(19) United States
(12) Patent Application Publication
(10) Pub. No.: US 2006/0030718 A1
(43) Pub. Date: Feb. 9, 2006

(54) Cobalt-based catalysts for the cyclization of alkenes

(75) Inventors: X. Peter Zhang, Knoxville, TN (US); Ying Chen, Knoxville, TN (US); Joshua V. Ruppel, Knoxville, TN (US); Jess E. Jones, Knoxville, TN (US); Jeremiah D. Harden, Knoxville, TN (US)

Correspondence Address: JENKINS, WILSON & TAYLOR, P. A., 3100 TOWER BLVD, SUITE 1400, DURHAM, NC, 27707, US

(73) Assignee: University of Tennessee Research Foundation

(21) Appl. No.: 11/215,347

(22) Filed: Aug. 30, 2005

Related U.S. Application Data
(63) Continuation-in-part of application No. 10/967,601, filed on Oct. 18, 2004.
Application No. 10/967,601 is a continuation-in-part of application No. 10/401,211, filed on Mar. 27, 2003, now Pat. No. 6,951,935.
(60) Provisional application No. 60/368,295, filed on Mar. 28, 2002.

(51) Int. Cl. C07D 203/04; C07D 487/22

(52) U.S. Cl. 548/954; 540/145

(57) ABSTRACT

Metal-ligand complexes, including cobalt-ligand complexes, such as a cobalt-porphyrin complex, and their use as catalysts in the cyclization of alkenes.

### CROSS REFERENCE TO RELATED APPLICATIONS

This application is a continuation-in-part of U.S. patent application Ser. No. 10/967,601, filed, Oct. 18, 2004, which is a continuation-in-part of U.S. patent application Ser. No. 10/401,211, filed Mar. 27, 2003, which claims the benefit of and priority to U.S. Provisional Patent Application Ser. No. 60/368,295, filed Mar. 28, 2002, the disclosures of which are incorporated herein by reference in their entireties.

### TECHNICAL FIELD

The presently disclosed subject matter relates to metal-ligand complexes, including cobalt-ligand complexes, such as a cobalt-porphyrin complex, and their use as catalysts in the cyclization of alkenes.

ABBREVIATIONS

| | |
|---|---|
| Ac | acetyl |
| t-BDA | t-butyl diazoacetate |
| BINAP | 2,2′-bis(diphenylphosphino)- |
| | 1,1′-binaphthyl |
| Bu | butyl |
| BT | bromamine-T |
| calc'd | calculated |
| CT | chloramine-T |
| dba | dibenzylidieneacetone |
| DDQ | 2,3-dichloro-5,6-dicyano-1,4- |
| | Benzoquinone |
| DMAP | 4-dimethylaminopyridine |
| DMF | N,N-dimethyl formamide |
| DMSO | dimethyl sulfoxide |
| DPEphos | bis(2-diphenylphosphinophenyl) |
| | ether |
| DPPA | diphenylphosphoryl azide |
| dppf | 1,1′-bis(diphenylphosphino) |
| | ferrocene] |
| EDA | ethyl diazoacetate |
| ee | enantiomeric excess |
| EI | electron impact |
| Et | ethyl |
| eV | electron volt |
| FT-IR | Fourier transform infrared |
| KOt-Bu | potassium tertiary butoxide |
| LDA | lithium diisopropyl amide |
| Me | methyl |
| Mg | milligram |
| MHz | megahertz |
| ML | milliliter |
| [M(Por)] | metalloporphyrin complex |
| [M(Por*)] | chiral metalloporphyrin complex |
| NaOt-Bu | sodium tertiary butoxide |
| NMR | nuclear magnetic resonance |
| Nu | nucleophile |
| Nu* | chiral nucleophile |
| OAc | acetate |
| OEP | octaethylporphyrin |
| OTf | trifluoromethanesulfonate |
| PDT | photodynamic therapy |
| Ph | phenyl |
| Por | porphyrin |
| Por* | chiral porphyrin |
| Pr | propyl |
| rt | room temperature |
| TFA | trifluoroacetatic acid |
| THF | tetrahydrofuran |
| TLC | thin layer chromatography |
| TOF | turnover frequency |
| TON | turnover number |
| TPFPP | tetrakis(pentafluorophenyl)porphyrin |
| TPP | 5,10,15,20-Tetraphenyl-21H,23H- |
| | Porphine |
| UV-Vis | ultraviolet-visible |
| vis | visible |

### BACKGROUND

Synthetic porphyrins and metalloporphyrins have become increasingly important in numerous and diverse technical fields. Their several practical applications include their use as sensitizers in photodynamic therapy (PDT) (Mody, (2000) J. Porphyrins Phthalocyanines 4: 362); in electron transfer (Lippard and Berg, (1994) Principles of Bioinorganic Chemistry, University Science Book: Mill Valley, Calif.); in DNA strand cleavage (Bennett et al., (2000) Proc. Natl. Acad. Sci. 97: 9476; Hashimoto et al., (1983) Tetrahedron Letters, 24: 1523); as carriers of cytotoxic anticancer drugs such as platinum (Song et al., (2002) Inorganic Biochemistry 83: 83; and Lottner et al., (2002) J. Med. Chem., 45, 2064); as components of synthetic receptors (Jain and Hamilton, (2002) Org. Lett. 2: 1721); and as oxidation catalysts (Guo et al., (2001) J. Mol. Catal. A Chem. 170: 43). Additionally, functionalized porphyrins have become important leads in current drug discovery techniques (See Mody, supra, and Priola et al., (2002) Science 287: 1503). Accordingly, the development of new methodologies and strategies to improve the synthesis of functionalized porphyrins has become highly desirable.

Numerous methods for the synthesis of porphyrins are known. The classical methods for porphyrin synthesis typically require harsh reaction conditions and can provide disappointingly low yields (Rothemund, (1935) J. Am. Chem. Soc., 57: 2010; Adler et al., (1967) J. Org. Chem. 32: 476). Newer methodologies, such as those developed by Lindsey and colleagues, have resolved certain issues regarding reaction conditions and yields (Lindsey et al., (1987) J. Org. Chem. 52: 827). More recently, transition metal-catalyzed organic synthesis methodologies (e.g., Suzuki coupling, Heck-type coupling, and Stille cross coupling), have been successfully employed with porphyrin systems, providing versatile and general synthetic approaches for the preparation of a variety of functionalized porphyrins and porphyrin analogs. See, e.g., DiMagno et al., (1993) J. Org. Chem., 58: 5983; DiMagno et al., (1993) J. Am. Chem. Soc. 115: 2513; Chan et al., (1995) Tetrahedron 51: 3129; Zhou et al., (1996) J. Org. Chem. 61: 3590; Risch and Rainer, (1997) Tetrahedron Letters 38: 223; Hyslop et al., (1998) J. Am. Chem. Soc. 120: 12676; Boyle and Shi, (2002) J. Chem. Soc. Perkin Trans., 1: 1397; and Pereira et al., (2002) J. Chem. Soc. Perkin Trans., 2: 1583. See also, Suzuki, (1998) Metal-Catalyzed Cross-Coupling Reactions, pp. 49-97, Wiley-VCH, Weinheim, Germany; Liu et al., (1998) J. Chem. Soc., Dalton Trans. 1805; Shi et al., (2000) J. Org. Chem. 65: 1650; Shanmugathasan et al., (2000) Porphyrins Phthalocyanines 4: 228; Lovine et al., (2000) J. Am. Chem. Soc. 122: 8717; Deng et al., (2000) Angew. Chem. Int. Ed. 39: 1066; and Chang et al., (2003) J. Org. Chem. 68: 4075; U.S. Pat. Nos. 5,550,236 and 5,756,804, which references are incorporated herein by reference. Further, porphyrin synthesis via palladium-catalyzed C—N bond formation, see Khan et al., (2001) Tetrahedron Lett. 42: 1615; Takanami et al., (2003) Tetrahedron Lett. 44: 7353, and metal-mediated C—C bond formation, see Sharman et al., (2000) Porphyr. Phthalocyanines 4: 441, has been reported.

Each of these foregoing methods, however, possesses undesirable aspects that should be mitigated, including incompatibilities between catalysts and reaction compounds, low turnover number (TON) and low turnover frequency (TOF). Thus, despite recent advances in porphyrin chemistry, a need still exists for facile and general syntheses for, in particular, heteroatom-substituted porphyrins and metalloporphyrins.

More particularly, a need exists for facile and general syntheses for heteroatom-substituted chiral porphyrins. Chiral porphyrins have found a range of applications in many areas, such as asymmetric catalysis, chiral recognition/sensing, and enzymatic mimicry. Of particular interest is the use of chiral porphyrins in asymmetric catalysis.

Biologically relevant porphyrins are among the most versatile ligands for transition metal complexes. See Brothers, (2001) Adv. Organometallic Chem. 46:223; Brothers, (2001) Adv. Organometallic Chem. 48: 289. Metalloporphyrins have found a diverse array of applications in areas ranging from chemistry to biology and from materials to medicine. Metalloporphyrins are known to catalyze a range of fundamentally and practically important chemical transformations, including an array of atom/group transfer reactions, such as oxene (epoxidation and hydroxylation), nitrene (aziridination and amination), and carbene (cyclopropanation and carbene insertion) transfers, that allow the direct conversion of abundant and inexpensive alkenes and alkanes into functional molecules. See The Porphyrin Handbook; Kadish, K. M., Smith, K. M., Guilard, R., Eds., Academic Press: San Diego, 2000-2003; Metalloporphyrins in Catalytic Oxidations; Sheldon, R. A., Ed.; Marcel Dekker: New York, 1994; Metalloporphyrins Catalyzed Oxidations: Montanari, F., Casella, L., Eds., Kluwer Academic Publishers: Boston, 1994. Due to the unique ligand environment and metal coordination mode of metalloporphyrins, unusual reaction selectivities and excellent catalyst turnovers have been observed for metalloporphyrin-based catalysts. Thus, there is a significant interest in designing and synthesizing chiral porphyrins for developing asymmetric versions of the abovementioned catalytic processes.

Since the first application of a chiral iron porphyrin complex for catalytic asymmetric epoxidation, see Groves et al., (1983) J. Am. Chem. Soc. 105: 5791, a number of chiral porphyrins have been synthesized as potential asymmetric catalysts. See Marchon, (2003) in The Porphyrin Handbook; supra, Vol. 11, pp 75-132; Simonneaux et al., (2002) Coord. Chem. Rev. 228: 43; Rose et al., (2000) Polyhedron 19: 581; Collman et al., (1999) Chemtracts 12: 299; Rose et al., (1998) Coord. Chem. Rev. 178-180: 1407; Collman et al., (1993) Science 261: 1404; Rose et al., (2004) Chem. Eur. J. 10: 224. Although significant progress has been made in this area, catalytic reactions based on metalloporphyrins have not been developed into practical methodologies that can be used in asymmetric synthesis. This lack of development can be attributed mainly to the expense and difficulty associated with chiral porphyrin synthesis.

Several approaches have been applied to chiral porphyrin synthesis. See Marchon, supra, Rose et al., (2000), supra, and Collman et al., (1993), supra. The most general and chirally economic scheme for synthesizing chiral porphyrins is to covalently attach suitable chiral building blocks to a preformed porphyrin synthon comprising peripheral functional groups. See Tani et al., (2002) Coord. Chem. Rev. 226: 219; Simonneaux et al., supra; Collman et al., (1999), supra; Rose et al., (1998), supra; Boschi, in Metalloporphyrins Catalyzed Oxidations: Montanari, F., Casella, L., Eds., Kluwer Academic Publishers: Boston, 1994; pp 239-267; and Naruta, (1994) in Metalloporphyrins in Catalytic Oxidations; Sheldon, R. A., Ed.; Marcel Dekker: New York, pp 241-259.

Representative porphyrin synthons that have been found to be useful for synthesizing chiral porphyrins include meso-tetrakis(o-aminophenyl)porphyrin (see Collman et al., (1975) J. Am. Chem. Soc. 97: 1427 and Leondiadis et al., (1989) J. Org. Chem. 54: 6135), meso-tetrakis(2,6-diaminophenyl)porphyrin (see Rose et al., (1996) J. Am. Chem. Soc. 118: 1567), meso-tetrakis (2,6-dihydroxyphenyl)porphyrin (see Collman et al., (1997) Inorg. Synth. 31: 117 and Tsuchida et al., (1990) J. Chem. Soc.-Dalton Trans. 2713), and meso-tetrakis(2,6-dicarboxyphenyl)porphyrin (see Nakagawa et al., (2001) Org. Lett. 3: 1805). These synthons allow the attachment of chiral acids, chiral amines, or chiral alcohols through amide or ester bond formation. To enhance the synthetic utility and flexibility of metalloporphyrin-based asymmetric catalysis, it is desirable to develop alternative synthons for the versatile construction of chiral porphyrins that could be employed in practical asymmetric catalysis.

Within this context, halogenated porphyrins, e.g., bromoporphyrins, have been shown to be versatile precursors for the synthesis of heteroatom-functionalized porphyrins via metal-catalyzed carbon-heteroatom cross-coupling reactions with soft, non-organometallic nucleophiles. See Chen et al., (2003) J. Org. Chem. 68: 4432; Gao et al., (2003) J. Org. Chem. 68: 6215; Gao et al., (2003) Org. Lett. 5: 3261; and Gao et al., (2004) Org. Lett. 6: 1837. These methods are based on metal-catalyzed carbon-heteroatom bond formations. See Ley et al., (2003) Angew. Chem.-Int. Edit. 42: 5400; Prim et al., (2002) Tetrahedron, 58: 2041; Muci et al., (2002) Top. Curr. Chem. 219: 131; Hartwig, (2002) in Handbook of Organopalladium Chemistry for Organic Synthesis; Negishi, E., Ed.; Wiley-Interscience: New York, pp 1051; Yang et al., (1999) J. Organomet. Chem. 576: 125; Wolfe et al., (1998) Acc. Chem. Res. 31: 805; Hartwig, (1998) Angew. Chem.-Int. Edit. 37: 2047; and Hartwig, (1997) Synlett, 329. Such syntheses can be performed under mild conditions with a wide range of nucleophiles, including amines, amides, alcohols, and thiols, leading to a family of novel porphyrins comprising otherwise inaccessible heteroatom functionalities in high yields.

For example, a general and efficient method has been developed for the synthesis of meso-arylamino- and meso-alkylamino-substituted porphyrins from reactions of meso-bromoporphyrins with amines. See Chen et al., (2003) J. Org. Chem. 68: 4432. Similar methodology also can be effectively applied to brominated diphenylporphyrins and tetraphenylporphyrins, leading to the versatile synthesis of porphyrin derivatives bearing multiple arylamino and alkylamino groups. See Gao et al., (2003) J. Org. Chem. 68: 6215. In addition, a convenient and general approach has been developed for the synthesis of meso-aryloxy- and meso-alkoxy-substituted porphyrins from reactions with alcohols via palladium-catalyzed etheration. See Gao et al., (2003) Org. Lett. 5: 3261. A general synthetic method for meso-amidoporphyrins from reactions with amides via palladium-catalyzed amidation also has been developed. See Gao et al., (2004) Org. Lett. 6: 1837. Expanding the synthetic strategy to palladium-mediated carbon-sulfur bond formation, a versatile procedure also has been developed for the synthesis of meso-arylsulfanyl- and meso-alkylsulfanyl-substituted porphyrins from reactions of the corresponding bromoporphyrin precursors and thiols. See Gao et al., (2004), J. Org. Chem. 69: 8886. There exists, however, a need in the art for improved methods for the synthesis of heteroatom-substituted chiral porphyrins.

Accordingly, the presently disclosed subject matter describes the use of haloporphyrins as a new class of synthons for the versatile syntheses of chiral porphyrins via metal catalyst-mediated carbon-heteroatom bond formation reactions with chiral nucleophiles, such as chiral amines, chiral amides, chiral alcohols, and chiral thiols, and the use of these chiral porphyrins as catalysts in asymmetric cyclopropanation, asymmetric aziridination, and asymmetric epoxidation reactions.

Further, there is a need in the art for improved catalysts for the cyclization of alkenes. Accordingly, in some embodiments, the presently disclosed subject matter describes a cobalt-based catalyst system for cyclization of alkenes, including but not limited to, the cyclopropanation of, alkenes and the aziridination of alkenes.

### SUMMARY

In some embodiments, the presently disclosed subject matter provides novel heteroatom-substituted porphyrin compounds. Novel compounds of the presently disclosed subject matter have the structure of Formula I, as follows:

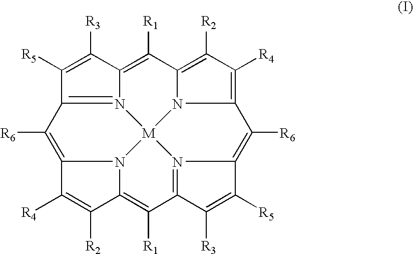

In Formula I, M is H₂ or a transition metal; each R₁, R₂, R₃, R₄, R₅ and R₆ are each independently selected from the group consisting of Y, H, alkyl, substituted alkyl, arylalkyl, aryl, and substituted aryl; Y is a heteroatom-containing moiety; and at least one of R₁, R₂, R₃, R₄, R₅ and R₆ is Y. In some embodiments, M is selected from the group consisting of H₂, Fe, Zn and Ni, although numerous other transition metals are useful in the presently disclosed subject matter. In some embodiments, Y is a heteroatom-containing moiety selected from the group consisting of NR₇R₈, NR₁₀, OR₁₀, PR₇R₈, SR₁₀, SiR₇R₈R₉, BR₇R₈, GeR₇R₈R₉, SnR₇R₈R₉ and SeR₁₀, wherein R₇, R₈, R₉, and R₁₀ are each independently selected from the group consisting of H, alkyl, substituted alkyl, arylalkyl, aryl, and substituted aryl. In some embodiments, Y is a selected from the group consisting of amino, substituted amino, imino, substituted imino, and phenoxy groups.

In some embodiments, the presently disclosed subject matter describes a method of synthesizing a heteroatom-substituted porphyrin compound, whereby a porphyrin precursor and a heteroatom reagent is reacted in the presence of a ligand, a metal compound, and a base to yield the substituted porphyrin. In some embodiments, the porphyrin precursor has the same general structure of Formula I, wherein M is H₂ or a transition metal; each R₁, R₂, R₃, R₄, R₅ and R₆ are each independently selected from the group consisting of X, H, alkyl, substituted alkyls, arylalkyls, aryls, and substituted aryls, and X is selected from the group consisting of halogen, trifluoromethanesulfonate (OTf), haloaryl and haloalkyl. In this embodiment, at least one of R₁, R₂, R₃, R₄, R₅ and R₆ is X. In some embodiments, M is selected from the group consisting of H₂, Zn, Fe and Ni. In some embodiments, the heteroatom reagents comprise moieties in which the heteroatom is selected from the group consisting of N, O, P, S, Si, B, Ge, Sn, and Se. In some embodiments, the heteroatom reagent is selected from one of N and O.

Accordingly, the presently disclosed subject matter provides a novel heteroatom-substituted porphyrin compound and a novel method of synthesizing a heteroatom-substituted porphyrin compound.

In some embodiments, the presently disclosed subject matter provides metal-ligand catalysts, including cobalt-ligand catalysts, such as cobalt-porphyrin catalysts, for the cyclization of alkenes, including but not limited to the aziridination of alkenes and intramolecular cyclopropanation of alkenes.

Accordingly, in some embodiments, the presently disclosed subject matter provides a method of synthesizing an aziridine compound, the method comprising reacting an alkene with a nitrene source in the presence of a cobalt-containing catalyst. In some embodiments, the presently disclosed subject matter provides a method of synthesizing an aziridine compound, the method comprising reacting an alkene with a nitrene source in the presence of a porphyrin metal complex, wherein the porphyrin metal complex has the structure of Formula (I): 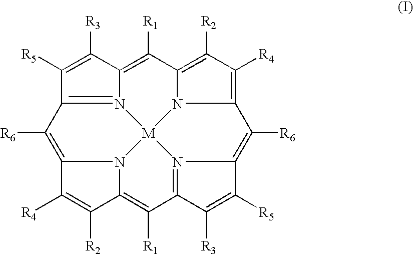
wherein M is a transition metal ion; and R₁, R₂, R₃, R₄, R₅ and R₆ are each independently selected from the group consisting of H, alkyl, substituted alkyl, arylalkyl, aryl, and substituted aryl and Y, wherein Y is a heteroatom-containing chiral moiety. In some embodiments, the transition metal ion is selected from the group consisting of zinc, rhodium, and cobalt. In some embodiments, the transition metal ion is cobalt.

In some embodiments, the presently disclosed subject matter provides for cobalt-porphyrin catalyzed intramolecular cyclopropanation. Accordingly, in some embodiments, the presently disclosed subject matter provides a method of synthesizing a cyclopropane compound, the method comprising reacting an alkene-substituted diazo compound with a porphyrin metal complex to form a cyclopropane compound, wherein the porphyrin metal complex has the structure of Formula (I) and the metal ion is cobalt.

Thus, an object of the presently disclosed subject matter is to provide cobalt-catalyzed aziridination. It is another object of the presently disclosed subject matter to provide cobalt-porphyrin catalyzed aziridination. It is another object of the presently disclosed subject matter to provide cobalt-catalyzed intramolecular cyclopropanation.

Certain objects of the presently disclosed subject matter having been stated hereinabove, which are addressed in whole or in part by the presently disclosed subject matter, other aspects and objects will become evident as the description proceeds when taken in connection with the accompanying Drawings and Examples as best described herein below.

### BRIEF DESCRIPTION OF THE DRAWINGS

FIG. 1 illustrates several schemes by which heteroatom substituents (e.g., nitrogen, oxygen, etc.) can be substituted into porphyrins by metal/ligand-catalyzed cross-coupling or amination reactions. In FIG. 1, “M” represents H₂, or a transition metal; “X” represents a reactive group such as, for example a halide, trifluoromethanesulfonate (OTf, haloalkyl or haloaryl; and “Y” is heteroatom moiety such as, for example, NR₇R₈, NR₁₀, OR₁₀, PR₇R₈, SR₁₀, SiR₇R₈R₉, BR₇R₈, GeR₇R₈R₉, SnR₇R₈R₉ and SeR₁₀, where R₇, R₈, R₉, and R₁₀ are each independently, for example, H, alkyl, substituted alkyl, arylalkyl, aryl, or substituted aryl.

FIG. 2 illustrates the chemical structures of eleven compounds that are representative, although not inclusive, of phosphine ligands useful in the presently disclosed subject matter.

FIGS. 3A and 3B are schemes of particular embodiments of the presently disclosed subject matter. FIG. 3A shows two generalized schemes of palladium-catalyzed amination reactions of meso-monobromoporphyrins (left-hand scheme) and meso-dibromoporphyrins (right-hand scheme). FIG. 3B shows the same two amination reactions with specific reaction components and conditions indicated. In FIG. 3B, the upper reaction scheme represents the amination of a meso-monobromoporphyrin, while the lower scheme represents the amination of a meso-dibromoporphyrin.

FIG. 4 illustrates the chemical structures of several compounds of the presently disclosed subject matter, which compounds are synthesized by the presently disclosed methods. The compound numbers shown in FIG. 4 (e.g., 3a, 3b, 4a, 4b, etc.) correspond to the compound numbers indicated in Table 1, below.

FIGS. 5A-5D are schemes of particular embodiments of the presently disclosed subject matter. FIG. 5A is a general scheme of the synthesis of aminophenylporphyrins by a palladium-catalyzed amination reaction of p-bromophenyl porphyrin and its zinc complex. FIG. 5B illustrates a particular embodiment of the general scheme of FIG. 5A, wherein specific reaction conditions and components are indicated. FIG. 5C illustrates yet another particular embodiment of the general reaction shown in FIG. 5A, wherein specific reaction conditions and components are indicated. FIG. 5D illustrates a generalized scheme of a palladium-catalyzed reaction of a tetrakis-p-bromophenyl porphyrin that yields a tetrakis-aminophenyl porphyrin.

FIG. 6 illustrates a general reaction scheme whereby [5-bromo-10,20-diphenylporphyrino]zinc(II) (indicated in the Figure as compound 1) and [5,15-dibromo-10,20-diphenylporphyrino]zinc(II) (indicated in the Figure as compound 2) undergo a palladium-catalyzed cross-coupling reaction to yield the corresponding meso-substituted phenoxyporphyrins (indicated in the Figure as compounds 3 and 4, respectively).

FIG. 7 shows the chemical structures of several heteroatom-substituted phenoxyporphyrin compounds of the presently disclosed subject matter, which compounds are synthesized via the methods described herein.

FIG. 8 provides general reaction schemes of the presently disclosed subject matter for the synthesis of heteroatom-substituted porphyrins via palladium-catalyzed cross-coupling reactions. FIG. 8A shows a generalized scheme of a palladium-catalyzed amination reaction of a meso-dibromoporphyrin to form meso-arylamino and meso-alkylamino substituted porphyrins. FIG. 8B shows a generalized scheme of a palladium-catalyzed amidation reaction of a meso-dibromoporphyrin to form meso-amido substituted porphyrins. FIG. 8C shows a generalized scheme of a palladium-catalyzed C—O cross coupling reaction of a meso-dibromoporphyrin and an alcohol to form meso-aryloxy- and meso-alkoxy-substituted porphyrins. FIG. 8D shows a generalized scheme of a palladium-catalyzed C—S bond formation reaction between a meso-dibromoporphyrin and a thiol to form meso-arylsulfanyl- and meso-alkylsulfanyl-substituted porphyrins. FIG. 8E shows a generalized scheme of a palladium-catalyzed amination reaction of a di(p-bromophenyl) porphyrin to form aminophenylporphyrins. FIG. 8F shows a generalized scheme of a palladium-catalyzed nucleophilic substitution reaction between a brominated porphyrin and a heteroatom nucleophile (H—Nu) to form a heteroatom-substituted porphyrin.

FIG. 9 provides reaction schemes for the synthesis of porphyrin triflates, e.g., compounds 22-1a, 22-1b, 22-1c and compounds 22-2a, 22-2b, 22-2c, which are representative, of the presently disclosed subject matter.

FIG. 10 illustrates generalized schemes A-F depicting the use of bromoporphyrins as synthons for the modular construction of chiral porphyrins of the presently disclosed subject matter.

FIG. 11 illustrates generalized schemes for the preparation of bromoporphyrin synthons S1 and S2 and the synthesis of meso-chiral porphyrins (meso-CP), e.g., compounds 15a-15f and compounds 17a-17c, and ortho-chiral porphyrins (ortho-CP), e.g., compounds 20a-20p, which are representative, of the presently disclosed subject matter.

FIG. 12 illustrates generalized schemes for the synthesis of amido-substituted ortho chiral porphyrins 20, e.g., compounds 20a-20p, which are representative, but not inclusive, of the presently disclosed subject matter, via a palladium-catalyzed amidation reaction and cobalt complexes 21 thereof and the chemical structures of particular chiral amide reagents 23a, 23b, 23c, and 23d of the presently disclosed subject matter.

FIG. 13 provides the chemical structures and synthetic yields of meso-chiral porphyrins, e.g., compounds 15a-15f and compounds 17a-17c, which are representative, of the presently disclosed subject matter.

FIG. 14 represents the X-ray structures of exemplary meso-aminoporphyrins of the presently disclosed subject matter. FIG. 14a represents the X-ray structure of a meso-aminoporphyrin comprising an N-methylaniline group. FIG. 14b represents the X-ray structure of a meso-aminoporphyrin comprising a diphenylamine group.

FIG. 15 provides the generalized chemical structures of meso-chiral porphyrins A, B, C, and D comprising C₂-symmetrical chiral secondary amine building blocks and which are representative of the presently disclosed subject matter.

FIG. 16 provides the chemical structures and synthetic yields of D₂-symmetric ortho-chiral porphyrins, e.g., compounds 20a-20d and compounds 20l-20o, which are representative, but not limiting, of the presently disclosed subject matter.

FIG. 17 is a generalized schematic representation of the synthesis of ortho-chiral porphyrins C comprising meso-heteroatom substituents, which are representative of the presently disclosed subject matter. More particularly, FIG. 17 illustrates the conversion of chiral porphyrins comprising hydrogen atoms at meso-positions (A) to meso-dibromoporphyrins (B) by selective bromination, followed by the conversion of meso-dibromoporphyrins B to the desired meso heteroatom-substituted ortho-chiral porphyrins C.

FIG. 18 provides the generalized chemical structures of ortho-chiral porphyrins D, E, F, and G, which are representative of the presently disclosed subject matter, and which can be prepared from various chiral building blocks.

FIG. 19 represents the structures of borate ester-containing chiral porphyrins 25a and 25b, which are representative of the presently disclosed subject matter and computer generated 3D structures of the same.

FIG. 20 is a schematic representation depicting the preparation of cobalt complexes of meso-chiral porphyrins, e.g., compounds 16a-16f and compounds 18a-18c, and ortho-chiral porphyrins, e.g., compounds 21a-21d and compounds 21l-21o, which are representative, of the presently disclosed subject matter.

FIG. 21 is a generalized schematic representation of a cobalt-porphyrin complex catalyzed cyclopropanation reaction with styrene as the limiting reagent providing cis-(1S,2R), cis-(1R,2S), trans-(1R,2R), and trans-(1S,2S) reaction products, which are representative of the presently disclosed subject matter.

FIG. 22 is a generalized schematic representation of a metalloporphyrin catalyzed intramolecular asymmetric cyclopropanation reaction comprising diazo compounds bearing a pendant alkene C═C bond, the reaction and the diazo compounds being representative of the presently disclosed subject matter.

FIG. 23 provides the structures of the cobalt, iron, ruthenium, and rhodium complexes of D2symmetric meso-chiral porphyrin 20l, which are representative of the presently disclosed subject matter.

FIG. 24 is a Hammett plot showing Log(kₓ/k_{H}) versus a for the cyclopropanation of para-substituted styrenes with EDA by [Co(20l)] and [Fe(20l)Cl].

FIG. 25 is an illustration of a possible cyclopropanation mechanism by cobalt porphyrins, which is representative of the presently disclosed subject matter.

FIG. 26 provides the chemical structures of various cobalt (II) porphyrin catalysts for aziridination.

FIG. 27 provides the chemical structures of alkene substrates, which are representative of the presently disclosed subject matter, for the aziridination by Fe(TPP)Cl with bromamine-T.

FIG. 28 provides the chemical structures of metal complexes of D₂-symmetric meso-chiral porphyrins, e.g., [M(21g)] and [M(21f)], bearing electron-withdrawing groups, which are representative of the presently disclosed subject matter.

FIG. 29 is a portion of a ¹H NMR spectrum showing the characteristic splitting pattern of the aziridine ring hydrogens of N-phosphorylated aziridines.

FIG. 30 is an illustration of a possible aziridination mechanism by cobalt porphyrins using diphenylphosphoryl azide (DPPA) as the nitrene source.

FIG. 31 provides the chemical structures of phosphoryl (24a & 24b), phosphinyl (24c & 24d), and phosphorodiamidic (24e & 24f) azides for use with the presently disclosed subject matter.

### DETAILED DESCRIPTION

In some embodiments the presently disclosed subject matter describes a modular approach for the versatile syntheses of porphyrins, including but not limited to chiral porphyrins, which can be utilized as supporting ligands for metal-based asymmetric and symmetric catalysis. In some embodiments the approach employs haloporphyrins as a new class of synthons to react with nucleophiles via metal catalyst (e.g., palladium)-mediated carbon-heteroatom bond formation reactions, thereby providing a diverse family of porphyrins.

Porphyrin synthesis can play a role in the development of metalloporphyrin-based asymmetric and symmetric catalysis. The synthetic approach described by the presently disclosed subject matter is modular and in some embodiments includes at least one of several attractive characteristics: (1) the haloporphyrin synthons are stable and readily accessible in large quantities from haloaldehydes or through selective halogenation; (2) the position and number of halide atoms can be varied, leading to porphyrins, including but not limited to chiral porphyrins, with different symmetries; (3) the metal (e.g. palladium)-catalyzed cross-coupling reactions have high yields, are reliable, and can be performed under mild conditions for which functional and sensitive groups are well tolerated; and (4) a wide range of readily available building blocks, including optically pure building blocks, such as chiral amines, chiral amides, chiral alcohols, and chiral thiols, can be coupled to form porphyrins with diverse characteristics to create a chemical library or “toolbox” of porphyrins for use in the asymmetric and symmetric cyclization of alkenes.

Recognizing the usefulness of the “toolbox” approach in asymmetric and symmetric catalysis, in some embodiments the presently disclosed subject matter couples various haloporphyrins with a diverse assortment of building blocks to construct a family of new porphyrins, including but not limited to chiral porphyrins, with tunable electronic, steric, and geometric characteristics. Accordingly, the presently disclosed subject matter can provide a “toolbox” of effective porphyrins, including but not limited to chiral porphyrins, for a variety of metal-based asymmetric and symmetric catalytic processes. Further, the presently disclosed subject matter can provide a class of catalysts that can be used for practical asymmetric and symmetrical syntheses of pharmaceutically and agriculturally important compounds. See Yoon et al., (2003) Science 299: 1691.

Accordingly, in some embodiments the presently disclosed subject matter describes the use of metal complexes of these porphyrins, including but not limited to chiral porphyrins, as catalysts for asymmetric and symmetric catalytic processes including a number of important atom/group transfer reactions. For example, the presently disclosed subject matter describes the use of cobalt-based catalysts, including cobalt porphyrins, as a catalyst for novel cyclopropanation and aziridination reactions. The presently disclosed subject matter demonstrates in some embodiments that both high diastereoselectivity and high enantioselectivity, as well as high chemical yields, can be achieved under practical conditions for each of the possible isomers by using the presently disclosed catalysts, including but not limited to the presently disclosed cobalt-based catalysts, under conditions comprising different environments.

Further, in some embodiments the presently disclosed subject matter describes the use of porphyrins, including but not limited to chiral porphyrins, as catalysts for asymmetric aziridination reactions with nitrene sources that are convenient and environmentally benign. For example, the presently disclosed subject matter describes the use of the easily accessible and highly stable bromamine-T and diphenylphosphoryl azide as nitrene sources for the catalytic aziridination by porphyrins, including but not limited to chiral porphyrins.

In sum, the presently disclosed subject matter provides efficient catalytic systems for asymmetric and symmetric cyclopropanation and aziridination under practical conditions.

The presently disclosed subject matter will now be described more fully hereinafter with reference to the accompanying Drawings and Examples, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

Unless otherwise defined, all technical and scientific terms used herein have the same meaning as commonly understood by one of ordinary skill in the art to which this presently described subject matter belongs. All publications, patent applications, patents, and other references mentioned herein are incorporated by reference in their entirety.

Throughout the specification and claims, a given chemical formula or name shall encompass all stereoisomers.

### I. DEFINITIONS

The term “independently selected” is used herein to indicate that the R groups, e.g., R₁, R₂, R₃ or R₄, can be identical or different (e.g., R₁, R₂ and R₃ can all be substituted alkyls, or R₁ and R₄ can be a substituted alkyl and R₃ can be an aryl, etc.). Moreover, “independently selected” means that in a multiplicity of R groups with the same name, each group can be identical to or different from each other (e.g., one R₁ can be an alkyl, while another R₁ group in the same compound can be aryl; one R₂ group can be H, while another R₂ group in the same compound can be alkyl, etc.).

A named R group will generally have the structure that is recognized in the art as corresponding to R groups having that name. For the purposes of illustration, representative R groups as enumerated above are defined herein. These definitions are intended to supplement and illustrate, not preclude, the definitions known to those of skill in the art.

As used herein, the term “alkyl” means C₁₋₂₀ inclusive, linear (i.e., “straight-chain”), branched, or cyclic, saturated or at least partially and in some cases fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups.

“Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

The alkyl group can be optionally substituted (i.e., a “substituted alkyl”) with one or more alkyl group substituents which can be the same or different, where “alkyl group substituent” includes but is not limited to alkyl, substituted alkyl, halo, arylamino, acyl, hydroxy, aryl, aryloxy, alkoxyl, alkylthio, arylthio, aralkyloxy, aralkylthio, carboxy, alkoxycarbonyl, oxo and cycloalkyl. Suitable substituted alkyls include, for example, benzyl, trifluoromethyl and the like. There can be optionally inserted along the alkyl chain one or more oxygen, sulfur or substituted or unsubstituted nitrogen atoms, wherein the nitrogen substituent is hydrogen, alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, hydroxy, and/or alkylene. There can be optionally inserted along the cyclic alkyl chain one or more oxygen, sulfur or substituted or unsubstituted nitrogen atoms, wherein the nitrogen substituent is hydrogen, alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl.

“Alkylene” refers to a straight or branched bivalent aliphatic hydrocarbon group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons.

The term “aryl” is used herein to refer to an aromatic substituent which can be a single aromatic ring or multiple aromatic rings which are fused together, linked covalently, or linked to a common group such as a methylene or ethylene moiety. The common linking group can also be a carbonyl as in benzophenone or oxygen as in diphenylether or nitrogen in diphenylamine. The term “aryl” specifically encompasses heterocyclic aromatic compounds. The aromatic ring(s) can include phenyl, naphthyl, biphenyl, diphenylether, diphenylamine and benzophenone among others. In particular embodiments, the term “aryl” means a cyclic aromatic comprising about 5 to about 10 carbon atoms, e.g., 5, 6, 7, 8, 9, or 10 carbon atoms, and including 5- and 6-membered hydrocarbon and heterocyclic aromatic rings.

Specific examples of aryl groups include but are not limited to cyclopentadienyl, phenyl, furan, thiophene, pyrrole, pyran, pyridine, imidazole, benzimidazole, isothiazole, isoxazole, pyrazole, pyrazine, triazine, pyrimidine, quinoline, isoquinoline, indole, carbazole, and the like.

The aryl group can be optionally substituted (i.e., a “substituted aryl”) with one or more aryl group substituents which can be the same or different, where “aryl group substituent” includes alkyl, substituted alkyl, aryl, substituted aryl, aralkyl, hydroxy, alkoxyl, aryloxy, aralkoxyl, carboxy, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene and —NR′R″, where R′ and R″ can be each independently hydrogen, alkyl, substituted alkyl, aryl, substituted aryl, and aralkyl.

Thus, as used herein, the term “substituted aryl” includes aryl groups, as defined herein, in which one or more atoms or functional groups of the aryl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

The term “arylalkyl” refers to the group-aryl-alkyl. The aryl group can be phenyl or napthyl or can be heteroaryl. The alkyl can be cyclic or branched or further substituted, for example, by a halo, hydroxy, or nitro group. Exemplary arylalkyl compounds include, but are not limited to 4-tert-butylphenyl, 3-methylphenyl, 2-isopropylphenyl, 2,6-di-isopropylphenyl, 2,6-dimethylphenyl, 3,5-di-tert-butylphenyl, and 2,4,6-trimethylphenyl.

The term “alkene” is used to denote a group containing a carbon-carbon double bond. Representative alkene groups include, but are not limited to, ethenyl, propenyl, butenyl, pentenyl, hexenyl, octenyl, and butadienyl. The carbon atoms of the double bond can be further substituted by substituents that can be the same or different and can include hydrogen, alkyl, substituted alkyl, aryl, hydroxyalkyl, aralkyl, arylalkyl, halo, arylamino, alkylamino, acyl, alkylthio, arylthio, cycloalkyl, carboxy, alkyloxycarbonyl, aryloxycarbonyl, alkylcarbamoyl, carbamoyl, dialkylcarbamoyl, and the like.

The term “di-substituted alkene” is used herein to refer to an alkene in which two of the substituents directly attached to the double-bonded carbon atoms are substituents that are other than hydrogen. Both of the non-hydrogen substituents can be attached to the same carbon of the carbon-carbon double bond. Alternatively, one non-hydrogen substituent can be attached to each of the double-bonded carbons. “Tri-substituted alkenes” are alkenes in which the two carbon atoms of the double bond are together substituted with three groups that are other than hydrogen. In “tetra-substituted alkenes,” all four of the groups directly attached to the two carbon atoms of the double bond are substituents that are other than hydrogen.

As in determining the stereochemistry of alkyl groups, the substituents of alkenes can be assigned priority numbers based upon the Cahn Ingold Prelog rules and the conventions of the International Union of Pure and Applied Chemistry (IUPAC). Alkenes substituted with at least one non-hydrogen substituent on each of the carbon atoms of the carbon-carbon double bond can be designated as “cis” or “trans” based upon the relationship between the highest priority substituents attached to each of the carbons. If the highest priority substituent (e.g., substituent S₁) on one of the carbons is on the same side of the plane of the double bond as the highest priority substituent (e.g., substituent S₂) attached to the other carbon, the alkene is referred to as a “cis-alkene”. 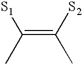
alkene with S₁ and S₂ cis

If the two substituents, e.g., S₁ and S₂, are attached on opposite sides of the plane of the double bond, the alkene is referred to as a “trans-alkene.” 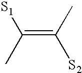
alkene with S₁ and S₂ trans

The term “acyclic alkene” is used herein to refer to an alkene that is not part of a cyclic moiety. The term “cyclic alkene” as used herein refers to an alkene in which the two carbons of the double bond are also part of a cyclic structure. Representative cyclic alkenes include, but are not limited to, cyclopropene, cyclobutene, cyclopentene, cyclohexene, cycloheptene, and the like.

The term “non-aromatic alkene” as used herein refers to an alkene that does not have an aromatic substituent. The term “aromatic alkene” refers to an alkene in which at least one of the alkene substituents contains an aromatic moiety. For example, styrene is an aromatic alkene.

A structure represented generally by a formula such as: 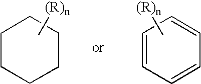
as used herein refers to a ring structure, for example, but not limited to a 3-carbon, a 4-carbon, a 5-carbon, a 6-carbon, and the like, aliphatic and/or aromatic cyclic compound comprising a substituent R group, wherein the R group can be present or absent, and when present, one or more R groups can each be substituted on one or more available carbon atoms of the ring structure. The presence or absence of the R group and number of R groups is determined by the value of the integer n. Each R group, if more than one, is substituted on an available carbon of the ring structure rather than on another R group. For example, the structure:

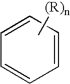

wherein n is an integer from 0 to 2 comprises compound groups including, but not limited to:

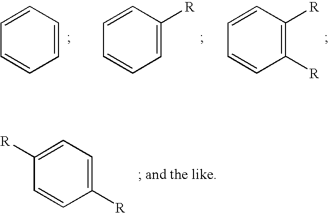

As used herein, the term “acyl” refers to an organic acid group wherein the —OH of the carboxyl group has been replaced with another substituent (i.e., as represented by RCO—, wherein R is an alkyl or an aryl group as defined herein). As such, the term “acyl” specifically includes arylacyl groups, such as an acetylfuran and a phenacyl group. Specific examples of acyl groups include acetyl and benzoyl.

The term “alkoxy” is used herein to refer to the —OZ₁ radical, where Z₁ is selected from the group consisting of alkyl, substituted alkyl, cycloalkyl, substituted cycloalkyl, heterocycloalkyl, substituted heterocycloalkyl, silyl groups and combinations thereof as described herein. Suitable alkoxy radicals include, for example, methoxy, ethoxy, benzyloxy, t-butoxy, and the like. A related term is “aryloxy” where Z₁ is selected from the group consisting of aryl, substituted aryl, heteroaryl, substituted heteroaryl, and combinations thereof. Examples of suitable aryloxy radicals include phenoxy, substituted phenoxy, 2-pyridinoxy, 8-quinalinoxy and the like.

The term “amino” is used herein to refer to the group —NZ₁Z₂, where each of Z₁ and Z₂ is independently selected from the group consisting of hydrogen; alkyl, substituted alkyl, cycloalkyl, substituted cycloalkyl, heterocycloalkyl, substituted heterocycloalkyl, aryl, substituted aryl, heteroaryl, substituted heteroaryl, alkoxy, aryloxy, silyl and combinations thereof. Additionally, the amino group can be represented as N⁺Z₁Z₂Z₃, with the previous definitions applying and Z₃ being either H or alkyl.

“Aralkyl” refers to an aryl-alkyl-group wherein aryl and alkyl are as previously described, and included substituted aryl and substituted alkyl. Exemplary aralkyl groups include benzyl, phenylethyl, and naphthylmethyl.

“Aralkyloxyl” refers to an aralkyl-O— group wherein the aralkyl group is as previously described. An exemplary aralkyloxyl group is benzyloxyl.

“Dialkylamino” refers to an —NRR′ group wherein each of R and R′ is independently an alkyl group and/or a substituted alkyl group as previously described. Exemplary alkylamino groups include ethylmethylamino, dimethylamino, and diethylamino.

“Alkoxycarbonyl” refers to an alkyl-O—CO— group. Exemplary alkoxycarbonyl groups include methoxycarbonyl, ethoxycarbonyl, butyloxycarbonyl, and t-butyloxycarbonyl.

“Aryloxycarbonyl” refers to an aryl-O—CO— group. Exemplary aryloxycarbonyl groups include phenoxy- and naphthoxy-carbonyl.

“Aralkoxycarbonyl” refers to an aralkyl-O—CO— group. An exemplary aralkoxycarbonyl group is benzyloxycarbonyl.

“Carbamoyl” refers to an H₂N—CO— group.

“Alkylcarbamoyl” refers to a R′RN—CO— group wherein one of R and R′ is hydrogen and the other of R and R′ is alkyl and/or substituted alkyl as previously described.

“Dialkylcarbamoyl” refers to a R′RN—CO— group wherein each of R and R′ is independently alkyl and/or substituted alkyl as previously described.

“Acyloxyl” refers to an acyl-O— group wherein acyl is as previously described.

“Acylamino” refers to an acyl-NH— group wherein acyl is as previously described.

“Aroylamino” refers to an aroyl-NH— group wherein aroyl is as previously described.

The term “carbonyl” refers to the —(C═O)— group.

The term “carboxyl” refers to the —COOH group.

The terms “halo”, “halide”, or “halogen” as used herein refer to fluoro, chloro, bromo, and iodo groups.

The term “hydroxyl” refers to the —OH group.

The term “hydroxyalkyl” refers to an alkyl group substituted with an —OH group.

The term “mercapto” refers to the —SH group.

The term “oxo” refers to a compound described previously herein wherein a carbon atom is replaced by an oxygen atom.

The term “nitro” refers to the —NO₂ group.

The term “thio” refers to a compound described previously herein wherein a carbon or oxygen atom is replaced by a sulfur atom.

The term “sulfate” refers to the —SO₄ group.

A “heteroatom,” as used herein, is an atom other than carbon. In some embodiments, the heteroatoms are selected from the group consisting of N, O, P, S, Si, B, Ge, Sn, and Se. In some embodiments of the presently disclosed subject matter, the heteroatoms are selected from one of N and O.

The term “stereoisomer” refers to molecules that are made up of the same atoms connected by the same sequence of bonds, but have different three dimensional structures. The term stereoisomer includes enantiomers, i.e., mirror image stereoisomers, cis-trans isomers, and diastereomers.

The term “chiral” refers to the stereochemical property of a molecule of being non-superimposible on its mirror image. A chiral molecule has no symmetry elements of the second kind, e.g., a mirror plane, a center of inversion, and a rotation-reflection axis. The two forms of a chiral molecule are known as enantiomers. A collection containing equal amounts of the two enantiomeric forms of a chiral molecule is referred to as a racemic mixture or racemate. In some embodiments, a chiral “R” group is represented by “R*.”

The term “diastereomer” refers to non-enantiomeric isomers which arise when more than one stereocenter is present in a molecule.

A collection of molecules containing only one enantiomeric form of a chiral molecule is referred to as “enantiopure,” “enantiomerically pure,” or “optically pure.” A mixture containing predominantly one enantiomer is referred to as enantiomerically enriched or enantioenriched. Enantiopurity is usually reported in terms of “enantiomeric excess” (e.e.), which is determined as:
% e.e.=(major−minor)*100/(major+minor)
wherein the term “major” refers to the more abundant enantiomer and the term “minor” refers to the less abundant enantiomer. For example, in some embodiments of the presently disclosed subject matter, an optically active compound can have an enantiopurity of greater than 50%; of greater than 75%; of greater than 90%; or of greater than 95%.

The term “nucleophile” or “nucleophilic reagent” refers to a reagent that forms a bond to its reaction partner, e.g., an “electrophile” by donating both bonding electrons.

The term “porphyrin” refers to a compound comprising a fundamental skeleton of four pyrrole nuclei united through the α-positions by four methane groups to form the following macrocyclic structure:

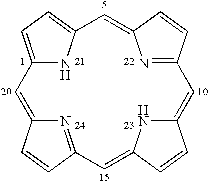

The term “meso” refers to the position on the porphyrin structure adjacent to the reduced pyrrole ring, i.e., positions 5, 10, 15, and 20. Said another way, a “meso-porphyrin” is a porphyrin compound comprising substituent groups at the 5, 10, 15, and 20 position, or combinations thereof.

The term “reflux” and grammatical derivations thereof refer to boiling a liquid, such as a solvent, in a container, such as a reaction flask, with which a condenser is associated, thereby facilitating continuous boiling without loss of liquid, due to the condensation of vapors on the interior walls of the condenser.

The term “aprotic solvent” refers to a solvent molecule which can neither accept nor donate a proton. Typical aprotic solvents include, but are not limited to, acetone, acetonitrile, benzene, butanone, butyronitrile, carbon tetrachloride, chlorobenzene, chloroform, 1,2-dichloroethane, dichloromethane, diethyl ether, dimethylacetamide, N,N-dimethylformamide (DMF), dimethylsulfoxide (DMSO), 1,4-dioxane, ethyl acetate, ethylene glycol dimethyl ether, hexane, N-methylpyrrolidone, pyridine, tetrahydrofuran (THF), and toluene. Certain aprotic solvents are polar solvents. Examples of polar aprotic solvents include, but are not limited to, acetone, acetonitrile, butanone, N,N-dimethylformamide, and dimethylsulfoxide. Certain aprotic solvents are non-polar solvents. Examples of nonpolar, aprotic solvents include, but are not limited to, diethyl ether, aliphatic hydrocarbons, such as hexane, aromatic hydrocarbons, such as benzene and toluene, and symmetrical halogenated hydrocarbons, such as carbon tetrachloride.

The term “protic solvent” refers to a solvent molecule which contains a hydrogen atom bonded to an electronegative atom, such as an oxygen atom or a nitrogen atom. Typical protic solvents include, but are not limited to, carboxylic acids, such as acetic acid, alcohols, such as methanol and ethanol, amines, amides, and water.

The term “azide” refers to compounds having the group N₃, —N═N⁺═N⁻. An azide can have the general formula of RN₃, wherein R is an organic radical, such as, but not limited to, alkyl, substituted alkyl, cycloalkyl, aryl, substituted aryl, phosphoryl, phosphinyl, and phosphorodiamidic. Thus, an “organic azide” refers to compounds, for example, including aryl azides, alkyl azides, acyl azides, sulfonyl azides, phosphoryl azides, phosphinyl azides and phosphorodiamidic azides.

The term “diazo” refers to compounds having the divalent diazo group, ═N⁺═N⁻, attached to a carbon atom. For example, the compound CH₂═N₂ is referred to as diazomethane. Exemplary diazo compound include, but are not limited to, diazomethane, trimethylsilyldiazomethane, diethyl diazomalonate, and diazoacetate, as defined herein below.

The term “diazoacetate” refers to compounds having the general formula: 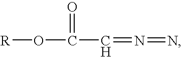
wherein R is an organic radical, such as, for example, alkyl, substituted alkyl, aryl, and substituted aryl. Exemplary diazoacetate compounds include, but are not limited to, ethyl diazoacetate, tert-butyl diazoacetate, 2,6-di-tert-butyl-4-methylphenyl diazoacetate, and methyl phenyldiazoacetate.

The term “nitrene” refers to reactive reaction intermediates having a univalent nitrogen and that can be represented by the general formula “RN:”. Sources of nitrenes, i.e., compounds that can provide a nitrene during the course of a reaction, include, but are not limited to [N-(p-toluenesulfonyl)imino]phenyliodinane; N-halo-p-toluenesulfonamides, such as bromamine T and chloramine T; and organic azides.

The term “allylic” refers to the group CH₂═CHCH₂— (allyl) and derivatives formed by substitution. The term “allylic position” or “allylic site” refers to the carbon immediately next to the carbon-carbon double bond. Thus, a substituent group, such as an —OH group, attached at an allylic site can be referred to as an “allylic hydroxyl” group. Exemplary allylic compounds include, but are not limited to 3-methyl-2-buten-1-yl diazoacetate, 2-propen-1-yl diazoacetate, trans-3-phenyl-2-propen-1-yl diazoacetate, trans-3-(para-chlorophenyl)-2-propen-1-yl diazoacetate, trans-3-(para-bromophenyl)-2-propen-1-yl diazoacetate, trans-3-(para-trifluoromethylphenyl)-2-propen-1-yl diazoacetate, trans-3-(para-methoxyphenyl)-2-propen-1-yl diazoacetate, trans-3-(para-tert-butylphenyl)-2-propen-1-yl diazoacetate, and trans-3-phenyl-2-buten-1-yl diazoacetate.

### II. SYNTHESIS OF HETERO-SUBSTITUTED PORPHYRINS

Heteroatom-substituted porphyrins and/or heteroatom-substituted chiral porphyrins of the presently disclosed subject matter are synthesized by reacting a porphyrin precursor and a heteroatom reagent and/or heteroatom chiral reagent in the presence of a metal compound, ligand and a base. Although applicants do not wish to be bound to any particular theory of the presently disclosed subject matter, it appears that the metal and ligand together (e.g., as a metal-ligand complex, or metal/ligand composition) function as a catalyst for the reaction, by which a heteroatom-substituted porphyrin and/or heteroatom-substituted chiral porphyrin is produced.

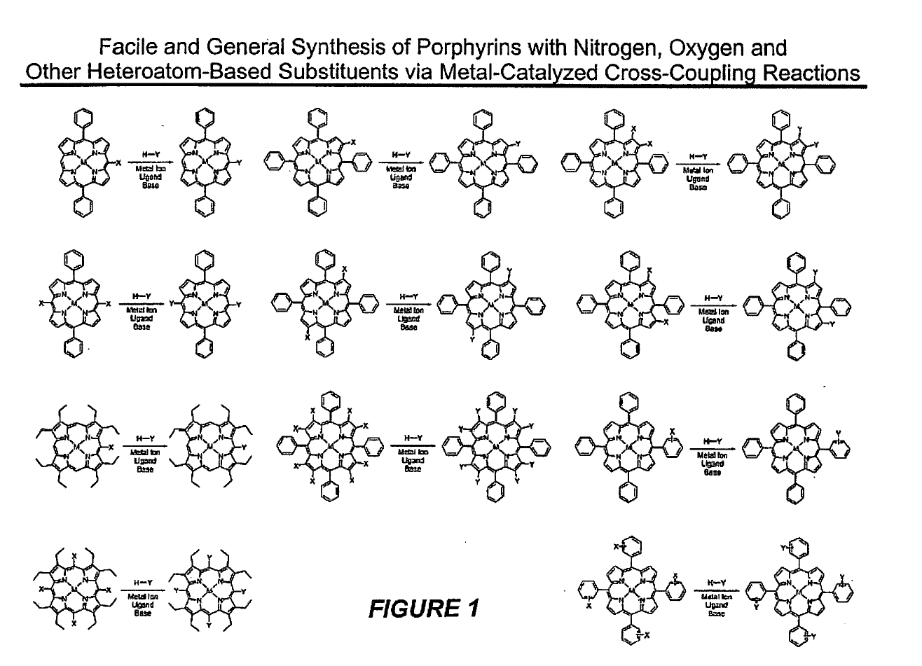

Depending on the heteroatom reagent, reactions of the presently disclosed subject matter can be, for example, cross-coupling reactions, amination reactions, or arylamination reactions. For example, in one embodiment, the metal compound and ligand together (in the configuration of a metal complex) catalyze the cross coupling reaction between the porphyrin precursor and the heteroatom reagent to yield the heteroatom-substituted porphyrin. Representative methods of the presently disclosed subject matter are generally illustrated in the several schemes shown in FIG. 1.

Porphyrin precursors of the presently disclosed subject matter have the structure of Formula I: 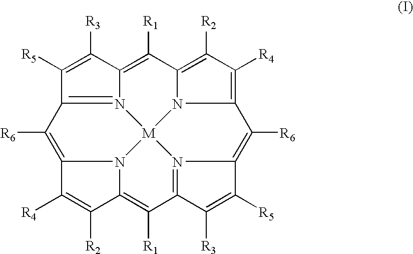
wherein:
M is H₂ or a transitional metal;
each R₁, R₂, R₃, R₄, R₅ and R₆ are each independently selected from the group consisting of X, H, alkyl, substituted alkyls, arylalkyls, aryls and substituted aryls;
X is selected from the group consisting of halogen, trifluoromethanesulfonate (OTf, haloaryl and haloalkyl, and at least one of R₁, R₂, R₃, R₄, R₅ and R₆ is X.

Transitional metals of the presently disclosed subject matter include any of the 30 metals in the 3d, 4d and 5d transition metal series of the Periodic Table of the Elements, including the 3d series that includes Sc, Ti, V, Cr, Mn, Fe, Co, Ni, Cu, and Zn; the 4d series that includes Y, Zr, Nb, Mo, Tc, Ru, Rh, Pd, Ag and Cd; and the 5d series that includes Lu, Hf, Ta, W, Re, Os, Ir, Pt, Au and Hg. In some embodiments, M is H₂ or a transition metal from the 3d series. In some embodiments, M is selected from the group consisting of H₂, Zn, Fe, and Ni. In some embodiments, M is selected from the group consisting of H₂ and Zn.

In some embodiments, the porphyrin precursor compound is halogenated, that is, at least one of R₁, R₂, R₃, R₄, R₅ and R₆ is halogen. In some embodiments, at least one meso-position of the porphyrin precursor compound is halogenated. In some embodiments, more than one meso-position of the porphyrin precursor compound is halogenated. When a porphyrin precursor compound of the presently disclosed subject matter is halogenated, one such halogen group is Br, although other halogen groups also are useful in the practice of the presently disclosed subject matter.

In some embodiments of the presently disclosed subject matter, the heteroatom reagent has the chemical structure Y—H, where Y is heteroatom-containing moiety comprising at least one of N, O, P, S, Si, B, Ge, Sn, and Se. Exemplary heteroatom-containing moieties include, but are not limited to, NR₇R₈, NR₁₀, OR₁₀, PR₇R₈, SR₁₀, SiR₇R₈R₉, BR₇R₈, GeR₇R₈R₉, SnR₇R₈R₉ and SeR₁₀, wherein R₇, R₈, R₉, and R₁₀ are each independently selected from the group consisting of H, alkyl, substituted alkyl, arylalkyl, aryl, and substituted aryl. In some embodiments, the heteroatom-containing moiety comprises one of N or O.

In some embodiments, the heteroatom reagent comprises at least one amino group. Suitable amino groups include, but are not limited to, primary amines, secondary amines, anilines, substituted aniline derivatives, aromatic amines, primary aliphatic amines, secondary aliphatic amines and cycloaliphatic amines. Specific amino groups useful in the presently disclosed subject matter include, but are not limited to, aniline, 4-nitroaniline, N-methylaniline, 4-trifluoromethylaniline, p-anisidine, 3,5-di-tert-butylaniline, n-hexylamine, benzylamine, diphenylamine, n-butylamine, 4-aminomethylpyridine, and o-toluidine. In some embodiments, the heteroatom reagent comprises an imino group. Suitable imino groups include but are not limited to benzophenone imino groups.

In some embodiments, the heteroatom reagent comprises an aryl or aryl halide group, which groups are sometimes referred to herein as phenol or substituted phenol groups. Suitable aryl groups include phenol, 4-methoxyphenol, 4-tert-butylphenol, 4-fluorophenol, 2-isopropylphenol, 3-cresol, 4-cresol, and 4-methoxyphenol.

Reactions of the presently disclosed subject matter involve a catalyst, which catalyst generally has the form of a metal complex. The metal complex comprises a metal compound of the presently disclosed subject matter complexed with a ligand. In some embodiments, the ligand comprises a phosphine ligand. Metal compounds of the presently disclosed subject matter can optionally be provided as metal precursors. Thus, as used herein, a “metal compound” can also be referred to as a “metal precursor,” a “metal precursor compound,” a “metal salt,” or a “metal ion.”

The metal precursor compounds can be characterized by the general formula M′(L)ₙ (also referred to as M′Lₙ or M′-Lₙ) where M′ is a metal selected from the group consisting of Groups 5, 6, 7, 8, 9 and 10 of the Periodic Table of Elements, L is independently each occurrence, a neutral or charged ligand, and n is a number 0, 1, 2, 3, 4, or 5, depending on M′. In some embodiments, M′ is selected from the group consisting of Ni, Pd, Fe, Pt, Ru, Rh, Co and Ir. In some embodiments, M′ is selected from the group consisting of Pd, Ni, Cu or Pt; in some embodiments, M′ is Pd. L is a compound chosen from the group consisting of halide, alkyl, substituted alkyl, cycloalkyl, substituted cycloalkyl, heterocycloalkyl, substituted heterocycloalkyl, aryl, substituted aryl, heteroaryl, substituted heteroaryl, alkoxy, aryloxy, hydroxy, boryl, silyl, hydrido, thio, seleno, phosphino, amino, and combinations thereof. When L is charged, L is selected from the group consisting of hydrogen, halogens, alkyl, substituted alkyl, cycloalkyl, substituted cycloalkyl, heteroalkyl, heterocycloalkyl, substituted heterocycloalkyl, aryl, substituted aryl, heteroaryl, substituted heteroaryl, alkoxy, aryloxy, silyl, boryl, phosphino, amino, thio, seleno, and combinations thereof. When L is neutral, L can be selected from the group consisting of carbon monoxide, isocyanide, nitrous oxide, PA₃, NA₃, OA₂, SA₂, SeA₂, and combinations thereof, wherein each A is independently selected from a group consisting of alkyl, substituted alkyl, heteroalkyl, cycloalkyl, substituted cycloalkyl, heterocycloalkyl, substituted heterocycloalkyl, aryl, substituted aryl, heteroaryl, substituted heteroaryl, alkoxy, aryloxy, silyl, and amino.

Specific examples of suitable metal precursor compounds include Pd(dba)₂, Pd₂(dba)₃, Pd(OAc)₂, PdCl₂, Pd(TFA)₂, (CH₃CN)₂PdCl₂, and the like. In some embodiments, the metal precursor compounds of the presently disclosed subject matter include Pd(OAc)₂ and Pd₂(dba)₃, where “Ac” means acetyl and “dba” means dibenzylidieneacetone.

In the practice of the presently disclosed subject matter, ligands of the presently disclosed subject matter can be combined with such a metal compound in order to provide a catalyst for the heteroatom-substitution reaction. For example, the ligand can be added to a reaction vessel at the same time as metal precursor compound along with the reactants. In other applications, the ligand will be mixed with a suitable metal precursor compound prior to or simultaneous with allowing the mixture to be contacted to the reactants. When the ligand is mixed with the metal precursor compound, a metal-ligand complex can be formed, which can be a catalyst.

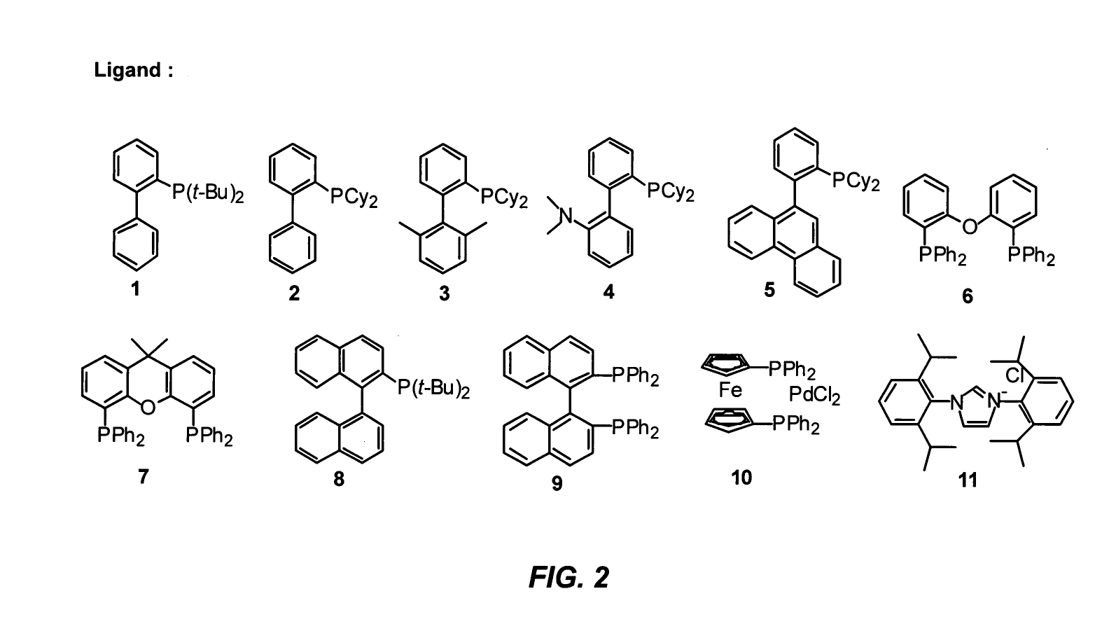

Generally, the ligands useful in the presently disclosed subject matter can be purchased or prepared by methods known to those of skill in the art. In some embodiments, the ligand comprises a phosphine ligand. Suitable phosphine ligand-metal complexes are disclosed in U.S. Pat. No. 6,268,513 to Guram et al., which patent is incorporated herein by reference in its entirety. Phosphine ligands can comprise dicycloalkylphenyl phosphine ligand or dialkylphenyl phosphine ligand, which can be in the form of a metal-ligand complex or a metal precursor/ligand composition. In some embodiments, the phosphine ligands useful in the presently disclosed subject matter comprise a cyclopentadienyl ring. Specific ligands that are useful in the practice of the presently disclosed subject matter include, but are not limited to, those whose structures are shown in FIG. 2. In some embodiments, the ligand is selected from the group consisting of DPEphos (FIG. 2, Ligand 6), BINAP (FIG. 2, Ligand 9) and 2-(Di-t-butylphosphino)-1,1-binaphthyl (FIG. 2, Ligand 8).

To carry out the process of the presently disclosed subject matter for one type of reaction, the porphyrin precursor, the heteroatom reagent, a base, a catalytic amount of metal precursor compound and a catalytic amount of the ligand are added to an inert solvent or inert solvent mixture. In a batch methodology, this mixture is stirred in some embodiments at a temperature from 0° C. to 200° C., in some embodiments from 30° C. to 170° C., in some embodiments from 50° C. to 150° C., and in some embodiments from 60° C. to 120° C. In some embodiments, the mixture is stirred at 68° C. The mixture is stirred in some embodiments for a period of from 5 minutes to 100 hours, in some embodiments from 15 minutes to 70 hours, in some embodiments from ½ hour to 50 hours, and in some embodiments from 1 hour to 30 hours. After the reaction is complete, the catalyst can be obtained as solid and separated off by filtration. The crude product is freed of the solvent or the solvents and is subsequently purified by methods known to those skilled in the art and matched to the respective product, e.g. by recrystallization, distillation, sublimation, zone melting, melt crystallization or chromatography.

Solvents suitable for the process of the presently disclosed subject matter are, for example, ethers (e.g., diethyl ether, dimethoxymethane, diethylene glycol, dimethyl ether, tetrahydrofuran (THF), dioxane, diisopropyl ether, tert-butyl methyl ether), hydrocarbons (e.g., hexane, iso-hexane, heptane, cyclohexane, benzene, toluene, xylene), alcohols (e.g., methanol, ethanol, 1-propanol, 2-propanol, ethylene glycol, 1-butanol, 2-butanol, tert-butanol), ketones (e.g., acetone, ethyl methyl ketone, iso-butyl methyl ketone), amides (e.g., dimethylformamide, dimethylacetamide, N-methylpyrrolidone), nitriles (e.g., acetonitrile, propionitrile, butyronitrile), water and mixtures thereof. In some embodiments, the solvents are selected from one of ethers (e.g., dimethoxyethane, THF), and hydrocarbons (e.g., cyclohexane, benzene, toluene, xylene). In some embodiments, the solvents are selected from one of toluene and THF.

Bases which are useful in the process of the presently disclosed subject matter are alkali metal and alkaline earth metal hydroxides, alkali metal and alkaline earth metal carbonates, alkali metal hydrogen carbonates, alkali metal and alkaline earth metal acetates, alkali metal and alkaline earth metal alkoxides, alkali metal and alkaline earth metal phosphates, primary, secondary and tertiary amines, alkali metal and alkaline earth fluorides, and ammonium fluorides. In some embodiments, the bases include but are not limited to n-BuLi, LDA, NaNH₂, NaOH, Et₃N, NaOAc, KOt-Bu, NaOt-Bu, Cs₂CO₃, K₂CO₃, K₃PO₄, carbonate-containing compounds, and phosphate-containing compounds. In some embodiments, the bases include, but are not limited to, Cs₂CO₃ and NaOt-Bu in some embodiments, the base is used in the process of the presently disclosed subject matter in an amount of from about 0.1 to about 100 equivalents, in some embodiments from about 0.5 to about 50 equivalents, in some embodiments from about 1.0 to about 10 equivalents, and in some embodiments from about 1.0 to about 1.5 equivalents.

The metal precursor compound used in this reaction is as described above and can be added to the process along with the other reactants. The metal portion of the catalyst (i.e., the metal precursor compound) is used in the process of the presently disclosed subject matter in some embodiments in a proportion of from about 0.01 to about 100 mol %, in some embodiments from about 0.1 to about 50 mol %, in some embodiments from about 0.5 to about 10 mol %, and in some embodiments from about 1 to about 5 mol %. The ligand component of the catalyst, which in some embodiments is complexed to the metal precursor compounds and in some embodiments is not complexed to the metal precursor compound, is used in the reaction in some embodiments in a proportion of from about 0.01 to about 100 mol %, in some embodiments from 0.1 to about 50 mol %, in some embodiments from about 0.5 to about 10 mol %, and in some embodiments from about 1 to about 5 mol %. These amounts can be combined to give metal precursor to ligand ratios useful in the process. It is also possible, if desired, to use mixtures of two or more different ligands.

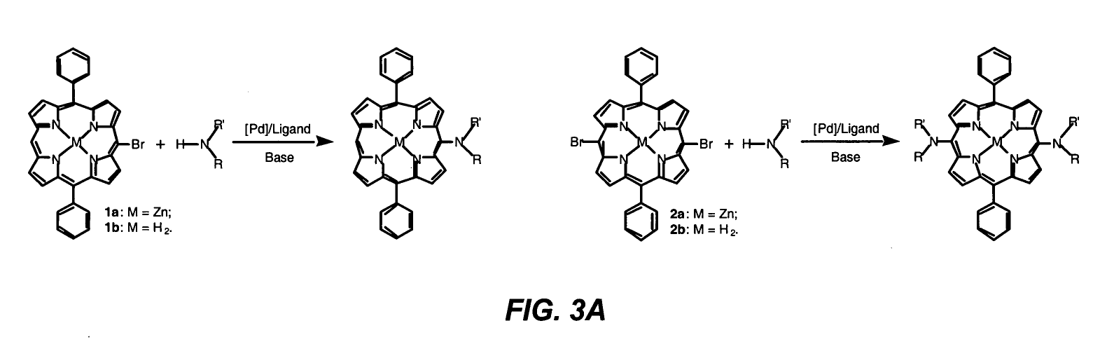

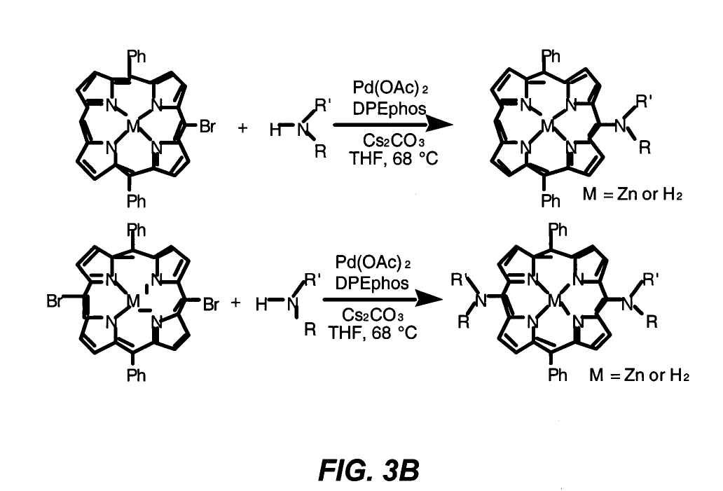

In some embodiments of the presently disclosed subject matter, at least one meso-position of the synthesized heteroatom-substituted porphyrin is substituted; that is, the heteroatom-substituted porphyrin is a meso-substituted porphyrin. In some embodiments of the presently disclosed subject matter, amino-substituted porphyrins are obtained from halogenated porphyrin precursors via palladium-catalyzed amination. Specifically, meso-arylamino- and alkylamino-substituted porphyrins are efficiently synthesized by reacting meso-halogenated porphyrins with amines via palladium-catalyzed amination. A general schematic of this embodiment is illustrated in FIG. 3A. FIG. 3B illustrates two particular embodiments of the presently disclosed subject matter. In the schematic on the left side of the figure, the porphyrin precursors 5-bromo-10,20-diphenylporphyrine and its corresponding zinc complex [5-bromo-10,20-diphenyl porphyrino]zinc(II) are each reacted with an amino group to yield the illustrated amino-substituted porphyrin. In the schematic on the right side of the picture, [5,15-dibromo-10,20-diphenylporphyrino]zinc(II) and its corresponding zinc complex [5,15-dibromo-10,20-diphenylporphyrino]zinc(II) are each reacted with an amino group to provide the indicated amino-substituted porphyrin. The precursors and amine reagents are reacted in the presence of palladium acetate and the commercially available phosphine ligand bis(2-diphenylphosphinophenyl) ether, or “DPEphos”.

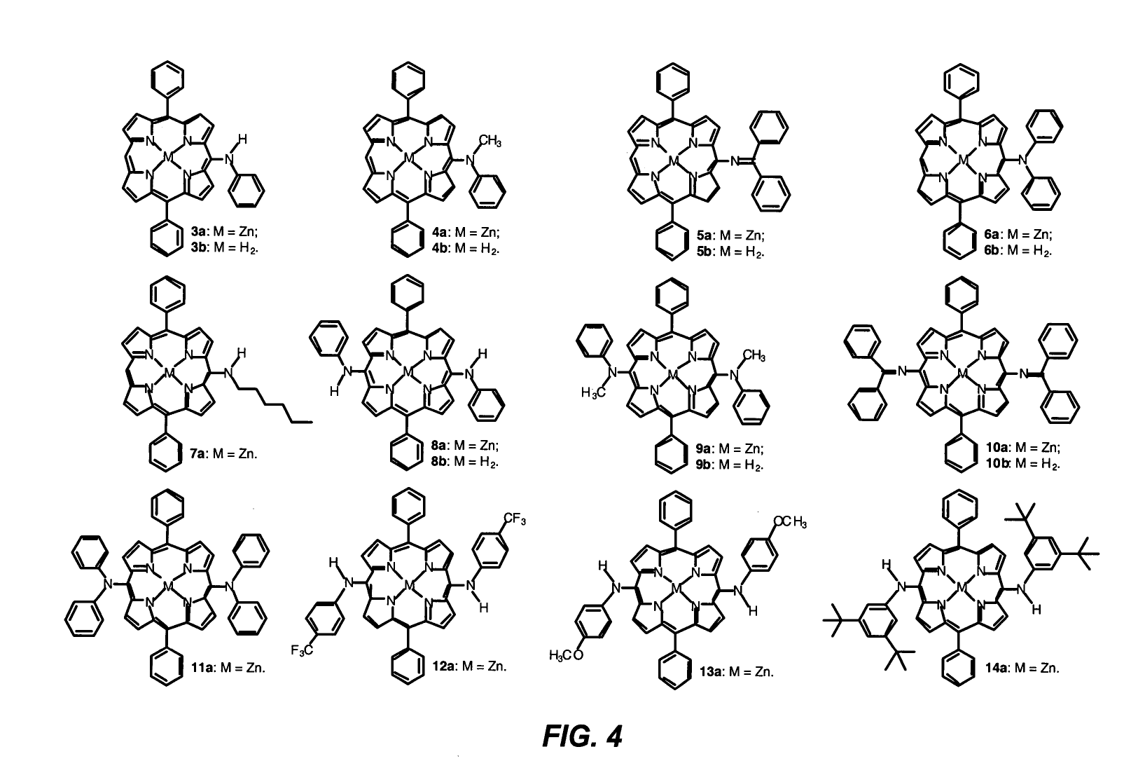

In some embodiments of the presently disclosed subject matter, a variety of different amines are efficiently coupled with the meso-brominated 10,20-diphenylporphyrins, 5-bromo-1 0,20-diphenylporphyrine, and 5,15-dibromo-10,20-diphenylporphyrine (compounds 1b and 2b in Table 1) as well as their corresponding zinc complexes [5-bromo-10,20-diphenyl porphyrino]zinc(II) and [5,15-dibromo-10,20-diphenylporphyrino]zinc(II) (compounds 1a and 2a in Table 1). The meso-arylamino- and alkylamino-substituted porphyrins that are obtained are summarized in Table 1, below, with the structures of the resulting compound being shown in FIG. 4. Specifically, both the primary aniline (Table 1, entry 1) and the secondary N-methylaniline (Table 1, entry 3) can be effectively coupled with 1a to give monoamino-substituted porphyrins 3a and 4a, respectively. When 2a is used, the corresponding diamino-substituted porphyrins 8a (Table 1, entry 10) and 9a (Table 1, entry 12) are synthesized via double amination reactions. Substituted aniline derivatives such as 4-trifluoromethylaniline (Table 1, entry 17), p-anisidine (Table 1, entry 18) and 3,5-di-tert-butylaniline (Table 1, entry 19) also give high yields of double amination products when reacted with 2a. Primary aliphatic amines can also be well-coupled, as demonstrated in the case of n-hexylamine with la (Table 1, entry 9).

In addition to primary and secondary amines, imines are also suitable coupling partners under similar reaction conditions. When benzophenone imine was employed, monoimino-substituted porphyrin 5a (Table 1, entry 5) and diimino-substituted porphyrin 10a (Table 1, entry 14) are obtained from its reactions with 1a and 2a, respectively.

TABLE 1
Palladium-Catalyzed Amination of meso-bromoporphyrins with amines

| | | | | | yield |
|---|---|---|---|---|---|
| entry | reactant^{b} | amine | time (h)^{c} | product^{d} | (%)^{e} |
| 1 | 1a | PhNH₂ | 13 | 3a | 95 |
| 2 | 1b | PhNH₂ | 19 | 3b | 98 |
| 3 | 1a | Ph(Me)NH | 13 | 4a | 99 |
| 4 | 1b | Ph(Me)NH | 16 | 4b | 94 |
| 5 | 1a | Ph₂C═NH | 22 | 5a | 94 |
| 6 | 1b | Ph₂C═NH | 24 | 5b | 84 |
| 7 | 1a | Ph₂NH | 25 | 6a | 61^{f} |
| 8 | 1b | Ph₂NH | 40 | 6b | 66 |
| 9 | 1a | n-HexNH₂ | 50 | 7a | 80 |
| 10 | 2a | PhNH₂ | 13 | 8a | 82 |
| 11 | 2b | PhNH₂ | 20 | 8b | 65 |
| 12 | 2a | Ph(Me)NH | 17 | 9a | 82 |
| 13 | 2b | Ph(Me)NH | 15 | 9b | 71 |
| 14 | 2a | Ph₂C═NH | 16 | 10a | 84 |
| 15 | 2b | Ph₂C═NH | 15 | 10b | 95 |
| 16 | 2a | Ph₂NH | 50 | 11a | 30 |
| 17 | 2a | (4-CF₃Ph)NH₂ | 17 | 12a | 90 |
| 18 | 2a | (4-CH₃OPh) NH₂ | 16 | 13a | 94 |
| 19 | 2a | (3,5-di-t-BuPh)NH₂ | 62 | 14a | 95 |
| Reactions were carried out at 68° C. in THF under N₂ with 1.0 equiv of bromoporphyrin, 3.6 equiv of amine for 1b and 2b or 4.8 equiv of amine for 1a and 2a, 5 mol % Pd(OAc)₂ and 7.5 mol % DPEphos in the presence of 1.4 equiv of Cs₂CO₃ per Br. Concentration: 0.05 mmol bromoporphyrin/5 mL THF. Yields represent isolated yields of >95% purity as determined by ¹H NMR. The reaction was conducted using 10 mol % Pd(OAc)₂ and 15 mol % | | | | | |
| # DPEphos in the presence of 2.8 equiv of NaOt-Bu. | | | | | |

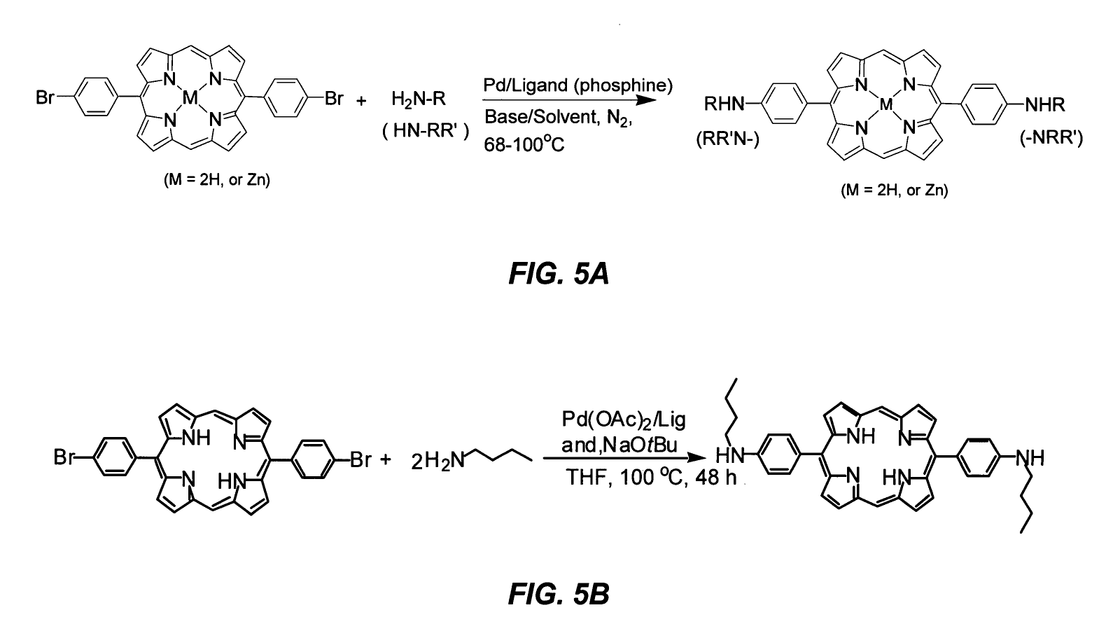

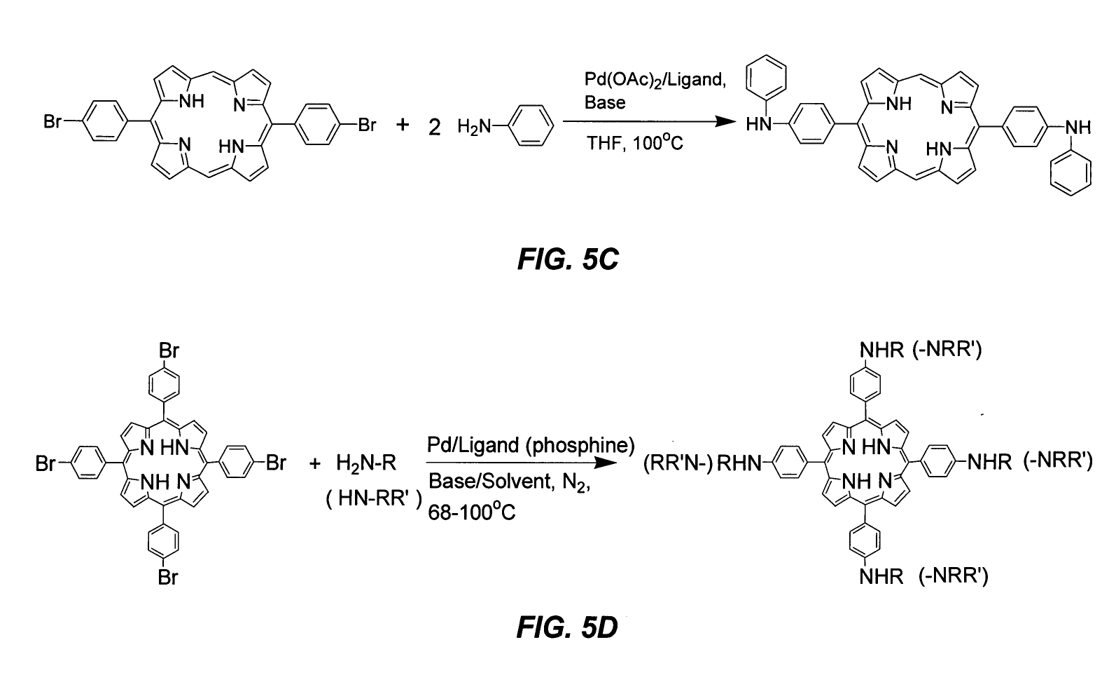

In embodiments of the presently disclosed subject matter, the methods of the presently disclosed subject matter are carried out to produce aminophenylporphyrins. In one such embodiment, the porphyrin precursors are p-bromophenyl porphyrin and its zinc complex, and the amination reaction is catalyzed by palladium. Schemes for this reaction are illustrated in FIGS. 5A-5D, with exemplary aminophenylporphyrins obtained in the presently disclosed subject matter being described in Tables 2 and 3, below.

TABLE 2
5,15-di-aminophenylporphyrin and zinc complex
synthesized via Pd catalyzed amination reaction

| | | Ligand | Base | | | Isolated yield (%) | |
|---|---|---|---|---|---|---|---|
| | Amine | (10%) | (8.0) | | | A | B |
| Entry | (8.0 equiv) | equiv | equiv | Solvent | Time | M = 2H | M = Zn (II) |
| 1 | 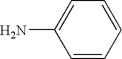 | 9 | Cs₂CO₃ | Toluene | 48 h | 70 | 66 |
| | | | NaOtBu | Toluene | 48 h | 88 | — |
| | | 9 | NaOtBu | THE | 24 h | 95 | — |
| | | 9 | NaOtBu | THF | 13 h | 83 | — |
| | | 9 | Cs₂CO₃ | THF | 48 h | 92 | — |
| | | 9 | Cs₂CO₃ | THF | 48 h | 85^{a} | — |
| | | 9 | NaOtBu | THF | 48 h | 92 | — |
| | | 9 | NaOtBu | THF | 48 h | — | |
| 2 | 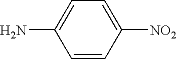 | 9 | Cs₂CO₃ | Toluene | 48 h | 76 | — |
| 3 | 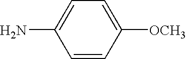 | 3 | NaOtBu | THF | 48 h | 93 | 68 |
| 4 | 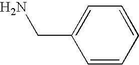 | 3 | NaOtBu | THF | 48 h | — | 83 |
| 5 | 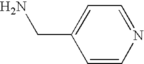 | 9 | NaOtBu | THF | 48 h | 88 | — |
| | | 8 | NaOtBu | THE | 48h | 80 | — |
| 6 | 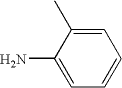 | 9 | Cs₂CO₃ | Toluene | 66.5 h | 45 | — |
| | | 3 | NaOtBu | THF | 48 h | 87 | 73 |
| 7 | 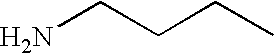 | 8 | NaOtBu | THF | 48 h | 83 | 93 |
| | | 8 | NaOtBu | THF | 24 h | 63 | — |
| | | 8 | NaOtBu | THF | 13 h | 76 | — |
| | | 8 | NaOtBu | THF | 48 h | 69^{b} | — |
| | | 1 | Cs₂CO₃ | THF | 48 h | 79 | — |
| | | 2 | NaOtBu | THF | 48 h | 66^{b} | — |
| | | 2 | NaOtBu | THF | 48 h | 99 | — |
| | | 3 | NaOtBu | THF | 48 h | 92 | — |
| | | 3 | NaOtBu | THF | 48 h | 93 | — |
| 8 | 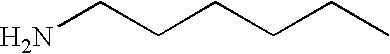 | 3 | NaOtBu | THF | 48 h | 90 | 53^{c} |
| 9 | 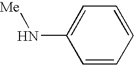 | 3 | NaOtBu | THF | 48 h | 88 | 73 |
| 10 | 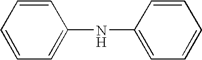 | 3 | NaOtBu | THF | 48 h | 81 | 57 |
| | | 9 | NaOtBu | THF | 48 h | 57 | — |
| 11 | 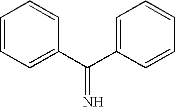 | 1 | NaOtBu | THF | 48 h | 52 | — |
| 12 | 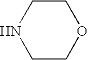 | 8 | Cs₂CO₃ | THF | 48 h | 76 | — |
| | | 8 | NaOtBu | THF | 48 h | 79 | — |

Note:
^{a}4.0 equiv aniline;
^{b}Pd(OAc)₂/Ligand = 10%/20%,
^{c}ligand 7

TABLE 3
Tetrakis-aminophenylporphyrins synthesized from
tetrakis-p-bromophenylporphyrin through
Pd catalyzed amination reaction

| | Amine | Pd | Ligand | Base | | | | |
|---|---|---|---|---|---|---|---|---|
| | 16.0 | (5%) | (10%) | (16.0) | | | | Isolated |
| Entry | equiv | equiv | equiv | equiv | Solvent | ° C. | Time | yield |
| 1 | 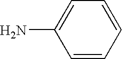 | Pd(OAc)₂ | 9 | NaOtBu | THF | 100 | 72 h | 91% |
| 2 | 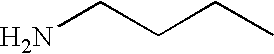 | Pd(OAc)₂ | 8 | NaOtBu | THF | 100 | 72 h | 86% |
| 3 | 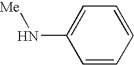 | Pd(OAc)₂ | 9 | NaOtBu | THF | 100 | 72 h | 82% |
| 4 | 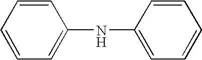 | Pd(OAc)₂ | 9 | NaOtBu | THF | 100 | 72 h | 81% |

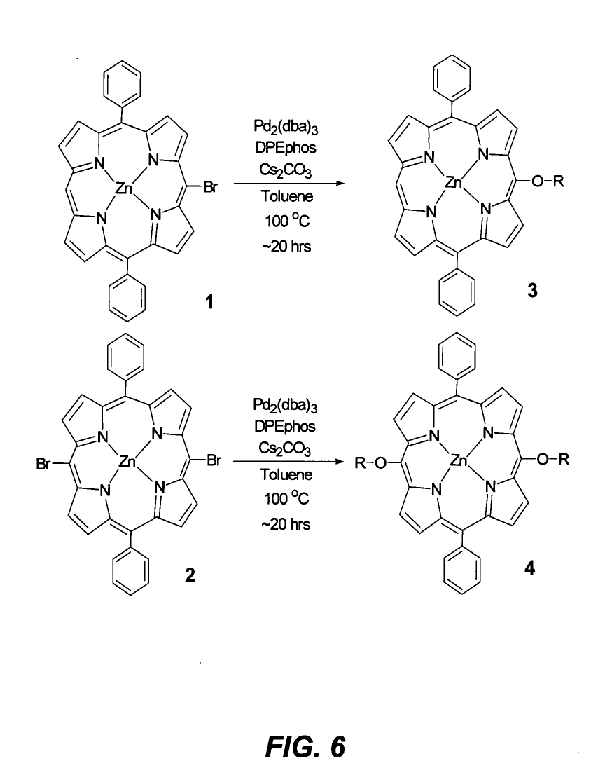

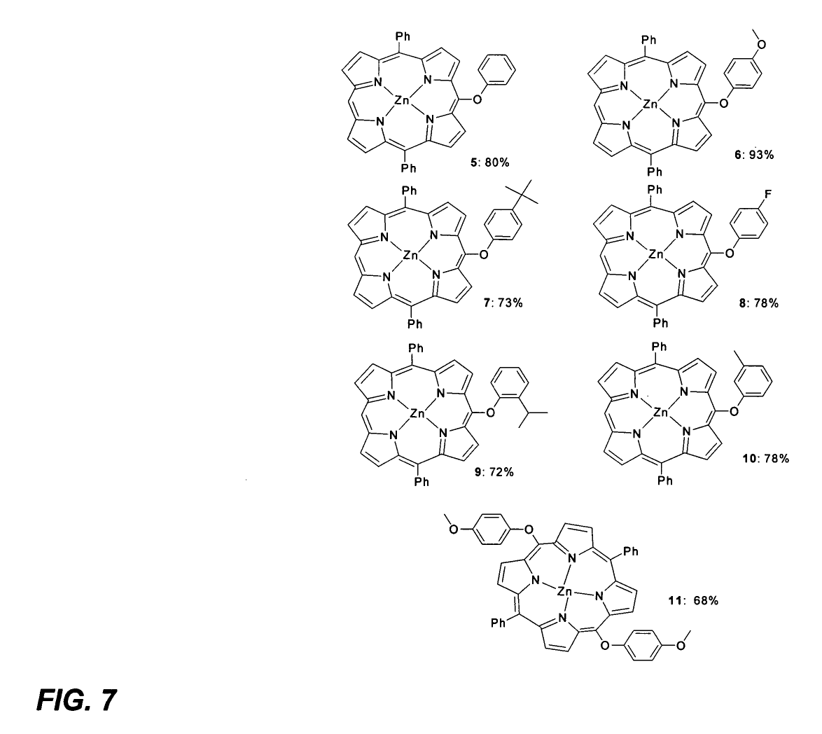

In some embodiments of the presently disclosed subject matter, monobromo-porphyrin [5-bromo-10,20-diphenylporphyrino]zinc(II) and the dibromoporphyrin [5,15-dibromo-10,20-diphenylporphyrino]zinc(II) can undergo efficient cross-coupling reactions with various phenols under mild conditions to yield desired phenoxy- and diphenoxy-substituted porphyrins. FIG. 6 illustrates the etheration of monobromo-porphyrin [5-bromo-10,20-diphenylporphyrino]zinc(II) and the dibromoporphyrin [5,15-dibromo-10,20-diphenylporphyrino]zinc(II) using a combination of Pd(OAc)₂ or Pd₂(dba)₃ and a phosphine ligand as the catalyst. FIG. 7 illustrates the chemical structures of a variety of phenoxy- and diphenoxy-substituted porphyrins that are obtained in the practice of the presently disclosed subject matter.

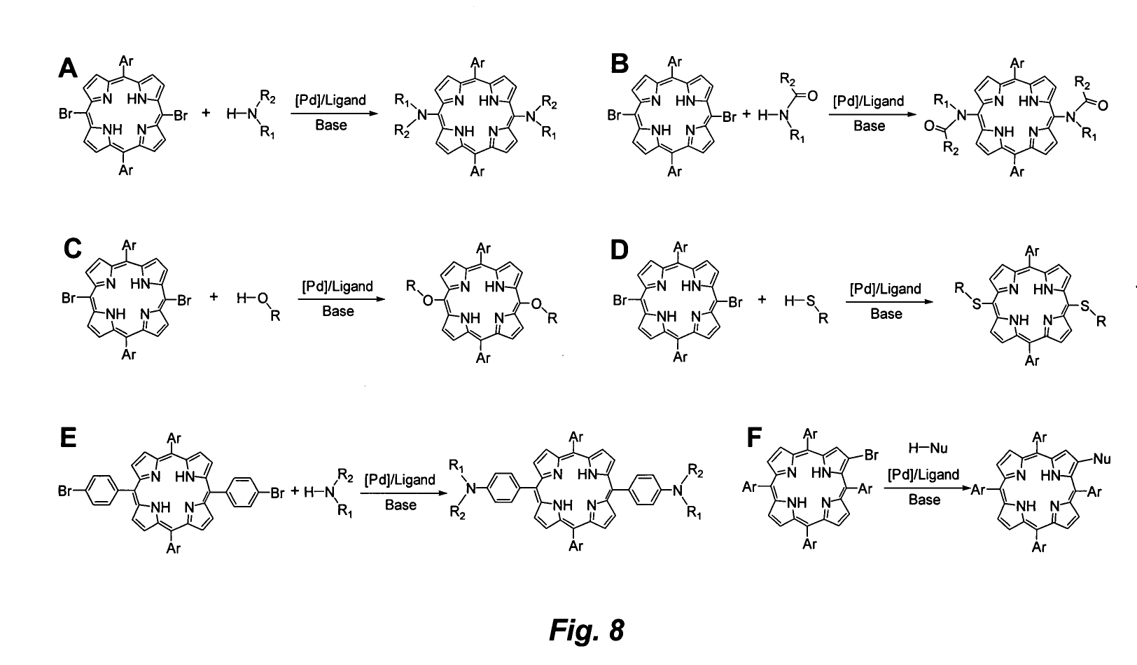

In summary, and as provided in FIG. 8, the presently disclosed subject matter demonstrates that halogenated porphyrins, e.g., bromoporphyrins, are versatile precursors for the synthesis of heteroatom-functionalized porphyrins via metal-catalyzed carbon-heteroatom cross-coupling reactions with soft, non-organometallic nucleophiles. See also Chen et al., (2003) J. Org. Chem. 68: 4432; Gao et al., (2003) J. Org. Chem. 68: 6215; Gao et al., (2003) Org. Lett. 5: 3261; and Gao et al., (2004) Org. Lett. 6: 1837, each of which is incorporated herein by reference in their entirety. As provided herein below, these methods also can be used to synthesize heteroatom chiral porphyrins.

### III. CHIRAL PORPHYRINS

In some embodiments, the presently disclosed subject matter describes a chiral porphyrin compound having the structure of Formula (I): 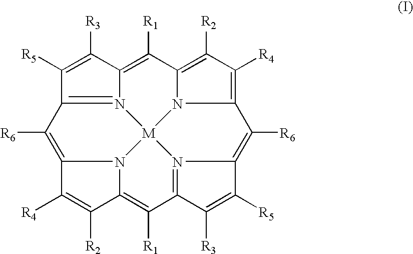
wherein: M is present or absent and when present is H₂ or a transition metal; R₁, R₂, R₃, R₄, R₅ and R₆ are each independently selected from the group consisting of Y, H, alkyl, substituted alkyl, arylalkyl, aryl, and substituted aryl, wherein Y is a heteroatom-containing chiral moiety; and at least one of R₁, R₂, R₃, R₄, R₅ and R₆ is Y. Representative chiral porphyrins of the presently disclosed subject matter and methods for preparing the same are provided herein below in Examples 59-146.

In some embodiments, M is present and is selected from the group consisting of H₂, Zn, Fe, Ni, Co, Mn, Ru, and Rh. In some embodiments, M is Co.

In some embodiments, Y comprises a heteroatom-containing chiral moiety selected from the group consisting of NR₇R₈, OR₉, SR₁₀, and BR₁₀ wherein R₇, R₈, R₉, and R₁₀ are each independently selected from the group consisting of H, alkyl, substituted alkyl, arylalkyl, alkoxyl, carboxyl, aryl, and substituted aryl. In some embodiments, Y is a selected from the group consisting of chiral amino, substituted chiral amino, chiral amido, substituted chiral amido, chiral alkoxy, substituted chiral alkoxy, chiral thio, substituted chiral thio, and chiral borate ester moieties. In some embodiments, Y is selected from the group consisting of (+)-estrone; (+)-dihydrocholesterol; R-(+)-1,1′-bi-2-naphthol; (R)-(+)-4-benzyl-2-oxazolidinone; (L)-phenylalanine methyl ester; 1-[1′-(R)-α-methylbenzyl]-aziridine-2(R)-carboxamide; (R)-(−)-2-methoxypropionamide; (S)-(+)-2-methoxypropionamide; (S)-(+)-2,2-dimethylcyclopropanecarboxamide; and L-(R)-lactamide.

In some embodiments, at least one of R₁ and R₆ is aryl, wherein the aryl group is bound to at least one heteroatom-containing chiral moiety Y. In some embodiments, the aryl group bound to at least one heteroatom-containing chiral moiety Y has the structure: 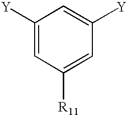
wherein R₁₁ is selected from the group consisting of H, alkyl, substituted alkyl, alkoxy, substituted alkoxy, halogen, arylalkyl, aryl, and substituted aryl.

In some embodiments, at least one of R₁ and R₆ is H. In some embodiments, at least one of R₁ and R₆ is alkyl. In some embodiments, at least one of R₁ and R₆ is n-heptyl. In some embodiments, at least one of R₁ and R₆ is aryl. In some embodiments, at least one of R₁ and R₆ is phenyl. In some embodiments, at least one of R₁ and R₆ is substituted aryl. In some embodiments, at least one of R₁ and R₆ is selected from the group consisting of 2,6-dimethylphenyl, 2,4,6-trimethylphenyl, 3,5-di-tert-butylphenyl, 2,6-dimethoxyphenyl, 3,5-dimethoxyphenyl, 4-t-butylphenyl, 4-acetylphenyl, 4-trifluoromethylphenyl, and pentafluorophenyl. In some embodiments, at least one of R₁ and R₆ is Y. In some embodiments, each R₆ is Y.

### IV. METHOD OF SYNTHESIZING A HETEROATOM-SUBSTITUTED CHIRAL PORPHYRIN

Based on the methods of synthesizing heteroatom-substituted porphyrins as provided hereinabove and as summarized in FIG. 8, in some embodiments, the presently disclosed subject matter describes the use of haloporphyrins as a new class of synthons for the modular construction of chiral porphyrins via palladium-mediated carbon-heteroatom bond formation reactions with chiral amines, chiral amides, chiral alcohols, and chiral thiols.

Accordingly, in some embodiments, a method of synthesizing a heteroatom-substituted chiral porphyrin compound is disclosed, the method comprising reacting a porphyrin precursor with a chiral reagent comprising a heteroatom, the porphyrin precursor having a structure of Formula II: 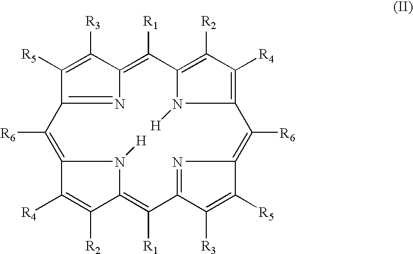
wherein R₁, R₂, R₃, R₄, R₅ and R₆ are each independently selected from the group consisting of X, H, alkyl, substituted alkyl, arylalkyl, aryl, and substituted aryl; X is selected from the group consisting of halogen, trifluoromethanesulfonate (OTf), OTf-substituted aryl, haloaryl and haloalkyl, and at least one of R₁, R₂, R₃, R₄, R₅ and R₆ is X; wherein the chiral reagent comprising a heteroatom has the structure H—Y and Y is a heteroatom-containing chiral moiety comprising at least one of N, O, and S; and wherein the porphyrin precursor and chiral reagent comprising a heteroatom are reacted in the presence of a metal compound, a ligand, and a base to produce a heteroatom-substituted chiral porphyrin.

In some embodiments, X is a halogen selected from the group consisting of Br, Cl, I and F. In some embodiments, X is Br. In some embodiments, at least one meso-position of the porphyrin precursor of Formula II is halogenated. In some embodiments, X is haloaryl. In some embodiments, the haloaryl is 2,6-dibromophenyl.

In some embodiments, the metal compound comprises a metal selected from the group consisting of Pd, Pt, Ni, or Cu. In some embodiments, the metal compound is a metal precursor compound selected from the group consisting of Pd(dba)₂, Pd₂(dba)₃, Pd(OAc)₂, PdCl₂, Pd(TFA)₂, and (CH₃CN)₂PdCl₂. In some embodiments, the base is selected from the group consisting of n-BuLi, LDA, NaNH₂, NaOH, Et₃N, NaOAc, KOt-Bu, NaOt-Bu, Cs₂CO₃, K₂CO₃, and K₃PO₄.

In some embodiments, the ligand is selected from the group of ligands provided in FIG. 2.

In some embodiments, Y comprises a heteroatom-containing chiral moiety selected from the group consisting of NR₇R₈, OR₉, and SR₁₀ wherein R₇, R₈, R₉, and R₁₀ are each independently selected from the group consisting of H, alkyl, substituted alkyl, arylalkyl, alkoxyl, carboxyl, aryl, and substituted aryl.

In some embodiments, Y is selected from the group consisting of chiral amino, substituted chiral amino, chiral amido, substituted chiral amido, chiral alkoxy, substituted chiral alkoxy, chiral thio, and substituted chiral thio moieties. In some embodiments, Y is selected from the group consisting of (+)-estrone; (+)-dihydrocholesterol; R-(+)-1,1′-bi-2-naphthol; (R)-(+)-4-benzyl-2-oxazolidinone; (L)-phenylalanine methyl ester; 1-[1′-(R)-α-methylbenzyl]-aziridine-2(R)-carboxamide; (R)-(−)-2-methoxypropionamide; (S)-(+)-2-methoxypropionamide; (S)-(+)-2,2-dimethylcyclopropanecarboxamide; and L-(R)-lactamide.

### V. METHOD OF SYNTHESIZING A CYCLOPROPANE COMPOUND

### V.A. Intermolecular Cyclopropanation

Transition metal complex-mediated cyclopropanation of alkenes with diazo compounds as shown in Equation 1 is an efficient and selective method for constructing synthetically and biologically important cyclopropanes.

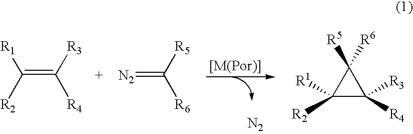

Among the various catalysts used in cyclopropanation reactions, metalloporphyrins are unique in their unusual selectivity and high catalytic turnover. The family of porphyrins described by the presently disclosed subject matter provides improved metal-based, catalytic systems for cyclopropanation.

Accordingly, in some embodiments, the presently disclosed subject matter discloses a method of synthesizing a cyclopropane compound, the method comprising reacting an alkene with a diazo compound in the presence of a porphyrin metal complex, wherein the porphyrin metal complex has the structure of Formula (I):

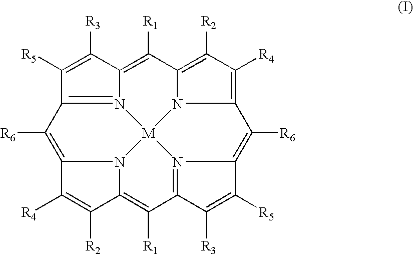

wherein: M is a transition metal; R₁, R₂, R₃, R₄, R₅ and R₆ are each independently selected from the group consisting of H, alkyl, substituted alkyl, arylalkyl, aryl, substituted aryl, and Y, wherein Y is a heteroatom-containing chiral moiety. Optionally, in some embodiments, at least one of R₁, R₂, R₃, R₄, R₅ and R₆ is Y.

In some embodiments, M is selected from the group consisting of Zn, Fe, Ni, Co, Mn, Ru, and Rh. In some embodiments, M is Co.

In some embodiments, the diazo compound is selected from the group consisting of ethyl diazoacetate, t-butyl diazoacetate, 2,6-di-tert-butyl-4-methylphenyl diazoacetate, methyl phenyldiazoacetate, ethyl diazoacetacetate, diethyl diazomalonate, and trimethylsilyldiazomethane. In some embodiments, the diazo compound is selected from one of ethyl diazoacetate and t-butyl diazoacetate.

In some embodiments, the alkene is selected from the group consisting of aromatic alkene, non-aromatic alkene, di-substituted alkene, tri-substituted alkene, tetra-substituted alkene, cis-alkene, trans-alkene, cyclic-alkene, and non-cyclic alkene. In some embodiments, the alkene is styrene.

In some embodiments, the method comprises an additive. In some embodiments, the additive is selected from the group consisting of 4-dimethylaminopyridine, nitrogen, phosphine, and sulfur coordinating ligands.

In some embodiments, the cyclopropane compound has an enantiomeric purity ranging from about 30% enantiomeric excess to about 99% enantiomeric excess. In some embodiments, the cyclopropane compound has an enantiomeric purity ranging from about 50% enantiomeric excess to about 99% enantiomeric excess. In some embodiments, the cyclopropane compound has an enantiomeric purity ranging from about 80 % enantiomeric excess to about 99% enantiomeric excess. In some embodiments, the cyclopropane compound has an enantiomeric purity ranging from about 90% enantiomeric excess to about 99% enantiomeric excess.

### V.B. Intramolecular Cyclopropanation

In some embodiments, the presently disclosed subject matter describes a method for porphyrin catalyzed intramolecular cyclopropanation of an alkene. Accordingly, in some embodiments, the method comprises contacting an alkene-substituted diazo compound with a porphyrin metal complex to form a cyclopropane compound, wherein the porphyrin metal complex has the structure of Formula (I):

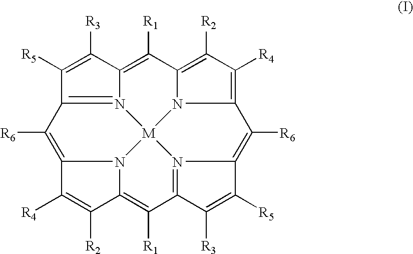

wherein:
M is Co;
R₁, R₂, R₃, R₄, R₅ and R₆ are each independently selected from the group consisting of H, alkyl, substituted alkyl, arylalkyl, aryl, substituted aryl, and Y, wherein Y is a heteroatom-containing chiral moiety.
   Optionally, in some embodiments, at least one of R₁, R₂, R₃, R₄, R₅ and R₆ is Y.

In some embodiments, the alkene-substituted diazo compound comprises an alkene-substituted diazoacetate compound. In some embodiments, the alkene-substituted diazoacetate compound comprises an allylic diazoacetate compound. In some embodiments, the alkene-substituted diazo compound is selected from the group consisting of 3-methyl-2-buten-1-yl diazoacetate, 2-propen-1-yl diazoacetate, trans-3-phenyl-2-propen-1-yl diazoacetate, trans-3-(para-chlorophenyl)-2-propen-1-yl diazoacetate, trans-3-(para-bromophenyl)-2-propen-1-yl diazoacetate, trans-3-(para-trifluoromethylphenyl)-2-propen-1-yl diazoacetate, trans-3-(para-methoxyphenyl)-2-propen-1-yl diazoacetate, trans-3-(para-tert butylphenyl)-2-propen-1-yl diazoacetate, and trans-3-phenyl-2-buten-1-yl diazoacetate.

In some embodiments, the method comprises contacting the alkene-substituted diazo compound with the porphyrin metal complex in the presence of an additive. In some embodiments, the additive is selected from the group consisting of 4-dimethylaminopyridine (DMAP), nitrogen, phosphine, and sulfur coordinating ligands.

In some embodiments, the cyclopropane compound has an enantiomeric purity ranging from about 25% enantiomeric excess to about 99% enantiomeric excess. In some embodiments, the cyclopropane compound has an enantiomeric purity ranging from about 50 % enantiomeric excess to about 99% enantiomeric excess. In some embodiments, the cyclopropane compound has an enantiomeric purity ranging from about 80% enantiomeric excess to about 99% enantiomeric excess. In some embodiments, the cyclopropane compound has an enantiomeric purity ranging from about 90 % enantiomeric excess to about 99% enantiomeric excess.

### VI. METHOD OF SYNTHESIZING AN AZIRIDINE COMPOUND

Aziridines are a class of synthetically and biologically important compounds that have found many applications. Among synthetic methodologies, transition metal complex-mediated aziridination represents a direct and powerful approach for the construction of the aziridine rings. Accordingly, in some embodiments, the presently disclosed subject matter provides a method of synthesizing an aziridine compound, the method comprising reacting an alkene with a nitrene source in the presence of a cobalt-containing catalyst.

Further, in some embodiments, the presently disclosed subject matter provides a metalloporphyrin (e.g., a cobalt porphyrin)-mediated aziridination of an alkene. An example of a metalloporphyrin mediated aziridination of an alkene is illustrated in Equation 2.

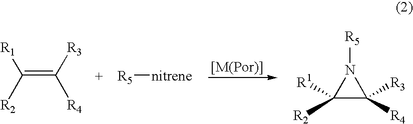

Accordingly, in some embodiments, the presently disclosed subject matter describes a method of synthesizing an aziridine compound, the method comprising reacting an alkene with a nitrene source in the presence of a porphyrin metal complex, wherein the porphyrin metal complex has the structure of Formula (I): 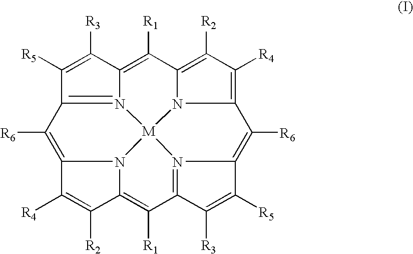
wherein: M is a transition metal; R₁, R₂, R₃, R₄, R₅ and R₆ are each independently selected from the group consisting of H, alkyl, substituted alkyl, arylalkyl, aryl, substituted aryl, and Y, wherein Y is a heteroatom-containing chiral moiety. Optionally, in some embodiments, at least one of R₁, R₂, R₃, R₄, R₅ and R₆ is Y.

In some embodiments, M is selected from the group consisting of Zn, Fe, Ni, Co, Mn, Ru, and Rh. In some embodiments, M is Co.

In some embodiments, the nitrene source is selected from the group consisting of bromamine-T, chloramines-T, and an organic azide. In some embodiments, the organic azide is selected from the group consisting of a phosphoryl azide, a phosphinyl azide, and a phosphorodiamidic azide. In some embodiments, the nitrene source is bromamine-T. In some embodiments, the nitrene source is diazo diphenylphosphoryl azide.

In some embodiments, the alkene is selected from the group consisting of aromatic alkene, non-aromatic alkene, di-substituted alkene, tri-substituted alkene, tetra-substituted alkene, cis-alkene, trans-alkene, cyclic-alkene, and non-cyclic alkene.

In some embodiments, R₁ and R₆ are independently selected from the group consisting of aryl and substituted aryl. In some embodiments, the substituted aryl is substituted with an electron-withdrawing group. In some embodiments, the electron-withdrawing group is selected from one of halogen and trihaloalkyl, such as trifluoromethyl.

In some embodiments, the porphyrin metal complex is selected from the group consisting of [Fe(TPP)Cl], [Fe(TPFPP)Cl], [Co(TDClPP)] and [Co(TPFPP)]. In some embodiments, the porphyrin metal complex is [Co(TPP)].

In some embodiments, the porphyrin is present in a concentration ranging from about 2 mol % to about 10 mol %. In some embodiments, the porphyrin is present in a concentration ranging from about 5 mol % to about 10 mol %.

In some embodiments, the alkene and the nitrene source are present in a ratio of about 1:2 alkene:nitrene. In some embodiments, the alkene and the nitrene source are present in a ratio of about 5:1 alkene:nitrene.

In some embodiments, the reacting of the alkene with the nitrene source takes place in a solvent. In some embodiments, the solvent is selected from the group consisting of acetonitrile and chlorobenzene.

In some embodiments, the reacting of the alkene and the nitrene source takes place at about room temperature, e.g., between about 20° C. to about 25° C. In some embodiments, the reacting of the alkene with the nitrene source takes place at a temperature of between about 80° C. and about 120° C. In some embodiments, the reacting of the alkene with the nitrene source takes place for between about 6 hours and about 46 hours.

### VII. METHOD OF SYNTHESIZING AN EPOXIDE COMPOUND

The chiral porphyrins of the presently disclosed subject matter also can be used as catalysts in asymmetric epoxidation reactions as illustrated in Equation 3.

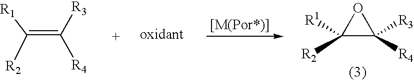

In some embodiments, the presently disclosed subject matter describes a method of synthesizing an epoxide compound, the method comprising reacting an alkene with a oxidant in the presence of a chiral porphyrin metal complex, wherein the chiral porphyrin metal complex has the structure of Formula (I): 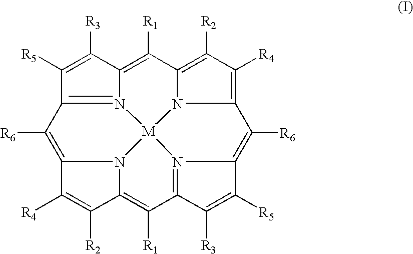
wherein: M is a transition metal; R₁, R₂, R₃, R₄, R₅ and R₆ are each independently selected from the group consisting of Y, H, alkyl, substituted alkyl, arylalkyl, aryl, and substituted aryl, wherein Y is a heteroatom-containing chiral moiety; and at least one of R₁, R₂, R₃, R₄, R₅ and R₆ is Y.

In some embodiments, M is selected from the group consisting of Zn, Fe, Ni, Co, Mn, Ru, and Rh. In some embodiments, M is Co.

In some embodiments, the oxidant is selected from the group consisting of sodium hypochlorite, potassium monopersulfate, hydrogen peroxide, alkylhydroperoxides, m-chloroperbenzoic acid, amines, N-oxides, iodosylbenzene, peroxycarboxylic acids, dioxiranes, hypochlorite, and oxygen. In some embodiments, the oxidant is oxygen.

In some embodiments, the alkene is selected from the group consisting of aromatic alkene, non-aromatic alkene, di-substituted alkene, tri-substituted alkene, tetra-substituted alkene, cis-alkene, trans-alkene, cyclic-alkene, and non-cyclic alkene.

### VIII. CHEMICAL LIBRARY OF CHIRAL METALLOPORPHYRINS AND METHODS OF USE THEREOF

In some embodiments, the presently disclosed subject matter describes a chemical library comprising a plurality of chiral metalloporphyrin compounds having the structure of Formula (I): 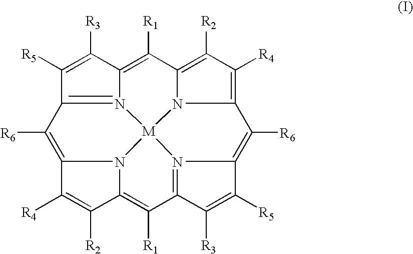
wherein M is a transition metal; R₁, R₂, R₃, R₄, R₅ and R₆ are each independently selected from the group consisting of Y, H, alkyl, substituted alkyl, arylalkyl, aryl, and substituted aryl, wherein Y is a heteroatom-containing chiral moiety; and at least one of R₁, R₂, R₃, R₄, R₅ and R₆ is Y.

In some embodiments, M is selected from the group consisting of Zn, Fe, Ni, Co, Mn, Ru, and Rh. In some embodiments, M is Co.

In some embodiments, the chiral metalloporphyrins are attached to a substrate. In some embodiments, the substrate comprises a microfluidic device.

In some embodiments, the presently disclosed subject matter describes a method of screening a chiral metalloporphyrin for catalytic activity, the method comprising: (a) providing a chemical library comprising a plurality of chiral metalloporphyrin compounds having the structure of Formula (I): 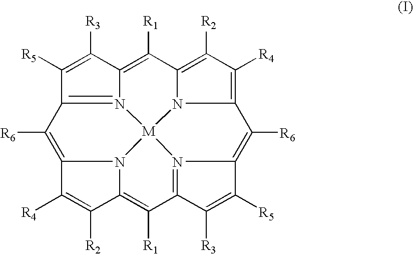
wherein: M is a transition metal; R₁, R₂, R₃, R₄, R₅ and R₆ are each independently selected from the group consisting of Y, H, alkyl, substituted alkyl, arylalkyl, aryl, and substituted aryl, wherein Y is a heteroatom-containing chiral moiety; and at least one of R₁, R₂, R₃, R₄, R₅ and R₆ is Y; (b) providing at least one target chemical reagent; (c) contacting the plurality of chiral metalloporphyrins with the target chemical reagent; and (d) detecting an interaction between and the plurality of chiral metalloporphyrins and the target chemical reagent, wherein the presence or the absence of the interaction is indicative of the catalytic activity of the chiral metalloporphyrin.

In some embodiments, M is selected from the group consisting of Zn, Fe, Ni, Co, Mn, Ru, and Rh. In some embodiments, M is Co.

### EXAMPLES

The following Examples have been included to illustrate modes of the presently disclosed subject matter. Certain aspects of the following Examples are described in terms of techniques and procedures found or contemplated to work well in the practice of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

Examples 1-146 relate to methods of the presently disclosed subject matter for the synthesis of porphyrins, metalloporphyrins, chiral porphyrins, and chiral metalloporphyrins.

### Example 1

### General Considerations

All reactions were carried out under a nitrogen atmosphere in oven-dried glassware using standard Schlenk techniques. Tetrahydrofuran was distilled under nitrogen from sodium benzophenone ketyl. 5-Bromo-10,20-diphenylporphyrin and 5,15-dibromo-10,20-diphenylporphyrin as well as their corresponding zinc complexes [5-bromo-10,20-diphenylporphyrino]zinc(II) and [5,15-dibromo-10,20-diphenylporphyrino]zinc(II) were synthesized by literature methods. Bis(2-diphenylphosphinophenyl)ether (DPEphos), palladium(II) acetate and tris(dibenzylideneacetone) dipalladium(0) were purchased from Strem Chemical Co. Cesium carbonate was obtained as a gift from Chemetall Chemical Products, Inc. Proton and carbon nuclear magnetic resonance spectra (¹H NMR and ¹³C NMR) were recorded on a Varian Mercury 300 spectrometer and referenced with respect to residual solvent. Infrared spectra were obtained using a Bomem B100 Series FT-IR spectrometer. Samples were prepared as films on a NaCl plate by evaporating THF solutions. UV-Vis spectra were obtained using a Hewlett-Packard 8452A diode array spectrophotometer. High-resolution mass spectroscopy was performed by the Mass Spectrometry Center located in the Chemistry Department of the University of Tennessee on a VG Analytical hybrid high performance ZAB-EQ (B-E-Q geometry) instrument using electron impact (EI) ionization technique with a 70 eV electron beam. Thin layer chromatography was carried out on E. Merck Silica Gel 60 F-254 TLC plates.

### Example 2

### General Procedures for Amination of Bromoporphyrin

The bromoporphyrin, palladium precursor, phosphine ligand and base were placed in an oven-dried, resealable Schlenk tube. The tube was capped with a Teflon screwcap, evacuated, and backfilled with nitrogen. The screwcap was replaced with a rubber septum, and amine was added via syringe, followed by solvent. The tube was purged with nitrogen for 2 min, and then the septum was replaced with the Teflon screwcap. The tube was sealed, and its contents were heated with stirring until the starting bromoporphyrin had been completely consumed as indicated by TLC analysis. The resulting mixture was cooled to room temperature, taken up in ethyl acetate (60 mL) and transferred to a separatory funnel. The mixture was washed with water (×2), dried over anhydrous sodium sulfate, filtered and concentrated in vacuo. The crude product was then purified.

### Example 3

### Synthesis of [5-(N-Phenylamino)-10,20-diphenylporphyrino]zinc(II) (Table 1, Product 3a)

The general procedure was used to couple [5-bromo-10,20-diphenylporphyrino]zinc(II) (30 mg, 0.050 mmol) with aniline (17 μL, 0.18 mmol), using palladium acetate (0.55 mg, 0.0025 mmol) as the palladium precursor, DPEphos (2.0 mg, 0.0038 mmol) as the phosphine ligand and cesium carbonate (22.8 mg, 0.070 mmol) as the base. The reaction was conducted in THF (5 mL) at 68° C. for 13 h. The title compound was isolated by flash column chromatography (silica gel, ethyl acetate:hexanes (v)=1:4) as purple solids (29 mg, 95%). ¹H NMR (300 MHz, THF-d₈): δ 10.08 (s, 1H), 9.48 (d, J=4.8 Hz, 2H), 9.31 (s, 1H), 9.29 (d, J=4.8 Hz, 2H), 8.92 (d, J=4.8 Hz, 2H), 8.81 (d, J=4.8 Hz, 2H), 8.22 (m, 4H), 7.75 (m, 6H), 7.04 (t, J=7.2 Hz, 2H), 6.87 (d, J=7.5 Hz, 2H), 6.65 (t, J=7.2 Hz, 1H). ¹³C NMR (75 MHz, THF-d₈): δ 164.8, 161.0, 160.8, 160.4, 160.2, 154.0, 145.1, 142.4, 141.7, 139.4, 139.2, 137.6, 136.8, 130.3, 130.2, 127.9, 124.6, 115.4. IR (film, cm⁻¹): 3383, 3050, 2953, 1599, 1493, 1307, 1061, 996, 793, 748. UV-vis (THF, λₘₐₓ, nm): 422, 554, 602. HRMS-EI ([M]⁺): calcd for C₃₈H₂₅N₅Zn, 615.1401; found: 615.1382 with an isotope distribution pattern that is same as the calculated one.

### Example 4

### Synthesis of 5-(N-Phenylamino)-10,20-diphenylporphyrin (Table 1, Product 3b)

The general procedure was used to couple 5-bromo-10,20-diphenylporphyrin (27 mg, 0.05 mmol) with aniline (17 μL, 0,18 mmol), using palladium acetate (0.55 mg, 0.0025 mmol) as the palladium precursor, DPEphos (2.0 mg, 0.0038 mmol) as the phosphine ligand and cesium carbonate (22.8 mg, 0.070 mmol) as the base. The reaction was conducted in THF (5 mL) at 68° C. for 19 h. The title compound was isolated by flash chromatography (silica gel, ethyl acetate:hexanes (v)=1:4) as red solids (27 mg, 98%). ¹H NMR (300 MHz, THF-d₈): δ 10.14 (s, 1H), 9.44(d, J=4.8 Hz, 2H), 9.42(s, 1H), 9.30(d, J=4.8 Hz, 2H), 8.90(d, J=4.8 Hz, 2H), 8.77(d, J=4.8 Hz, 2H), 8.21 (m, 4H), 7.78 (m, 6H), 7.06 (t, J=7.4, 2H), 6.86 (d, J=7.4 Hz, 2H), 6.69 (J=7.4 Hz, 1H), −2.54 (s, 2H). ¹³C NMR (75 MHz, THF-d₈): δ 154.8, 147.7, 142.7, 135.5, 132.1, 131.9, 131.1, 129.7, 128.5, 127.7, 120.6, 120.1, 119.0, 115.5, 105.1. IR (film, cm⁻¹): 3302, 3043, 1599, 1495, 1476, 1338, 1309, 1255, 1064, 973, 958, 797, 748. UV-vis(THF, λₘₐₓ, nm): 412, 512, 582, 660. HRMS-EI ([M]+): calcd for C₃₈H₂₇N₅, 553.2266; found: 553.2274 with an isotope distribution pattern that is same as the calculated one.

### Example 5

### Synthesis of [5-(N-Methyl-N-phenylamino)-10,20-diphenylporphyrino]zinc(II) (Table 1. Product 4a)

The general procedure was used to couple [5-bromo-10,20-diphenylporphyrino]zinc(II) (30 mg, 0.050 mmol) with N-methylaniline (20 μL, 0.18 mmol), using palladium acetate (0.55 mg, 0.0025 mmol) as the palladium precursor, DPEphos (2.0 mg, 0.0038 mmol) as the phosphine ligand and cesium carbonate (22.8 mg, 0.070 mmol) as the base. The reaction was conducted in THF (5 mL) at 68° C. for 13 h. The title compound was isolated by flash column chromatography (silica gel, THF:hexanes (v)=1:8) as purple solids (31 mg, 99%). ¹H NMR (300 MHz, THF-d₈): δ 10.20 (s, 1H), 9.36 (d, J=4.8 Hz, 2H), 9.19 (d, J=4.8 Hz, 2H), 8.97 (d, J=4.8 Hz, 2H), 8.87 (d, J=4.8 Hz, 2H), 8.23 (m, 4H), 7.77 (m, 6H), 7.05 (broad, 2H), 6.69 (broad, 2H), 6.61 (t, J=7.2 Hz, 1H), 4.28 (s, 3H). ¹³C NMR (75 MHz, THF-d₈): δ 156.0, 152.0, 151.2, 150.9, 150.7, 144.2, 135.5, 133.0, 132.8, 132.4, 130.0, 129.3, 128.1, 127.2, 125.3, 120.8, 116.7, 114.1, 106.9, 45.7. IR (film, cm⁻¹): 3054, 3023, 2978, 2876, 2807, 1596, 1498, 1341, 1120, 994, 793, 747. UV-vis (THF, λₘₐₓ, nm): 416, 552, 598. HRMS-EI ([M]⁺): calcd for C₃₉H₂₇N₅Zn, 629.1558; found: 629.1549 with an isotope distribution pattern that is same as the calculated one.

### Example 6

### Synthesis of 5-(N-Methyl-N-phenylamino)-10,20-diphenylporphyrin (Table 1, Product 4b)

The general procedure was used to couple 5-bromo-10,20-diphenylporphyrin (54 mg, 0.10 mmol) with N-methylaniline (40 μL, 0.36 mmol), using palladium acetate (1.1 mg, 0.005 mmol) as the palladium precursor, DPEphos (4.0 mg, 0.0075 mmol) as the phosphine ligand and cesium carbonate (45.6 mg, 0.014 mmol) as the base. The reaction was conducted in THF (5 mL) at 68° C. for 16 h. The title compound was isolated by flash column chromatography (silica gel, ethyl acetate:hexanes (v)=1:4) as purple solids (53 mg, 94%). ¹H NMR (300 MHz, CDCl₃): δ 10.18(s, 1H), 9.30(d, J=4.8 Hz, 2H), 9.19(d, J=4.8 Hz, 2H), 9.00(d, J=4.8 Hz, 2H), 8.90(d, J=4.8 Hz, 2H), 8.23(m, 4H), 7.78(m, 6H), 7.19 (broad, 2H), 6.73 (broad, 3H), 4.26 (s, 3H), −2.82(s, 2H). ¹³C NMR (75 MHz, CDCl₃): δ 154.9, 141.6, 134.9, 131.9, 131.7, 131.6, 129.6, 129.1, 128.0, 127.2, 124.2, 119.8, 116.9, 113.9, 105.8, 45.5. IR (film, cm⁻¹): 3303, 3055, 3026, 2875, 2810, 1596, 1498, 1351, 1113, 971, 796, 731. UV-vis (CHCl₃, λₘₐₓ, nm): 410, 512, 548, 592. HRMS-EI ([M]⁺): C₃₉H₂₉N₅, 567.2423; found: 567.2419 with an isotope distribution pattern that is same as the calculated one.

### Example 7

### Synthesis of [5-Benzophenoeimino-10,20-diphenylporphyrino]zinc(II) (Table 1. Product 5a)

The general procedure was used to couple [5-bromo-10,20-diphenylporphyrino]zinc(II) (30 mg, 0.050 mmol) with benzophenone imine (31 μL, 0.18 mmol), using palladium acetate (0.55 mg, 0.0025 mmol) as the palladium precursor, DPEphos (2.0 mg, 0.0038 mmol) as the phosphine ligand and cesium carbonate (22.8 mg, 0.070 mmol) as the base. The reaction was conducted in THF (5 mL) at 68° C. for 22 h. The title compound was isolated by flash column chromatography (silica gel, ethyl acetate:hexanes (v)=1:4) as purple solids (33 mg, 94%). ¹H NMR (300 MHz, THF-d₈): δ 9.80 (s, 1H), 9.23 (d, J=4.8 Hz, 2H), 9.13 (d, J=4.8 Hz, 2H), 8.79 (d, J=4.8 Hz, 2H), 8.71 (d, J=4.8 Hz, 2H), 8.19 (broad, 6H), 7.73 (m, 6H), 7.66 (broad, 3H), 7.36 (broad, 2H), 6.65 (broad, 3H). ¹³C NMR (75 MHz, THF-d₈): δ 170.8, 152.0, 150.3, 149.9, 144.5, 142.5, 135, 4, 133.0, 131.6, 131.1, 130.9, 130.0, 129.4, 128.8, 127.9, 127.2, 120.6, 103.7. IR (film, cm⁻¹): 3056, 3023, 2962, 1618, 1596, 1578, 1490, 1439, 1124, 1061, 994, 794. UV-vis (THF, λₘₐₓ, nm): 428, 562, 610. HRMS-EI ([M]⁺): calcd for C₄₅H₂₉N₅Zn, 703.1714; found: 703.1699 with an isotope distribution pattern that is same as the calculated one.

### Example 8

### Synthesis of 5-Benzophenoeimino-10,20-diphenylporphyrin (Table 1, Product 5b)

The general procedure was used to couple 5-bromo-10,20-diphenylporphyrin (27 mg, 0.05 mmol) with benzophenone imine (31 μL, 0.18 mmol), using palladium acetate (0.55 mg, 0.0025 mmol) as the palladium precursor, DPEphos. (2.0 mg, 0.0038 mmol) as the phosphine ligand and cesium carbonate (22.8 mg, 0.070 mmol) as the base. The reaction was conducted in THF (5 mL) at 68° C. for 24 h. The title compound was isolated by flash column chromatography (silica gel, ethyl acetate:hexanes (v)=1:8) as purple solids (27 mg, 84%). ¹H NMR (300 MHz, CDCl₃): δ 9.78(s, 1H), 9.23(d, J=4.8 Hz, 2H), 9.08 (d, J=4.8 Hz, 2H), 8.85(d, J=4.8 Hz, 2H), 8.75 (d, J=4.8 Hz, 2H), 8.26 (broad, 6H), 7.76 (broad, 9H), 7.18 (broad, 2H), 6.61 (broad, 3H), -2.34 (s, 2H). ¹³C NMR (75 MHz, CDCl₃): δ 171.6, 146.0, 141.7, 134.6, 133.6, 131.6, 130.7, 129.8, 127.9, 127.5, 126.8, 119.4, 102.4. IR (film, cm⁻¹): 3306, 3057, 3026, 1808, 1616, 1595, 1576, 1476, 1442, 1405, 1316, 1241, 1097, 976, 954, 845, 797, 745. UV-vis (CHCl₃, λₘₐₓ, nm): 424, 526, 564, 604, 658. HRMS-EI ([M]⁺): calcd for C₄₅H₃₁N₅, 641.2579; found: 641.2591 with an isotope distribution pattern that is same as the calculated one.

### Example 9

### Synthesis of [5-(N-Diphenylamino)-10,20-diphenylporphyrino]zinc(II) (Table 1, Product 6a)

The general procedure was used to couple [5-bromo-10,20-diphenylporphyrino]zinc(II) (30 mg, 0.05 mmol) with diphenylamine (0.031 g, 0.18 mmol), using palladium acetate (1.1 mg, 0.005 mmol) as the palladium precursor, DPEphos (4.0 mg, 0.0075 mmol) as the phosphine ligand and sodium tert-butoxide (13.5 mg, 0.14 mmol) as the base. The reaction was conducted in THF (5 mL) at 68° C. for 25 h. The title compound was isolated by flash column chromatography (silica gel, THF:hexanes (v)=1:6) as purple solids (21 mg, 61%). ¹H NMR (300 MHz, THF-d₈): δ 10.17(s, 1H), 9.33(m, 4H), 8.93(d, J=4.8 Hz, 2H), 8.80(d, J=4.8 Hz, 2H), 8.20(m, 4H), 7.75(m, 6H), 7.33 (m, 8H), 7.12(t, J=7.8 Hz, 8H), 6.80(t, J=7.2 Hz, 4H). ¹³C NMR (75 MHz, CDCl₃): δ 153.7, 153.0, 151.3, 151.0, 150.1, 144.1, 135.4, 133.3, 132.8, 132.4, 130.9, 129.8, 129.6, 128.1, 127.2, 122.9, 121.1, 120.9, 107.0. IR (film, cm⁻¹): 3055, 2961, 2361, 1598, 1587, 1490, 1293, 1273, 1062, 1003, 994, 794, 752. UV-vis (THF, λₘₐₓ, nm): 412, 558, 604. HRMS-EI ([M]⁺): calcd for C₄₄H₂₉N₅Zn, 691.1714; found: 691.1712 with an isotope distribution pattern that is same as the calculated one.

### Example 10

### Synthesis of 5-(N-Diphenylamino)-10,20-diphenylporphyrin (Table 1. Product 6b)

The general procedure was used to couple 5-bromo-10,20-diphenylporphyrin (54 mg, 0.1 mmol) with diphenylamine (0.061 g, 0.36 mmol), using palladium acetate (1.1 mg, 0.005 mmol) as the palladium precursor, DPEphos (4.0 mg, 0.0075 mmol) as the phosphine ligand and cesium carbonate (45.6 mg, 0.014 mmol) as the base. The reaction was conducted in THF (5 mL) at 68° C. for 40 h. The title compound was isolated by flash column chromatography (silica gel, THF:hexanes (v)=1:8) as purple solids (41 mg, 66%). ¹H NMR (300 MHz, CDCl₃): δ 10.13(s, 1H), 9.33(d, J=4.8 Hz, 2H), 9.26(d, J=4.8 Hz, 2H), 8.96(d, J=4.8 Hz, 2H), 8.83(d, J=4.8 Hz, 2H), 8.20(m, 4H), 7.76(m, 6H), 7.35 (m, 4H), 7.20(t, J=7.2 Hz, 4H), 6.89(t, J=7.2 Hz, 2H), −2.69(s, 2H). ¹³C NMR (75 MHz, CDCl₃): δ 152.5, 141.3, 134.8, 134.6, 132.0, 131.4, 130.1, 129.1, 127.8, 126.8, 122.3, 120.8, 119.6, 105.6. IR (film, cm⁻¹): 3307, 3055, 3029, 1591, 1491, 1342, 1184, 973, 796, 750, 731, 695. UV-vis (CHCl₃, λₘₐₓ, nm): 407, 523, 577, 656. HRMS-EI ([M]⁺): calcd for C₄₄H₃₁N₅, 629.2579; found: 629.2576 with an isotope distribution pattern that is same as the calculated one.

### Example 11

### Synthesis of [5-(N-Hexylamino)-10,20-diphenylporphyrino]zinc(II) (Table 1, Product 7a)

The general procedure was used to couple [5-bromo-10,20-diphenylporphyrino]zinc(II) (30 mg, 0.05 mmol) with hexylamine (0.024 mL, 0.18 mmol), using palladium acetate (0.55 mg, 0.0025 mmol) as the palladium precursor, DPEphos (2.0 mg, 0.0038 mmol) as the phosphine ligand and cesium carbonate (22.8 mg, 0.070 mmol) as the base. The reaction was conducted in THF (5 mL) at 68° C. for 50 h. The title compound was isolated by flash column chromatography (silica gel, THF:hexanes (v)=1:8) as purple solids (25 mg, 80%). ¹H NMR (300 MHz, THF-d₈): δ 9.63(s, 1H), 9.43(d, J=4.8 Hz, 2H), 9.05(d, J=4.8 Hz, 2H), 8.76(d, J=4.8 Hz, 2H), 8.65(d, J=4.8 Hz, 2H), 8.18(m, 4H), 7.75(m, 6H), 7.33 (m, 8H), 6.78(s, 1H), 4.38(m, 2H), 2.04(m, 2H), 1.58(m, 2H), 1.37(m, 4H), 0.87(t, J=7.2 Hz, 3H). ¹³C NMR (75 MHz, THF-d₈): δ 152.7, 149.9, 149.5, 147.0, 144.7, 135.3, 133.0, 131.4, 130.2, 127.8, 127.2, 126.9, 120.5, 102.4, 60.2, 32.8, 32.5, 28.0, 23.5,14.4. IR (film, cm⁻¹): 3330, 3053, 2954, 2925, 2854, 1584, 1542, 1489, 1440, 1213, 1062, 1010, 1002, 992, 836, 789, 780, 750. UV-vis (THF, λₘₐₓ, nm): 428, 606. HRMS-EI ([M]⁺): calcd for C₃₈H₃₃N₅Zn, 623.2027; found: 623.2009 with an isotope distribution pattern that is same as the calculated one.

### Example 12

### Synthesis of [5,15-Bis(N-phenylamino)-10,20-diphenylporphyrino]zinc(II) (Table 1, Product 8a)

The general procedure was used to couple [5,15-dibromo-10,20-diphenylporphyrino]zinc(II) (34 mg, 0.050 mmol) with aniline (22 μL, 0.24 mmol), using palladium acetate (1.1 mg, 0.0050 mmol) as the palladium precursor, DPEphos (4.0 mg, 0.0075 mmol) as the phosphine ligand and cesium carbonate (45.6 mg, 0.14 mmol) as the base. The reaction was conducted in THF (5 mL) at 68° C. for 13 h. The title compound was isolated by flash column chromatography (silica gel, THF:hexanes (v)=1:4) as purple solids (29 mg, 82%). ¹H NMR (300 MHz, THF-d₈): δ 9.36 (d, J=4.8 Hz, 4H), 9.17 (s, 2H), 8.69 (d, J=4.8 Hz, 4H), 8.16 (m, 4H), 7.72 (m, 6H), 7.03 (t, J=6.9, 7.2 Hz, 4H), 6.84 (d, J=8.4 Hz, 4H), 6.64 (t, J=7.2 Hz, 2H). ¹³C NMR (75 MHz, THF-d₈): δ 155.1, 152.0, 150.5, 144.4, 135.3, 132.2, 129.7, 129.6, 128.0, 127.2, 121.0, 119.9, 118.2, 115.0. IR (film, cm⁻¹): 3380, 3047, 3020, 2953, 1599, 1492, 1339, 1308, 1063, 1003, 795, 747. UV-vis (THF, λₘₐₓ, nm): 440, 564, 620. HRMS-EI ([M]⁺): calcd for C₄₄H₃₀N₆Zn, 706.1823; found: 706.1840 with an isotope distribution pattern that is same as the calculated one.

### Example 13

### Synthesis of 5,15-Bis(N-phenylamino)-10,20-diphenylporphyrin (Table 1, Product 8b)

The general procedure was used to couple 5,15-dibromo-10,20-diphenylporphyrin (31 mg, 0.05 mmol) with aniline (22 μL, 0.24 mmol), using palladium acetate (1.1 mg, 0.0050 mmol) as the palladium precursor, DPEphos (4.0 mg; 0.0075 mmol) as the phosphine ligand and cesium carbonate (45.6 mg, 0.14 mmol) as the base. The reaction was conducted in THF (5 mL) at 68° C. for 20 h. The title compound was isolated by flash column chromatography (silica gel, ethyl acetate:hexanes (v)=1:4) as purple solids (21 mg, 65%). ¹H NMR (300 MHz, THF-d₈): δ 9.32(d, J=4.8 Hz, 4H), 9.29(s, 2H), 8.65(d, J=4.8 Hz, 4H), 8.17(m, 4H), 7.75(m, 6H), 7.07(t, J=8.1 Hz, 4H), 6.86(d, J=8.1 Hz, 4H), 6.69(t, J=7.4 Hz, 2H), −2.03(s, 2H). ¹³C NMR (75 MHz, THF-d₈): δ 154.5, 142.9, 137.1, 135.3, 129.7, 128.5, 127.6, 120.5, 119.7, 118.9, 115.4. IR (film, cm⁻¹): 3307, 1599, 1496, 1474, 1340, 1306, 1258, 1071, 974, 797, 732. UV-vis (THF, λₘₐₓ, nm): 438, 526, 592, 680. HRMS-EI ([M]⁺): calcd for C₄₄H₃₂N₆, 644.2688; found: 644.2704 with an isotope distribution pattern that is same as the calculated one.

### Example 14

### Synthesis of [5,15-Bis(N-methyl-N-phenylamino)-10,20-diphenylporphyrino]zinc(II) (Table 1. Product 9a)

The general procedure was used to couple [5,15-dibromo-10,20-diphenylporphyrino]zinc(II) (34 mg, 0.050 mmol) with N-methylaniline (26 μL, 0.24 mmol), using palladium acetate (1.1 mg, 0.0050 mmol) as the palladium precursor, DPEphos (4.0 mg, 0.0075 mmol) as the phosphine ligand and cesium carbonate (45.6 mg, 0.14 mmol) as the base. The reaction was conducted in THF (5 mL) at 68° C. for 17 h. The title compound was isolated by flash column chromatography (silica gel, THF:hexanes (v)=1:8) as purple solids (30 mg, 82%). ¹H NMR (300 MHz, THF-d₈): δ 9.10 (d, J=4.8 Hz, 4H), 8.75 (d, J=4.8 Hz, 4H), 8.15 (m, 4H), 7.73 (m, 6H), 7.04 (broad, 4H), 6.69 (broad, 4H), 6.59 (t, J=7.2 Hz, 2H), 4.25 (s, 6H). ¹³C NMR (75 MHz, THF-d₈): δ 155.8, 152.4, 150.9, 144.0, 135.2, 133.1, 130.1, 129.3, 128.2, 127.2, 125.7, 121.2, 116.8, 114.2, 45.6. IR (film, cm⁻¹): 3054, 2985, 2883, 2807, 1597, 1496, 1346, 1118, 1000, 796, 747. UV-vis (THF, λₘₐₓ, nm): 422, 562, 608. HRMS-EI ([M]⁺): calcd for C₄₆H34N₆Zn, 734.2136; found: 734.2128 with an isotope distribution pattern that is same as the calculated one.

### Example 15

### Synthesis of 5,15-Bis(N-methyl-N-phenylamino)-10,20-diphenylporphyrin (Table 1. Product 9b)

The general procedure was used to couple 5,15-dibromo-10,20-diphenylporphyrin (31 mg, 0.05 mmol) with N-methylaniline (26 μL, 0.24 mmol), using palladium acetate (1.1 mg, 0.0050 mmol) as the palladium precursor, DPEphos (4.0 mg, 0.0075 mmol) as the phosphine ligand and the cesium carbonate (45.6 mg, 0.14 mmol) as the base. The reaction was conducted in THF (5 mL) at 68° C. for 15 h. The title compound was isolated by flash column chromatography (silica gel, ethyl acetate:hexanes (v)=1:4) as red solids (24 mg, 71%). ¹H NMR (300 MHz, CDCl₃): δ 9.08 (d, J=4.8 Hz, 4H), 8.77 (d, J=4.8 Hz, 4H), 8.16 (m, 4H), 7.72(m, 6H), 7.14(m, 4H), 6.72(m, 6H), 4.23(s, 6H), −2.54(s, 2H). ¹³C NMR (75 MHz, CDCl3): δ 154.4, 141.3, 134.5, 131.9, 128.9, 128, 127.8, 126.8, 124.3, 119.9, 116.7, 113.8, 45.1. IR (film, cm⁻¹): 3315, 3026, 2359, 1596, 1498, 1475, 1354, 1114, 972, 798. UV-vis (CHCl₃, λₘₐₓ, nm): 412, 522, 562, 596, 608. HRMS-EI ([M]⁺): calcd for C₄₆H₃₆N₆, 672.3001; found: 672.3003 with an isotope distribution pattern that is same as the calculated one.

### Example 16

### Synthesis of [5,15-Bis(benzophenoeimino)-10,20-diphenylporphyrino]zinc(II) (Table 1, Product 10a)

The general procedure was used to couple [5,15-dibromo-10,20-diphenylporphyrino]zinc(II) (34 mg, 0.050 mmol) with benzophenoe imine (41 μL, 0.24 mmol), using palladium acetate (1.1 mg, 0.0050 mmol) as the palladium precursor, DPEphos (4.0 mg, 0.0075 mmol) as the phosphine ligand and cesium carbonate (45.6 mg, 0.14 mmol) as the base. The reaction was conducted in THF (5 mL) at 68° C. for 16 h. The title compound was isolated by flash column chromatography (silica gel, ethyl acetate:hexanes (v)=1:4) as purple solids (37 mg, 84%). ¹H NMR (300 MHz, CDCl₃): δ 9.06 (d, J=4.8 Hz, 4H), 8.57 (d, J=4.8 Hz, 4H), 8.19 (m, 4H), 8.07 (m, 4H), 7.68 (m, 6H), 7.61 (m, 6H), 7.33 (m, 4H), 6.62 (m, 6H). ¹³C NMR (75 MHz, CDCl₃): δ 170.8, 149.4, 144.7, 143.8, 135.4, 135.2, 132.7, 131.9, 131.5, 130.8, 129.9, 129.3, 128.4, 128.0, 127.7, 127.2, 127.1, 126.9, 120.9. IR (film, cm⁻¹): 3054, 3027, 2976, 1618, 1597, 1485, 1442, 1338, 1212, 1118, 1004, 793, 753. UV-vis (THF, λₘₐₓ, nm): 438, 652. HRMS-EI ([M]⁺): calcd for C₅₈H₃₈N₆Zn, 882.2449, found: 882.2464 with an isotope distribution pattern that is same as the calculated one.

### Example 17

### Synthesis of 5,15-Bis(benzophenoeimino)-10,20-diphenylporphyrin (Table 1. Product 10b)

The general procedure was used to couple 5,15-dibromo-10,20-diphenylporphyrin (31 mg, 0.05 mmol) with benzophenone imine (41 μL, 0.24 mmol), using palladium acetate (1.1 mg, 0.0050 mmol) as the palladium precursor, DPEphos (4.0 mg, 0.0075 mmol) as the phosphine ligand and cesium carbonate (45.6 mg, 0.14 mmol) as the base. The reaction was conducted in THF (5 mL) at 68° C. for 15 h. The title compound was isolated by flash column chromatography (silica gel, ethyl acetate:hexanes (v)=1:4) as purple solids (39 mg, 95%). ¹H NMR(300 MHz, THF-d₈): δ 9.09(d, J=4.8 Hz, 4H), 8.57(d, J=4.8 Hz, 4H), 8.10(m, 8H), 7.64(m, 12H), 7.23(broad, 4H), 6.62(broad, 6H), −1.87(s, 2H). ¹³C NMR (75 MHz, THF-d₈): δ 172.3, 143.2, 140.8, 137.9, 135.4, 135.1, 132.1, 131.0, 129.3, 128.9, 128.3, 128.2, 127.5, 120.3, 108.4. IR (film, cm⁻¹) 3316, 3056, 3022, 1614, 1596, 1575, 1465, 1443, 1351, 1316, 1278, 1244, 1105, 1066, 976, 950, 798, 725. UV-vis (THF, λₘₐₓ, nm): 434, 592, 700. HRMS-EI ([M]⁺): calcd for C₅₈H₄₀N₆, 820.3314; found: 820.3308 with an isotope distribution pattern that is same as the calculated one.

### Example 18

### Synthesis of [5,15-Bis(N-diphenylamino)-10,20-diphenylporphyrino]zinc(II) (Table 1, Product 11a)

The general procedure was used to couple [5,15-dibromo-10,20-diphenylporphyrino]zinc(II) (34 mg, 0.05 mmol) with diphenylamine (0.041 g, 0.24 mmol), using palladium acetate (1.1 mg, 0.0050 mmol) as the palladium precursor, DPEphos (4.0 mg, 0.0075 mmol) as the phosphine ligand and sodium tert-butoxide (13.5 mg, 0.14 mmol) as the base. The reaction was conducted in THF (5 mL) at 68° C. for 50 h. The title compound was isolated by flash column chromatography (silica gel, THF:hexanes (v)=1:6) as purple solids (13 mg, 30%). ¹H NMR (300 MHz, CDCl₃): δ 9.25(d, J=4.8 Hz, 4H), 8.75(d, J=4.8 Hz, 4H), 8.09(m, 4H), 7.66(m, 6H), 7.29 (m, 8H), 7.15(t, J=7.8 Hz, 8H), 6.85(t, J=7.4 Hz, 4H). ¹³C NMR (75 MHz, CDCl₃): δ 152.6, 152.3, 149.7, 142.1, 134.3, 133.3, 130.5, 129.1, 127.6, 126.5, 122.8, 122.1, 121.0, 120.7. IR (film, cm⁻¹): 3056, 2360, 1595, 1590, 1490, 1341, 1294, 1249, 1002, 794, 750. UV-vis (CHCl₃, λₘₐₓ, nm): 406, 460, 572, 628. HRMS-EI ([M]⁺): calcd for C₅₆H₃₈N₆Zn, 858.2449; found: 858.2436 with an isotope distribution pattern that is same as the calculated one.

### Example 19

### Synthesis of [5,15-Bis(N-4-trifluoromethylphenylamino)-10,20-diphenylporphyrino]zinc(II) (Table 1, Product 12a)

The general procedure was used to couple [5,15-dibromo-10,20-diphenylporphyrino]zinc(II) (34 mg, 0.05 mmol) with 4-trifluoromethyllaniline (0.030 mL, 0.24 mmol), using palladium acetate (1.1 mg, 0.0050 mmol) as the palladium precursor, DPEphos (4.0 mg, 0.0075 mmol) as the phosphine ligand and cesium carbonate (45.6 mg, 0.14 mmol) as the base. The reaction was conducted in THF (5 mL) at 68° C. for 17 h. The title compound was isolated by flash column chromatography (silica gel, ethyl acetate:hexanes (v)=1:2) as purple solids (38 mg, 90%). ¹H NMR (300 MHz, THF-d₈): δ 9.84(s, 2H), 9.43(d, J=4.8 Hz, 4H), 8.84(d, J=4.8 Hz, 4H), 8.22(m, 4H), 7.78(m, 6H), 7.41(d, J=8.2 Hz, 4H), 6.93(d, J=8.2 Hz, 4H). ¹³C NMR (75 MHz, THF-d₈): δ 157.5, 151.7, 151.0, 144.1, 135.4, 132.9, 129.7, 128.2, 127.3, 127.2, 127.1, 121.6, 119.3, 118.1, 114.1. IR (film, cm⁻¹): 3376, 1614, 1522, 1322, 1110, 1065, 1003, 828, 797. UV-vis (THF, λₘₐₓ, nm): 435, 562, 612. HRMS-EI ([M]⁺): calcd for C₄₆H₂₈N₆F₆Zn, 842.1571; found: 842.1590 with an isotope distribution pattern that is same as the calculated one.

### Example 20

### [5,15-Bis(N-4-methoxyphenylamino)-10,20-diphenylporphyrino]zinc(II) (Table 1, Product 13a)

The general procedure was used to couple [5,15-dibromo-10,20-diphenylporphyrino]zinc(II) (34 mg, 0.05 mmol) with p-anisidine (30 mg, 0.24 mmol), using palladium acetate (1.1 mg, 0.0050 mmol) as the palladium precursor, DPEphos (4.0 mg, 0.0075 mmol) as the phosphine ligand and cesium carbonate (45.6 mg, 0.14 mmol) as the base. The reaction was conducted in THF (5 mL) at 68° C. for 16 h. The title compound was isolated by flash column chromatography (silica gel, ethyl acetate:hexanes (v)=1:3 as purple solids (36 g, 94%). ¹H NMR (300 MHz, THF-d₈): δ 9.34(d, J=4.8 Hz, 4H), 8.88(s, 2H), 8.66(d, J=4.8 Hz, 4H), 8.17(m, 4H), 7.73(m, 6H), 6.87(d, J=9.0 Hz, 4H), 6.69(d, J=9.0 Hz, 4H), 3.65(s, 6H). ¹³C NMR (75 MHz, THF-d₈): δ 153.5, 151.9, 150.2, 149.6, 144.6, 135.3, 131.9, 129.3, 127.8, 127.1, 121.2, 120.7, 116.6, 115.0, 55.6. IR (film, cm⁻¹): 3372, 1597, 1507, 1489, 1339, 1234, 1036, 1002, 797. UV-vis (THF, λₘₐₓ, nm): 447, 571, 629.

### Example 21

### Synthesis of [5,15-Bis(N-3,5-di-tert-butylphenylamino)-10,20-diphenylporphyrino]zinc(II) (Table 1. Product 14a)

The general procedure was used to couple [5,15-dibromo-10,20-diphenylporphyrino]zinc(II) (34 mg, 0.05 mmol) with 3,5-di-tert-butylaniline (0.050 g, 0.24 mmol), using palladium acetate (1.1 mg, 0.0050 mmol) as the palladium precursor, DPEphos (4.0 mg, 0.0075 mmol) as the phosphine ligand and cesium carbonate (45.6 mg, 0.14 mmol) as the base. The reaction was conducted in THF (5 mL) at 68° C. for 62 h. The title compound was isolated by flash column chromatography (silica gel, ethyl acetate:hexanes (v)=1:4) as purple solids (44 mg, 95%). ¹H NMR (300 MHz, CDCl₃): δ 9.28(d, J=4.8 Hz, 4H), 8.68(d, J=4.8 Hz, 4H), 8.14(m, 4H), 7.71(m, 8H), 6.87(m, 6H), 1.21(s, 36H). ¹³C NMR (75 MHz, CDCl₃): δ 152.1, 151.5, 150.6, 149.4, 143.0, 134.8, 131.7, 128.5, 127.1, 126.4, 120.4, 118.8, 113.4, 109.6, 34.7, 31.3. IR (film, cm⁻¹): 3383, 3055, 2961, 2902, 2867, 1595, 1488, 1436, 1340, 1064, 1004, 796. UV-vis (THF, λₘₐₓ, nm): 448, 576, 634. HRMS-EI ([M]⁺): calcd for C₆₀H₆₂N₆Zn, 930.4327; found: 930.4354 with an isotope distribution pattern that is same as the calculated one.

Examples 22 through 47 relate to methods of synthesizing aminophenylporphyrins, and novel aminophenylporphyrins, according to the presently disclosed subject matter. In Example 2247, ligands referred to by number refer to the numbered ligands shown in FIG. 2.

### Example 22

### General Considerations

All reactions were carried out under a nitrogen atmosphere in oven-dried Schlenk tube. All amines were purchased from Acros Organics or Aldrich Chemical Co. and used without further purification. Tetrahydrofuran and toluene were continuously refluxed and freshly distilled from sodium benzophenone ketyl under nitrogen. Sodium tert-butoxide was purchased from Aldrich Chemical Co.; Cesium carbonate was obtained as a gift from Chemetall Chemical Products, Inc.

Potassium phosphate, potassium carbonate, palladium(II) acetate, tris(dibenzylideneacetone)dipalladium(0), 2-(di-t-butylphosphino)biphenyl (FIG. 2, Ligand 1), 2-(dicyclohexylphosphino)biphenyl (FIG. 2, Ligand 2), 2-dicyclohexylphosphino-2′-(N,N-di-methylamino)biphenyl (FIG. 2, Ligand 4), bis(2-diphenylphosphinophenyl)ether (DPEphos, FIG. 2, Ligand 6), Xantphos (FIG. 2, Ligand 7), racemic-2-(di-t-butylphosphino)-1,1′-binaphthyl (FIG. 2, Ligand 8), (±)BINAP (FIG. 2, Ligand 9), dichloro[1,1′-bis(diphenylphosphino)ferrocene]palladium(II) dichloride ((dppf)PdCl₂, FIG. 2, Ligand 10) and 1,3-bis(2,6-di-i-propylphenyl)imidazolium chloride (FIG. 2, Ligand 11) were purchased from Strem Chemical Co.; 2-(dicyclohexylphosphino)-2′6′dimethyl-biphenyl (FIG. 2, Ligand 3) and the ligand shown in FIG. 2 as Ligand 5 were synthesized according to literature methods. All ligands and palladium precursors and bases were stored in desiccators filled with anhydrous calcium sulfate, and weighed in the air. 5,15-di-p-bromophenylporphyrin as well as its zinc complex, and tetrakis-p-bromophenylporphyrin were prepared according to the method described in literatures. ¹H NMR and ¹³C NMR were recorded on Varian Mercury 300 spectrometer with TMS as an internal standard. UV-Vis spectra were measured on Hewlett-Packard 8452 diode array spectrometer. High resolution mass spectroscopy was determined on a VG analytical hybrid high performance ZAB-EQ(B-E-Q geometry) instrument by the Mass Spectrometry Center (Department of Chemistry, University of Tennessee). All solvents were supplied by Fisher Scientific, Inc. with HPLC grade and used as received. Thin layer chromatography was performed on Silica Gel 60F-254 precasted aluminum TLC plate.

### Example 23

### General Procedures for Amination of Bromophenylporphyrin

An oven-dried Schlenk tube equipped with stirring bar was degassed on vacuum line and purged with nitrogen. The tube was charged with Pd(OAc)₂ or Pd₂(dba)₃ (5 mole %), phosphine ligand (10 mole %), bromophenylporphyrin or zinc complex (0.05 mmole), base (NaOtBu or Cs₂CO_{3,} 4.0 equiv for 1.0 equiv Br) and solid amine, if any. The tube was capped with a Teflon screw cap, evacuated on vacuum line for 40-50 min and backfilled with nitrogen. The Teflon screw cap was then replaced with a rubber septum, 2-3 mL of freshly redistilled and dried solvent, and amine (4.0 equiv for 1.0 equiv Br) was added via syringe successively. An additional 2-3 mL of solvent was added against the wall of the tube to wash down the possible reactants on the wall. The tube was purged with nitrogen for 1-2 min, and the septum was then replaced by Teflon screw cap. The tube was tightly sealed and immersed in a 100° C. oil bath. The reaction was preceded under this condition with stirring for 48 h (72 h for tetra-bromophenylporphyrin), and cooled to room temperature. The aliquot of the solution was detected on TLC (methylene chloride:hexanes=8:2 or ethyl acetate:hexanes=5:5 ) to monitor the result.

### Example 24

### General Workup Procedures for Amination of Bromophenylporphyrin

The reaction solution was transferred with a long glass pipette to a small round-bottom flask, the residue was washed with acetone or chloroform and pooled to the flask as well. The solution was concentrated on a rotary evaporator to remove the solvent. The residue was redissolved in ethyl acetate and transferred to a separatory funnel, washed with deionized water three times to remove the base and salts. The organic layer was concentrated on a rotary evaporator to dryness. The residue was dissolved in minimal acetone (or methylene chloride, or THF), and small amount of hexanes was added to recrystallize the product. The product gradually precipitated or crystallized from the solution, filtered on funnel, washed with small amount of hexanes to afford the pure product (purity 98-99%). Extra pure compound can be obtained through flash chromatography on silica gel column (methylene chloride:hexanes (8:2 to 10:0) as elute).

### Example 25

### Synthesis of 5,15-di-p-(N-phenylamino)phenylporphyrin (Table 2, entry 1, A)

The general procedure using 5,15-di-p-bromophenylporphyrin (31.0 mg, 0.05 mmol), Pd(OAc)₂ (1.12 mg, 0.005 mmol), (±)BINAP (6.2 mg, 0.01 mmol, 9), Cs₂CO₃ (130.33 mg, 0.4 mmol), aniline (36.5 μL, 0.4 mmol) and toluene, the reaction proceeded at 100° C. for 48 h. After workup with general procedure, the title compound was obtained as dark-purple solid (22.6 mg, 70%). ¹H NMR (CDCl₃, 300 MHz) δ 10.29 (s, meso-2H), 9.39 (d, J=4.5 Hz, β-4H), 9.17 (d, J=4.8 Hz, β-4H), 8.14 (d, J=8.7 Hz, 4H), 7.50 (d, J=8.4 Hz, 4H), 7.38-7.46 (m, 8H), 7.06 (m, 2H), 6.13 (s, 2H), −3.05 (s, 2H). ¹³C NMR (CDCl₃, 75 MHz) δ 142.9, 142.6, 135.9, 131.5, 131.0, 129.6, 121.6, 118.6, 115.7, 105.1. UV-vis (λₘₐₓ, nm) 421, 508, 548, 580, 637. HRMS-EI ([M+1]⁺): calc'd for C₄₄H₃₃N₆, 645.2767; found 645.2734

### Example 26

### Synthesis of 5,15-di-D-(N-phenylamino)phenylporphyrin (Zn II) (Table 2. entry 1, B)

The reactants were as the same as entry 1 A except 5,15-di-p-bromophenylporphyrin was replaced by its zinc complex (34.5 mg, 0.05 mmol). After workup with general procedure, the title compound was obtained as brown solid (23.5 mg, 66%). ¹H NMR (CDCl₃, 300 MHz) δ 10.31 (s, meso-2H), 9.45 (d, J=4.2 Hz, β-4H), 9.24 (d, J=4.5 Hz, β-4H), 8.14 (d, J=8.1 Hz, 4H), 7.50 (d, J=8.4 Hz, 4H), 7.40-7.47 (m, 8H), 7.06 (m, 2H), 6.11 (s, 2H). ¹³C NMR (CDCl₃, 75 MHz) δ 150.4, 149.3, 135.7, 132.5, 131.6, 129.5, 121.5, 118.4, 115.5, 106.1. UV-vis (λₘₐₓ, nm) 419, 542, 583. HRMS-EI ([M]⁺): calc'd for C₄₄H₃₀N₆Zn, 706.1823; found 706.1845

### Example 27

### Synthesis of 5,15-di-p-[N-(4-nitrophenyl)amino]phenylporphyrin (Table 2, entry 2, A)

The general procedure using 5,15-di-p-bromophenylporphyrin (31.0 mg, 0.05 mmol), Pd(OAc)₂ (1.12 mg, 0.005 mmol), (±)BINAP (6.2 mg, 0.01 mmol, 9), Cs₂CO₃ (130.33 mg, 0.4 mmol), 4-nitroaniline (55.3 mg, 0.4 mmol) and toluene, the reaction proceeded at 100° C. for 48 h. After workup with general procedure, the title compound was obtained as brown solid (28.0 mg, 76%). ¹H NMR (DMSO-d₆, 300 MHz) δ 10.64 (s, meso-2H), 9.82 (s, 2H), 9.67 (d, J=4.2 Hz, β-4H), 9.15 (d, J=4.2 Hz, β-4H), 8.24-8.29 (m, 8H), 7.75 (d, J=7.5 Hz, 4H), 7.45 (d, J=9.0 Hz, 4H), −3.19 (s, 2H). ¹³C NMR (DMSO-d₆, 75 MHz) δ 150.5, 146.7, 144.7, 140.2, 138.4, 135.9, 134.9, 132.7, 130.9, 126.4, 118.9, 114.3, 105.8. UV-vis (λₘₐₓ, nm) 413, 506, 542, 579, 635. HRMS-EI ([M+1]⁺): calc'd for C₄₄H₃₁N₈O₄, 735.2463; found 725.2436.

### Example 28

### Synthesis of 5,15-di-p-[N-(4-methoxyphenyl)amino]phenylporphyrin (Table 2, entry 3, A)

The general procedure using 5,15-di-p-bromophenylporphyrin (31.0 mg, 0.05 mmol), Pd(OAc)₂ (1.12 mg, 0.005 mmol), ligand 3 ( 3.8 mg, 0.01 mmol), NaOtBu (38.22 mg, 0.4 mmol), p-anisidine (49.3 mg, 0.4 mmol) and THF, the reaction proceeded at 100° C. for 48 h. After workup with general procedure, the title compound was obtained (32.6 mg, 93%). ¹H NMR (DMSO-d₆, 300 MHz) δ 10.56 (s, meso-2H), 9.62 (d, J=4.2 Hz, β-4H), 9.15 (d, J=4.8 Hz, β-4H), 8.44 (s, 2H), 8.07 (d, J=7.8 Hz, 4H), 7.37 (d, J=9.0 Hz, 4H), 7.41 (d, J=8.7 Hz, 4H), 7.02 (d, J=8.4 Hz, 4H), 3.78(s, 6H), −3.09 (s, 2H). ¹³C NMR (DMSO-d₆, 75 MHz) δ 154.3, 147.0, 145.2, 144.4, 136.1, 135.7, 134.7, 134.6, 132.3, 129.9, 121.4, 119.3, 114.7, 113.3, 55.3. UV-vis (λₘₐₓ, nm) 418, 510, 552, 583, 640. HRMS-EI ([M+1]⁺): calc'd for, C₄₆H₃₇N₆O₂, 705.2978; found 705.3018.

### Example 29

### Synthesis of 5,15-di-p-[N-(4-methoxyphenyl)amino]phenylporphyrin (Zn II) (Table 2, entry 3, B)

The general procedure using 5,15-di-p-bromophenylporphyrin (Zn II)(34.5 mg, 0.05 mmol), Pd(OAc)₂ (1.12 mg, 0.005 mmol), ligand 8 (3.98 mg, 0.01 mmol), NaOtBu (38.22 mg, 0.4 mmol), p-anisidine (49.3 mg, 0.4 mmol) and THF, the reaction proceeded at 100° C. for 48 h. After workup with general procedure, the title compound was obtained (26 mg, 68%). ¹H NMR (DMSO-d₆, 300 MHz) δ 10.29 (s, meso-2H), 9.47 (d, J=4.5 Hz, β-4H), 9.07 (d, J=4.2 Hz, β-4H), 8.36 (s, 2H), 8.02 (d, J=8.1 Hz, 4H), 7.39 (d, J=8.1 Hz, 4H), 7.37 (d, J=8.1 Hz, 4H), 7.01 (d, J=8.1 Hz, 4H), 3.78(s, 6H). ¹³C NMR (DMSO-d₆, 75 MHz) δ 154.1, 149.8, 148.7, 144.6, 136.1, 135.7, 134.7, 132.3, 131.9, 131.8, 121.0, 119.5, 114.7, 113.0, 105.8, 55.3. UV-vis (λₘₐₓ, nm) 419, 545, 585.

### Example 30

### Synthesis of 5,15-di-p-(N-benzylamino)phenylporphyrin (Zn II) (Table 2, entry 4, B)

The general procedure using 5,15-di-p-bromophenylporphyrin (Zn II) (34.2 mg, 0.05 mmol), Pd(OAc)₂ (1.12 mg, 0.005 mmol), ligand 8 (3.98 mg, 0.01 mmol), NaOtBu (38.22 mg, 0.4 mmol), benzylamine (43.7 μL, 0.4 mmol) and THF, the reaction proceeded at 100° C. for 48 h. After workup with general procedure, the title compound was obtained (30.7 mg, 83%). ¹H NMR (CDCl₃, 300 MHz) δ 10.17 (s, meso-2H), 9.35 (d, J=4.2 Hz, β-4H), 9.17 (d, J=4.8 Hz, β-4H), 8.04 (d, J=7.2 Hz, 4H), 7.58 (d, J=7.5 Hz, 4H), 7.35-7.49(m, 6H), 7.02 (d, J=7.5 Hz, 4H), 5.5(s, 2H). UV-vis (λₘₐₓ, nm) 419, 543, 584.

### Example 31

### Synthesis of 5,15-di-p-[N-(4-methylpyridyl)amino]phenylporphyrin (Table 2, entry 5, A)

The general procedure using 5,15-di-p-bromophenylporphyrin (31.0 mg, 0.05 mmol), Pd(OAc)₂ (1.12 mg, 0.005 mmol), (±)BINAP (6.2 mg, 0.01 mmol, 9), NaOtBu (38.22 mg, 0.4 mmol), 4-aminomethylpyridine (41 μL, 0.4 mmol) and THF, the reaction proceeded at 100° C. for 48 h. After workup with general procedure, the title compound was obtained (29.6 mg, 88%). Different yield was observed by using other conditions (table 1). ¹H NMR (DMSO-d₆, 300 MHz) δ 10.52 (s, meso-2H), 9.58 (d, J=4.5 Hz, β-4H), 9.07 (d, J=4.2 Hz, β-4H), 8.63 (d, J=5.7 Hz, 4H), 7.98 (d, J=8.4 Hz, 4H), 7.58 (d, J=5.7 Hz, 4H), 7.05 (d, J=8.7 Hz, 4H), 6.97(t, 2H), 4.61 (d, J=5.7, 4H), −3.10 (s, 2H). ¹³C NMR (DMSO-d₆, 75 MHz) δ 149.8, 148.2, 147.1, 144.6, 136.0, 134.6, 134.4, 134.3, 132.2, 130.8, 128.1, 122.5, 111.4, 105.4, 45.6. UV-vis (λₘₐₓ, nm) 416, 508, 548, 581, 638.

### Example 32

### Synthesis of 5,15-di-p-[N-(o-methylphenyl)amino]phenylporphyrin (Table 2, entry 6, A)

The general procedure using 5,15-di-p-bromophenylporphyrin (31.0 mg, 0.05 mmol), Pd(OAc)₂ (1.12 mg, 0.005 mmol), ligand 3 (3.8 mg, 0.01 mmol), NaOtBu (38.22 mg, 0.4 mmol), o-toluidine (43 μL, 0.4 mmol) and THF, the reaction proceeded at 100° C. for 48 h. After workup with general procedure, the title compound was obtained (29.1 mg, 87%). ¹H NMR (DMSO-d₆, 300 MHz) δ 10.56 (s, meso-2H), 9.62 (d, J=4.8 Hz, β-4H), 9.16 (d, J=4.8 Hz, β-4H), 8.08 (d, J=8.7 Hz, 4H), 7.98 (s, 2H), 7.59 (d, J=7.5 Hz, 2H), 7.38 (d, J=8.7 Hz, 4H), 7.27-7.38 (m, 4H), 7.04 (m, 2H), 2.44 (s, 6H), −3.10 (s, 2H). ¹³C NMR (DMSO-d₆, 75 MHz) δ 147.5, 144.9, 142.6, 140.9, 135.9, 131.4, 131.2, 131.0, 128.9, 126.9, 122.6, 119.6, 115.5, 105.1, 18.0. UV-vis (λₘₐₓ, nm) 419, 510, 552, 583, 640. HRMS-EI ([M+1]⁺): calc'd for, C₄₆H₃₇N₆, 673.3080; found 673.3107.

### Example 33

### Synthesis of 5,15-di-p-[N-(o-methylphenyl)amino]phenylporphyrin(Zn(II)) (Table 2, entry 6, B)

The general procedure using 5,15-di-p-bromophenylporphyrin(Zn(II)) (34.2 mg, 0.05 mmol), Pd(OAc)₂ (1.12 mg, 0.005 mmol), ligand 3 (3.8 mg, 0.01 mmol), NaOtBu (38.22 mg, 0.4 mmol), o-toluidine (43 μL, 0.4 mmol) and THF, the reaction proceeded at 100° C. for 48 h. After workup with general procedure, the title compound (29.1 mg, 87%) was obtained. ¹H NMR (DMSO-d₆, 300 MHz) δ 10.23 (s, meso-2H), 9.39 (d, J=4.8 Hz, β-4H), 9.20 (d, J=4.8 Hz, β-4H), 8.11 (d, J=8.1 Hz, 4H), 7.63 (d, J=7.5 Hz, 2H), 7.36 (d, J=8.4 Hz, 4H), 7.27-7.35 (m, 4H), 7.04 (dd, J=7.8 Hz 2H), 5.76 (s, 2H), 2.48 (s, 6H). ¹³C NMR (CDCl₃, 75 MHz) δ 150.3, 149.3, 139.7, 135.8, 132.3, 131.4, 131.1, 122.2, 115.4, 105.8, 18.2. UV-vis (λₘₐₓ, nm) 421, 542, 583. HRMS-EI ([M−Zn+1]⁺): calc'd for, C₄₆H₃₅N₆, 673.3080; found 673.3075.

### Example 34

### Synthesis of 5,15-di-n-butylaminophenylporphyrin (Table 2, entry 7, A)

The general procedure using 5,15-di-p-bromophenylporphyrin (31.0 mg, 0.05 mmol), Pd(OAc)₂ (1.12 mg, 0.005 mmol), ligand 3 (3.78 mg, 0.01 mmol), NaOtBu (38.22 mg, 0.4 mmol), n-butylamine (40 μL, 0.4 mmol) and THF (4-6 mL), the reaction proceeded at 100° C. for 48 h. After workup with general procedure, the title compound (27.7 mg, 92%) was obtained. By using other ligand or other condition, the same product with different yield was obtained (table 1, entry 7). ¹H NMR (CDCl₃, 300 MHz) δ 10.25 (s, meso-2H), 9.35 (d, J=4.6 Hz, β-4H), 9.16 (d, J=4.5 Hz, β-4H), 8.06 (d, J=8.4 Hz, 4H), 7.03 (d, J=8.4 Hz, 4H), 3.40 (t, J=6.6, 7.2 Hz, 4H), 1.83 (m, 4H), 1.59 (m, 4H), 1.08 (m, 6H), −3.00 (s, 2H). ¹³C NMR (CDCl₃, 75 MHz) δ 148.1, 147.8, 144.8, 136.1, 131.2, 131.1, 130.0, 119.7, 111.3, 104.9, 44.9, 31.8, 20.5, 14.1. UV-vis (λₘₐₓ, nm) 419, 511, 553, 586, 641. HRMS-EI ([M+1]⁺): calc'd for, C₄₀H₄₁N₆, 605.3393; found 605.3395.

### Example 35

### Synthesis of 5,15-di-n-butylaminophenylporphyrin (Zn II) (Table 2, entry 7, B)

The general procedure using 5,15-di-p-bromophenylporphyrin (Zn II) (34.2 mg, 0.05 mmol), Pd(OAc)₂ (1.12 mg, 0.005 mmol), ligand 8 (3.98 mg, 0.01 mmol), NaOtBu (38.22 mg, 0.4 mmol), n-butylamine (40 μL, 0.4 mmol) and THF (4-6 mL), the reaction proceeded at 100° C. for 48 h. After workup with general procedure, the title compound (31.1 mg, 93%) was obtained. ¹H NMR (DMSO-d₆, 300 MHz) δ 10.26 (s, meso-2H), 9.44 (d, J=4.8 Hz, β-4H), 9.04 (d, J=4.8 Hz, β-4H), 7.92 (d, J=8.1 Hz, 4H), 7.01 (d, J=8.1 Hz, 4H), 6.04 (t, 2H), 3.28 (m, 4H), 1.76 (m, 4H), 1.55 (m, 4H), 1.05 (m, 6H). ¹³C NMR (DMSO-d₆, 75 MHz) δ 149.9, 148.5, 135.6, 134.5, 134.3, 131.6, 129.5, 110.4, 42.8, 31.2, 20.1, 14.0. UV-vis (λₘₐₓ, nm419, 545, 586. HRMS-EI ([(M−Zn)+1]⁺): calc'd for, C₄₀H₄₁N₆, 605.3393; found 605.3360.

### Example 36

### Synthesis of 5,15-di-n-hexylaminophenylporphyrin (Table 2, entry 8, A)

The general procedure using 5,15-di-p-bromophenylporphyrin (31.0 mg, 0.05 mmol), Pd(OAc)₂ (1.12 mg, 0.005 mmol), ligand 3 (3.78 mg, 0.01 mmol), NaOtBu (38.22 mg, 0.4 mmol), n-hexylamine (52.8 μL, 0.4 mmol) and THF (4-6 mL), the reaction proceeded at 100° C. for 48 h. After workup with general procedure, the title compound (29.7 mg, 90%) was obtained. ¹H NMR (CDCl₃, 300 MHz) δ 10.25 (s, meso-2H), 9.35 (d, J=4.8 Hz, β-4H), 9.16 (d, J=4.2 Hz, β-4H), 8.05 (d, J=8.4 Hz, 4H), 7.03 (d, J=8.4 Hz, 4H), 4.05 (br, s, 2H), 3.40 (t, J=7.2 Hz, 4H), 1.84 (m, 4H), 1.54 (m, 4H), 1.43 (m, 4H), 0.97 (m, 6H), −3.00 (s, 2H). ¹³C NMR (CDCl₃, 75 MHz) δ 148.1, 147.8, 144.8, 136.1, 131.2, 131.1, 130.0, 119.7, 111.4, 104.9, 44.3, 31.8, 29.7, 27.0, 22.7, 14.1. UV-vis (λₘₐₓ, nm) 421, 509, 549, 583, 638.

### Example 37

### 5,15-di-n-hexylaminophenylporphyrin (Zn II) (Table 2, entry 8, B)

The general procedure using 5,15-di-p-bromophenylporphyrin (Zn II) (34.2 mg, 0.05 mmol), Pd(OAc)₂ (1.12 mg, 0.005 mmol), ligand 7 (5.78 mg, 0.01 mmol), NaOtBu (38.22 mg, 0.4 mmol), n-hexylamine (52.8 μL, 0.4 mmol) and THF (4-6 mL), the reaction proceeded at 100° C. for 48 h. After workup with general procedure, the title compound (17 mg, 53%) was obtained. ¹H NMR (CDCl₃, 300 MHz) δ 10.22 (s, meso-2H), 9.38 (d, J=4.8 Hz, β-4H), 9.18 (d, J=4.2 Hz, β-4H), 7.95 (d, J=8.1 Hz, 4H), 6.70 (d, J=8.1 Hz, 4H), 3.44 (m, 4H), 2.95 (m, 4H), 1.76 (m, 4H), 1.61 (m, 4H), 1.36 (m, 8H), 0.94 (m, 6H). ¹³C NMR (DMSO-d₆, 75 MHz) δ 150.5, 149.2, 139.4, 135.4, 132.5, 131.1, 111.4, 104.9, 44.1, 31.5, 28.0, 26.6, 22.7, 14.1. UV-vis (λₘₐₓ, nm) 419, 543, 584.

### Example 38

### 5,15-di-p-(N-methyl, N-phenylamino)phenylporphyrin (Table 2, entry 9, A)

The general procedure using 5,15-di-p-bromophenylporphyrin (31.0 mg, 0.05 mmol), Pd(OAc)₂ (1.12 mg, 0.005 mmol), ligand 3 (3.78 mg, 0.01 mmol), NaOtBu (38.22 mg, 0.4 mmol), N-methylaniline (43.7 μL, 0.4 mmol) and THF (4-6 mL), the reaction proceeded at 100° C. for 48 h. After workup with general procedure, the title compound (29.5 mg, 88%) was obtained. ¹H NMR (CDCl₃, 300 MHz) δ 10.28 (s, meso-2H), 9.39 (d, J=4.8 Hz, β-4H), 9.20 (d, J=4.8 Hz, β-4H), 8.14 (d, J=8.7 Hz, 4H), 7.37-7.50 (m, 12H), 7.13 (dd, J=2.1, 6.6 Hz, 2H), 3.62 (s, 6H), −3.02 (s, 2H). ¹³C NMR (CDCl₃, 75 MHz) δ 148.9, 148.5, 147.6, 144.9, 135.8, 133.0, 131.4, 131.1, 129.6, 122.8, 122.7, 119.2, 116.9, 105.1, 40.6. UV-vis (λₘₐₓ, nm) 413, 510, 552, 583, 640. HRMS-EI ([M]⁺): calc'd for C₄₆H₃₆N₆, 672.3001; found 672.3010.

### Example 39

### Synthesis of 5,15-di-p-(N-methyl, N-phenylamino)phenylporphyrin (Zn II) (Table 2, entry 9, B)

The general procedure using 5,15-di-p-bromophenylporphyrin (Zn II) (34.2 mg, 0.05 mmol), Pd(OAc)₂ (1.12 mg, 0.005 mmol), ligand 3 (3.78 mg, 0.01 mmol), NaOtBu (38.22 mg, 0.4 mmol), N-methylaniline (43.7 μL, 0.4 mmol) and THF (4-6 mL), the reaction proceeded at 100° C. for 48 h. After workup with general procedure, the title compound (27 mg, 73%) was obtained. ¹H NMR (CDCl₃, 300 MHz) δ 10.24 (s, meso-2H), 9.39 (d, J=4.2 Hz, β-4H), 9.22 (d, J=4.8 Hz, β-4H), 8.12 (d, J=8.1 Hz, 4H), 7.37-7.49 (m, 12H), 7.13 (m, 2H), 3.63 (s, 6H). ¹³C NMR (CDCl₃, 75 MHz) δ 150.4, 149.2, 148.3, 135.6, 134.5, 132.6, 131.5, 129.5, 122.4, 122.2, 117.0, 106.0, 40.6. UV-vis (λₘₐₓ, nm) 413, 544, 587. HRMS-EI ([M−Zn+1]⁺): calc'd for C₄₆H₃₅N₆, 673.3080; found 673.3104.

### Example 40

### Synthesis of 5,15-di-p-diphenylaminophenylporphyrin (Table 2, entry 10, A)

The general procedure using 5,15-di-p-bromophenylporphyrin (31.0 mg, 0.05 mmol), Pd(OAc)₂ (1.12 mg, 0.005 mmol), ligand 3 (3.78 mg, 0.01 mmol), NaOtBu (38.22 mg, 0.4 mmol), diphenylamine (67.7 mg, 0.4 mmol) and THF (4-6 mL), the reaction proceeded at 100° C. for 48 h. After workup with general procedure, the title compound (32.4 mg, 81%) was obtained. ¹H NMR (CDCl₃, 300 MHz) δ 10.28 (s, meso-2H), 9.39 (d, J=4.8 Hz, β-4H), 9.20 (d, J=4.8 Hz, 8-4H), 8.14 (d, J=8.7 Hz, 4H), 7.37-7.50 (m, 12H), 7.13 (dd, J=2.1, 6.6 Hz, 2H), 3.62 (s, 6H), −3.04 (s, 2H). ¹³C NMR (CDCl₃, 75 MHz) δ 147.8, 135.8, 131.5, 131.0, 129.5, 124.9, 123.3, 121.6, 105.2. UV-vis (λₘₐₓ, nm) 410, 510, 552, 583, 640. HRMS-EI ([M+1]⁺): calc'd for C₅₆H₄₁N₆, 797.3393; found 797.3398.

### Example 41

### Synthesis of 5,15-di-n-diphenylaminophenylporphyrin (Zn II) (Table 2, entry 10, B)

The general procedure using 5,15-di-p-bromophenylporphyrin (Zn II) (34.2 mg, 0.05 mmol), Pd(OAc)₂ (1.12 mg, 0.005 mmol), ligand 8 (3.98 mg, 0.01 mmol), NaOtBu (38.22 mg, 0.4 mmol), diphenylamine (67.7 mg, 0.4 mmol) and THF (4-6 mL), the reaction proceeded at 100° C. for 48 h. After workup with general procedure, the title compound (24.5 mg, 57%) was obtained. ¹H NMR (DMSO-d₆, 300 MHz) δ 10.35 (s, meso-2H), 9.52 (d, J=4.8 Hz, β-4H), 9.08 (d, J=4.5 Hz, β-4H), 8.12 (d, J=8.1 Hz, 4H), 7.37-7.51 (m, 12H), 7.17 (m, 4H). ¹³C NMR (DMSO-d₆, 75 MHz) δ 149.4, 148.9, 147.4, 146.6, 136.6, 135.6, 132.1, 129.9, 124.5, 123.4, 121.1, 118.8, 116.7, 106.1. UV-vis (λₘₐₓ, nm) 416, 543, 584. HRMS-EI ([M−Zn+1]⁺): calc'd for C₅₆H₄₁N₆Zn, 797.3393; found 797.3408.

### Example 42

### Synthesis of 5,15-di-n-benzophenone iminophenylporphyrin (Table 2, entry 11, A)

The general procedure using 5,15-di-p-bromophenylporphyrin (31.0 mg, 0.05 mmol), Pd₂(dba)₃ (4.58 mg, 0.005 mmol), ligand 1 (2.98 mg, 0.01 mmol), NaOtBu (38.22 mg, 0.4 mmol), benzophenone imine (67.1 μL, 0.4 mmol) and THF (4-6 mL), the reaction proceeded at 100° C. for 48 h. After workup with general procedure, the title compound (21.4 mg, 81%) was obtained. ¹H NMR (CDCl₃, 300 MHz) δ 10.27 (s, meso-2H), 9.36 (d, J=4.8 Hz, β-4H), 9.95 (d, J=4.8 Hz, β-4H), 8.0 (d, J=7.5 Hz, 4H), 7.95 (d, J=8.7 Hz, 4H ), 7.52 (m, 12H), 7.43 (m, 4H), 7.13 (d, J=7.5 Hz, 4H), −3.18 (s, 2H). ¹³C NMR (CDCl₃, 75 MHz) δ 142.9, 142.6, 135.9, 131.5, 131.0, 129.6, 121.6, 118.6, 115.7, 105.1. UV-vis (λₘₐₓ, nm) 412, 506, 541, 578, 634. HRMS-EI ([M+1]⁺): calc'd for C₅₈H₄₁N₆, 821.3393; found 821.3370.

### Example 43

### Synthesis of 5,15-di-p-morpholinophenylporphyrin (Table 2, entry 12, A)

The general procedure using 5,15-di-p-bromophenylporphyrin (31.0 mg, 0.05 mmol), Pd(OAc)₂ (1.12 mg, 0.005 mmol), ligand 8 (3.98 mg, 0.01 mmol), Cs₂CO₃ (130.33 mg, 0.4 mmol), morpholine (35 μL, 0.4 mmol) and THF (4-6 mL), the reaction proceeded at 100° C. for 48 h. After workup with general procedure, the title compound (25 mg, 76%) was obtained.

### Example 44

### Synthesis of Tetrakis-p-(N-phenylamino)phenylporphyrin (Table 3, entry 1)

The general procedure using tetrakis-p-bromophenylporphyrin (46.5 mg, 0.05 mmol), Pd(OAc)₂ (2.24 mg, 0.01 mmol), (±)BINAP (12.4 mg, 0.02 mmol, 9), NaOtBu (76.44 mg, 0.8 mmol), aniline (73 μL, 0.8 mmol) and THF (4-6 mL), the reaction proceeded at 100° C. for 72 h. After workup with general procedure, the title compound (44.6 mg, 91%) was obtained. ¹H NMR (CDCl₃, 300 MHz) δ 8.95 (s, β-8H), 8.08 (d, J=8.1 Hz, 8H), 7.34-7.42 (m, 24H), 7.04 (t, J=6.6, 7.2 Hz, 4H), 6.05 (s, 4H), −2.66 (s, 2H). ¹³C NMR (CDCl₃, 75 MHz) δ 142.9, 142.7, 135.7, 134.6, 129.5, 121.5, 119.9, 118.5, 115.3. UV-vis (λₘₐₓ, nm) 433, 524, 566, 657. HRMS-EI ([M+1]⁺): calc'd for C₆₈H₅₁N₈, 979.4237; found 979.4218.

### Example 45

### Synthesis of Tetrakis-p-(n-butylamino)phenylporphyrin (Table 3, entry 2)

The general procedure using tetrakis-p-bromophenylporphyrin (46.5 mg, 0.05 mmol), Pd(OAc)₂ (2.24 mg, 0.01 mmol), ligand 8 (7.96 mg, 0.02 mmol),, NaOtBu (76.44 mg, 0.8 mmol), n-butylamine (80 μL, 0.8 mmol) and THF (4-6 mL), the reaction proceeded at 100° C. for 72 h. After workup with general procedure, the title compound (38.5 mg, 86%) was obtained. ¹H NMR (CDCl₃, 300 MHz) δ 8.91 (s, β-8H), 8.01 (d, J=8.1 Hz, 8H), 6.95 (d, J=8.1 Hz, 8H), 3.95 (s, 4H), 3.60 (t, J=7.2, 8.4 Hz, 8H), 1.79 (m, 8H), 1.59 (m, 8H), 1.06 (t, J=6.9, 7.2 Hz, 12H), −2.64 (s, 2H). ¹³C NMR (CDCl₃, 75 MHz) δ 147.9, 135.8, 131.3, 120.3, 110.9, 43.9, 31.9, 20.5, 14.0. UV-vis (λₘₐₓ, nm) 434, 527, 571, 661. HRMS-EI ([M+1]⁺): calc'd for C₆₀H₆₇N₈, 899.5489; found 899.5507.

### Example 46

### Synthesis of Tetrakis-p-(N-methyl, N-phenylamino)phenylporphyrin (Table 3, entry 3)

The general procedure using tetrakis-p-bromophenylporphyrin (46.5 mg, 0.05 mmol), Pd(OAc)₂ (2.24 mg, 0.01 mmol), (±)BINAP (12.4 mg, 0.02 mmol, 9), NaOtBu (76.44 mg, 0.8 mmol), N-methylaniline (87.4 μL, 0.8 mmol) and THF (4-6 mL), the reaction proceeded at 100° C. for 72 h. After workup with general procedure, the title compound (42.4 mg, 82%) was obtained. ¹H NMR (CDCl₃, 300 MHz) δ 8.78 (s, 8i-8H), 7.92 (d, J=7.8 Hz, 8H), 7.21-7.30 (m, 16H), 7.16 (d, J=8.1 Hz, 8H), 7.05 (s, 2H), 6.95(t, 4H), 3.42 (s, 12H), −2.81 (s, 2H). ¹³C NMR (CDCl₃, 75 MHz) δ 148.9, 148.4, 135.6, 134.1, 129.5, 122.7, 122.6, 122.5, 120.1, 116.6, 116.5, 40.5. UV-vis (λₘₐₓ, nm) 435, 525, 567, 657. HRMS-EI ([M+1]⁺): Calc'd for C₇₂H₅₉N₈, 1035.4863; found 1035.4836.

### Example 47

### Synthesis of Tetrakis-p-(diphenylamino)phenylporphyrin (Table 3, entry 4)

The general procedure using tetrakis-p-bromophenylporphyrin (46.5 mg, 0.05 mmol), Pd(OAc)₂ (2.24 mg, 0.01 mmol), ligand 3 (7.56 mg, 0.02 mmol), NaOtBu (76.44 mg, 0.8 mmol), diphenylamine (135.4 mg, 0.8 mmol) and THF (4-6 mL), the reaction proceeded at 100° C. for 72 h. After workup with general procedure, the title compound (52.2 mg, 81%) was obtained. ¹H NMR (CDCl₃, 300 MHz) δ 9.02 (s, β-8H), 8.12 (d, J=8.7 Hz, 8H), 7.47 (d, J=8.4 Hz, 8H), 7.43 (s, 16H), 7.41 (m, 4H), 7.15 (s, 8H), −2.66 (s, 2H). ¹³C NMR (CDCl₃, 75 MHz) δ 147.8, 147.4, 135.9, 135.7, 129.5, 124.8, 123.3, 121.3, 119.9, 117.7. UV-vis (λₘₐₓ, nm) 439, 526, 570, 659. HRMS-EI ([M+1]⁺): calc'd for C₉₂H₆₇N₈, 1283.5489; found 1283.5478.

Examples 48 through 58 relate to methods for synthesizing meso-substituted phenoxyporphyrins, and the phenoxyporphyrin compounds so made, according to the presently disclosed subject matter.

### Example 48

### General Considerations

All reactions were carried out under a nitrogen atmosphere in oven-dried glassware using standard Schlenk techniques. Toluene was distilled under nitrogen from sodium benzophenone ketyl. Deuterated solvents were purchased from Cambridge Isotope Laboratories and were used as supplied. All other solvents were of liquid chromatography grade, which were purchased from Fisher Scientific and used as supplied. Phenols were purchased from Acros Organics or Aldrich Chemical Co. and used without further purification. [5-bromo-10,20-diphenylporphyrino]zinc(II) and [5,15-dibromo-10,20-diphenylporphyrino]zinc(II) were synthesized according to the literature. Phosphine ligands notably, bis(2-diphenylphosphinophenyl)ether (DPEphos), were purchased from Strem along with the metal precursors; palladium(II) acetate and tris(dibenzylideneacetone)dipalladium(0). Cesium carbonate was obtained as a gift from Chemetall Chemical Products, Inc. Proton and carbon nuclear magnetic resonance spectra (¹H NMR and ¹³C NMR) were recorded on a Varian Mercury 300 spectrometer and referenced with respect to residual solvent. Infrared spectra were obtained using a Bomem B100 Series FT-IR spectrometer. Samples were prepared as films on a NaCl plate by evaporating THF solutions. UV-Vis spectra were obtained using a Hewlett-Packard 8452A diode array spectrophotometer. High-resolution mass spectroscopy was performed by the Mass Spectrometry Center located in the Chemistry Department of the University of Tennessee on a VG Analytical hybrid high performance ZAB-EQ (B-E-Q geometry) instrument using electron impact (EI) ionization technique with a 70 eV electron beam. Thin layer chromatography was carried out on E. Merck Silica Gel 60 F-254 TLC plates.

### Example 49

### General Procedures for Catalytic C—O Coupling of Bromoporphyrin

The bromoporphyrin, palladium precursor, phosphine ligand and base were placed in an oven-dried, resealable Schlenk tube. The tube was sealed with a Teflon screw cap, evacuated, and backfilled with nitrogen. The screw cap was replaced with a rubber septum; the phenol was then added via syringe, followed by solvent. The tube was purged with nitrogen for 2 min, and then the septum was replaced with the Teflon screw cap. The tube was sealed, and its contents were placed in a heated oil-bath with constant stirring until the starting bromoporphyrin had been completely consumed as indicated by TLC analysis. The resulting mixture was cooled to room temperature, taken up in ethyl acetate (60 mL) and transferred to a separatory funnel. The mixture was then washed with water (×2), dried over anhydrous sodium sulfate, filtered and dried in vacuo. The crude product was then purified.

### Example 50

### Synthesis of 5-phenoxy-10,20-diphenylporphinato zinc(II)

The general procedure was used to couple 5-bromo-10,20-diphenylporphinato zinc(II) (30 mg, 0.05 mmol) with phenol (17 mg, 0.018 mmol), using palladium acetate (1 mg, 0.005 mmol) as the palladium precursor, DPEphos (4.0 mg, 0.015 mmol) as the phosphine ligand and cesium carbonate (24 mg, 0.07 mmol) as the base. The reaction was conducted in toluene (5 mL) at 100° C. for 23 hours. Isolated via flash chromatography (silica gel, THF:hexanes (v)=1:8 as a red solid (24 mg, 80%). ¹H NMR (300 MHz, CDCl₃): δ 10.13 (s, 1H), 9.39 (d, J=4.5 Hz, 2H), 9.31 (d, J=4.8 Hz, 2H), 9.09 (d J=4.5 Hz, 2H), 8.92 (d, J=4.8 Hz, 2H), 8.19 (m, 4H), 7.74 (m, 6H), 7.23 (m, 2H), 7.02 (m, 3H). ¹³C NMR (75 MHz, CDCl₃): δ 165.9, 150.3, 150.1, 149.7, 145.8, 142.3, 134.5, 132.9, 132.2, 131.8, 129.6, 128.0, 127.5, 126.6, 121.5, 120.7, 116.6, 107.7, 105.6. UV-vis (CHCl₃, λₘₐₓ, nm): 218, 418. IR (film, cm⁻¹): 3609, 3583, 3047, 2362, 1591, 1544, 1486, 1440, 1384, 1361, 1319, 1295, 1214, 1163, 1147, 1062, 996, 851, 790, 750, 721, 701. HRMS-EI ([M]⁺): C₃₈H₂₄N₄OZn, 616.124; found: 616.125.

### Example 51

### Synthesis of 5-(4-methoxyphenoxy)-10,20-diphenylporphinato zinc(II)

The general procedure was used to couple 5-bromo-10,20-diphenylporphinato zinc(II) (30 mg, 0.05 mmol) with 4-methoxyphenol (22 mg, 0.18 mmol), using palladium acetate (1 mg, 0.005 mmol) as the palladium precursor, DPEphos (4.0 mg, 0.015 mmol) as the phosphine ligand and cesium carbonate (24 mg, 0.07 mmol) as the base. The reaction was conducted in toluene (5 mL) at 100° C. for 17 hours. Isolated via flash chromatography (silica gel, THF:hexanes (v)=1:8 as a red solid (29.9 mg, 93%). ¹H NMR (300 MHz, CDCl₃): δ 10.02 (s, 1H), 9.35 (d, J=4.2 Hz, 2H), 9.24 (d, J=3.9 Hz, 2H), 8.98 (d, J=4.2 Hz, 2H), 8.8 (d, J=3.9 Hz, 2H), 8.18 (m, 4H), 7.75 (m, 6H), 6.92 (d, J=8.4 Hz, 2H), 6.67 (d, J=8.4 Hz, 2H), 3.60 (s, 3H). ¹³C NMR (75 MHz, CDCl₃): δ 160.8, 153.9, 150.2, 150.0, 149.5, 145.9, 142.6, 134.6, 132.7, 132.1, 132.0, 131.6, 127.9, 127.4, 126.5, 120.4, 117.1, 114.6, 105.2, 55.6. UV-vis (CHCl₃, λₘₐₓ, nm): 418, 548. IR (film, cm⁻¹): 3291, 3054, 2973, 2954, 2877, 2833, 2738, 1808, 1721, 1595, 1538, 1502, 1459, 1440, 1385, 1360, 1322, 1294, 1243, 1147, 1103, 1061, 1037, 994, 881, 846, 827, 793, 751, 724, 701. HRMS-EI ([M]⁺): C₃₉H₂₆N₄O₂Zn, 646.135; found: 646.137.

### Example 52

### Synthesis of 5-(4-t-butylphenoxy)-10,20-diphenylporphinato zinc(II)

The general procedure was used to couple 5-bromo-10,20-diphenylporphinato zinc(II) (30 mg, 0.05 mmol) with 4-t-butylphenol (27 mg, 0.18 mmol), using palladium acetate (1 mg, 0.005 mmol) as the palladium precursor, DPEphos (4.0 mg, 0.015 mmol) as the phosphine ligand and cesium carbonate (24 mg, 0.07 mmol) as the base. The reaction was conducted in toluene (5 mL) at 100° C. for 18 hours. Isolated via flash chromatography (silica gel, THF:hexanes (v)=1:8 as a red solid (23.8 mg, 73%). ¹H NMR (300 MHz, CDCl₃): δ 10.16 (s, 1H), 9.45 (d, J=4.8 Hz, 2H), 9.34 (d, J=4.2 Hz, 2H), 9.06 (d, J=4.5 Hz, 2H), 8.93 (d, J=4.8 Hz, 2H), 8.22 (m, 4H), 7.77 (m, 6H), 7.24 (d, J=9.9 Hz, 2H), 6.96 (d, J=8.7 Hz, 2H), 1.26 (S, 9H). ¹³C NMR (75 MHz, CDCl₃): δ 164.0, 159.9, 150.4, 150.1, 149.7, 146.0, 144.1, 142.4, 134.5, 132.9, 132.1, 131.7, 128.1, 127.5, 126.7, 126.3, 120.66, 115.9, 105.5, 31.5, 29.7. UV-vis (CHCl₃, λₘₐₓ, nm): 418, 548. IR (film, cm⁻¹): 3297, 3054, 3027, 2961, 2872, 1806, 1599, 1542, 1505, 1488, 1460, 1386, 1362, 1322, 1295, 1266, 1220, 1173, 1150, 1110, 1062, 1041, 995, 883, 846, 832, 792, 750, 723, 701. HRMS-EI ([M]⁺): C₄₂H₃₂N₄OZn, 672.187; found: 672.186.

### Example 53

### Synthesis of 5-(4-fluorophenoxy)-10,20-diphenylporphinato zinc(II)

The general procedure was used to couple 5-bromo-10,20-diphenylporphinato zinc(II) (30 mg, 0.05 mmol) with 4-fluorophenol (20 mg, 0.18 mmol), using palladium acetate (1 mg, 0.005 mmol) as the palladium precursor, DPEphos (4.0 mg, 0.015 mmol) as the phosphine ligand and cesium carbonate (24 mg, 0.07 mmol) as the base. The reaction was conducted in toluene (5 mL) at 100° C. for 17 hours. Isolated via flash chromatography (silica gel, THF:hexanes (v)=1:8 as a red solid (25.4 mg, 78%). ¹H NMR (300 MHz, CDCl₃): δ 10.10 (s, 1H), 9.35 (d, J=4.5 Hz, 2H), 9.29 (d, J=4.2 Hz, 2H), 9.02 (d, J=4.8 Hz, 2H), 8.91 (d, J=4.8 Hz, 2H), 8.19 (m, 4H), 7.76 (m, 6H), 6.93 (m, 4H). ¹³C NMR (75 MHz, CDCl₃): δ 150.4, 150.2, 149.70, 145.4, 134.5, 132.9, 132.2, 131.8, 127.7, 127.5, 126.6, 120.6, 117.4, 117.2, 116.1, 115.8, 105.6. UV-vis (CHCl₃, λₘₐₓ, nm): 418, 546. IR (film, cm⁻¹): 3273, 3101, 3073, 3054, 3023, 2974, 2933, 2875, 2740, 2951, 2582, 2552, 1807, 1719, 1597, 1541, 1520, 1498, 1459, 1440, 1386, 1360, 1322, 1295, 1260, 1195, 1145, 1091, 1062, 1041, 995, 885, 847, 832, 793, 751, 724, 701. HRMS-EI ([M]⁺): C₃₈H₂₃N₄OFZn, 634.115; found: 634.113.

### Example 54

### Synthesis of 5-(2-isopropylphenoxy)-10,20-diphenylporphinato zinc(II)

The general procedure was used to couple 5-bromo-10,20-diphenylporphinato zinc(II) (30 mg, 0.05 mmol) with 2-isopropylphenol (25 μL, 0.018 mmol), using palladium acetate (1 mg, 0.005 mmol) as the palladium precursor, DPEphos (4.0 mg, 0.015 mmol) as the phosphine ligand and cesium carbonate (24 mg, 0.07 mmol) as the base. The reaction was conducted in toluene (5 mL) at 100° C. for 17 hours. Isolated via flash chromatography (silica gel, THF:hexanes (v)=1:8 as a red solid (23 mg, 72%). ¹H NMR (300 MHz, CDCl₃): δ 10.05 (s, 1H), 9.33 (d, J=4.8 Hz, 2H), 9.26 (d, J=4.5 Hz, 2H), 9.01 (d, J=4.8 Hz, 2H), 8.90 (d, J=4.5 Hz, 2H), 8.19 (m, 4H), 7.75 (m, 6H), 7.60 (d, J=7.8 Hz, 1H), 6.96 (t, J=7.5 Hz, 1H), 6.67 (t, J=7.2 Hz, 1H), 6.02 (d, J=8.1 Hz, 1H), 4.40 (m, 1H), 1.82 (d, J=6.9 Hz, 6H). ¹³C NMR (75 MHz, CDCl₃): δ 163.86, 150.3, 150.0, 149.6, 145.8, 142.5, 135.8, 134.5, 132.8, 132.1, 131.6, 127.9, 127.4, 126.6, 126.5, 121.3, 120.4, 116.4, 105.2, 28.0, 23.3. UV-vis (CHCl₃, λₘₐₓ, nm): 418, 546. IR (film, cm⁻¹): 3293, 3055, 3026, 2961, 2873, 1805, 1596, 1542, 1483, 1441, 1385, 1360, 1322, 1294, 1261, 1218, 1191, 1154, 1061, 1039, 994, 885, 847, 824, 793, 750, 723, 701. HRMS-EI ([M]⁺): C₄₁H₃₀N₄OZn, 658.171; found: 658.168.

### Example 55

### Synthesis of 5-(3-methylphenoxy)-10,20-diphenylporphinato zinc(II)

The general procedure was used to couple 5-bromo-10,20-diphenylporphinato zinc(II) (30 mg, 0.05 mmol) with 3-cresol (20 μL, 0.018 mmol), using palladium DBA (1.5 mg, 0.0075 mmol) as the palladium precursor, DPEphos (9.6 mg, 0.036 mmol) as the phosphine ligand and cesium carbonate (34 mg, 0.1 mmol) as the base. The reaction was conducted in toluene (5 mL) at 100° C. for 16 hours. Isolated via flash chromatography (silica gel, THF:hexanes (v)=1:8 as a red solid (25 mg, 78%). ¹H NMR (300 MHz, CDCl₃): δ 10.03 (s, 1H), 9.36 (d, J=4.5 Hz, 2H), 9.25 (d, J=4.5 Hz, 2H), 9.00 (d, J=4.2 Hz, 2H), 8.89 (d, J=4.5 Hz, 2H), 8.2 (m, 4H), 7.76 (m, 6H), 7.10 (t, J=7.5 Hz, 1H), 6.80 (m, 3H), 2.15 (s, 3H). ¹³C NMR (75 MHz, CDCl₃): δ 166.1, 150.0, 150.2, 149.6, 145.8, 142.7, 139.7, 134.6, 132.7, 132.0, 131.6, 129.3, 127.9, 127.4, 126.5, 122.2, 120.3, 117.3, 113.7, 105.2, 21.4. UV-vis (CHCl₃, λₘₐₓ, nm): 418, 546. IR (film, cm⁻¹): 3053, 3024, 2922, 2877, 1587, 1542, 1484, 1458, 1440, 1384, 1360, 1321, 1294, 1248, 1217, 1188, 1158, 1061, 1039, 995, 911, 881, 848, 793, 781, 752, 723, 700. HRMS-EI ([M]⁺): C₃₉H₂₆N₄OZn, 630.140; found: 630.139.

### Example 56

### Synthesis of 5-(4-methylphenoxy)-10,20-diphenylporphinato zinc(II)

The general procedure was used to couple 5-bromo-10,20-diphenylporphinato zinc(II) (30 mg, 0.05 mmol) with 4-cresol (20 mg, 0.018 mmol), using palladium acetate (1 mg, 0.005 mmol) as the palladium precursor, DPEphos (4.0 mg, 0.015 mmol) as the phosphine ligand and cesium carbonate (24 mg, 0.07 mmol) as the base. The reaction was conducted in toluene (5 mL) at 100° C. for 16 hours. Isolated via flash chromatography (silica gel, toluene:hexanes (v)=3:1 as a red solid (21 mg, 65%). ¹H NMR (300 MHz, CDCl₃): δ 9.97 (s, 1H), 9.30 (d, J=4.5 Hz, 2H), 9.21 (d, J=4.5 Hz, 2H), 8.92 (d, J=4.5 Hz, 2H), 8.8 (d, J=4.5 Hz, 2H), 8.13 (m, 4H), 8.13 (m, 6H), 6.97 (d, J=9.0 Hz, 2H), 6.86 (d, J=8.7 Hz, 2H), 2.2 (s, 3H). ¹³C NMR (75 MHz, CDCl₃): δ 150.3, 145.8, 143.4, 142.7, 134.6, 132.7, 132.0, 131.6, 130.4, 130.0, 128.9, 128.4, 127.9, 127.4, 126.5, 125.2, 120.3, 116.3, 105.2, 24.9. UV-vis (CHCl₃, λₘₐₓ, nm): 416, 546. IR (film, cm⁻¹): 3324, 2988, 1557, 1505, 1453, 1440, 1384, 1358, 1321, 1294, 1215, 1167, 1145, 1060, 993, 846, 820, 793, 753, 723. HRMS-EI ([M]⁺): C₃₉H₂₆N₄OZn, 630.140; found: 630.141.

### Example 57

### Synthesis of 5-(2-methylphenoxy)-10,20-diphenylporphinato zinc(II)

The general procedure was used to couple 5-bromo-10,20-diphenylporphinato zinc(II) (30 mg, 0.05 mmol) with 2-cresol (20 mg, 0.018 mmol), using palladium DBA (1.5 mg, 0.0075 mmol) as the palladium precursor, DPEphos (9.6 mg, 0.036 mmol) as the phosphine ligand and cesium carbonate (24 mg, 0.07 mmol) as the base. The reaction was conducted in toluene (5 mL) at 100° C. for 17 hours. Isolated via flash chromatography (silica gel, toluene:hexanes (v)=3:1 as a red solid (27.8 mg, 89%). ¹H NMR (300 MHz, CDCl₃): δ 9.94 (s, 1H), 9.33 (d, J=4.8 Hz, 2H), 9.19 (d, J=4.5 Hz, 2H), 8.97 (d, J=4.2 Hz, 2H), 8.90 (d, J=4.8 Hz, 2H), 8.18 (m, 4H), 7.74 (m, 6H), 7.47 (d, J=6.9 Hz, 1H), 6.90 (t, J=7.2 Hz, 7.5 Hz 1H), 6.68 (t, J=7.5 Hz, 7.2 Hz 1H), 6.02 (d, J=8.4 Hz, 1H), 3.10 (s, 3H). ¹³C NMR (75 MHz, CDCl₃): δ 150.1, 149.9, 149.6, 145.7, 142.3, 140.7, 140.6, 140.6, 140.5, 134.5, 132.8, 132.1, 131.6, 131.0, 127.8, 127.5, 126.8, 126.6, 121.1, 116.2, 105.3, 17.0. UV-vis (CHCl₃, λₘₐₓ, nm): 415, 546. IR (film, cm⁻¹): 3047, 3024, 2922, 2877, 1587, 1542, 1484, 1458, 1440, 1384, 1359, 1321, 1294, 1217, 1188, 1158, 1061, 991, 908, 877, 851, 793, 779, 751, 723. HRMS-EI ([M]⁺): C₃₉H₂₆N₄OZn, 630.140; found: 630.139.

### Example 58

### Synthesis of bis-5,15-(4-methoxyphenoxy)-10,20-diphenylporphinato zinc(II)

The general procedure was used to couple 5,15-dibromo-10,20-diphenylporphinato zinc(II) (34 mg, 0.05 mmol) with 4-methoxyphenol (22 mg, 0.018 mmol), using palladium DBA (1.5 mg, 0.0075 mmol) as the palladium precursor, DPEphos (9.6 mg, 0.036 mmol) as the phosphine ligand and cesium carbonate (47 mg, 0.14 mmol) as the base. The reaction was conducted in toluene (5 mL) at 100° C. for 18 hours. Isolated via flash chromatography (silica gel, THF:hexanes (v)=1:8 as a purple solid (26 mg, 68%). ¹H NMR (300 MHz, THF-d₈): δ 9.28 (m, 4H), 8.77 (m, 4H), 8.17 (m, 4H), 7.73 (m, 6H), 8.8 (d, J=4.5 Hz, 2H), 8.13 (m, 4H), 7.73 (m, 6H), 6.95 (d, J=9.3 Hz, 4H), 6.77 (d, J=9.6 Hz, 4H), 3.67 (s, 6H). ¹³C NMR (75 MHz, CDCl₃): δ 155.4, 150.4, 147.8, 144.0, 135.3, 132.7, 128.4, 128.2, 127.2, 117.8, 115.3, 55.7. UV-vis (CHCl₃, λₘₐₓ, nm):426, 554. IR (film, cm⁻¹): 3056, 2950, 2903, 2833, 2353, 1812, 1722, 1596, 1502, 1490, 1461, 1439, 1332, 1302, 1243, 1198, 1166, 1144, 1103, 1063, 1035, 1003, 920, 884, 827, 796, 751, 735, 722, 702. HRMS-EI ([M]⁺): C₄₆H₃₂N₄O₄Zn, 768.162; found: 768.164.

### Example 59

### General Considerations for the Synthesis of meso-Chiral Porphyrins via Palladium-Mediated C—N and C—O Bond Formations

All reactions were carried out under a nitrogen atmosphere in oven-dried glassware following standard Schienk techniques. Tetrahydrofuran and toluene were distilled under nitrogen from sodium benzophenone ketyl. 5,15-dibromo-10,20-diphenylporphyrin, 5,15-dibromo-10,20-di(3′,5′-di-tert-butylphenyl)porphyrin, 5,15-dibromo-10,20-di(2′,6′-dimethylphenyl)porphyrin and 5,15-dibromo-10,20-di(2′,4′,6′-trimethylphenyl)porphyrin were synthesized by literature methods. See Lindsey et al., (1987) 52: 827; DiMagno et al., (1993) J. Org. Chem. 58: 5983. Thin-layer chromatography was carried out on E. Merck Silica Gel 60 F-254 TLC plates.

### Example 60

### General Procedures for the Etheration and the Amidation of a Bromoporphyrin

The general procedures for the etheration and amidation of bromoporphyrin follow those described by Gao et al., (2003) Org. Lett. 5: 3261; and Gao et al., (2004) Org. Lett. 6: 1837. The bromoporphyrin, chiral alcohol or amide, palladium precursor, phosphine ligand, and base were placed in an oven-dried, resealable Schlenk tube. The tube was capped with a Teflon screwcap, evacuated, and backfilled with nitrogen. The screwcap was replaced with a rubber septum, and solvent was added via syringe. The tube was purged with nitrogen for 2 min, and then the septum was replaced with the Teflon screwcap. The tube was sealed, and its contents were heated with stirring until the starting bromoporphyrin had been completely consumed as indicated by TLC analysis. The resulting mixture was cooled to room temperature, taken up in ethyl acetate, and transferred to a separatory funnel. The mixture was washed with water and concentrated in vacuo. The crude product was then purified by flash chromatography.

### Example 61

### General Procedures for the Synthesis of a Cobalt Porphyrin Complex

The general procedures for the synthesis of cobalt porphyrin follow those described by Tsuchida et al., (1990) Chem. Lett. 3: 389; Tsuchida et al., (1990) J. Chem. Soc.-Dalton Trans. 2713; and Komatsu et al., (1990) J. Chem. Soc.-Chem. Commun. 66. Free base porphyrin and anhydrous CoCl₂ were placed in an oven-dried, resealable Schlenk tube. The tube was capped with a Teflon screwcap, evacuated, and backfilled with nitrogen. The screwcap was replaced with a rubber septum, 2,6-lutidine and dry THF were added via syringe. The tube was purged with nitrogen for 2 minutes, and then the septum was replaced with the Teflon screwcap. The tube was sealed, and its contents were heated with stirring. The resulting mixture was cooled to room temperature, taken up in ethyl acetate and transferred to a separatory funnel. The mixture was washed with water 3 times and concentrated in vacuo.

### Example 62

### meso-Chiral Porphyrin 15a

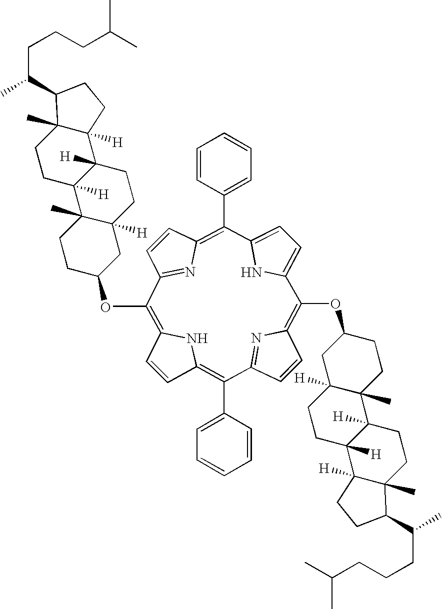

The general procedure was used to couple 5,15-dibromo-10,20-diphenylporphyrin (31.0 mg, 0.05 mmol) with (+)-dihydrocholesterol (77.8 mg, 0.2 mmol), using Pd₂(dba)₃ (4.6 mg, 0.005 mmol) and DPEphos (10.7 mg, 0.02 mmol) in the presence of Cs₂CO₃ (65.2 mg, 0.2 mmol). The reaction was conducted in toluene at 100° C. for 17 h. The title compound was isolated by flash chromatography (silica gel, methylene chloride:hexanes (v/v)=8:2) as a purple solid (27.5 mg, 45%). ¹H NMR (300 MHz, CDCl₃): δ 9.43 (d, J=4.8 Hz, 4H), 8.78 (d, J=4.8 Hz, 4H), 8.18 (m, 4H), 7.74 (m, 6H), 4.95 (s, 2H), 0.62-2.3 (m, 92H), −2.59 (s, 2H). UV-vis (CH₂Cl₂, λₘₐₓ, nm): 418, 520, 557, 602, 660. HRMS-MALDI ([M+H]⁺): calcd for C₈₆H₁₁₅N₄O₂, 1235.9015, found 1235.90110 with an isotope distribution pattern that is the same as calculated one.

### Example 63

### Cobalt Complex 16a

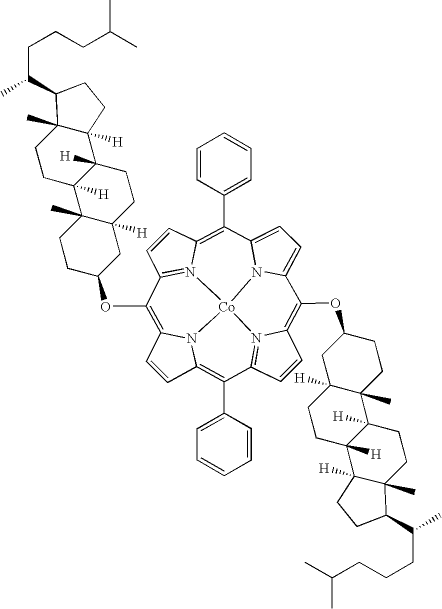

The general procedure was used for cobalt ion insertion. meso-Chiral porphyrin 15a (0.040 g), anhydrous CoCl₂ (0.030 g), 2,6-lutidine (0.012 mL), and dry THF (8 mL) were heated at 70° C. under N₂ for 15 hours. The resulting mixture was cooled to room temperature, taken up in ethyl acetate and transferred to a separatory funnel. The mixture was washed with water 3 times and concentrated in vacuo. The title compound was obtained as a red solid (0.030 g, 71%). UV-vis (CHCl₃, λₘₐₓ, nm): 412, 535, 577. HRMS-EI ([M−1Cholestane+H]⁺): calcd for C₅₉H₆₆CoN₄O₂, 921.4518, found 921.4487 with an isotope distribution pattern that is the same as calculated one.

### Example 64

### meso-Chiral Porphyrin 15b

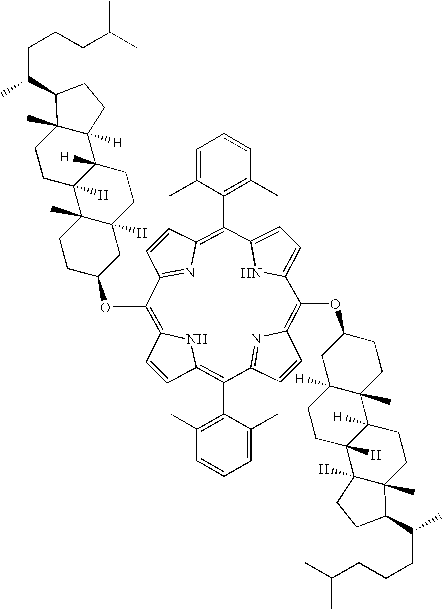

The general procedure was used to couple 5,15-dibromo-10,20-di(2′,6′-dimethylphenyl)porphyrin (0.034 g, 0.05 mmol) with (+)-dihydrocholesterol (0.1556 g, 0.4 mmol), using Pd₂(dba)₃ (0.0046 g, 0.005 mmol) and DPEphos (0.0107 g, 0.02 mmol) in the presence of Cs₂CO₃ (0.0652 g, 0.2 mmol). The reaction was conducted in toluene (5 mL) at 100° C. for 20 h. The title compound was isolated by flash column chromatography (silica gel, ethyl acetate:hexanes (v/v)=1:10) as purple solids (0.053 g, 82%). ¹H NMR (300 MHz, CDCl₃): δ 9.43 (d, J=4.8 Hz, 4H), 8.65 (d, J=4.8 Hz, 4H), 7.63 (t, J=7.5 Hz, 2H), 7.49 (d, J=7.5 Hz, 4H), 5.02 (m, 2H), 2.32-0.55 (m, 92H), 1.93 (s, 12H), −2.42 (s, 2H). ¹³C NMR (75 MHz, CDCl₃): δ 141.0, 139.6, 135.6, 128.2, 127.0, 117.5, 91.8, 56.4, 56.2, 54.3, 45.0, 42.5, 39.9, 39.5, 37.1, 36.1, 35.8, 35.7, 35.4, 32.0, 29.4, 28.7, 28.2, 28.0, 24.2, 23.8, 22.8, 22.6, 21.8, 21.2, 18.6, 12.6, 12.1. UV-vis (CHCl₃, λₘₐₓ, nm): 418, 518, 555, 600, 659. HRMS-MALDI ([M+H]⁺): calcd for C₉₀H₁₂₃N₄O₂, 1291.9641; found: 1291.9650 with an isotope distribution pattern that is the same as calculated one.

### Example 65

### Cobalt Complex 16b

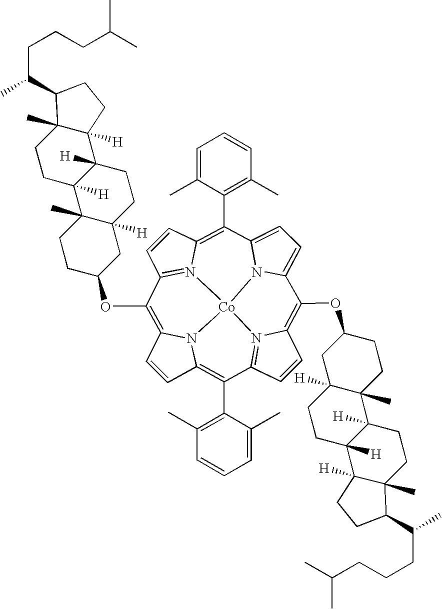

The general procedure was used for cobalt ion insertion. meso-Chiral porphyrin 15b (0.030 g), anhydrous COCl₂ (0.020 g), 2,6-lutidine (0.008 mL) and dry THF (5 mL) were heated at 70° C. under N₂ for 14 hours. The resulting mixture was cooled to room temperature, taken up in ethyl acetate and transferred to a separatory funnel. The mixture was washed with water 3 times and concentrated in vacuo. The title compound was obtained as a red solid (0.030 g, 96%). UV-vis (CHCl₃, λₘₐₓ, nm): 411, 533, 571. HRMS-MALDI ([M−2Cholestane+2H]⁺): calcd for C₃₆H₂₈CoN₄O₂, 607.1539, found 607.1570 with an isotope distribution pattern that is the same as calculated one.

### Example 66

### meso-Chiral Porphyrin 15c

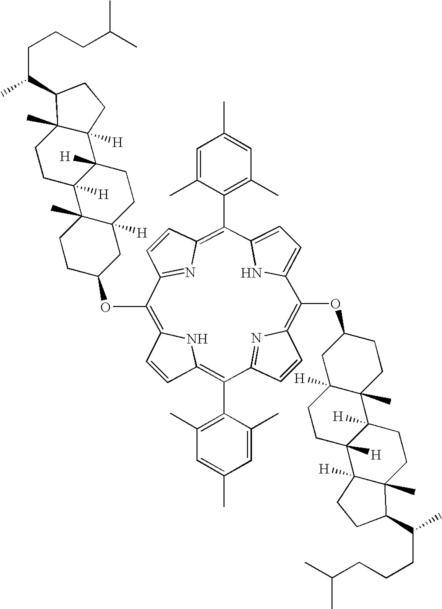

The general procedure was used to couple 5,15-dibromo-10,20-di(2′,4′,6′-trimethylphenyl)porphyrin (0.035 g, 0.05 mmol) with (+)-dihydrocholesterol (0.1556 g, 0.4 mmol), using Pd₂(dba)₃ (0.0046 g, 0.005 mmol) and DPEphos (0.0107 g, 0.02 mmol) in the presence of Cs₂CO₃ (0.0652 g, 0.2 mmol). The reaction was conducted in toluene (5 mL) at 100° C. for 20 h. The title compound was isolated by flash column chromatography (silica gel, ethyl acetate:hexanes (v/v)=1:10) as purple solids (0.052 g, 80%). ¹H NMR (300 MHz, CDCl₃): δ 9.42 (d, J=4.8 Hz, 4H), 8.67 (d, J=4.8 Hz, 4H), 7.32 (s, 4H), 5.02 (m, 2H), 2.31-0.55 (m, 92), 2.67 (s, 6H) 1.90 (s, 12H), −2.43 (s, 2H). ¹³C NMR (75 MHz, CDCl₃): δ 139.4, 138.1, 137.6, 135.5, 127.8, 117.6, 91.7, 56.4, 56.2, 54.3, 45.0, 42.5, 40.0, 39.5, 37.1, 36.1, 35.8, 35.4, 32.0, 29.4, 28.7, 28.2, 28.0, 24.2, 23.8, 22.8, 22.6, 21.7, 18.6, 12.6, 12.1. UV-vis (CHCl₃, λₘₐₓ, nm): 418, 519, 557, 601, 659. HRMS-MALDI ([M+H]⁺): calcd for C₉₂H₁₂₇N₄O₂, 1319.9954; found: 1320.0008 with an isotope distribution pattern that is the same as calculated one.

### Example 67

### Cobalt Complex 16c

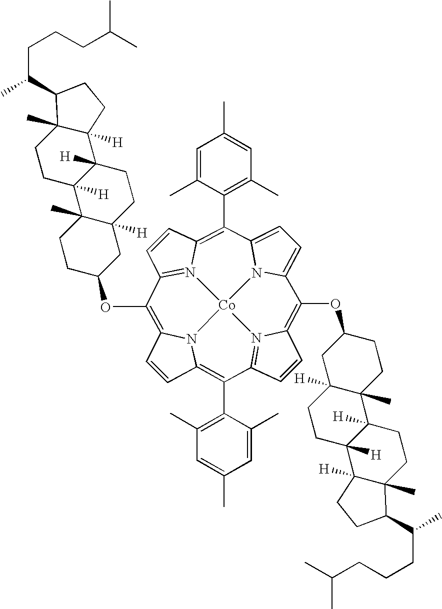

The general procedure was used for cobalt ion insertion. meso-Chiral porphyrin 15c (0.023 g), anhydrous COCl₂ (0.020 g), 2,6-lutidine (0.008 mL) and dry THF (5 mL) were heated at 70° C. under N₂ for 14 hours. The resulting mixture was cooled to room temperature, taken up in ethyl acetate and transferred to a separatory funnel. The mixture was washed with water 3 times and concentrated in vacuo. The title compound was obtained as a red solid (0.021 g, 88%). UV-vis (CHCl₃, λₘₐₓ, nm): 412, 534, 574. HRMS-MALDI ([M−2Cholestane+2H]⁺): calcd for C₃₈H₃₂CoN₄O₂, 635.1852, found 635.1844 with an isotope distribution pattern that is the same as calculated one.

### Example 68

### meso-Chiral Porphyrin 15d

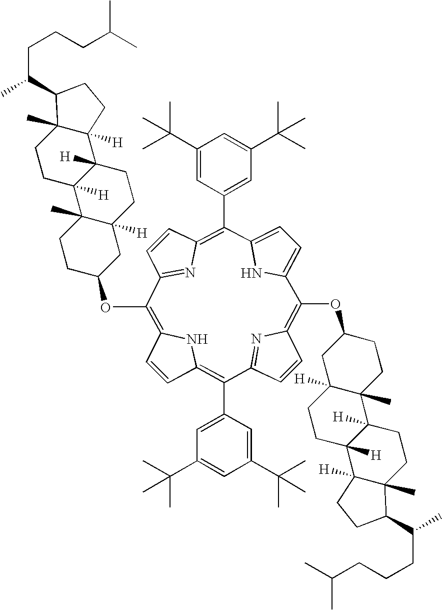

The general procedure was used to couple 5,15-dibromo-10,20-di(3,5-di-tert-butylphenyl)porphyrin (0.043 g, 0.05 mmol) with (+)-dihydrocholesterol (0.1556 g, 0.4 mmol), using Pd₂(dba)₃ (0.0046 g, 0.005 mmol) and DPEphos (0.0107 g, 0.02 mmol) in the presence of Cs₂CO₃ (0.0652 g, 0.2 mmol). The reaction was conducted in toluene (5 mL) at 100° C. for 18 h. The title compound was isolated by flash column chromatography (silica gel, ethyl acetate:hexanes (v/v)=1:20) as purple solids (0.059 g, 79%). ¹H NMR (300 MHz, CDCl₃): δ 9.46 (d, J=4.8 Hz, 4H), 8.87 (d, J=4.8 Hz, 4H), 8.09 (d, J=2.1 Hz, 4H), 7.81 (t, J=1.8 Hz, 2H), 5.01 (m, 2H), 2.26-0.61 (m, 92H), 1.57 (s, 36H), −2.48 (s, 2H). ¹³C NMR (75 MHz, CDCl₃): δ 148.9, 140.8, 135.8, 130.6, 129.9, 127.4, 121.0, 120.8, 91.6, 56.3, 56.1, 54.2, 44.9, 42.5, 39.9, 39.5, 37.0, 36.1, 35.8, 35.7, 35.4, 35.0, 31.8, 29.3, 28.7, 28.2. 28.0, 24.1, 23.8, 22.8, 22.5, 21.9, 18.6, 12.6, 12.0. UV-vis (CHCl₃, λₘₐₓ, nm): 420, 521, 560, 604, 661. HRMS-MALDI ([M+H]⁺): calcd for C₁₀₂H₁₄₇N₄O₂, 1460.1519, found 1460.1522 with an isotope distribution pattern that is the same as calculated one.

### Example 69

### Cobalt Complex 16d

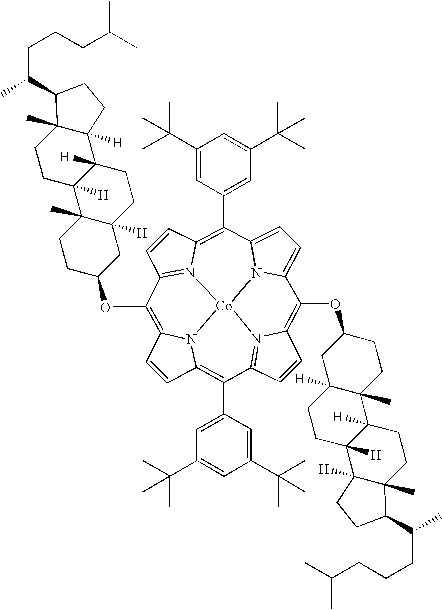

The general procedure was used for cobalt ion insertion. meso-Chiral porphyrin 15d (0.027 g), anhydrous COCl₂ (0.020 g), 2,6-lutidine (0.008 mL) and dry THF (5 mL) were heated at 70° C. under N₂ for 14 hours. The resulting mixture was cooled to room temperature, taken up in ethyl acetate and transferred to a separatory funnel. The mixture was washed with water 3 times and concentrated in vacuo. The title compound was obtained after flash chromatography (silica gel, ethyl acetate: hexanes (v/v)=1:8) as a red solid (0.025 g, 89%). UV-vis (CHCl₃, λₘₐₓ, nm): 413, 534, 578. HRMS-MALDI ([M−2Cholestane+2H]⁺): calcd for C₄₈H₅₂CoN₄O₂, 775.3422, found 775.3425 with an isotope distribution pattern that is the same as calculated one.

### Example 70

### meso-Chiral Porphyrin 15e

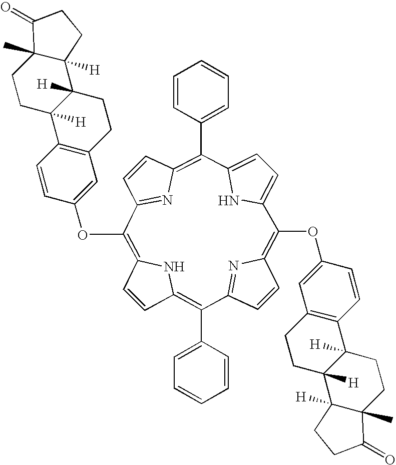

The general procedure was used to couple 5,15-dibromo-10,20-diphenylporphyrin (31.0 mg, 0.05 mmol) with (+)-estrone (108 mg, 0.2 mmol), using Pd₂(dba)₃ (4.6 mg, 0.005 mmol) and DPEphos (10.7 mg, 0.02 mmol) in the presence of Cs₂CO₃ (65.2 mg, 0.2 mmol). The reaction was conducted in toluene at 100° C. for 40 h. The title compound was isolated by flash chromatography (silica gel, methylene chloride:ethyl acetate (v/v)=9:1) as a purple solid (49.1 mg, 98%). ¹H NMR (300 MHz, CDCl₃): δ 9.30 (d, J=4.5 Hz, 4H), 8.78 (d, J=4.8 Hz, 4H), 8.16 (m, 4H), 7.74 (m, 6H), 7.16 (d, J=8.4 Hz, 1H), 7.13 (d, J=8.4 Hz, 1H), 6.89 (d, J=8.4, 2.7 Hz, 1H), 6.86 (d, J=8.4, 3.0 Hz, 1H), 6.65 (d, J=3.0 Hz, 2H), 1.20-2.8 (m, 30H), 0.88 (s, 3H), 0.86 (s, 3H), −2.47 (s, 2H). ¹³C NMR (75 MHz, CDCl₃): δ 141.1, 138.1, 134.6, 133.1, 132.0, 131.3, 131.0, 127.8, 126.9, 120.2, 116.3, 114.0, 50.3, 47.9, 44.0, 38.3, 38.1, 35.9, 31.5, 29.4, 26.3, 25.8, 21.5, 13.8. UV-vis (CH₂Cl₂, λₘₐₓ, nm): 420, 517, 555, 598, 655. HRMS-EI ([M]⁺): calcd for C₆₈H₆₂N₄O₄, 998.4771, found 998.4773 with an isotope distribution pattern that is the same as calculated one.

### Example 71

### Cobalt Complex 16e

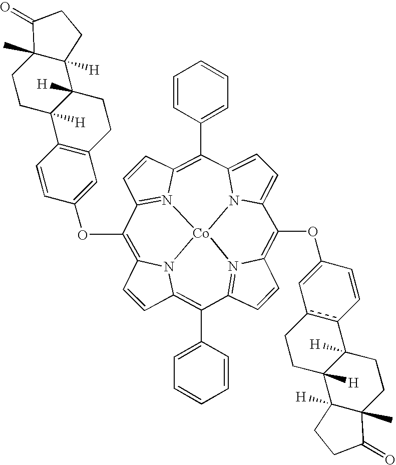

To 100 mg meso-Chiral porphyrin 15e in DMF (40 mL) was added cobalt acetate tetrahydrate (150 mg, 0.8 mmol). The solution was purged with nitrogen and heated at 160° C. for 2 h, cooled to room temperature, and poured into water. The crude product was extracted with ethyl acetate and concentrated to dry. The pure compound obtained after flash chromatography (silica gel, methylene chloride: hexanes (v/v)=8:2) as a red solid (81 mg, 77%). UV-vis (CH₂Cl₂, λₘₐₓ, nm): 413, 436, 531. HRMS-EI ([M]⁺): calcd for C₆₈H₆₀CoN₄O₄, 1055.3947, found 1055.3937 with an isotope distribution pattern that is the same as calculated one.

### Example 72

### meso-Chiral Porphyrin 15f (Mixture of α,α and α,β)

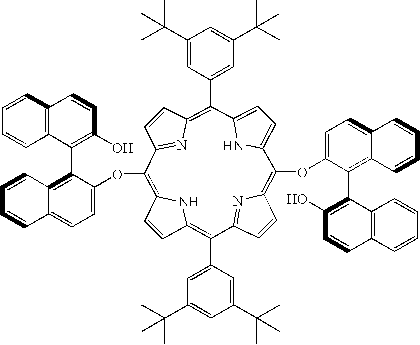

The general procedure was used to couple 5,15-dibromo-10,20-di(3,5-di-tert-butylphenyl)porphyrin (0.043 g, 0.05 mmol) with R-(+)-1,1′-bi-2-naphthol (0.167 g, 0.58 mmol), using Pd₂(dba)₃ (0.0046 g, 0.005 mmol) and DPEphos (0.0107 g, 0.02 mmol) in the presence of Cs₂CO₃ (0.0652 g, 0.2 mmol). The reaction was conducted in toluene (5 mL) at 100° C. for 20 h. The title compound was isolated by flash column chromatography (silica gel, ethyl acetate:hexanes (v/v)=1:5) as purple solids (0.022 g, 35%). ¹H NMR (300 MHz, CDCl₃): δ 9.21 (d, J=4.8 Hz, 4H), 8.77 (d, J=4.8 Hz, 4H), 7.37-8.05 (m, 26H), 7.07 (d, J=16.2 Hz, 2H), 6.49 (d, J=9.6 Hz, 2H), 5.66 (s, 2H), 1.51 (m, 36H), −2.45 (s, 2H). ¹³C NMR (75 MHz, CDCl₃): δ 161.2, 151.9, 149.0, 140.0, 134.4, 133.9, 131.7, 130.8, 130.3, 129.7, 129.6, 128.9, 128.6, 128.4, 127.9, 127.4, 127.0, 125.2, 125.0, 123.7, 121.3, 117.8, 117.2, 114.5, 35.0, 31.7. UV-vis (CHCl₃, λₘₐₓ, nm): 425, 521, 558, 598, 655. HRMS-MALDI ([M+H]⁺): calcd for C₈₈H₇₉N₄O₄, 1255.6096, found 1255.6045 with an isotope distribution pattern that is the same as calculated one.

### Example 73

### Cobalt Complex 16f

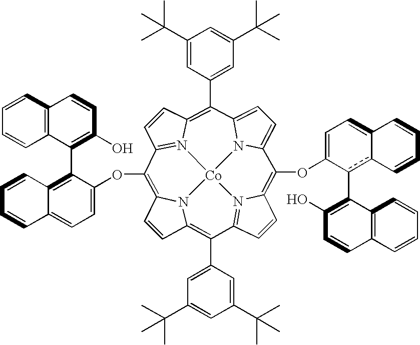

The general procedure was used for cobalt ion insertion. meso-Chiral porphyrin 15f (0.020g), anhydrous CoCl₂ (0.017 g), 2,6-lutidine (0.006 mL) and dry THF (4 mL) were heated at 70° C. under N₂ for 14 hours. The resulting mixture was cooled to room temperature, taken up in ethyl acetate and transferred to a separatory funnel. The mixture was washed with water 3 times and concentrated in vacuo. The title compound was obtained as a red solid (0.020 g, 96%). UV-vis (CHCl₃, λₘₐₓ, nm): 417, 532, 564. HRMS-MALDI ([M]⁺): calcd for C₈₈H₇₆CoN₄O₄ 1311.5199, found 1311.5225 with an isotope distribution pattern that is the same as calculated one.

### Example 74

### Meso-Chiral Porphyrin 17a (Mixture of α,α and α,β)

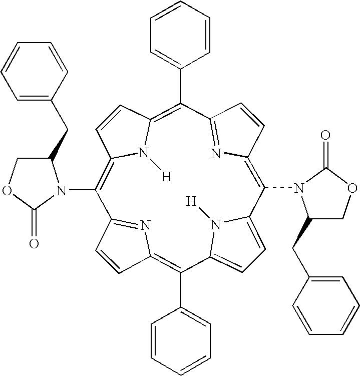

The general procedure described by Gao et al., (2004) Org. Lett., 6:1837, was used to couple 5,15-dibromo-10,20-diphenylporphyrin (31.0 mg, 0.05 mmol) with (R)-(+)-4-benzyl-2-oxazolidinone (70.8 mg, 0.4 mmol), using Pd₂(dba)₃ (2.3 mg, 0.0025 mmol) and Xantphos (5.78 mg, 0.01 mmol) in the presence of Cs₂CO₃ (65.2 mg, 0.2 mmol). The reaction was conducted in THF at 68° C. for 22 h. The title compound was isolated by flash chromatography (silica gel, methylene chloride: ethyl acetate (v/v)=9:1) as a purple mixture of two atropic isomers (25 mg, 62%, α,α/α,β=50%/50%). ¹H NMR (300 MHz, CDCl₃): δ 9.46 (d, J=5.1 Hz, 4H), 9.42 (d, J=4.8 Hz, 4H), 9.24 (t, J=4.2 Hz, 4H), 9.00 (d, J=4.8 Hz, 2H), 8.96 (d, J=4.2 Hz, 4H), 8.93 (d, J=4.8 Hz, 2H), 8.32 (d, J=4.8 Hz, 2H), 8.20 (t, J=7.2 Hz, 4H), 8.06 (d, J=4.8 Hz, 2H), 7.79 (m, 12H), 7.07 (m, 6H), 7.02 (m, 6H), 6.83 (m, 8H), 5.35 (m, 2H), 5.23 (m, 2H), 5.00 (dd, J=9.0 Hz, 4H), 4.82 (dd, J=9.0 Hz, 4H), 3.13-3.30 (m, 4H), 2.94 (dd, J=13.2, 3.3 Hz, 2H), 2.70 (dd, J=13.2, 3.3 Hz, 2H), −2.84 (s, 2H), −2.89 (s, 2H); ¹³C NMR (75 MHz, CDCl₃): δ 147.1, 140.1, 140.9, 135.0, 134.6, 128.8, 128.7, 128.2, 127.1, 126.9, 126.8, 121.4, 111.5, 68.3, 66.4, 40.4, 40.3. UV-vis (CH₂Cl₂, λₘₐₓ, nm): 412, 526, 558. HRMS-MALDI ([M+H]⁺): calcd for C₅₂H₄₁N₆O₄, 813.3184, found 813.3194 with an isotope distribution pattern that is the same as calculated one.

### Example 75

### Cobalt Complex 18a

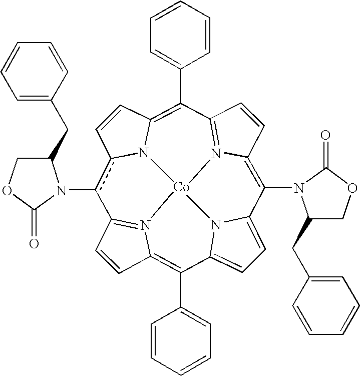

The general procedure was used for cobalt ion insertion. meso-Chiral porphyrin 17a (0.030 g), anhydrous COCl₂ (0.038 g), 2,6-lutidine (0.015 mL) and dry THF (5 mL) were heated at 70° C. under N₂ for 14 hours. The resulting mixture was cooled to room temperature, taken up in ethyl acetate and transferred to a separatory funnel. The mixture was washed with water for 3 times and concentrated in vacuo. The title compound was obtained as a red solid (0.028 g, 87%). UV-vis (CHCl₃, λₘₐₓ, nm): 408, 528, 560. HRMS-MALDI ([M+H]⁺): calcd for C₅₂H₃₉CoN₆O₄ 870.2359, found 870.2332 with an isotope distribution pattern that is the same as calculated one.

### Example 76

### meso-Chiral Porphyrin 17b (Mixture of α,α and α,β)

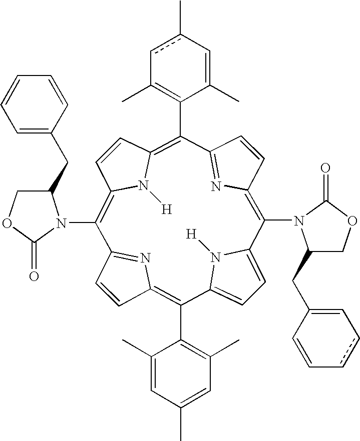

The general procedure was used to couple 5,15-dibromo-10,20-di(2′,4′,6′-trimethylphenyl)porphyrin (0.035 g, 0.05 mmol) with (R)-(+)-4-benzyl-2-oxazolidinone (0.0708 g, 0.4 mmol), using Pd₂(dba)₃ (0.0046 g, 0.005 mmol) and Xantphos (0.0116 g, 0.02 mmol) in the presence of Cs₂CO₃ (0.0652 g, 0.2 mmol). The reaction was conducted in THF (5 mL) at 80° C. for 20 h. The title compound was isolated by flash column chromatography (silica gel, ethyl acetate:hexanes (v/v)=1:2) as purple mixture of two atropic isomers (0.032 g, 72%, α,α and α,β=50%/50%). ¹H NMR (300 MHz, CDCl₃): δ 9.45 (d, J=4.8 Hz, 2H), 9.41 (d, J=4.8 Hz, 2H), 9.22 (m, 4H), 8.88 (d, J=4.8 Hz, 2H), 8.82 (m, 6H), 7.35(m, 8H), 7.10 (m, 8H), 6.95 (m, 4H), 6.86 (m, 8H), 5.36 (m, 2H), 5.20 (m, 2H), 5.03 (m, 4H), 4.86 (m, 4H), 3.28 (m, 4H), 3.07 (dd, J=13.2, 3.6 Hz, 2H), 2.75 (dd, J=13.2, 3.3 Hz, 2H), 2.67 (s, 12H), 2.04 (s, 6H), 1.89 (s, 6H), 1.86 (s, 6H), 1.73 (s, 6H), −2.67 (s, 2H), −2.76 (s, 2H). ¹³C NMR (75 MHz, CDCl₃): δ 159.5, 139.5, 139.3, 139.1, 138.2, 137.3, 137.2, 135.0, 128.8, 128.6, 128.5, 128.1, 127.9, 127.7, 127.1, 126.9, 119.9, 111.1, 110.8, 68.8, 66.3, 40.4, 21.9, 21.7, 21.6, 21.5. UV-vis (CHCl₃, λₘₐₓ, nm): 416, 512, 544, 590, 646. HRMS-MALDI ([M+H]⁺): calcd for C₅₈H₅₃N₆O₄, 897.4123, found 897.4109 with an isotope distribution pattern that is the same as calculated one.

### Example 77

### Cobalt Complex 18b

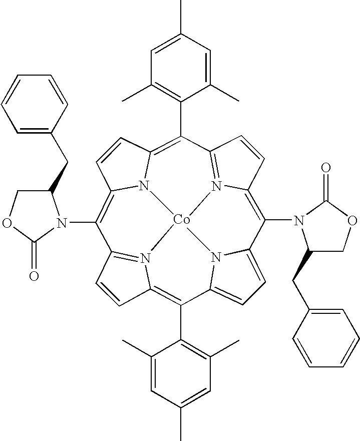

The general procedure was used for cobalt ion insertion. meso-Chiral porphyrin 17b (0.020 g), anhydrous COCl₂ (0.020 g), 2,6-lutidine (0.008 mL) and dry THF (4 mL) were heated at 70° C. under N₂ for 14 hours. The resulting mixture was cooled to room temperature, taken up in ethyl acetate and transferred to a separatory funnel. The mixture was washed with water for 3 times and concentrated in vacuo. The title compound was obtained as a red solid (0.020 g, 94%). UV-vis (CHCl₃, λₘₐₓ, nm): 409, 528, 559. HRMS-EI ([M]⁺): calcd for C₅₈H₅₀CoN₆O₄ 953.3226, found: 953.3254 with an isotope distribution pattern that is the same as calculated one.

### Example 78

### meso-Chiral Porphyrin 17c (Mixture of α,α and α,β)

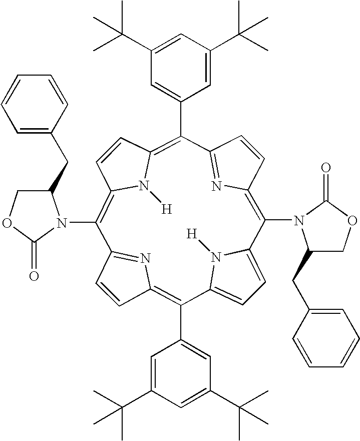

The general procedure was used to couple 5,15-dibromo-10,20-di(3′,5′-di-tert-butylphenyl)porphyrin (0.043 g, 0.05 mmol) with (R)-(+)-4-benzyl-2-oxazolidinone (0.0708 g, 0.4 mmol), using Pd₂(dba)₃ (0.0023 g, 0.0025 mmol) and Xantphos (0.0058 g, 0.01 mmol) in the presence of Cs₂CO₃ (0.0652 g, 0.2 mmol). The reaction was conducted in THF (5 mL) at 80° C. for 22 h. The title compound was isolated by flash column chromatography (silica gel, ethyl acetate:hexanes (v/v)=1:3) as purple mixture of two atropic isomers (0.039 g, 79%, α,α/α,β=50%/50%). ¹H NMR (300 MHz, CDCl₃): δ 9.48 (d, J=4.8 Hz, 2H), 9.46 (d, J=4.8 Hz, 2H), 9.27 (d, J=4.8 Hz, 2H), 9.25 (d, J=4.8 Hz, 2H), 9.08 (d, J=4.8 Hz, 2H), 9.04 (d, J=4.8 Hz, 2H), 9.02 (d, J=4.8 Hz, 2H), 9.00 (d, J=4.8 Hz, 2H), 8.24 (s, 2H), 8.12 (d, J=0.9 Hz, 2H), 8.07 (d, J=0.9 Hz, 2H), 7.93 (s, 2H), 7.86(s, 4H), 7.08 (m, 12H), 6.88 (m, 8H), 5.40 (m, 2H), 5.27 (m, 2H), 5.03 (q, J=9.0 Hz, 4H), 4.84 (q, J=7.8 Hz, 4H), 3.27 (m, 4H), 3.00 (m, 2H), 2.77 (m, 2H), 1.61 (s, 18H), 1.59 (s, 18H), 1.56 (s, 18H), 1.53 (s, 18H), −2.76 (s, 2H), −2.81 (s, 2H). ¹³C NMR (75 MHz, CDCl₃): δ 159.5, 159.4, 149.3, 149.1, 149.0, 140.2, 140.0, 135.1, 130.1, 130.1, 129.8, 129.8, 128.8, 128.7, 127.1, 127.0, 122.9, 121.5, 111.4, 111.2, 68.9, 68.8, 66.3, 40.5, 40.4, 35.1, 31.8, 31.7. UV-vis (CHCl₃, λₘₐₓ, nm): 419, 514, 549, 591, 646. HRMS-MALDI ([M+H]⁺): calcd for C₆₈H₇₃N₆O₄, 1037.5688; found: 1037.5677 with an isotope distribution pattern that is the same as calculated one.

### Example 79

### Cobalt Complex 18c

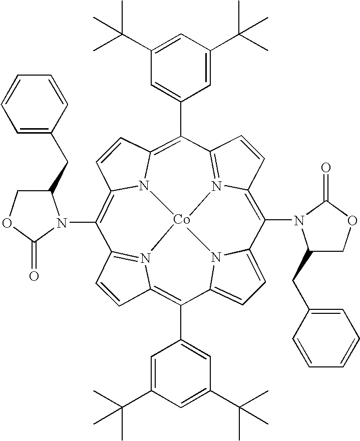

The general procedure was used for cobalt ion insertion. meso-Chiral porphyrin 17c (0.029 g), anhydrous COCl₂ (0.029 g), 2,6-lutidine (0.010 mL) and dry THF (4 mL) were heated at 70° C. under N₂ for 14 hours. The resulting mixture was cooled to room temperature, taken up in ethyl acetate and transferred to a separatory funnel. The mixture was washed with water 3 times and concentrated in vacuo. The title compound was obtained as a red solid (0.029 g, 95%). UV-vis (CHCl₃, λₘₐₓ, nm): 409, 530, 561. HRMS-MALDI ([M+H]⁺): calcd for C₆₈H₇₁CoN₆O₄, 1094.4863, found: 1094.4838 with an isotope distribution pattern that is the same as calculated one.

### Example 80

### General Considerations for the Synthesis of Chiralporphyrins via Palladium-Catalyzed C—O and C—N Bond Formation

All reactions were carried out under a nitrogen atmosphere in oven-dried glassware following standard Schlenk techniques. Toluene and THF were distilled under nitrogen from sodium benzophenone ketyl. All chiral building blocks and chemicals were purchased from Acros Organics or Aldrich Chemical Co. and used without further purification. Cesium carbonate was obtained as a gift from Chemetall Chemical Products, Inc. Palladium(II) acetate, tris(dibenzylideneacetone)dipalladium(0), bis(2-diphenylphosphinophenyl)ether (DPEphos) and 9,9-dimethyl-4,5-bis(diphenylphosphino)xanthene (Xantphos) were purchased from Strem Chemical Co. All ligands, palladium precursors and bases were stored in desiccators filled with anhydrous calcium sulfate, and weighed in the air. All bromoporphyrins were prepared according to the method described in the literature. See Lindsey et al., (1987) J. Org. Chem. 52: 827; DiMagno et al., (1993) J. Org. Chem. 58: 5983; Shi et al., (2000) J. Org. Chem. 65: 1650; Shanmugathasan et al., (2000) Porphyrins Phthalocyanines 4: 228. Porphyrin triflate was synthesized based on the procedure described below.

### Example 81

### General Procedure for the Synthesis of Porphyrin Triflates (22-1a, 22-1b, 22-1c and 22-2a, 22-2b, 22-2c)

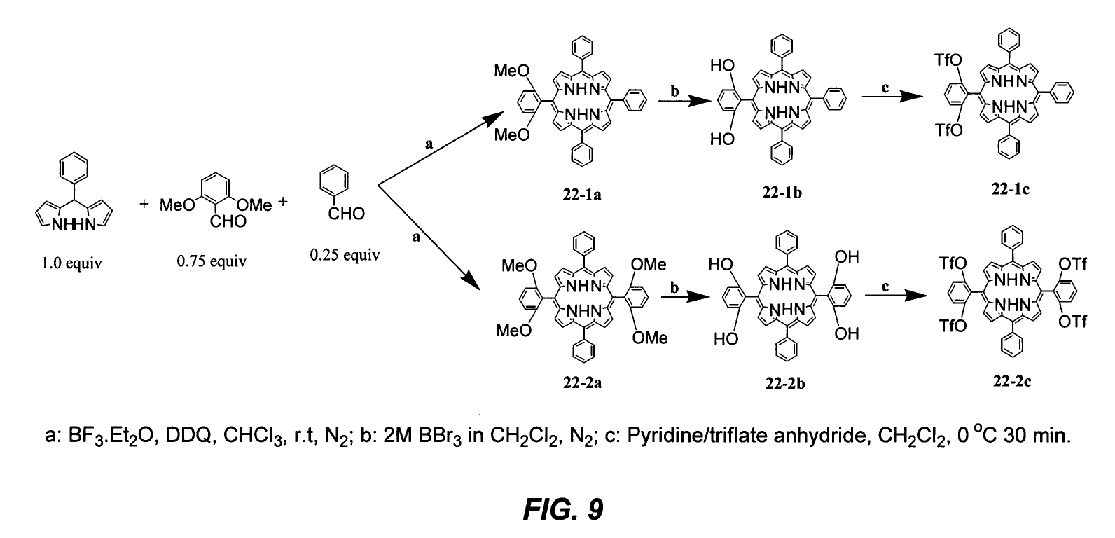

The general procedure for the synthesis of porphyrin triflates following Lindsey's method is provided in FIG. 9. See Lindsey et al., (1987) J. Org. Chem. 52: 827; DiMagno et al., (1993) J. Org. Chem. 58:5983; Shi et al., (2000) J. Org. Chem. 65: 1650; and Shanmugathasan et al., (2000) J. Porphyrins Phthalocyanines 4: 228.

### Example 82

### 5-(2,6-Dimethoxy-phenyl)-10,15,20-triphenylporphyrin (22-1a) and 5,15-bis(2,6-Dimethoxy-phenyl)-10,20-diphenylporphyrin (22-2a)

Compounds 22-1a and 22-2a were prepared based on Lindsey's method as provided in FIG. 9. Both 22-1a (12.2% yield) and 22-2a (12.2% yield) were obtained after flash chromatography (silica gel: methylene chloride). For 22-1a, ¹H NMR (300 MHz, CDCl₃) δ 8.80 (d, J=5.4 Hz, 8H), 8.20 (m, 6H), 7.72 (m, 10H), 7.01 (d, J=8.4 Hz, 2H), 3.51 (s, 6H), −2.71 (s, 2H); ¹³C NMR (CDCl₃, 75 MHz) δ 160.5, 142.3, 134.5, 130.2, 127.5, 126.6, 119.5, 104.1, 56.0. UV-vis (CH₂Cl₂, λₘₐₓ, nm): 417, 514, 546, 590, 645. HRMS-EI ([M]⁺): calcd for C₄₆H₃₄N₄O₂, 674.2682; found: 674.2690. For 22-2a, ¹H NMR (300 MHz, CDCl₃) δ 8.76 (s, 8H), 8.20 (m, 4H), 7.71 (m, 8H), 6.98 (dd, J=8.1, 1.8 Hz, 4H), 3.49 (s, 12H), −2.65 (s, 2H); ¹³C NMR (CDCl₃, 75 MHz) δ 160.5, 142.4, 134.5, 130.1, 127.4, 126.5, 119.9, 118.8, 112.1, 104.1, 56.1. UV-vis (CH₂Cl₂, λₘₐₓ, nm): 417, 513, 547, 590, 643. HRMS-EI ([M]⁺): calcd for C₄₈H₃₈N₄O₄, 734.2893; found: 734.2906.

### Example 83

### 5-(2,6-Dihydroxy-phenyl)-10,15,20-triphenylporphyrin (22-1b) and 5,15-bis(2,6-Dihydroxy-phenyl)-10,20-diphenylporphyrin (22-2b)

To a solution of 22-1a (or 22-2a) in anhydrous methylene chloride, BBr₃ was added dropwise slowly under N₂ until the concentration of BBr₃ reached 2M. The mixture was stirred under N₂ in room temperature for 4-5 h. A small amount of water was then added carefully. The product was extracted with ethyl acetate and washed with water to neutral. The ethyl acetate solution was concentrated to dry and the residue was recrystallized in hexanes to give pure title compound as a purple solid (90-95% yield, in general). For 22-1b, ¹H NMR (300 MHz, CDCl₃) δ 8.91 (m, 4H), 8.87 (m, 4H), 8.20 (m, 6H), 7.77 (m, 9H), 7.59 (t, J=8.1 Hz, 1H), 6.96 (d, J=8.1 Hz, 2H), 4.72 (s, 2H), −2.74 (s, 2H); ¹³C NMR (CDCl₃, 75 MHz) δ 156.2, 141.8, 141.5, 134.5, 130.9, 127.9, 126.8, 126.7, 122.0, 120.8, 115.5, 107.7, 103.2. UV-vis (CH₂Cl₂, λₘₐₓ, nm): 417, 514, 549, 588, 642. HRMS-EI ([M]⁺): calcd for C₄₄H₃₀N₄O₂, 646.2369; found: 646.2362. For 22-2b, ¹H NMR (300 MHz, CDCl₃) δ 8.95 (d, J=4.8 Hz, 4H), 8.92 (d, J=4.8 Hz, 4H), 8.17 (m, 4H), 7.78 (m, 6H), 7.61 (t, J=8.1 Hz, 2H), 6.97 (d, J=8.7 Hz, 4H), 4.66 (s, 4H), −2.77 (s, 2H); ¹³C NMR (CDCl₃, 75 MHz) δ 156.2, 141.0, 134.5, 131.2, 128.1, 126.9, 126.8, 121.3, 115.4, 107.9. UV-vis (CH₂Cl₂, λₘₐₓ, nm): 417, 513, 548, 588, 642. HRMS-EI ([M]⁺): calcd for C₄₄H₃₀N₄O₄, 678.2267; found: 678.2257.

To a solution of 22-1b (or 22-2b) in anhydrous methylene chloride at 0° C., pyridine (2.0 equiv per OH) and triflic anhydride (1.5 equiv per OH) was added dropwise successively under 0° C. The mixture was stirred in 0° C. for 0.5 h and continued at room temperature for another 4-5 h. The solution 20 was diluted with methylene chloride and washed with water to neutral. The methylene chloride solution was concentrated to dry and the residue was recrystallized in hexanes to give pure title compound as a purple solid (85-95% yield, in general). For 22-1c, ¹H NMR (300 MHz, CDCl₃) δ 8.89 (d, J=4.8 Hz, 2H), 8.85 (m, 4H), 8.64 (d, J=4.8 Hz, 2H), 8.19-8.25 (m, 6H), 8.02 (t, J=8.1 Hz, 1H), 7.86 (d, J=8.1 Hz, 2H), 7.77 (m, 9H), -2.72 (s, 2H); ¹³C NMR (CDCl₃, 75 MHz) δ 150.2, 141.9, 141.8, 134.7, 134.5, 131.3, 131.0, 127.9, 127.8, 126.7, 122.1, 1201.3, 120.8, 107.7, 103.2. ¹⁴F NMR (CDCl₃, 75 MHz) δ −74.9. UV-vis (CH₂Cl₂, λₘₐₓ, nm): 417, 514, 548, 589, 643. HRMS-EI ([M]⁺): calcd for C₄₆H₂₈F₆N₄O₆S₂, 910.1354; found: 910.1356. For 22-2c, ¹H NMR (300 MHz, CDCl₃) δ 8.88 (d, J=4.8 Hz, 4H), 8.65 (d, J=4.8 Hz, 4H), 8.25 (m, 4H), 8.04 (t, J=8.1 Hz, 2H), 7.87 (m, 4H), 7.77 (m, 6H), −2.79 (s, 2H); ¹³C NMR (CDCl₃, 75 MHz) δ 150.2, 141.5, 134.9, 134.5, 131.5, 130.8, 127.9, 126.7, 121.5, 121.4, 121.2, 119.6, 115.4, 104.2. ¹⁴F NMR (CDCl₃, 75 MHz) δ −74.9. UV-vis (CH₂Cl₂, λₘₐₓ, nm): 416, 512, 546, 589, 644. HRMS-EI ([M]⁺): calcd for C₄₈H₂₆F₁₂N₄O₁₂S₄, 1206.0238; found: 1206.0235.

### Example 84

### General Procedures for Synthesis of Chiralporphyrin via Palladium-Catalyzed C—O— and C—N Bond Formation (Examples 85-95)

An oven-dried Schlenk tube equipped with a stirring bar was degassed on vacuum line and purged with nitrogen. The tube was then charged with palladium precursor (5 mol % per Br or triflate), phosphine ligand (10 mol % per Br or triflate), chiral building block (2-4 equiv per Br or triflate), bromoporphyrin or porphyrin triflate (0.05 mmol) and base (2.0-4.0 equiv per Br). The tube was capped with a Teflon screwcap, evacuated and backfilled with nitrogen. After the Teflon screwcap was replaced with a rubber septum, solvent (5 mL) was added. The tube was purged with nitrogen (1-2 min) and the septum was then replaced with the Teflon screwcap and sealed. The reaction mixture was heated in an oil bath with stirring and monitored by TLC. After cooling to room temperature, the reaction mixture was diluted with ethyl acetate, washed with water (3×) and concentrated to dryness. The solid residue was purified by flash chromatography.

### Example 85

### (R)-(+)-4-Benzyl-3-(10′, 20′-diphenyl-porphyrin-5′-yl)-oxazolidin-2-one

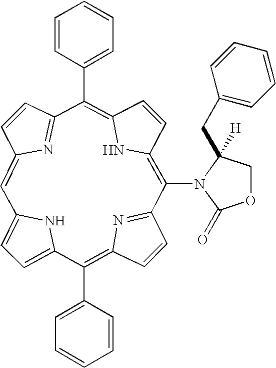

The general procedure was used to couple 5-bromo-10,20-diphenylporphyrin (27.1 mg, 0.05 mmol) with (R)-(+)-4-benzyl-2-oxazolidinone (35.4 mg, 0.2 mmol), using Pd₂(dba)₃ (2.3 mg, 0.0025 mmol) and Xantphos (5.78 mg, 0.01 mmol) in the presence of Cs₂CO₃ (37.6 mg, 0.1 mmol). The reaction was conducted in THF at 80° C. for 19 h. The title compound was isolated by flash chromatography (silica gel, ethyl acetate: hexanes (v/v)=4:6) as a purple-red solid (19 mg, 60%). ¹H NMR (300 MHz, CDCl₃ ) δ 10.26 (s, 1H), 9.48 (d, J=5.4 Hz, 1H), 9.35 (d, J=4.5 Hz, 1H), 9.32 (d, J=4.8 Hz, 1H), 9.28 (d, J=4.8 Hz, 1H), 9.01-9.06 (m, 3H), 8.98 (d, J=5.2 Hz, 1H), 8.29 (t, bro., 2H), 8.17 (d, J=5.1 Hz, 2H), 7.79 (s, bro., 6H), 7.06 (s, b, 3H), 6.82 (d, b, 2H), 5.30 (m, b, 1H), 5.00 (t, J=8.1 Hz, 1H), 4.84 (t, J=9.0 Hz, 1H), 3.21 (t, J=11.7 Hz, 2H), 3.21 (t, J=11.7 Hz, 2H), 2.80 (d, J=13.2 Hz, 1H), −3.03 (s, 2H); ¹³C NMR (CDCl₃, 75 MHz) δ 146.4, 141.2, 135.2, 134.7, 132.0, 131.0, 128.8, 128.7, 128.0, 127.0, 126.9, 106.7, 68.9, 66.5, 40.4. UV-vis (THF, λₘₐₓ, nm): 417, 476, 506, 539, 582, 638. HRMS-MALDI ([M+H]⁺): calcd for C₄₂H₃₂N₅O₂, 638.2551; found: 638.2544.

### Example 86

### 10-[17-(1,5-Dimethyl-hexyl)-10,13-dimethyl-hexadecahydro cyclopenta[a]phenanthren-3-yloxyl-5,15-diphenyl-porphprin

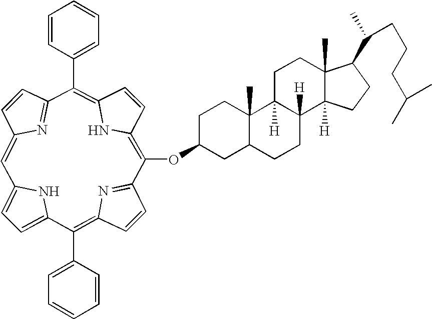

The general procedure was used to couple 5-bromo-10,20-diphenylporphyrin (27.1 mg, 0.05 mmol) with (+)-dihydrocholesterol (38.9 mg, 0.1 mmol), using Pd₂(dba)₃ (2.3 mg, 0.0025 mmol) and DPEphos (5.38 mg, 0.01 mmol) in the presence of Cs₂CO₃ (37.6 mg, 0.1 mmol). The reaction was conducted in THF at 100° C. for 24 h. The title compound was isolated by flash chromatography (silica gel, methylene chloride:hexanes (v/v)=8:2) as a purple-red solid (24 mg, 56%). ¹H NMR (300 MHz, CDCl₃) δ 10.01 (s, 1H), 9.56 (d, J=4.8 Hz, 1H), 9.21 (d, J=4.8 Hz, 1H), 8.94 (d, J=4.8 Hz, 1H), 8.88 (d, J=4.8 Hz, 1H), 8.23 (m, 4H), 7.76 (m, 6H), 5.05 (s, bro., 1H), 2.26 (m, bro., 2H), 2.08 (q, 1H), 1.69-1.88 (m, 5H), 0.61-1.52 (m, 44), −2.76 (s, 2H); ¹³C NMR (CDCl₃, 75 MHz) δ 146.4, 143.1, 141.6, 137.7, 134.7, 131.5, 131.1, 129.9, 127.8, 127.7, 126.9, 119.3, 103.4, 92.3, 56.3, 56.1, 54.2, 44.9, 42.5, 39.9, 39.5, 37.1, 36.1, 35.8, 35.7, 35.4, 31.9, 29.4, 28.7, 28.2, 27.9, 24.1, 23.8, 22.8, 22.5, 21.2, 18.6, 12.6, 12.0. UV-vis (CH₂Cl₂, λₘₐₓ, nm): 412, 511, 546, 587, 643. HRMS-MALDI ([M+H]⁺): calcd for C₅₉H₆₉N₄O, 849.5466; found: 849.5470.

### Example 87

### 2-(10,20-Diphenyl-porphyrin-5-ylamino)-3-phenyl-propionic acid methyl ester

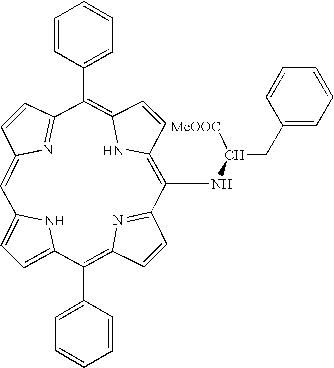

The general procedure was used to couple 5-bromo-10,20-diphenylporphyrin (27.1 mg, 0.05 mmol) with (L)-phenylalanine methyl ester hydrochloride (43 mg, 0.2 mmol), using Pd (OAc)₂ (1.12 mg, 0.005 mmol) and DPEphos (5.38 mg, 0.01 mmol) in the presence of Cs₂CO₃ (65.2 mg, 0.2 mmol). The reaction was conducted in THF at 100° C. for 24 h. The title compound was isolated by flash chromatography (silica gel, methylene chloride: hexanes (v/v)=8:2) as a purple-red solid (12 mg, 36%). ¹H NMR (300 MHz, CDCl₃) δ 9.79 (s, 1H), 9.14 (d, J=4.8 Hz, 1H), 9.09 (d, J=4.8 Hz, 1H), 8.84 (d, J=4.8 Hz, 1H), 9.28 (d, J=4.8 Hz, 1H), 9.01-9.06 (m, 3H), 8.98 (d, J=4.8 Hz, 1H), 8.67 (d, J=4.8 Hz, 2H), 8.18 (m,4H), 7.77 (m, 6H), 7.49 (dd, J=1.5, 8.4 Hz, 2H), 7.34-7.43 (m, 3H), 6.60 (d, bro., 1H), 5.52 (d, bro, 1H), 3.63 (t, J=6.9 Hz, 1H), 3.32 (s, 3H), 2.16 (s, 2H), −2.32 (s, 2H); ¹³C NMR (CDCl₃, 75 MHz) δ 174.2, 141.7, 136.8, 134.5, 131.8, 130.8, 129.8, 128.8, 128.5, 127.6, 127.2, 126.8, 126.1, 119.4, 102.2, 72.1, 51.7, 40.8. UV-vis (CH₂Cl₂, λₘₐₓ, nm): 420, 521, 562, 660. HRMS-MALDI ([M+H]⁺): calcd for C₄₂H₃₄N₅O₂, 640.2707; found: 640.2714.

### Example 88

### 3-(10,20-Diphenyl-porphyrin-5-yloxy)-13-methyl-6,7,8,9,11,12,13,14,15,16-decahydro-cyclopenta[a]phenanthren-17-one

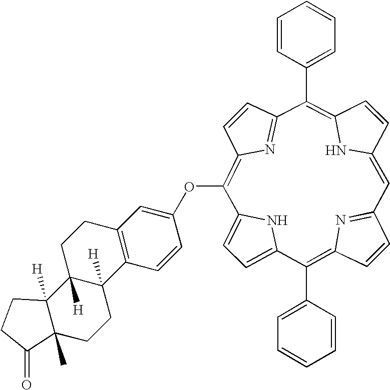

The general procedure was used to couple 5-bromo-10,20-diphenylporphyrin (27.1 mg, 0.05 mmol) with estrone (27 mg, 0.1 mmol), using Pd₂(dba)₃ (2.3 mg, 0.0025 mmol) and DPEphos (5.38 mg, 0.01 mmol) in the presence of Cs₂CO₃ (37.6 mg, 0.1 mmol). The reaction was conducted in THF at 100° C. for 24 h. The title compound was isolated by flash chromatography (silica gel, methylene chloride: hexanes (v/v)=8:2) as a purple-red solid (20 mg, 55%). ¹H NMR (300 MHz, CDCl₃) δ 10.13 (s, 1H), 9.39 (d, J=4.8 Hz, 2H), 9.29 (d, J=4.8 Hz, 4H), 8.98 (d, J=4.8 Hz, 4H), 8.86 (d, J=4.8 Hz, 4H), 8.22 (m, 4H), 7.75 (m, 6H), 7.14 (d, J=9.0 Hz, 1H), 6.88 (d, J=8.4 Hz, 1H), 6.64 (d, J=1.8 Hz, 1H), 1.86-2.62 (m, H from estrone), 1.26-1.52 (m, H from estrone), 0.85 (s, 3H), −2.77 (s, 2H). ¹³C NMR (75 MHz, CDCl₃): δ 141.2, 138.1, 134.7, 133.0, 131.5, 130.8, 127.9, 127.8, 126.9, 126.5, 120.2, 119.7, 116.6, 114.1, 104.6, 50.3, 47.9, 44.0, 38.1, 35.8, 31.5, 29.4, 26.3, 25.8, 21.5, 13.8. UV-vis (CH₂Cl₂, λₘₐₓ, nm): 413, 510, 544, 585, 641. HRMS-EI ([M]⁺): calcd for C₅₀H₄₂N₄O₂, 730.3308, found 730.3294.

### Example 89

### 2′-(10,20-Diphenyl-porphyrin-5-yloxy)-[1,1′]binaphthalenyl-2-ol

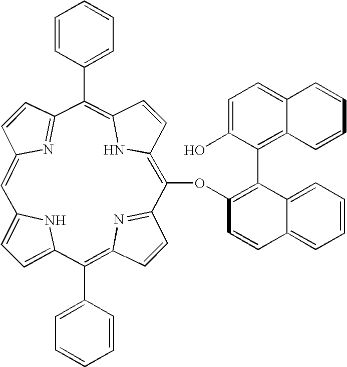

The general procedure was used to couple 5-bromo-10,20-diphenylporphyrin (54.2 mg, 0.1 mmol) with R-(+)-1,1′-Bi-2-naphthol (14.3 mg, 0.05 mmol), using Pd₂(dba)₃ (4.6 mg, 0.005 mmol) and DPEphos (10.8 mg, 0.02 mmol) in the presence of Cs₂CO₃ (37.6 mg, 0.1 mmol). The reaction was conducted in toluene at 100° C. for 24 h. The title compound was isolated by flash chromatography (silica gel, methylene chloride:hexanes (v/v)=8:2) as a purple-red solid (10 mg, 17%). ¹H NMR (300 MHz, CDCl₃) δ 10.12 (s, 1H), 9.27 (d, J=4.8 Hz, 4H), 8.97 (d, J=4.8 Hz, 4H), 8.78 (d, J=4.8 Hz, 4H), 8.17 (m, 4H), 7.99 (d, J=9.0 Hz, 1H), 7.94 (d, J=7.5 Hz, 1H), 7.86 (d, J=8.7 Hz, 1H), 7.74 (m, 6H), 7.40-7.66 (m, 8H), 6.42 (d, J=9.6 Hz, 1H), −2.83 (s, 2H). ¹³C NMR (75 MHz, CDCl₃): δ 141.2, 134.7, 131.6, 131.0, 130.8, 130.4, 129.5, 128.6, 128.3, 127.9, 127.8, 127.6, 126.9, 125.2, 124.9, 124.6, 123.7, 119.8, 117.8, 117.2, 104.7, 88.5. UV-vis (CH₂Cl₂, λₘₐₓ, nm): 414, 511, 545, 586, 641. HRMS-EI ([M]⁺): calcd for C₅₂H₃₄N₄O₂, 746.2682, found 746.2680.

### Example 90

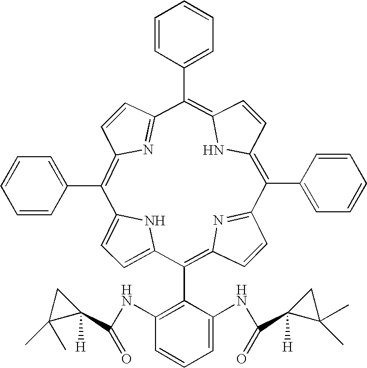

The general procedure was used to couple 3-trifluoromethanesulfonyloxy-2-(10,15,20-triphenyl-porphyrin-5-yl)-phenyl ester (45.5 mg, 0.05 mmol) with (s)-(+)-2,2-dimethylcyclopropanecarboxamide (45.2 mg, 0.4 mmol), using Pd₂(OAc)₂ (2.2 mg, 0.01 mmol) and Xantphos (11.6 mg, 0.02 mmol) in the presence of Cs₂CO₃ (130.3 mg, 0.4 mmol). The reaction was conducted in THF at 100° C. for 20 h. The title compound was isolated by flash chromatography (silica gel, methylene chloride: ethyl acetate (v/v)=9:1) as a purple solid (14 mg, 34%). ¹H NMR (300 MHz, CDCl₃) δ 8.89 (m, 6H), 8.81 (d, J=4.8 Hz, 2H), 8.45 (s, bro., 2H), 8.19 (d, J=6.6 Hz, 1H), 7.94 (d, J=7.5 Hz, 1H), 7.86 (d, J=8.7 Hz, 1H), 7.74 (m, 6H), 7.73-7.83 (m, 10H), 6.51 (s, 2H), 0.85 (s, 6H), 0.63 (s, 2H), 0.13 (s, 6H), 0.06 (s, 2H), −0.096 (s, 2H), −2.69 (s, 2H). ¹³C NMR (75 MHz, CDCl₃): δ 141.7, 141.4, 139.3, 134.5, 134.4, 130.2, 128.1, 126.9, 126.8, 122.0, 120.8, 29.0, 26.2, 22.3, 20.3, 18.3. UV-vis (CH₂Cl₂, λₘₐₓ, nm): 419, 515, 549, 590, 644. HRMS-EI ([M]⁺): calcd for C₅₆H₄₈N₆O₂, 837.3839, found 837.3860.

### Example 91

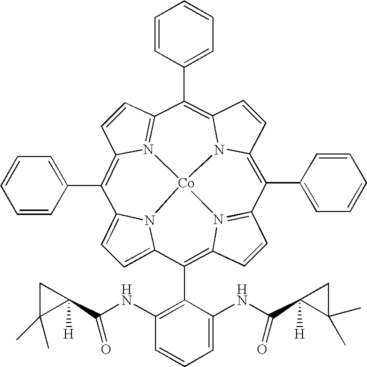

To the solution of free-base chiral porphyrin (Example 90, 21 mg, 0.0025 mmol) in 5 mL THF was added 2,6-lutidine (8.6 μL, 0.073 mmol) and CoCl₂ (26 mg, 0.2 mmol). The mixture was refluxed 16 h, concentrated to dry, re-dissolved in methylene chloride and washed with water (3×), the organic layer was concentrated and the product was obtained after recrystallization in hexanes (22 mg, 98%). Since the integration of ¹H NMR of cobalt complex was difficult to assign accurately, only the signals are provided herein. ¹H NMR (300 MHz, CDCl₃) δ 15.7 (s, bro.), 12.8 (s, bro.), 10.5 (s, bro.), 9.74 (m, bro.), 7.76 (s, bro.), 1.43 (s, bro.), 1.25 (s, bro.), 0.86 (s), 0.20 (s), −1.35 (s, bro.), −3.8 (s, bro.), −5.34 (s, bro.), −6.25 (s, bro). UV-vis (CH₂Cl₂, λₘₐₓ, nm): 411, 440, 530, 554, 600. HRMS-EI ([M]⁺): calcd for C₅₆H₄₆CoN₆O₂, 893.3014, found 893.3047.

### Example 92

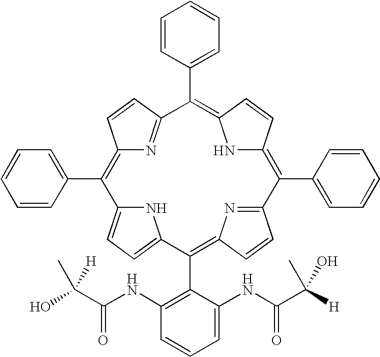

The general procedure was used to couple 3-trifluoromethanesulfonyloxy-2-(10,15,20-triphenyl-porphyrin-5-yl)-phenyl ester (45.5 mg, 0.05 mmol) with L-(R)-lactamide (36 mg, 0.4 mmol), using Pd₂(OAc)₂ (2.2 mg, 0.01 mmol) and Xantphos (11.6 mg, 0.02 mmol) in the presence of Cs₂CO₃ (130.3 mg, 0.4 mmol). The reaction was conducted in THF at 100° C. for 21 h. The title compound was isolated by flash chromatography (silica gel, methylene chloride: THF (v/v)=9:1) as a purple solid (14 mg, 34%). ¹H NMR (300 MHz, CDCl₃) δ 8.89 (m, 6H), 8.83 (m, 6H), 8.68 (d, J=4.8 Hz, 2H), 8.38 (d, J=8.7 Hz, 2H), 8.13 (m, 6H), 7.67-7.82 (m, 10H), 7.55 (s, 2H), 3.19 (q, J=6.0, 3.9 Hz, 2H), 0.40 (d, J=6.6 Hz 6H), 0.06 (s, 2H), −0.10 (s, 2H), −2.69 (s, 2H). ¹³C NMR (75 MHz, CDCl₃): δ 172.1, 141.3, 138.4, 134.6, 134.4, 130.4, 128.0, 126.9, 126.7, 120.9, 117.4, 67.7, 20.1. UV-vis (CH₂Cl₂, λₘₐₓ, nm): 418, 515, 550, 589, 644. HRMS-EI ([M]⁺): calcd for C₅₀H₄₀N₆O₄, 788.3111, found 788.3127.

### Example 93

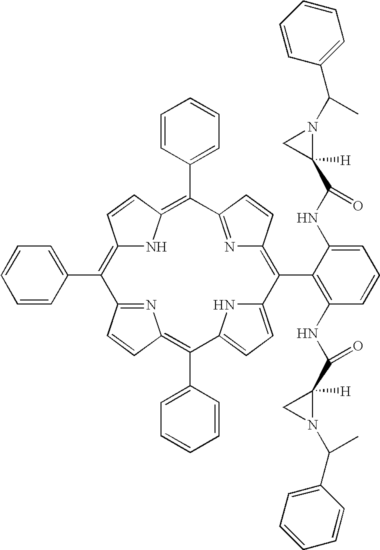

The general procedure was used to couple 3-trifluoromethanesu lfonyloxy-2-(10,15,20-triphenyl-porphyrin-5-yl)-phenyl ester (45.5 mg, 0.05 mmol) with 1-[1′-(R)-α-methylbenzyl]-aziridine-2(R)-carboxamide (64 mg, 0.4 mmol), using Pd₂(OAc)₂ (2.2 mg, 0.01 mmol) and Xantphos (11.6 mg, 0.02 mmol) in the presence of Cs₂CO₃ (130.3 mg, 0.4 mmol). The reaction was conducted in THF at 100° C. for 22 h. The title compound was isolated by flash chromatography (silica gel, methylene chloride: ethyl acetate (v/v)=9:1) as a purple solid (39 mg, 80%). ¹H NMR (300 MHz, CDCl₃) δ 8.84 (t, J=4.8 Hz, 4H), 8.78 (d, J=4.8 Hz, 2H), 8.73 (d, J=4.8 Hz, 2H), 8.62 (d, J=8.1 Hz, 2H), 8.18-8.23 (m, 6H), 7.72-7.88 (m, 9H), 7.62-7.68 (m, 1 H), 5.80 (t, J=7.5 Hz, 2H), 4.33 (t, J=7.5 Hz, 2H), 3.78 (d, J=7.2 Hz, 4H), 2.03 (s, 1H), 1.52 (dd, J=7.2, 3.0 Hz, 2H), 0.57 (d, J=6.3 Hz, 2H), 0.53 (dd, J=7.2, 3.0 Hz, 4H), −0.76 (d, J=5.7 Hz, 6H), −2.35 (s, 2H). ¹³C NMR (75 MHz, CDCl₃): δ 168.2, 142.0, 141.5, 140.9, 138.9, 134.3, 130.5, 127.8, 126.7, 125.9, 125.4, 124.2, 120.8, 120.7, 115.1, 106.2, 66.7, 39.4, 33.7, 20.4. UV-vis (CH₂Cl₂, λₘₐₓ, nm): 418, 513, 547, 588, 643. HRMS-EI ([M]⁺): calcd for C₆₆H₅₄N₈O₂, 990.4370, found 990.4412.

### Example 94

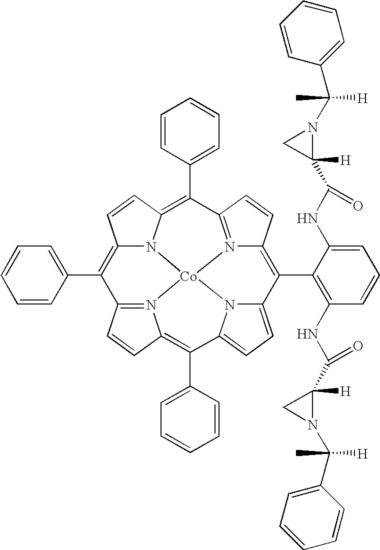

To the solution of free-base chiral porphyrin (Example 93, 12 mg, 0.0125 mmol) in 5 mL DMF was added Co(OAc)₂.4H₂O (25 mg, 0.1 mmol). The mixture was refluxed 2 h, concentrated to dryness, re-dissolved in ethyl acetate and washed with water (3×), the organic layer was concentrated and the product was obtained after recrystallization in hexanes (10.7 mg, 82%). Since the integration of ¹H NMR of cobalt complex was difficult to assign accurately, only the signals are provided herein. ¹H NMR (300 MHz, CDCl₃) δ 17.0 (s, bro.), 14.2 (s, bro.), 11.2 (s, bro.), 10.19-10.40 (m), 8.74 (m, bro.), 7.76 (s, bro.), 1.25 (s, bro.), −0.61 (s, bro.), −1.98 (s, bro.), −5.89 (s, bro.), −6.09 (s, bro), −12.1 (s, bro). UV-vis (CH₂Cl₂, λₘₐₓ, nm): 409, 526. HRMS-EI ([M]⁺): calcd for C₆₆H₅₂CoN₈O₂, 1047.3545, found 1047.3489.

### Example 95

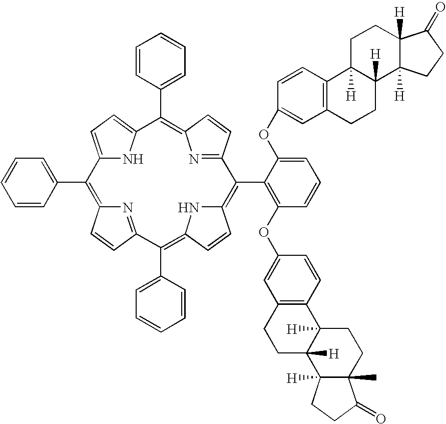

The general procedure was used to couple 3-trifluoromethanesulfonyloxy-2-(10,15,20-triphenyl-porphyrin-5-yl)-phenyl ester (45.5 mg, 0.05 mmol) with estrone (54 mg, 0.2 mmol), using Pd₂(dba)₃ (4.6 mg, 0.005 mmol) and Xantphos (5.8 mg, 0.01 mmol) in the presence of Cs₂CO₃ (65.2 mg, 0.2 mmol). The reaction was conducted in toluene at 80° C. for 24 h. The title compound was isolated by flash chromatography (silica gel, methylene chloride) as a purple solid (39 mg, 67%). ¹H NMR (300 MHz, CDCl₃) δ 8.85 (m, 8H), 8.28 (s, bro., 2H), 8.13 (m, 4H), 8.01 (s, bro, 1H), 7.62-7.82 (m, 11 H), 7.56 (m, 2H), 7.38 (m, 4H), 2.78 (m, 1H), 2.13 (s, 6H), 0.08-1.76 (m, H from estrone), −2.75 (s, 2H). ¹³C NMR (75 MHz, CDCl₃): δ 157.7, 149.6, 143.3, 141.8, 134.6, 134.1, 131.1, 130.5, 128.9, 128.3, 127.9, 127.1, 126.8, 126.7, 125.2, 122.5, 122.0, 121.1, 120.8, 118.2, 115.7, 113.1, 102.8, 49.3, 47.2, 43.3, 37.1, 35.0, 30.9, 30.4, 29.7, 29.2, 25.5, 25.1, 21.0, 13.3. UV-vis (CH₂Cl₂, λₘₐₓ, nm): 421, 474, 513, 547, 5988, 643. MS-El ([M−estrone−2]⁺): 880.4

### Example 96

### General Considerations for Bromoporphyrins as Versatile Synthons for Modular Construction of Chiral Porphyrins: Cobalt-Catalyzed Highly Enantioselective and Diastereoselective Cyclopropanation

All cross-coupling reactions were carried out under a nitrogen atmosphere in oven-dried glassware following standard Schlenk techniques. Tetrahydrofuran (THF) and toluene were distilled under nitrogen from sodium benzophenone ketyl. Chiral amides were purchased from Aldrich Chemical Co. and Acros Organics, used without further purification. Anhydrous cobalt(II) chloride, cobalt acetate tetrahydrate, palladium(II) acetate, and 9,9-dimethyl-4,5-bis(diphenylphosphino)xanthene (Xantphos) were purchased from Strem Chemical Co. Cesium carbonate was obtained as a gift from Chemetall Chemical Products, Inc.

2,6-Dibromobenzaldehyde was synthesized according to the literature (Luliński et al., (2003) J. Org. Chem. 68: 5384). ¹H NMR (300 MHz, CDCl₃): δ 10.24 (s, 1H), 7.63 (d, J=8.1 Hz, 2H), 7.20 (t, J=8.1 Hz, 1H).

2,6-Dibromo-4-trimethylsilylbenzaldehyde was synthesized according to the literature (Luliński et al., (2003) J. Org. Chem. 68: 5384). ¹H NMR (300 MHz, CDCl₃): δ 10.26 (s, 1H), 7.70 (s, 2H), 0.31 (s, 9H).

meso-(2,6-dibromophenyl)dipyrromethane was synthesized according to literature method (Naik et al., (2003) Tetrahedron 59: 2207) using Amberlyst 15 resin. ¹H NMR (300 MHz, CDCl₃): δ 8.29 (s, 2H), 7.56 (d, J=8.1 Hz, 2H), 6.95 (t, J=8.1 Hz, 1H), 6.72 (s, 2H), 6.53 (s, 1 H), 6.19 (m, 2H), 6.13 (s, 2H).

### Example 97

### General Procedures for Synthesis of Brominated Porphyrins

The brominated porphyrins were prepared according to the method described in literature. See Lindsey et al., (1989) J. Org. Chem. 54: 828; Lindsey (2000) In The Porphyrin Handbook; Kadish, K. M., Smith, K. M., Guilard, R., Eds., Academic Press: San Diego, Calif., Vol. 1; pp 45-118. A mixture of meso-(2,6-dibromophenyl)dipyrromethane (1 mmol), aldehyde (1 mmol), and molecular sieves (4A, 0.300 g) in chloroform (150 mL) was purged with nitrogen for 10 min. Boron trifluoride diethyl etherate (0.1 mL) was added dropwise via a syringe and the flask was wrapped with aluminum foil to shield it from light. The solution was stirred under a nitrogen atmosphere at room temperature for 3 h, and 2,3-dichloro-5,6-dicyano-1,4-benzoquinone (DDQ) (1.2 mmol) was added as powder at one time. After 30 min, 1 mL of triethylamine was added. The reaction solution was then directly poured on the top of a silica gel column that was packed with dichloromethane. The column was eluted with dichloromethane. The fractions containing product were collected and concentrated on a rotary evaporator. The residue was washed several times with hexanes to afford the pure compound.

### Example 98

### 5,15-Bis(2,6-dibromophenyl)-10,20-diphenylporphyrin 19a

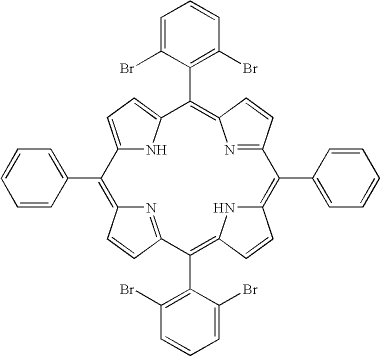

Purple solid. Yield: 41%. ¹H NMR (300 MHz, CDCl₃): δ 8.84 (d, J=4.8 Hz, 4H), 8.63 (d, J=4.8 Hz, 4H), 8.22 (m, 4H), 8.01 (d, J=8.1 Hz, 4H), 7.73 (m, 6H), 7.52 (t, J=8.1 Hz, 2H), −2.61 (s, 2H). UV-vis (CH₂Cl₂), λₘₐₓ nm (log ε): 419(5.65), 515(4.31), 548(3.76), 590(3.81), 646(3.41). HRMS-MALDI ([M+H]⁺): calcd for C₄₄H₂₇Br₄N₄ 926.8964; found: 926.8992 with an isotope distribution pattern that is the same as the calculated one.

### Example 99

### 5,15-Bis(2,6-dibromophenyl)-10,20-bis[4-(tert-butyl)phenyllporphyrin 19b

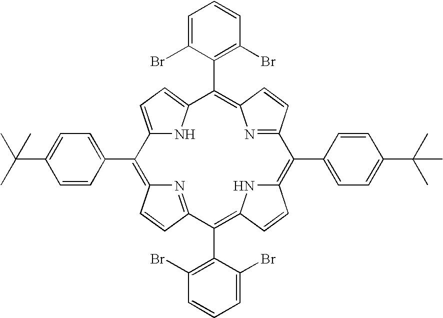

Purple solid. Yield: 61%. ¹H NMR (300 MHz, CDCl₃): δ 8.88 (d, J=4.8 Hz, 4H), 8.61 (d, J=4.8 Hz, 4H), 8.14 (d, J=8.1 Hz, 4H), 8.01 (d, J=8.1 Hz, 4H), 7.73 (d, J=8.1 Hz, 4H), 7.52 (t, J=8.1 Hz, 2H), 1.52 (s, 18H), −2.57 (s, 2H). UV-vis (CH₂Cl₂), λₘₐₓ nm (log ε): 421(5.68), 516(4.31), 551(3.82), 592(3.81), 647(3.47). HRMS-MALDI ([M+H]⁺): calcd for C₅₂H₄₃Br₄N₄ 1039.0216; found: 1039.0236 with an isotope distribution pattern that is the same as the calculated one.

### Examnle 100

### 5,15-Bis(2,6-dibromophenyl)-10,20-bis(4-trifluoromethvlphenyl)porphyrin 19c

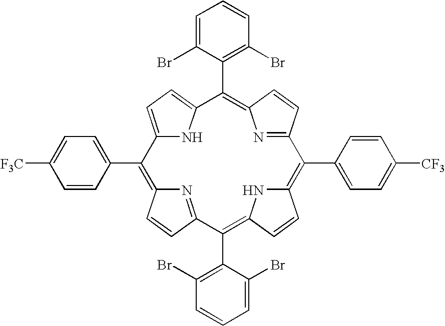

Purple solid. Yield: 45%. ¹H NMR (300 MHz, CDCl₃): δ 8.77 (d, J=4.8 Hz, 4H), 8.66 (d, J=4.8 Hz, 4H), 8.36 (d, J=8.1 Hz, 4H), 8.03 (d, J=8.1 Hz, 4H), 8.01 (d, J=8.1 Hz, 4H), 7.54 (t, J=8.1 Hz, 2H), −2.63 (s, 2H). UV-vis (CH₂Cl₂), λₘₐₓ nm (log ε): 419(5.65), 514(4.31), 547(3.67), 589(3.82), 644(3.20). HRMS-EI ([M]⁺): calcd for C₄₆H₂₄Br₄F₆N₄ 1061.8639; found: 1061.8623 with an isotope distribution pattern that is the same as the calculated one.

### Example 101

### 5,15-Bis(2,6-dibromorhenyl)-10,20-bis(2,3,4,5,6-pentafluorolhenyl)porphyrin 19d

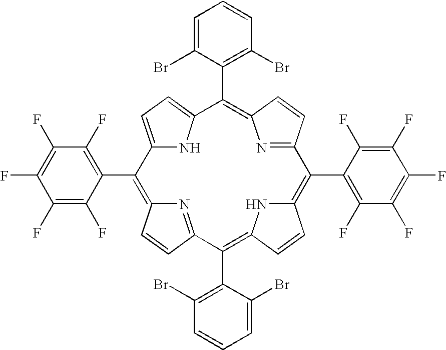

Purple solid. Yield: 19%. ¹H NMR (300 MHz, CDCl₃): δ 8.78 (d, J=4.8 Hz, 4H), 8.73 (d, J=4.8 Hz, 4H), 8.04 (d, J=8.1 Hz, 4H), 7.57 (t, J=8.1 Hz, 2H), −2.68 (s, 2H). UV-vis (CH₂Cl₂), λₘₐₓ nm (log ε): 416(5.62), 511(4.40), 547(3.72), 588(4.09), 643(3.35). HRMS-MALDI ([M+H]⁺): calcd for C₄₄H₁₇Br₄F₁₀N₄ 1106.8022; found: 1106.8009 with an isotope distribution pattern that is the same as the calculated one.

### Example 102

### 5,15-Bis(2,6-dibromophenyl)-10,20-bis(4-acetylphenyl)porphyrin 19e

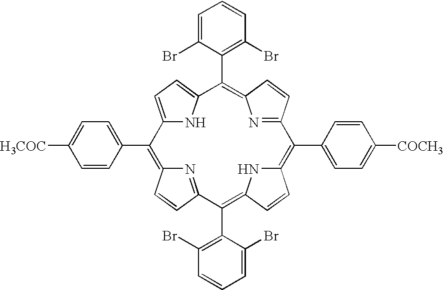

Purple solid. Yield: 45%. ¹H NMR (300 MHz, CDCl₃): δ 8.79 (d, J=4.8 Hz, 4H), 8.66 (d, J=4.8 Hz, 4H), 8.35 (s, 8H), 8.02 (d, J=8.1 Hz, 4H), 7.54 (t, J=8.1 Hz, 2H),2.89 (s, 6H), −2.61 (s, 2H). UV-vis (CH₂Cl₂), λₘₐₓ nm (log ε): 421(5.59), 516(4.37), 549(3.79), 591(3.87), 646(3.44). HRMS-MALDI ([M+H]⁺): calcd for C₄₈H₃₁Br₄N₄O₂ 1010.9175 found: 1010.9171 with an isotope distribution pattern that is the same as the calculated one.

### Example 103

### 5,15-Bis(2,6-dibromophenyl)-10,20-dimesitylporphyrin 19f

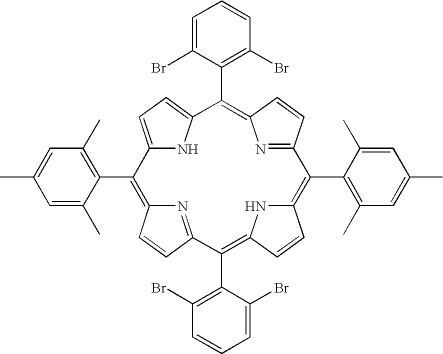

Purple solid. Yield: 41%. ¹H NMR (300 MHz, CDCl₃): δ 8.66 (d, J=4.8 Hz, 4H), 8.53 (d, J=4.8 Hz, 4H), 8.00 (d, J=8.1 Hz, 4H), 7.50 (t, J=8.1 Hz, 2H), 7.25 (s, 4H), 2.60 (s, 6H), 1.84 (s, 12H), −2.49 (s, 2H). UV-vis (CH₂Cl₂), λₘₐₓ nm (log ε): 419(5.75), 515(4.39), 547(3.79), 591(3.90), 647(3.46). HRMS-MALDI ([M+H]⁺): calcd for C₅₀H₃₉Br₄N₄ 1010.9903; found: 1010.9907 with an isotope distribution pattern that is the same as the calculated one.

### Example 104

### 5,15-Bis(2,6-dibromophenyl)-10,20-bis(2,6-dimethoxvphenyl)porphyrin 19g

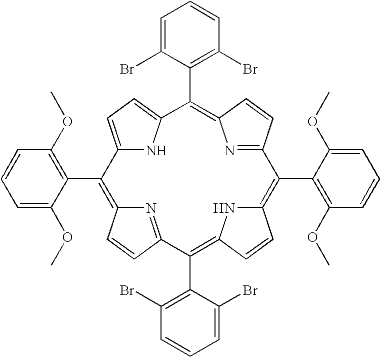

Purple solid. Yield: 69%. ¹H NMR (300 MHz, CDCl₃): δ 8.73 (d, J=4.8 Hz, 4H), 8.53 (d, J=4.8 Hz, 4H), 7.98 (d, J=8.1 Hz, 4H), 7.69 (t, J=8.1 Hz, 2H), 7.48 (t, J=8.1 Hz, 2H), 6.97 (d, J=8.7 Hz, 4H), 3.51 (s, 12H), −2.49 (s, 2H). UV-vis (CH₂Cl₂), λₘₐₓ nm (log ε): 420(5.79), 515(4.45), 545(3.72), 590(3.95), 644(3.26). HRMS-MALDI ([M+H]⁺): calcd for C₄₈H₃₅Br₄N₄O₄ 1046.9386; found: 1046.9434 with an isotope distribution pattern that is the same as the calculated one.

### Example 105

### 5,15-Bis(2,6-dibromophenyl)-10,20-bis(3,5-dimethoxyphenyl)porphyrin 19h

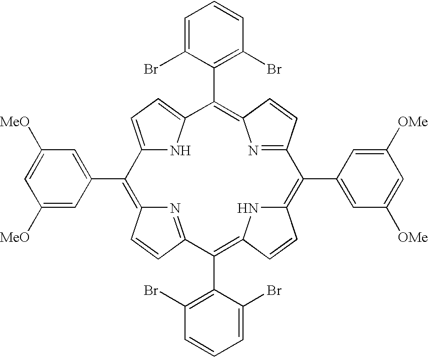

Purple solid. Yield: 55%. ¹H NMR (300 MHz, CDCl₃): δ 8.92 (d, J=4.8 Hz, 4H), 8.61 (d, J=4.8 Hz, 4H), 8.01 (d, J=8.1 Hz, 4H), 7.52 (t, J=8.1 Hz, 2H), 7.40 (t, J=2.4 Hz, 4H), 6.87 (d, J=2.4 Hz, 2H), 3.94 (s, 12H), −2.65 (s, 2H). UV-vis (CH₂Cl₂), λₘₐₓ nm (log ε): 421(5.79), 515(4.44), 548(3.80), 590(3.96), 645(3.50). HRMS-MALDI ([M+H]⁺): calcd for C₄₈H₃₅Br₄N₄O₄ 1046.9386; found: 1046.9423 with an isotope distribution pattern that is the same as the calculated one.

### Example 106

### 5,15-Bis(2,6-dibromophenyl)-10,20-bis[3,5-di(tert-butyl)phenyl]porphyrin 19i

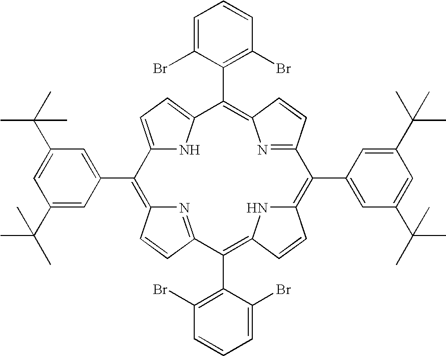

Purple solid. Yield: 69%. ¹H NMR (300 MHz, CDCl₃): δ 8.90 (d, J=4.8 Hz, 4H), 8.65 (d, J=4.8 Hz, 4H), 8.11 (d, J=1.5 Hz, 4H), 8.01 (d, J=8.1 Hz, 4H), 7.79 (t, J=1.8 Hz, 2H), 7.51 (t, J=8.1 Hz, 2H), 1.53 (s, 36H), −2.52 (s, 2H). ¹³HC NMR (75 MHz, CDCl₃): δ 148.8, 143.6, 140.6, 132.3, 131.4, 131.0, 129.9, 129.3, 128.5, 121.6, 121.1, 118.1, 35.1, 31.7. UV-vis (CH₂Cl₂), λₘₐₓ nm (log ε): 421(5.69), 516(4.30), 551(3.79), 592(3.79), 648(3.50). HRMS-MALDI ([M+H]⁺): calcd for C₆₀H₅₉Br₄N₄ 1151.1468; found: 1151.1459 with an isotope distribution pattern that is the same as the calculated one.

### Example 107

### 5,15-Bis(2,6-dibromophenyl)-10,20-bisheptylporphyrin 19j

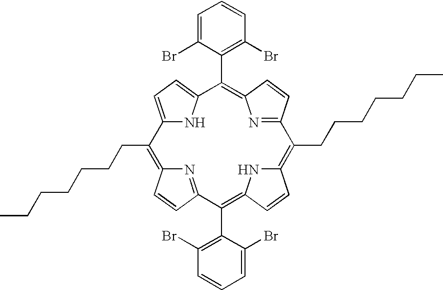

Purple solid. Yield: 53%. ¹H NMR (300 MHz, CDCl₃): δ 9.40 (d, J=4.8 Hz, 4H), 8.66 (d, J=4.8 Hz, 4H), 8.03 (d, J=8.1 Hz, 4H), 7.54 (t, J=8.1 Hz, 2H), 4.89. t, J=7.8 Hz, 4H), 2.54 (m, 4H), 1.81(m,4H), 1.52(m, 4H), 1.33(m, 8H), 0.90 (m, 6H), −2.45 (s, 2H). UV-vis (CH₂Cl₂), λₘₐₓ nm (log ε): 419(5.61), 518(4.31), 553(3.92), 596(3.80), 654(3.74). HRMS-MALDI ([M+H]⁺): calcd for C₄₆H₄₇Br₄N₄, 971.0529; found: 971.0510 with an isotope distribution pattern that is the same as the calculated one.

### Example 108

### 5,15-Bis(2,6-dibromophenyl)porphyrin 19k

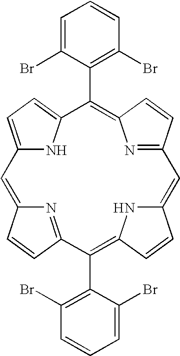

A mixture of dipyrromethane (0.146 g, 1 mmol), 2,6-dibromobenzaldehyde (0.264 g, 1 mmol) and molecular sieves (4A, 1.0 g) in chloroform (100 mL) was purged with nitrogen for 10 min. Boron trifluoride diethyl etherate (0.1 mL) was added dropwise via a syringe and the flask was wrapped with aluminum foil to shield it from light. The solution was stirred under a nitrogen atmosphere at room temperature for 16 h, and 2,3-dichloro-5,6-dicyano-1,4-benzoquinone (DDQ) (0.287 g, 1.2 mmol) was added as powder at one time. After 30 min, 1 mL of triethylamine was added. The reaction solution was then directly poured on the top of a silica gel column that was packed with dichloromethane. The column was eluted with dichloromethane. The fractions containing product were collected and concentrated on a rotary evaporator. The residue was washed several times with hexanes to afford the title compound as a purple solid. Yield: 0.055 g (14%). ¹H NMR (300 MHz, CDCl₃): δ 10.26 (s, 2H), 9.36 (d, J=4.8 Hz, 4H), 8.84 (d, J=4.8 Hz, 4H), 8.01 (d, J=8.1 Hz, 4H), 7.57 (t, J=8.1 Hz, 2H), −3.07 (s, 2H). UV-vis (CH₂Cl₂), λₘₐₓ nm (log ε): 407(5.73), 502(4.44), 534(3.95), 576(3.99), 630(3.53). HRMS-MALDI ([M+H]⁺): calcd for C₃₂H₁₉Br₄N₄ 774.8338; found: 774.8476 with an isotope distribution pattern that is the same as the calculated one.

### Example 109

### General Procedures for Amidation of Bromoporphyrin

The general procedures for amidation of bromoporphyrin follow those described in Gao et al., (2004) Org. Lett. 6: 1837. The bromoporphyrin, chiral amide, Pd(OAc)₂, Xantphos, and Cs₂CO₃ were placed in an oven-dried, resealable Schlenk tube. The tube was capped with a Teflon screwcap, evacuated, and backfilled with nitrogen. The screwcap was replaced with a rubber septum, and THF was added via syringe. The tube was purged with nitrogen for 2 min, and then the septum was replaced with the Teflon screwcap. The tube was sealed, and its contents were heated with stirring. The resulting mixture was cooled to room temperature, taken up in ethyl acetate and concentrated in vacuo. The crude product was then purified by flash chromatography.

### Example 110

### Porphyrin 20a (Table 4, entry 1)

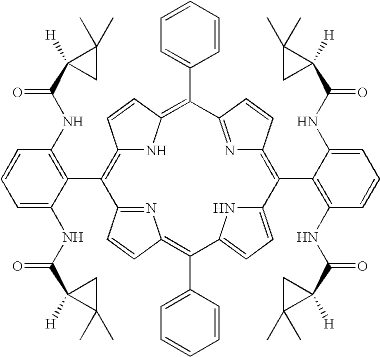

The general procedure was used to couple 5,15-bis(2,6-dibromophenyl)-10,20-diphenylporphyrin (0.093 g, 0.1 mmol) with (S)-(+)-2,2-dimethyl cyclopropanecarboxamide (0.362 g, 3.2 mmol), using Pd(OAc)₂ (0.009 g, 0.04 mmol), Xantphos (0.046 g, 0.08 mmol), and Cs₂CO₃ (0.522 g, 1.6 mmol). The reaction was conducted in THF (6 mL) at 100° C. for 60 h. The pure compound was isolated by flash column chromatography (silica gel, ethyl acetate:hexanes (v/v)=1:2) as purple solids (0.083 g, 78%). ¹H NMR (300 MHz, CDCl₃): δ 8.95 (d, J=4.8 Hz, 4H), 8.87 (d, J=4.8 Hz, 4H), 8.44 (broad, 4H), 8.18 (d, J=6.0 Hz, 4H), 7.83 (m, 8H), 6.45 (broad, 4H), 0.87 (s, 12H), 0.69 (broad, 4H), −0.08-0.18 (m, 20H), −2.65 (s, 2H). ¹³C NMR (75 MHz, CDCl₃): δ 169.6, 140.7, 139.3, 134.4, 133.6, 130.4, 128.5, 127.1, 121.4, 117.8, 28.9, 26.3, 22.4, 20.4, 18.2. UV-vis (CH₂Cl₂), λₘₐₓ nm (log ε): 420(5.33), 516(4.09), 549(3.62), 589(3.59), 644(3.33). HRMS-EI ([M]⁺): calcd for C₆₈H₆₆N₈O₄ 1058.5207, found 1058.5184 with an isotope distribution pattern that is the same as the calculated one.

### Example 111

### Porphyrin 20b (Table 4, entry 2)

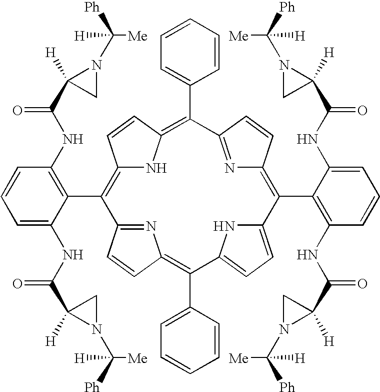

The general procedure was used to couple 5,15-bis(2,6-dibromophenyl)-10,20-diphenylporphyrin (0.023 g, 0.025 mmol) with 1-[1′(R)-a-methyl benzyl]-aziridine-2(R)-carboxamide (0.076 g, 0.4 mmol), using molecular sieves (4A, 0.05 g), Pd(OAC)₂ (0.002 g, 0.01 mmol), Xantphos (0.012 g, 0.02 mmol), and CS₂CO₃ (0.130 g, 0.4 mmol). The reaction was conducted in THF (2 mL) at 100° C, for 60 h. The pure compound was isolated by flash column chromatography (silica gel, ethyl acetate:hexanes (v/v)=1:1) as purple solids (0.022 g, 64%). ¹H NMR (300 MHz, CDCl₃): δ 8.89 (m, 8H), 8.58 (m, 8H), 7.93 (t, J=8.7 Hz, 2H), 7.55 (t, J=7.2 Hz, 2H), 7.34 (t, J=7.8 Hz, 4H), 6.75 (d, J=7.2 Hz, 4H), 5.87 (t, J=7.8 Hz, 4H), 4.59 (m, 16H), 1.63 (m, 8H), 0.53 (d, J=6.3 Hz, 12H), 0.27 (m, 8H), −2.08 (s, 2H). ¹³C NMR (75 MHz, CDCl₃): δ 168.7, 141.5, 140.6, 139.0, 133.2, 130.9, 127.7, 126.1, 125.3, 123.8, 121.8, 120.3, 116.0, 108.7, 66.6, 39.4, 34.0, 23.5. UV-vis (CH₂Cl₂), λₘₐₓ nm (log ε): 421(5.60), 514(4.35), 548(3.92), 590(3.84), 646(3.73). HRMS-MALDI ([M]⁺): calcd for C₈₈H₇₉N₁₂O₄ 1367.6342, found 1367.6343 with an isotope distribution pattern that is the same as the calculated one.

### Example 112

### Porphyrin 20c (Table 4, entry 3)

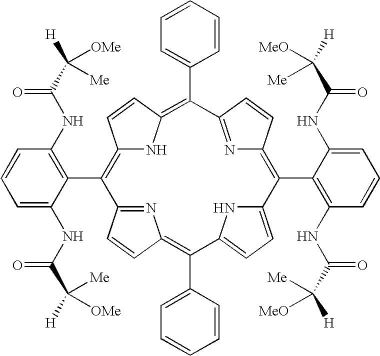

The general procedure was used to couple 5,15-bis(2,6-dibromophenyl)-10,20-diphenylporphyrin (0.046 g, 0.05 mmol) with (R)-(+)-2-methoxy propionamide (0.082 g, 0.8 mmol), using molecular sieves (4A, 0.100 g), Pd(OAc)₂ (0.004 g, 0.02 mmol), Xantphos (0.023 g, 0.04 mmol), and Cs₂CO₃ (0.261 g, 0.8 mmol). The reaction was conducted in THF (4 mL) at 100° C. for 64 h. The pure compound was isolated by flash column chromatography (silica gel, ethyl acetate:hexanes (v/v)=1:1) as purple solids (0.038,g, 75%). ¹H NMR (300 MHz, CDCl₃): δ 8.86 (d, J=4.8 Hz, 4H), 8.79 (d, J=4.8 Hz, 4H), 8.53 (d, J=8.1 Hz, 4H), 8.09 (d, J=7.2 Hz, 4H), 7.88 (t, J=8.1 Hz, 2H), 7.77 (m, 1OH), 3.03 (q, J=6.6 Hz, 4H), 1.22 (s, 12H), 0.62 (d, J=6.6 Hz, 12H), −2.56 (s, 2H). ¹³C NMR (75 MHz, CDCl₃): δ171.1, 140.9, 138.4, 134.4, 130.7, 128.3, 127.1, 122.4, 121.2, 117.2, 108.2, 78.0, 55.8, 17.7. UV-vis (CH₂Cl₂), λₘₐₓ nm (log ε): 419(5.51), 514(4.24), 547(3.76), 589(3.72), 644(3.49). HRMS-MALDI ([M]⁺): calcd for C₆₀H₅₉N₈O₈ 1019.4450, found 1019.4462 with an isotope distribution pattern that is the same as the calculated one.

### Example 113

### Porphyrin 20d (Table 4, entry 4)

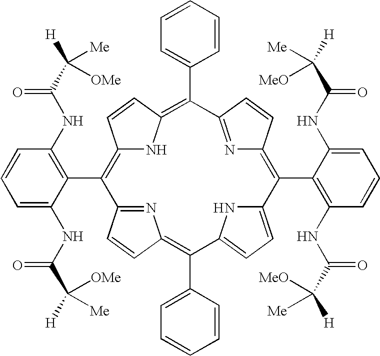

The general procedure was used to couple 5,15-bis(2,6-dibromophenyl)-10,20-diphenylporphyrin (0.046 g, 0.05 mmol) with (S)-(−)-2-methoxy propionamide (0.082 g, 0.8 mmol), using molecular sieves (4A, 0.1 g), Pd(OAc)₂ (0.004 g, 0.02 mmol), Xantphos (0.023 g, 0.04 mmol), and Cs₂CO₃ (0.261 g, 0.8 mmol). The reaction was conducted in THF (4 mL) at 80° C. for 62 h. The pure compound was isolated by flash column chromatography (silica gel, ethyl acetate:hexanes (v/v)=1:1) as purple solids (0.036 g, 71%). ¹H NMR (300 MHz, CDCl₃): δ 8.86 (d, J=4.8 Hz, 4H), 8.79 (d, J=4.8 Hz, 4H), 8.53 (d, J=8.1 Hz, 4H), 8.09 (d, J=6.9 Hz, 4H), 7.88 (t, J=8.1 Hz, 2H), 7.77 (m, 1OH), 3.03 (q, J=7.2 Hz, 4H), 1.22 (s, 12H), 0.62 (d, J=6.6 Hz, 12H), −2.57 (s, 2H). ¹³C NMR (75 MHz, CDCl₃): δ171.1, 140.9, 138.4, 134.4, 130.7, 128.3, 127.1, 122.3, 121.2, 117.1, 108.1, 78.0, 55.8, 17.7. UV-vis (CH₂Cl₂), λₘₐₓ nm (log ε): 419(5.50), 514(4.23), 547(3.74), 587(3.70), 644(3.44). HRMS-MALDI ([M]J): calcd for C₆₀H₅₉N₈O₈ 1019.4450, found 1019.4497 with an isotope distribution pattern that is the same as the calculated one.

### Example 114

### Porphyrin 20e (Table 4, entry 5)

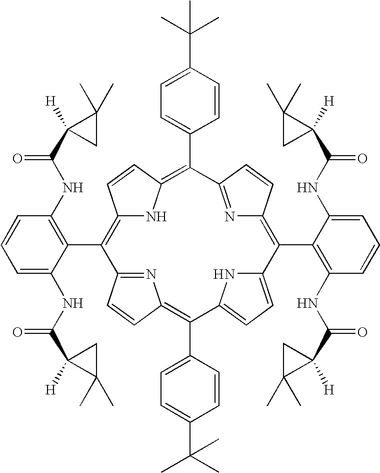

The general procedure was used to couple 5,15-bis(2,6-dibromophenyl)-10,20-bis[4-(tert-butyl)phenyl]porphyrin (0.078 g, 0.075 mmol) with (S)-(+)-2,2-dimethylcyclopropanecarboxamide (0.136 g, 1.2 mmol), using Pd(OAc)₂ (0.007 g, 0.03 mmol), Xantphos (0.035 g, 0.06 mmol), and Cs₂CO₃ (0.391 g, 1.2 mmol). The reaction was conducted in THF (6 mL) at 100° C. for 40 h. The pure compound was isolated by flash column chromatography (silica gel, ethyl acetate:hexanes (v/v)=1:4) as purple solids (0.076 g, 86%). ¹H NMR (300 MHz, CDCl₃): δ 8.99 (d, J=4.8 Hz, 4H), 8.85 (d, J=4.8 Hz, 4H), 8.45 (broad, 4H), 8.10 (d, J=8.1 Hz, 4H), 7.81 (m, 6H), 6.46 (broad, 4H), 1.61 (s, 18H), 0.87 (s, 12H), 0.67 (broad, 4H), 0.11-0.17 (m, 20H), −2.63 (s, 2H). ¹³C NMR (75 MHz, CDCl₃): δ 169.7, 151.4, 139.3, 137.7, 134.0, 130.3, 124.1, 121.68, 117.5, 35.0, 31.6, 29.1, 26.3, 22.4, 20.4, 18.2. UV-vis (CH₂Cl₂), λₘₐₓ nm (log ε): 421(5.38), 516(4.10), 552(3.69), 591(3.59), 648(3.49). HRMS-EI ([M]⁺): calcd for C₇₆H₈₂N₈O₄ 1170.6459, found 1170.6451 with an isotope distribution pattern that is the same as the calculated one.

### Example 115

### Porphyrin 20f (Table 4 entry 6)

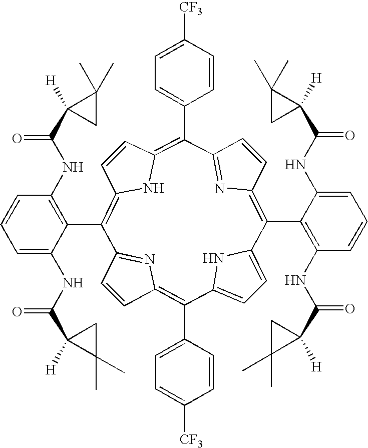

The general procedure was used to couple 5,15-bis(2,6-dibromophenyl)-10,20-bis(4-trifluoromethylphenyl)porphyrin (0.053 g, 0.05 mmol) with (S)-(+)-2,2-dimethylcyclopropanecarboxamide (0.184 g, 1.6 mmol), using Pd(OAc)₂ (0.004 g, 0.02 mmol), Xantphos (0.023 g, 0.04 mmol), and Cs₂CO₃ (0.261 g, 0.8 mmol). The reaction was conducted in THF (4 mL) at 100° C. for 60 h. The pure compound was isolated by flash column chromatography (silica gel, ethyl acetate:hexanes (v/v)=1:2) as purple solids (0.046 g, 77%). ¹H NMR (300 MHz, CDCl₃): δ 8.89 (m, 8H), 8.41 (broad, 4H), 8.31 (d, J=8.1 Hz, 4H), 8.08 (d, J=8.1 Hz, 4H), 7.83 (t, J=8.1 Hz, 2H), 6.41 (broad, 4H), 0.85 (s, 12H), 0.69 (broad, 4H), −0.07-0.19 (m, 20H), −2.68 (s, 2H). ¹³C NMR (75 MHz, CDCl₃): δ 189.8, 170.2, 144.4, 139.3, 134.5, 130.6, 124.2, 119.6, 29.0, 26.3, 22.5, 20.4, 18.2. UV-vis (CH₂Cl₂), λₘₐₓ nm (log ε): 420(5.53), 514(4.33), 547(3.77), 588(3.82), 643(3.43). HRMS-MALDI ([M+H]⁺): calcd for C₇₀H₆₅F₆N₈O₈ 1195.5027, found 1195.5085 with an isotope distribution pattern that is the same as the calculated one.

### Example 116

### Porphyrin 20g (Table 4, entry 7)

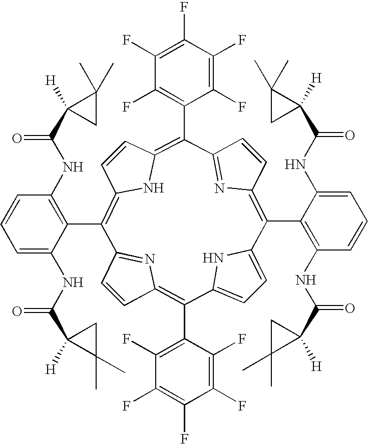

The general procedure was used to couple 5,15-bis(2,6-dibromophenyl)-10,20-bis(2,3,4,5,6-pentafluorophenyl)porphyrin (0.028 g, 0.025 mmol) with (S)-(+)-2,2-dimethylcyclopropanecarboxamide (0.091 g, 0.8 mmol), using Pd(OAc)₂ (0.002 g, 0.01 mmol), Xantphos (0.012 g, 0.02 mmol), and CS₂CO₃ (0.130 g, 0.4 mmol). The reaction was conducted in THF (2 mL) at 100° C. for 60 h. The pure compound was isolated by flash column chromatography (silica gel, ethyl acetate:hexanes (v/v)=1:4) as purple solids (0.015 g, 46%). ¹H NMR (300 MHz, CDCl₃): δ 8.99 (d, J=4.8 Hz, 4H), 8.91 (d, J=4.8 Hz, 4H), 8.36 (broad, 4H), 7.84 (t, J=8.1 Hz, 2H), 6.37 (broad, 4H), 0.81 (s, 12H), 0.69 (broad, 4H), −0.02-0.14 (m, 20H), −2.71 (s, 2 H). ¹³C NMR (75 MHz, CDCl₃): δ 169.8, 139.1, 130.8, 118.7, 110.8, 103.5, 28.8, 26.1, 22.5, 20.4, 18.1. UV-vis (CH₂Cl₂), λₘₐₓ nm (log ε): 419(5.54), 511(4.46), 544(3.64), 585(3.98), 639(3.15). HRMS-MALDI ([M+H]⁺): calcd for C₆₈H₅₇F₁₀N₈O₄ 1239.4338, found 1239.4335 with an isotope distribution pattern that is the same as the calculated one.

### Example 117

### Porphyrin 20h (Table 4, entry 8)

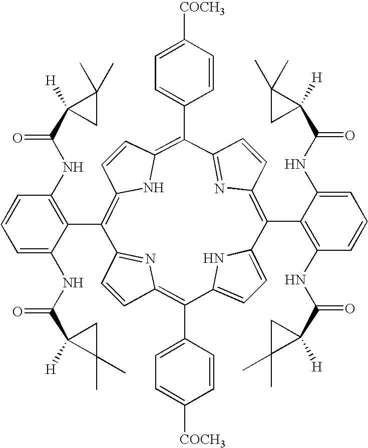

The general procedure was used to couple 5,15-bis(2,6-dibromophenyl)-10,20-bis(4-acetylphenyl)porphyrin (0.051 g, 0.05 mmol) with (S)-(+)-2,2-dimethylcyclopropanecarboxamide (0.184 g, 1.6 mmol), using Pd(OAc)₂ (0.004 g, 0.02 mmol), Xantphos (0.023 g, 0.04 mmol), and CS₂CO₃ (0.261 g, 0.8 mmol). The reaction was conducted in THF (4 mL) at 100° C. for 60 h. The pure compound was isolated by flash column chromatography (silica gel, ethyl acetate:methylene chloride (v/v)=1:3) as purple solids (0.038 g, 66%). ¹H NMR (300 MHz, CDCl₃): δ 8.92 (m, 8H), 8.30-8.42 (m, 12H), 7.83 (t, J=8.1 Hz, 2H), 6.46 (broad, 4H), 2.89 (s, 6H), 0.71-0.88 (m, 16H), −0.04-0.20 (m, 20H), -2.63 (s, 2H). ¹³C NMR (75 MHz, CDCl₃): δ 197.9, 171.0, 169.6, 145.5, 139.2, 136.8, 134.6, 130.5, 127.0, 118.0, 28.9, 27.0, 26.3, 22.4, 20.4, 18.2. UV-vis (CH₂Cl₂), λₘₐₓ nm (log ε): 412(5.55), 516(4.32), 550(3.82), 589(3.79), 644(3.40). HRMS-MALDI ([M+H]⁺): calcd for C₇₂H₇₁N₈O₆ 1143.5491, found 1143.5467 with an isotope distribution pattern that is the same as the calculated one.

### Example 118

### Porphyrin 20i (Table 4, entry 9)

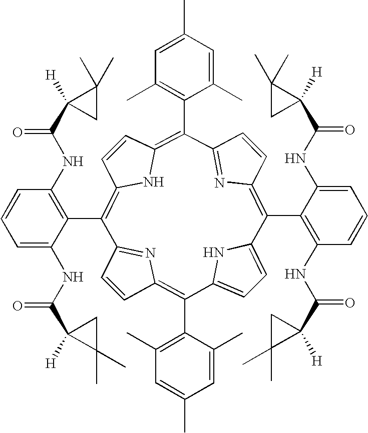

The general procedure was used to couple 5,15-bis(2,6-dibromophenyl)-10,20-dimesitylporphyrin (0.051 g, 0.05 mmol) with (S)-(+)-2,2-dimethyl cyclopropanecarboxamide (0.181 g, 1.6 mmol), using Pd(OAc)₂ (0.004 g, 0.02 mmol), Xantphos (0.023 g, 0.04 mmol), and Cs₂CO₃ (0.261 g, 0.8 mmol). The reaction was conducted in THF (6 mL) at 100° C. for 56 h. The pure compound was isolated by flash column chromatography (silica gel, ethyl acetate:hexanes (v/v)=1:4) as purple solids (0.048 g, 84%). ¹H NMR (300 MHz, CDCl₃): δ 8.80 (m, 8H), 8.42 (broad, 4H), 7.80 (t, J=8.1 Hz, 2H), 7.30 (s, 4H), 6.52 (broad, 4H), 2.63 (s, 6H), 1.82 (s, 12H), 0.86 (s, 12H), 0.68 (broad, 4H), −0.06-0.20 (m, 20H), −2.54 (s, 2H). ¹³C NMR (75 MHz, CDCl₃): δ 169.6, 147.3, 139.2, 138.9, 138.6, 136.9, 130.4, 128.2, 119.9, 117.6, 28.9, 26.4, 22.4, 21.7, 21.5, 20.5, 18.2. UV-vis (CH₂Cl₂), λₘₐₓ nm (log ε): 421(5.42), 515(4.19), 549(3.68), 590(3.68), 645(3.48). HRMS-EI ([M]⁺): calcd for C₇₄H₇₈N₈O₄ 1142.6146, found 1142.6115 with an isotope distribution pattern that is the same as the calculated one.

### Example 119

### Porphyrin 20i (Table 4, entry 10)

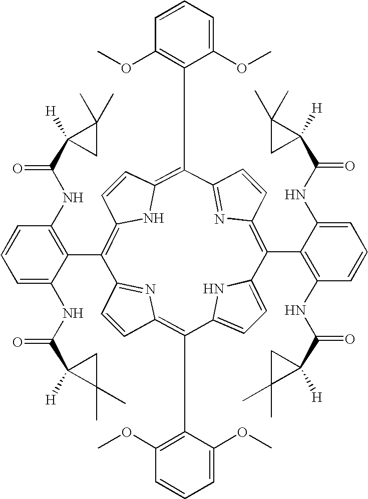

The general procedure was used to couple 5,15-bis(2,6-dibromophenyl)-10,20-bis(2,6-dimethoxyphenyl)porphyrin (0.105 g, 0.1 mmol) with (S)-(+)-2,2-dimethylcyclopropanecarboxamide (0.362 g, 3.2 mmol), using Pd(OAc)₂ (0.009 g, 0.04 mmol), Xantphos (0.046 g, 0.08 mmol), and Cs₂CO₃ (0.522 g, 1.6 mmol). The reaction was conducted in THF (6 mL) at 100° C. for 60 h. The pure compound was isolated by flash column chromatography (silica gel, ethyl acetate:hexanes (v/v)=1:2) as purple solids (0.069 g, 59%). ¹H NMR (300 MHz, CDCl₃): δ 8.87 (d, J=4.8 Hz, 4H), 8.79 (d, J=4.8 Hz, 4H), 8.47 (broad, 4H), 7.81 (t, J=8.7 Hz, 4H), 7.06 (d, J=8.4 Hz, 4H), 6.58 (broad, 4H), 3.55 (s, 12H), 0.88 (s, 12H), 0.65 (broad, 4H), 0.04-0.21 (m, 20H), −2.47 (s, 2H). ¹³C NMR (75 MHz, CDCl₃): δ 169.6, 160.2, 139.2, 130.9, 130.1, 118.2, 117.0, 113.6, 107.1, 104.1, 55.9, 29.0, 26.2, 22.2, 20.1, 18.2. UV-vis (CH₂Cl₂), λₘₐₓ nm (log ε): 412(5.50), 514(4.28), 547(3.67), 589(3.77), 643(3.38). HRMS-MALDI ([M+H]⁺): calcd for C₇₂H₇₅N₈O₈ 1179.5702, found 1179.5758 with an isotope distribution pattern that is the same as the calculated one.

### Example 120

### Porphyrin 20k (Table 4, entry 11)

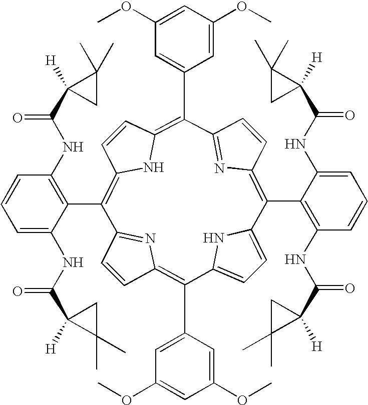

The general procedure was used to couple 5,15-bis(2,6-dibromophenyl)-10,20-bis(3,5-dimethoxyphenyl)porphyrin (0.053 g, 0.05 mmol) with (S)-(+)-2,2-dimethylcyclopropanecarboxamide (0.181 g, 1.6 mmol), using Pd(OAc)₂ (0.004 g, 0.02 mmol), Xantphos (0.023 g, 0.04 mmol), and Cs₂CO₃ (0.261 g, 0.8 mmol). The reaction was conducted in THF (4 mL) at 100° C. for 48 h. The pure compound was isolated by flash column chromatography (silica gel, ethyl acetate:hexanes (v/v)=1:1) as purple solids (0.052 g, 88%). ¹H NMR (300 MHz, CDCl₃): δ 9.04 (d, J=4.8 Hz, 4H), 8.84 (d, J=4.8 Hz, 4H), 8.44 (broad, 4H), 7.83 (t, J=8.7 Hz, 2H), 7.34 (d, J=1.8 Hz, 4H), 6.93 (t, J=1.8 Hz, 2H), 6.45 (broad, 4H), 3.98 (s, 12H), 0.88 (s, 12H), 0.69 (broad, 4H), −0.07-0.17 (m, 20H), −2.68 (s, 2H). ¹³C NMR (75 MHz, CDCl₃): δ 169.6, 159.1, 142.5, 139.3, 133.7, 130.4, 121.0, 117.7, 114.1, 100.1, 55.6, 29.0, 26.3, 22.4, 20.4, 18.3. UV-vis (CH₂Cl₂), λₘₐₓ nm (log ε): 423(5.53), 515(4.34), 549(3.81), 589(3.85), 643(3.55). HRMS-MALDI ([M+H]⁺): calcd for C₇₂H₇₅N₈O₈ 1179.5702, found 1179.5736 with an isotope distribution pattern that is the same as the calculated one.

### Example 121

### Porphyrin 201 (Table 4, entry 12)

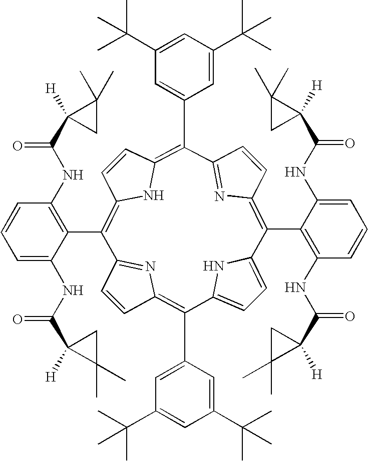

The general procedure was used to couple 5,15-bis(2,6-dibromophenyl)-10,20-bis[3,5-di(tert-butyl)phenyl]porphyrin (0.231 g, 0.2 mmol) with (S)-(+)-2,2-dimethylcyclopropanecarboxamide (0.362 g, 3.2 mmol), using Pd(OAc)₂ (0.018 g, 0.08 mmol), Xantphos (0.093 g, 0.16 mmol), and Cs₂CO₃ (1.045 g, 3.2 mmol). The reaction was conducted in THF (4 mL) at 100° C. for 48 h. The pure compound was isolated by flash column chromatography (silica gel, ethyl acetate:hexanes (v/v)=1:4) as purple solids (0.217 g, 85%). ¹H NMR (300 MHz, CDCl₃): δ 8.99 (d, J=4.8 Hz, 4H), 8.87 (d, J=4.8 Hz, 4H), 8.44 (broad, 4H), 8.04 (d, J=1.5 Hz, 4H), 7.83 (m, 4H), 6.50 (broad, 4H), 1.53 (s, 36H), 0.87 (s, 12H), 0.69 (broad, 4H), −0.05-0.14 (m, 20H), −2.34 (s, 2H). ¹³C NMR (75 MHz, CDCl₃): δ 169.6, 149.3, 139.8, 139.2, 133.6, 130.3, 129.8, 122.7, 121.8, 117.4, 35.0, 31.7, 29.0, 26.3, 22.3, 20.2, 18.3. UV-vis (CH₂Cl₂), λₘₐₓ nm (log ε): 422(5.46), 517(4.17), 552(3.77), 591(3.66), 646(3.53). HRMS-EI ([M]⁺): calcd for C₈₄H₉₈N₈O₄ 1282.7711, found 1282.7715 with an isotope distribution pattern that is the same as the calculated one.

### Example 122

### Porphyrin 20m (Table 4, entry 13)

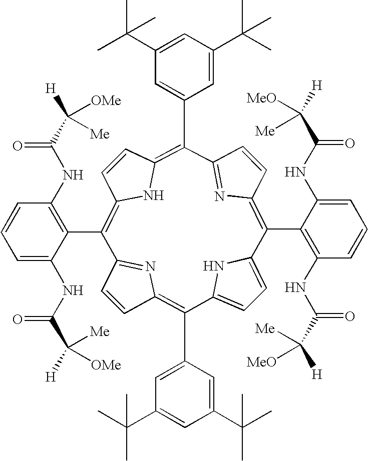

The general procedure was used to couple 5,15-bis(2,6-dibromophenyl)-10,20-bis[3,5-di(tert-butyl)phenyl]porphyrin (0.058 g, 0.05 mmol) with (R)-(+)-2-methoxypropionamide (0.082 g, 0.8 mmol), using molecular sieves (4A, 0.100 g), Pd(OAc)₂ (0.004 g, 0.02 mmol), Xantphos (0.023 g, 0.04 mmol), and Cs₂CO₃ (0.261 g, 0.8 mmol). The reaction was conducted in THF (4 mL) at 100° C. for 64 h. The pure compound was isolated by flash column chromatography (silica gel, ethyl acetate:hexanes (v/v)=1:2) as purple solids (0.049 g, 79%). ¹H NMR (300 MHz, CDCl₃): δ 8.90 (d, J=4.8 Hz, 4H), 8.79 (d, J=4.8 Hz, 4H), 8.57 (d, J=8.7 Hz, 4H), 7.95 (s, 4H), 7.89 (t, J=8.7 Hz, 2H), 7.83 (s, 6H), 3.08 (q, J=6.6 Hz, 4H), 1.52 (s, 36H), 1.36 (s, 12H), 0.66 (d, J=6.6 Hz, 12H), −2.48 (s, 2H). ¹³C NMR (75 MHz, CDCl₃): δ 171.2, 149.2, 140.0, 138.5, 130.6, 129.8, 122.6, 122.3, 121.7, 117.0, 107.8, 78.1, 55.9, 35.0, 31.6, 17.7. UV-vis (CH₂Cl₂), λₘₐₓ nm (log ε): 421(5.59), 516(4.29), 551(3.89), 592(3.76), 647(3.62). HRMS-MALDI ([M]⁺): calcd for C₇₆H₉₁N₈O₈ 1243.6954, found 1243.6894 with an isotope distribution pattern that is the same as the calculated one.

### Example 123

### Porphyrin 20n (Table 4, entry 14)

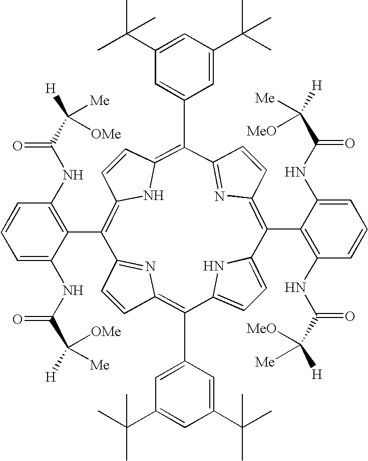

The general procedure was used to couple 5,15-bis(2,6-dibromophenyl)-10,20-bis[3,5-di(tert-butyl)phenyl]porphyrin (0.058 g, 0.05 mmol) with (S)-(−)-2-methoxypropionamide (0.082 g, 0.8 mmol), using molecular sieves (4A, 0.1 g), Pd(OAc)₂ (0.004 g, 0.02 mmol), Xantphos (0.023 g, 0.04 mmol), and Cs₂CO₃ (0.261 g, 0.8 mmol). The reaction was conducted in THF (4 mL) at 100° C. for 48 h. The pure compound was isolated by flash column chromatography (silica gel, ethyl acetate:hexanes (v/v)=1:2) as purple solids (0.045 g, 72%). ¹H NMR (300 MHz, CDCl₃): δ 8.90 (d, J=4.8 Hz, 4H), 8.79 (d, J=4.8 Hz, 4H), 8.57 (d, J=8.7 Hz, 4H), 7.94 (s, 4H), 7.88 (t, J=8.7 Hz, 2H), 7.82 (s, 6H), 3.08 (q, J=6.9 Hz, 4H), 1.52 (s, 36H),1.35 (s, 12H), 0.65 (d, J=7.2 Hz, 12H), −2.48 (s, 2H). ¹³C NMR (75 MHz, CDCl₃): δ 171.2, 149.2, 140.0, 138.5, 130.6, 129.8, 122.6, 122.3, 121.7, 117.0, 107.8, 78.1, 55.9, 35.0, 31.6, 17.8. UV-vis (CH₂Cl₂), λₘₐₓ nm (log ε): 421(5.61), 516(4.31), 551(3.91), 592(3.80), 648(3.68). HRMS-MALDI ([M]⁺): calcd for C₇₆H₉₁N₈O₈ 1243.6954, found 1243.6991 with an isotope distribution pattern that is the same as the calculated one.

### Example 124

### Porphyrin 20o (Table 4, entry 15)

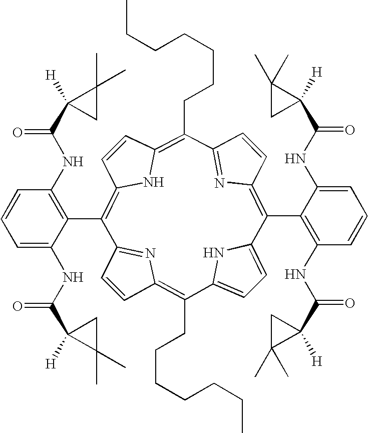

The general procedure was used to couple 5,15-bis(2,6-dibromophenyl)-10,20-bisheptylporphyrin (0.049 g, 0.05 mmol) with (S)-(+)-2,2-dimethylcyclopropanecarboxamide (0.184 g, 1.6 mmol), using Pd(OAc)₂ (0.004 g, 0.02 mmol), Xantphos (0.023 g, 0.04 mmol), and Cs₂CO₃ (0.261 g, 0.8 mmol). The reaction was conducted in THF (4 mL) at 100° C. for 60 h. The pure compound was isolated by flash column chromatography (silica gel, ethyl acetate:hexanes (v/v)=1:3) as purple solids (0.043 g, 74%). ¹H NMR (300 MHz, CDCl₃): δ 9.56 (d, J=4.8 Hz, 4H), 8.95 (d, J=4.8 Hz, 4H), 8.51 (broad, 4H), 7.87 (t, J=8.1 Hz, 2H), 6.50 (broad, 4H), 5.03 (m, 4H), 2.55 (m, 4H), 1.86 (m, 4H), 1.37 (m, 6H), 0.91 (s, 12H), 0.70 (broad, 4H), −0.04-0.19 (m, 20H), −2.48 (s, 2H). ¹³C NMR (75 MHz, CDCl₃): δ 169.7, 139.3, 131.2, 130.3, 130.0, 121.4, 117.4, 107.5, 39.0, 35.2, 31.8, 30.5, 29.3, 28.9, 26.3, 22.7, 22.3, 20.4, 18.2, 14.1. UV-vis (CH₂Cl₂), λₘₐₓ nm (log ε): 421(5.40), 517(4.16), 553(3.82), 594(3.62), 651(3.71). HRMS-MALDI ([M+H]⁺): calcd for C₇₀H₈₇N₈O₄ 1103.6845, found 1103.6871 with an isotope distribution pattern that is the same as the calculated one.

### Example 125

### Porphyrin 20p (Table 4, entry 16)

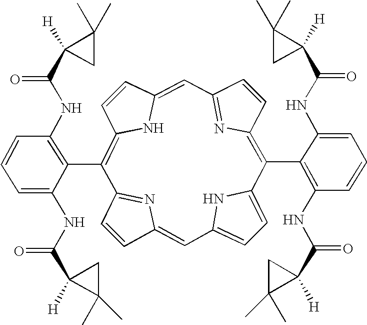

The general procedure was used to couple 5,15-bis(2,6-dibromophenyl)porphyrin (0.039 g, 0.05 mmol) with (S)-(+)-2,2-dimethylcyclopropane carboxamide (0.181 g, 1.6 mmol), using Pd(OAc)₂ (0.004 g, 0.02 mmol), Xantphos (0.023 g, 0.04 mmol), and Cs₂CO₃ (0.261 g, 0.8 mmol). The reaction was conducted in THF (6 mL) at 100° C. for 60 h. The pure compound was isolated by flash column chromatography (silica gel, ethyl acetate:hexanes (v/v)=1:1) as purple solids (0.036 g, 79%). ¹H NMR (300 MHz, CDCl₃): δ 10.44 (s, 2H), 9.50 (d, J=4.8 Hz, 4H), 9.08 (d, J=4.8 Hz, 4H), 8.48 (broad, 4H), 7.86 (t, J=8.7 Hz, 2H), 6.47 (broad, 4H), 0.88 (s, 12H), 0.67 (broad, 4H), −0.14-0.13 (m, 20H), -3.05 (s, 2H). ¹³C NMR (75 MHz, CDCl₃): δ 169.7, 147.2, 146.4, 139.3, 133.7, 130.7, 130.5, 117.7, 108.0, 106.3, 28.9, 26.2, 22.4, 20.4, 18.2. UV-vis (CH₂Cl₂), λₘₐₓ nm (log ε): 409(5.31), 503(4.12), 536(3.74), 575(3.67), 628(3.40). HRMS-MALDI ([M+H]⁺): calcd for C₅₆H₅₉N₈O₄ 907.4654, found 907.4640 with an isotope distribution pattern that is the same as the calculated one.

### Example 126

### 5,15-Bis(2,6-dibromo-4-trimethylsilanylphenyl)porphyrin

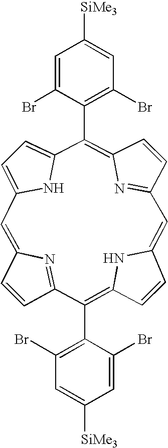

A mixture of dipyrromethane (0.146 g, 1 mmol), 2,6-dibromo-4-trimethylsilanyl-benzaldehyde (0.336 g, 1 mmol) and molecular sieves (4A, 0.3 g) in chloroform (150 mL) was purged with nitrogen for 10 min. Boron trifluoride diethyl etherate (0.1 mL) was added dropwise via a syringe and flask was wrapped with aluminum foil to shield it from light. The solution was stirred under nitrogen atmosphere at room temperature for 16 h, and 2,3-dichloro-5,6-dicyano-1,4-benzoquinone (DDQ) (0.287 g, 1.2 mmol) was added as powder at one time. After 30 min, 1 mL of triethylamine was added in. The reaction solution was then directly poured on the top of a silica gel column that was packed with dichloromethane. The column was eluted with dichloromethane. The fractions containing product were collected and concentrated on a rotary evaporator. The residue was washed several times with hexanes to afford the title compound as a purple solid. Yield: 0.058 g (14%). ¹H NMR (300 MHz, CDCl₃): δ 10.25 (s, 2H), 9.35 (d, J=4.8 Hz, 4H), 8.85 (d, J=4.8 Hz, 4H), 8.11 (s, 4H), 0.54 (s, 18H), −3.09 (s, 2H). UV-vis (CH₂Cl₂): 407(5.62), 502(4.34), 534(3.88), 576(3.90), 630(3.45). HRMS-MALDI ([M+H]⁺): calcd for C₃₈H₃₅Br₄N₄Si₂ 918.9128; found: 918.9124.

### Example 127

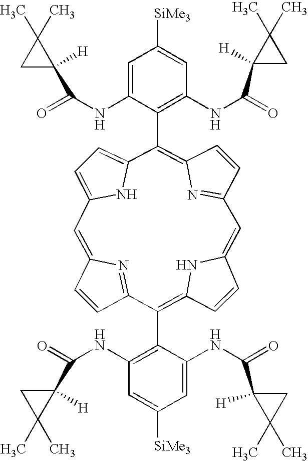

The general procedure was used to couple 5,15-bis(2,6-dibromo-4-trimethylsilanylphenyl)porphyrin (0.023 g, 0.025 mmol) with (S)-(+)-2,2-dimethylcyclopropanecarboxamide (0.091 g, 0.8 mmol), using Pd(OAc)₂ (0.002 g, 0.01 mmol), Xantphos (0.012 g, 0.02 mmol) and Cs₂CO₃ (0.130 g, 0.4 mmol). The reaction was conducted in THF (4 mL) at 100° C. for 41 h. The pure compound was isolated by flash column chromatography (silica gel, ethyl acetate:hexanes (v/v)=1:2) as purple solids (0.019 g, 72%). ¹H NMR (300 MHz, CDCl₃): δ 10.44 (s, 2H), 9.50 (d, J=4.8 Hz, 4H), 9.12 (d, J=4.8 Hz, 4H), 8.66 (broad, 4H), 6.50 (broad, 4H), 0.86 (s, 12H), 0.69 (broad, 4H), 0.55 (s, 18H), −0.10-0.08 (m, 20H), −3.05 (s, 2H). ¹³C NMR (75 MHz, CDCl₃): δ 169.6, 147.0, 146.4, 138.3, 133.5, 130.7, 130.8, 122.3, 108.3, 106.2, 29.0, 26.3, 22.3, 20.3, 18.2, −0.87. UV-vis (CH₂Cl₂): 410(5.53), 503(4.33), 537(3.97), 575(3.88), 629(3.61). HRMS-MALDI ([M+H]⁺): calcd for C₆₂H₇₅N₈O₄Si₂ 1051.5444, found 1051.5458.

### Example 128

### General Procedures for Synthesis of Cobalt Porphyrin Complex

The general procedures for the synthesis of cobalt porphyrin complex follow those described by Tsuchida et al., 1990 Chem. Lett. 3:389; Tsuchida et al., (1990) J. Chem. Soc.-Dalton Trans. 2713; Komatsu et al., (1990) J. Chem. Soc.-Chem. Commun. 66. Free base porphyrin and anhydrous CoCl₂ were placed in an oven-dried, resealable Schlenk tube. The tube was capped with a Teflon screwcap, evacuated, and backfilled with nitrogen. The screwcap was replaced with a rubber septum, 2,6-lutidine and dry THF were added via syringe. The tube was purged with nitrogen for 2 minutes, and then the septum was replaced with the Teflon screwcap. The tube was sealed, and its contents were heated with stirring. The resulting mixture was cooled to room temperature, taken up in ethyl acetate, and transferred to a separatory funnel. The mixture was washed with water 3 times and concentrated in vacuo.

### Example 129

### Cobalt porphyrin 21a (Table 5, entry 1)

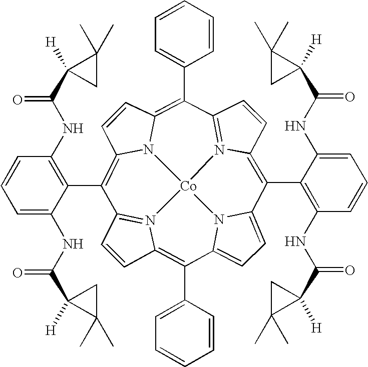

The general procedure was used for cobalt ion insertion. Free-base porphyrin (0.066 g), anhydrous CoCl₂ (0.073 g), 2,6-lutidine (0.025 mL), and dry THF (5 mL) were heated at 70° C. under N₂ for 16 hours. The resulting mixture was cooled to room temperature, taken up in ethyl acetate and transferred to a separatory funnel. The mixture was washed with water 3 times and concentrated in vacuo. The pure compound was obtained after flash column chromatography (silica gel, ethyl acetate:hexanes (v/v)=1:2) as a red solid (0.061 g, 88%). UV-vis (CH₂Cl₂), λₘₐₓ nm (log ε): 412(5.29), 529(4.06), 556(3.71). HRMS-EI ([M]⁺): calcd for C₆₈H₆₄CoN₈O₄, 1115.4383, found 1115.4376 with an isotope distribution pattern that is the same as the calculated one.

### Example 130

### Cobalt porphyrin 21b (Table 5, entry 2)

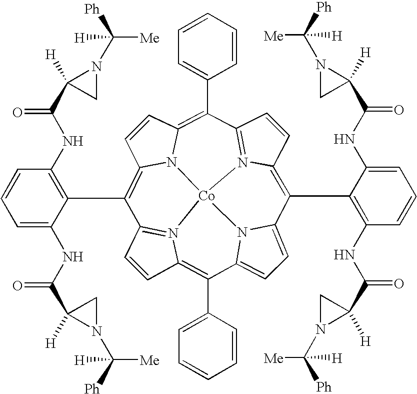

Free-base porphyrin (0.019 g), cobalt acetate tetrahydrate (0.028 g), and dry DMF (2 mL) were heated at 160° C. under N₂ for 3 hours. The resulting mixture was cooled to room temperature, taken up in ethyl acetate and transferred to a separatory funnel. The mixture was washed with water 3 times and concentrated in vacuo. The pure compound was obtained as a red solid (0.017 g, 86%). UV-vis (CH₂Cl₂), λₘₐₓ nm (log ε): 412(5.43), 528(4.30), 556(4.11), 615(3.71). HRMS-MALDI ([M+H]⁺): calcd for C₈₀H₆₉CoN₁₂O₄ 1320.4891, found 1320.3267 with an isotope distribution pattern that is the same as the calculated one.

### Example 131

### Cobalt porphyrin 21c (Table 5, entry 3)

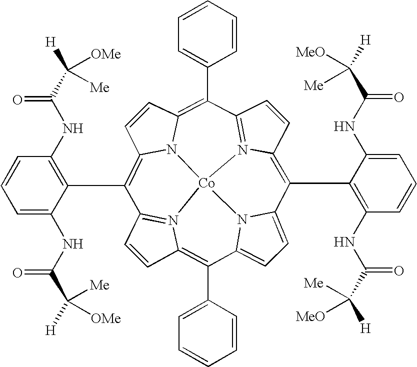

The general procedure was used for cobalt ion insertion. Free-base porphyrin (0.025 g), anhydrous CoCl₂ (0.029 g), 2,6-lutidine (0.010 mL), and dry THF (3 mL) were heated at 70° C. under N₂ for 15 hours. The resulting mixture was cooled to room temperature, taken up in ethyl acetate, and transferred to a separatory funnel. The mixture was washed with water 3 times and concentrated in vacuo. The pure compound was obtained as a red solid (0.025 g, 95%). UV-vis (CH₂Cl₂), λₘₐₓ nm (log ε): 412(5.49), 528(4.25), 556(3.91). HRMS-MALDI ([M]⁺): calcd for C₆₀H₅₆CoN₈O₈ 1075.3548, found 1075.3518 with an isotope distribution pattern that is the same as the calculated one.

### Example 132

### Cobalt porphyrin 21d (Table 5, entry 4)

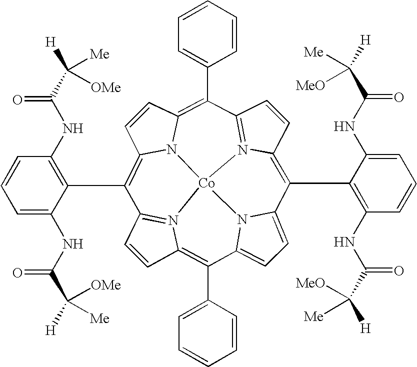

The general procedure was used for cobalt ion insertion. Free-base porphyrin (0.020 g), anhydrous CoCl₂ (0.023 g), 2,6-lutidine (0.008 mL), and dry THF (4 mL) were heated at 70° C. under N₂ for 14 hours. The resulting mixture was cooled to room temperature, taken up in ethyl acetate, and transferred to a separatory funnel. The mixture was washed with water 3 times and concentrated in vacuo. The pure compound was obtained as a red solid (0.020 g, 95%). UV-vis (CH₂Cl₂), λₘₐₓ nm (log ε): 412(5.65), 528(4.39), 556(3.85). HRMS-MALDI ([M]⁺): calcd for C₆₀H₅₆CoN₈O₈ 1075.3548, found 1075.3544 with an isotope distribution pattern that is the same as the calculated one.

### Example 133

### Cobalt porphyrin 21e (Table 5, entry 5)

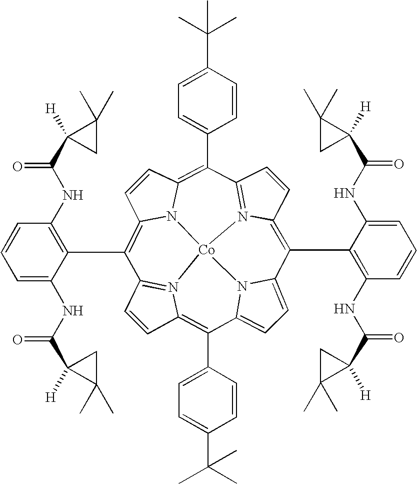

The general procedure was used for cobalt ion insertion. Free-base porphyrin (0.045 g), anhydrous CoCl₂ (0.045 g), 2,6-lutidine (0.014 mL), and dry THF (5 mL) were heated at 70° C. under N₂ for 12 hours. The resulting mixture was cooled to room temperature, taken up in ethyl acetate, and transferred to a separatory funnel. The mixture was washed with water 3 times and concentrated in vacuo. The pure compound was obtained after flash column chromatography (silica gel, ethyl acetate:hexanes (v/v)=1:4) as a red solid (0.034 g, 72%). UV-vis (CH₂Cl₂), λₘₐₓ nm (log ε): 413(5.48), 529(4.25), 554(3.92). HRMS-EI ([M]⁺): calcd for C₇₆H₈₀CoN₈O₄ 1227.5635, found 1227.5593 with an isotope distribution pattern that is the same as the calculated one.

### Example 134

### Cobalt porphyrin 21f (Table 5, entry 6)

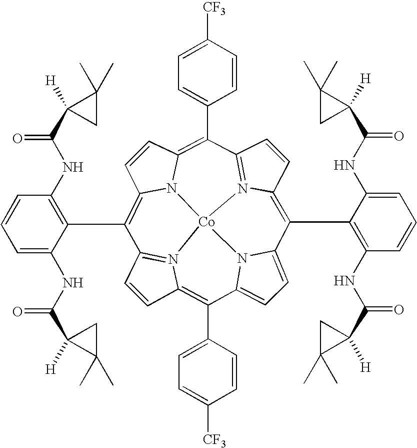

The general procedure was used for cobalt ion insertion. Free-base porphyrin (0.025 g), anhydrous CoCl₂ (0.022 g), 2,6-lutidine (0.008 mL), and dry THF (2 mL) were heated at 70° C. under N₂ for 17 hours. The resulting mixture was cooled to room temperature, taken up in ethyl acetate, and transferred to a separatory funnel. The mixture was washed with water 3 times and concentrated in vacuo. The pure compound was obtained as a red solid (0.025 g, 95%). UV-vis (CH₂Cl₂), λₘₐₓ nm (log ε): 420(4.85), 444(4.95), 523(4.11), 550(4.20). HRMS-EI ([M]⁺): calcd for C₇₀H₆₂CoF₆N₈O₄ 1251.4125, found 1251.4085 with an isotope distribution pattern that is the same as the calculated one.

### Example 135

### Cobalt porphyrin 21a (Table 5, entry 7)

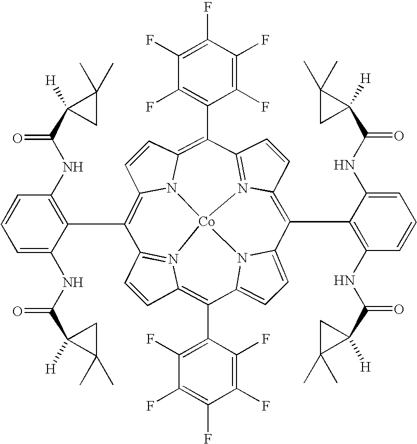

The general procedure was used for cobalt ion insertion. Free-base porphyrin (0.010 g), anhydrous CoCl₂ (0.009 g), 2,6-lutidine (0.005 mL), and dry THF (2 mL) were heated at 70° C. under N₂ for 14 hours. The resulting mixture was cooled to room temperature, taken up in ethyl acetate, and transferred to a separatory funnel. The mixture was washed with water 3 times and concentrated in vacuo. The pure compound was obtained as a red solid (0.009 g, 86%). UV-vis (CH₂Cl₂), λₘₐₓ nm (log ε): 410(5.57), 443(4.70), 527(4.41), 556(4.23). HRMS-MALDI ([M]⁺): calcd for C₆₈H₅₄CoF₁₀N₈O₄ 1295.3435, found 1295.3459 with an isotope distribution pattern that is the same as the calculated one.

### Example 136

### Cobalt porphyrin 21h (Table 5, entry 8)

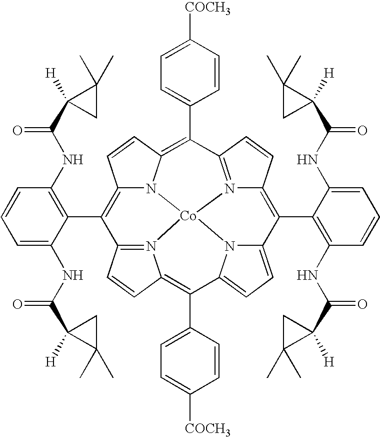

The general procedure was used for cobalt ion insertion. Free-base porphyrin (0.030 g), anhydrous CoCl₂ (0.031 g), 2,6-lutidine (0.010 mL), and dry THF (3 mL) were heated at 70° C. under N₂ for 12 hours. The resulting mixture was cooled to room temperature, taken up in ethyl acetate, and transferred to a separatory funnel. The mixture was washed with water 3 times and concentrated in vacuo. The pure compound was obtained after flash column chromatography (silica gel, ethyl acetate:hexanes (v/v)=1:1) as a red solid (0.026 g, 83%). UV-vis (CH₂Cl₂), λₘₐₓ nm (log ε): 413(5.53), 528(4.31), 553(3.98). HRMS-MALDI ([M]⁺): calcd for C₇₂H₆₈CoN₈O₆ 1199.4588, found 1199.4572 with an isotope distribution pattern that is the same as the calculated one.

### Example 137

### Cobalt porphyrin 21i (Table 5, entry 9)

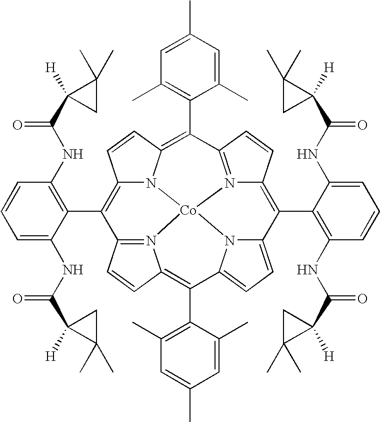

The general procedure was used for cobalt ion insertion. Free-base porphyrin (0.070 g), anhydrous CoCl₂ (0.071 g), 2,6-lutidine (0.024 mL), and dry THF (5 mL) were heated at 70° C. under N₂ for 16 hours. The resulting mixture was cooled to room temperature, taken up in ethyl acetate, and transferred to a separatory funnel. The mixture was washed with water 3 times and concentrated in vacuo. The pure compound was obtained after flash column chromatography (silica gel, ethyl acetate:hexanes (v/v)=1:3) as a red solid (0.067 g, 91%). UV-vis (CH₂Cl₂), λₘₐₓ nm (log ε): 413(5.33), 528(4.09), 558(3.73). HRMS-EI ([M]⁺): calod for C₇₄H₇₆CoN₈O₄, 1199.5322, found 1199.5320 with an isotope distribution pattern that is the same as the calculated one.

### Example 138

### Cobalt porphyrin 21j (Table 5, entry 10)

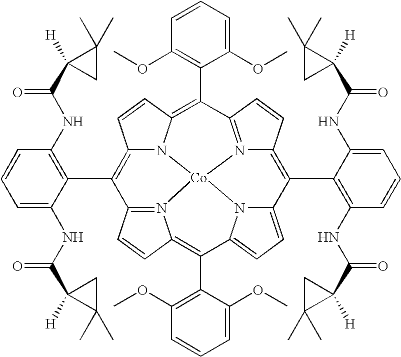

The general procedure was used for cobalt ion insertion. Free-base porphyrin (0.050 g), anhydrous CoCl₂ (0.044 g), 2,6-lutidine (0.015 mL), and dry THF (3 mL) were heated at 70° C. under N₂ for 19 hours. The resulting mixture was cooled to room temperature, taken up in ethyl acetate, and transferred to a separatory funnel. The mixture was washed with water 3 times and concentrated in vacuo. The pure compound was obtained as a red solid (0.050 g, 95%). UV-vis (CH₂Cl₂), λₘₐₓ nm (log ε): 413(5.18), 439(4.53), 532(4.09), 551(4.00). HRMS-EI ([M]⁺): calcd for C₇₂H₇₂CoN₈O₈ 1235.4805, found 1235.4794 with an isotope distribution pattern that is the same as the calculated one.

### Example 139

### Cobalt porphyrin 21k (Table 5 entry 11)

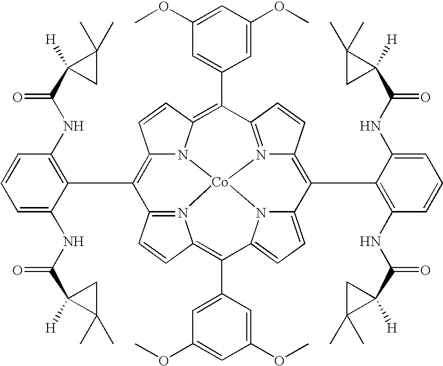

The general procedure was used for cobalt ion insertion. Free-base porphyrin (0.023 g), anhydrous CoCl₂ (0.022 g), 2,6-lutidine (0.007 mL), and dry THF (3 mL) were heated at 70° C. under N₂ for 15 hours. The resulting mixture was cooled to room temperature, taken up in ethyl acetate, and transferred to a separatory funnel. The mixture was washed with water 3 times and concentrated in vacuo. The pure compound was obtained as a red solid (0.023 g, 96%). UV-vis (CH₂Cl₂), λₘₐₓ nm (log ε): 414(4.92), 445(4.66), 530(4.13), 553(4.11). HRMS-MALDI ([M]⁺): calcd for C₇₂H₇₂CoN₈O₈ 1235.4800, found 1235.4749 with an isotope distribution pattern that is the same as the calculated one.

### Example 140

### Cobalt porphyrin 21l (Table 5, entry 12)

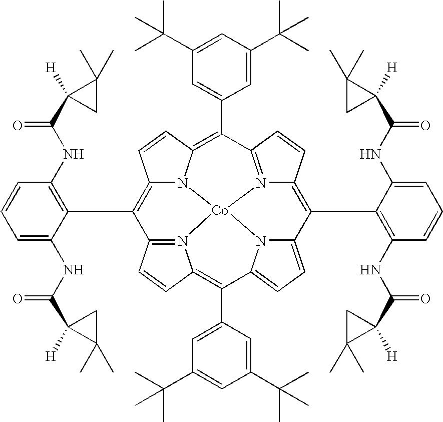

The general procedure was used for cobalt ion insertion. Free-base porphyrin (0.100 g), anhydrous CoCl₂ (0.080 g), 2,6-lutidine (0.027 mL), and dry THF (5 mL) were heated at 70° C. under N₂ for 9 hours. The resulting mixture was cooled to room temperature, taken up in ethyl acetate, and transferred to a separatory funnel. The mixture was washed with water 3 times and concentrated in vacuo. The pure compound was obtained after flash column chromatography (silica gel, ethyl acetate:hexanes (v/v)=1:4) as a red solid (0.099 g, 91%). UV-vis (CH₂Cl₂), λₘₐₓ nm (log ε): 414(5.37), 529(4.14), 549(3.84). HRMS-EI ([M]⁺): calcd for C₈₄H₉₆CoN₈O₄ 1339.6887, found 1339.6909 with an isotope distribution pattern that is the same as the calculated one.

### Example 141

### Cobalt porphyrin 21m (Table 5, entry 13)

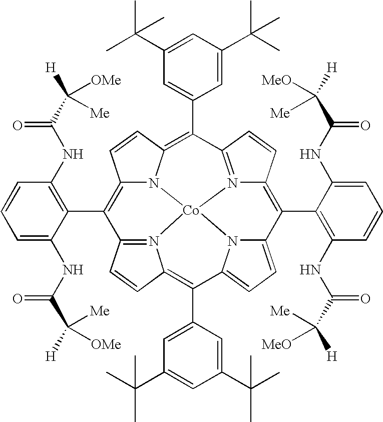

The general procedure was used for cobalt ion insertion. Free-base porphyrin (0.029 g), anhydrous CoCl₂ (0.026 g), 2,6-lutidine (0.010 mL), and dry THF (3 mL) were heated at 70° C. under N₂ for 15 hours. The resulting mixture was cooled to room temperature, taken up in ethyl acetate, and transferred to a separatory funnel. The mixture was washed with water 3 times and concentrated in vacuo. The pure compound was obtained as a red solid (0.029 g, 96%). UV-vis (CH₂Cl₂), λₘₐₓ nm (log ε): 414(5.52), 529(4.23), 558(3.96). HRMS-MALDI ([M]⁺): calcd for C₇₆H₈₈CoN₈O₈ 1299.6052, found 1299.6082 with an isotope distribution pattern that is the same as the calculated one.

### Example 142

### Cobalt porphyrin 21n (Table 5, entry 14)

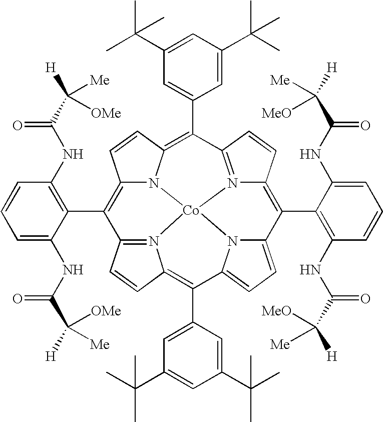

The general procedure was used for cobalt ion insertion. Free-base porphyrin (0.030 g), anhydrous CoCl₂ (0.028 g), 2,6-lutidine (0.010 mL), and dry THF (3 mL) were heated at 70° C. under N₂ for 15 hours. The resulting mixture was cooled to room temperature, taken up in ethyl acetate, and transferred to a separatory funnel. The mixture was washed with water 3 times and concentrated in vacuo. The pure compound was obtained as a red solid (0.029 g, 92%). UV-vis (CH₂Cl₂), λₘₐₓ nm (log ε): 414(5.54), 529(4.29), 557(3.94). HRMS-MALDI ([M]⁺): calcd for C₇₆H₈₈CoN₈O₈ 1299.6052, found 1299.6070 with an isotope distribution pattern that is the same as the calculated one.

### Example 143

### Cobalt porphyrin 21o (Table 5, entry 15)

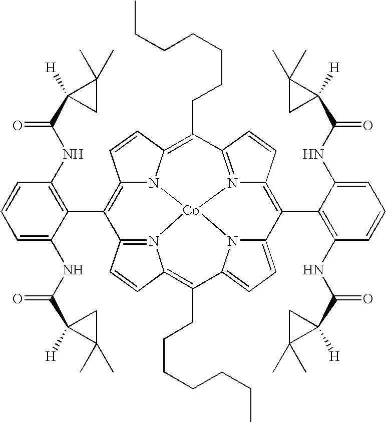

The general procedure was used for cobalt ion insertion. Free-base porphyrin (0.030 g), anhydrous CoCl₂ (0.030 g), 2,6-lutidine (0.009 mL), and dry THF (4 mL) were heated at 70° C. under N₂ for 16 hours. The resulting mixture was cooled to room temperature, taken up in ethyl acetate, and transferred to a separatory funnel. The mixture was washed with water 3 times and concentrated in vacuo. The pure compound was obtained as a red solid (0.030 g, 95%). UV-vis (CH₂Cl₂), λₘₐₓ nm (log ε): 414(5.35), 443(4.37), 533(4.18), 560(3.89). HRMS-MALDI ([M]⁺): calcd for C₇₀H₈₄CoN₈O₄ 1159.5942, found 1159.5927 with an isotope distribution pattern that is the same as the calculated one.

### Example 144

### Cobalt porphvrin 21p (Table 5, entry 16)

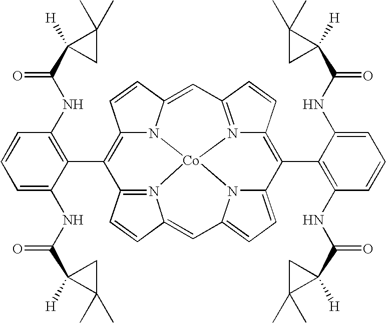

The general procedure was used for cobalt ion insertion. Free-base porphyrin (0.017 g), anhydrous CoCl₂ (0.022 g), 2,6-lutidine (0.007 mL), and dry THF (3 mL) were heated at 70° C. under N₂ for 15 hours. The resulting mixture was cooled to room temperature, taken up in ethyl acetate, and transferred to a separatory funnel. The mixture was washed with water 3 times and concentrated in vacuo. The pure compound was obtained as a red solid (0.017 g, 91%). UV-vis (CH₂Cl₂), λₘₐₓ nm (log ε): 404(5.27), 428(4.93), 461(4.62), 521(4.39), 547(4.41). HRMS-MALDI ([M]⁺): calcd for C₅₆H₅₆CoN₈O₄ 963.3751, found 963.3726 with an isotope distribution pattern that is the same as the calculated one.

### Example 145

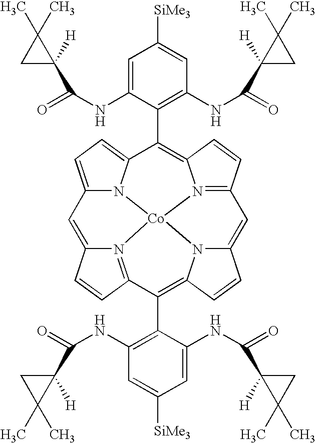

The general procedure was used for cobalt ion insertion. Free-base porphyrin (0.011 g), anhydrous CoCl₂ (0.012 g), 2,6-lutidine (0.004 mL) and dry THF (2 mL) were heated at 70° C. under N₂ for 16 hours. The resulting mixture was cooled to room temperature, taken up in ethyl acetate and transferred to a separatory funnel. The mixture was washed with water 3 times and concentrated in vacuo. The pure compound was obtained as a red solid (0.009 g, 78%). UV-vis (CH₂Cl₂): 404(5.51), 430(4.81), 454(4.39), 518(4.39), 549(4.28). HRMS-MALDI ([M]⁺): calcd for C₆₂H₇₂CoN₈O₄Si₂ 1107.4542, found 1 107.4579.

### Example 146

### Synthesis of the Iron Complex of Chiral Porphyrin 20l

Free base chiral porphyrin 20l (0.043 g) and anhydrous FeCl₂ (0.030 g) were placed in an oven-dried, resealable Schlenk tube. The tube was capped with a Teflon screwcap, evacuated, and backfilled with nitrogen. The screwcap was replaced with a rubber septum, 2,6-lutidine (0.020 mL) and dry DMF (4 mL) were added via syringe. The tube was purged with nitrogen for 2 minutes, and then the septum was replaced with the Teflon screwcap. The tube was sealed, and its contents were heated at 160° C. with stirring. The resulting mixture was cooled to room temperature, taken up in CH₂Cl₂ and transferred to a separatory funnel. The mixture was washed with 0.1 M HCl, then washed with water 3 times and concentrated in vacuo. The pure compound was obtained as a red solid (0.043 g, 93%). UV-vis (CH₂Cl₂): 419(5.34), 508(4.36), 580(4.08). HRMS-EI ([M]⁺): calcd for C₈₄H₉₆ClFeN₈O₄ 1371.6592, found 1371.6650.

### Example 147

### General Procedures for Cyclopropanation of Styrene

Catalyst (1 mol %) and DMAP were placed in an oven-dried, resealable Schlenk tube. The tube was capped with a Teflon screwcap, evacuated, and backfilled with nitrogen. The screwcap was replaced with a rubber septum, and 1.0 equivalent of styrene (0.25 mmol) was added via syringe, followed by toluene (0.5 mL), 1.2 equivalents of diazo compound and toluene again (0.5 mL). The tube was purged with nitrogen for 1 min and its contents were stirred at room temperature. After the reaction finished, the resulting mixture was concentrated and the residue was purified by flash silica gel chromatography to give the product. Chiral HPLC measurements (if necessary) were carried out on a Hewlett-Packard HP1100 system with Whelk-O1 or Chiralcel OD-H columns. Chiral GC measurements were carried out on a Hewlett-Packard G1800B GCD system equipped with Chirasil-Dex CB or Chiraldex G-TA columns.

### Example 148

### Ethyl 2-phenylcyclopropane-1-carboxylate

¹H NMR (300 MHz, CDCl₃) trans-isomer: δ 7.09-7.31 (m, 5H), 4.17 (q, J=7.2 Hz, 2H), 2.52 (ddd, J=9.3, 6.6, 4.2 Hz, 1H), 1.90 (ddd, J=8.7, 5.4, 4.5 Hz, 1H), 1.60 (ddd, J=9.0, 5.1, 4.2 Hz, 1H), 1.30 (ddd, J=8.4, 6.6, 4.8 Hz, 1H), 1.28 (t, J=7.2 Hz, 3H). ¹³C NMR (75 MHz, CDCl₃) trans-isomer: δ 173.4, 140.1, 128.4, 126.4, 126.1, 60.7, 26.2, 24.2, 17.1, 14.3. ¹H NMR (300 MHz, CDCl₃) cis-isomer: δ 7.18-7.28 (m, 5H), 3.88 (q, J=7.2 Hz, 2H), 2.59 (m, 1H), 2.08 (ddd, J=9.0, 7.8, 5.6 Hz, 1H), 1.72 (ddd, J=6.3, 4.9, 4.4 Hz, 1H), 1.32 (ddd, J=8.9, 7.9, 5.0 Hz, 1H), 0.97 (t, J=7.2 Hz, 3H). ¹³C NMR (75 MHz, CDCl₃) cis-isomer: δ 170.9, 136.5, 129.2, 127.8, 126.6, 60.1, 25.4, 21.7, 14.0, 11.1.

### Example 149

### tert-Butyl 2-phenylcyclopropane-1-carboxylate

¹H NMR (300 MHz, CDCl₃) trans-isomer: δ 7.07-7.29 (m, 5H), 2.44 (m, 1H), 1.82 (m, 1H), 1.53 (m, 1H), 1.46 (s, 9H), 1.21 (m, 1H). ¹³C NMR (75 MHz, CDCl₃) trans-isomer: δ 172.5, 140.5, 128.4, 126.3, 126.0, 80.5, 28.1, 26.0, 25.3, 17.0. ¹H NMR (300 MHz, CDCl₃) cis-isomer: δ 7.17-7.27 (m, 5H), 2.52 (m, 1H), 1.99 (m, 1H), 1.65 (m, 1H), 1.24 (m, 1H), 1.13 (s, 9H). ¹³C NMR (75 MHz, CDCl₃) cis-isomer: δ 170.1, 136.8, 129.5, 127.8, 126.5, 80.0, 27.7, 25.0, 22.7, 10.5.

### Example 150

### Ethyl 2-(4-methoxyphenyl)cyclopropane-1-carboxylate

Synthesized from 4-methoxystyrene with EDA. ¹H NMR (300 MHz, CDCl₃) trans-isomer: δ 7.03 (d, J=8.7 Hz, 2H), 6.82 (d, J=8.7 Hz, 2H), 4.16 (q, J=7.2 Hz, 2H), 3.78 (s, 3H), 2.48 (m, 1H), 1.82 (m, 1H), 1.55 (m, 1H), 1.28 (t, J=7.2 Hz, 3H), 1.25 (m, 1 H). ¹³C NMR (75 MHz, CDCl₃) trans-isomer: δ 173.5, 158.3, 132.0, 127.3, 113.8, 60.6, 55.3, 25.6, 23.8, 16.7, 14.2.

### Example 151

### tert-Butyl 2-(4-methoxvphenyl)cyclopropane-1-carboxylate

Synthesized from 4-methoxystyrene with t-BDA. ¹H NMR (300 MHz, CDCl₃) trans-isomer: δ 7.02 (d, 2H), 6.81 (d, 2H), 3.77 (s, 3H), 2.40 (m, 1H), 1.76 (m, 1H), 1.56 (m, 1H), 1.46 (s, 9H), 1.17 (m, 1H). ¹³C NMR (75 MHz, CDCl₃) trans-isomer: δ 172.7, 158.1, 132.4, 127.1, 113.8, 80.4, 55.2, 28.1, 25.1, 24.9, 16.7.

### Example 152

### Ethyl 2-(4-methylphenyl)cyclopropane-1-carboxylate

Synthesized from 4-methylstyrene with EDA. ¹H NMR (300 MHz, CDCl₃) trans-isomer: δ 7.08 (d, J=8.1 Hz, 2H), 6.99 (d, J=8.1 Hz, 2H), 4.16 (q, J=7.2 Hz, 2H), 2.48 (m, 1H), 2.31(s, 3H), 1.86 (m, 1H), 1.57 (m, 1H), 1.29 (m, 1H), 1.27 (t, J=7.2 Hz, 3H). ¹³C NMR (75 MHz, CDCl₃) trans-isomer: δ 173.5, 137.0, 136.0, 129.1, 126.0, 60.6, 25.9, 24.0, 20.9, 16.9, 14.2.

### Example 153

### tert-Butyl 2-(4-methylphenyl)cyclopropane-1-carboxylate

Synthesized from 4-methylstyrene with t-BDA. ¹H NMR (300 MHz, CDCl₃) trans-isomer: δ 7.08 (d, J=8.1 Hz, 2H), 6.98 (d, J=8.1 Hz, 2H), 2.41 (m, 1H), 2.31 (s, 3H), 1.79 (m, 1H), 1.50 (m, 1H), 1.46 (s, 9H), 1.20 (m, 1H). ¹³C NMR (75 MHz, CDCl₃) trans-isomer: δ 173.0, 137.7, 136.2, 129.3, 126.2, 80.7, 28.4, 25.8, 25.4, 21.2, 17.2.

### Example 154

### Ethyl 2-(3-methylphenyl)cyclopropane-1-carboxylate

Synthesized from 3-methylstyrene with EDA. ¹H NMR (300 MHz, CDCl₃) trans-isomer: δ 6.88-7.19 (m, 4H), 4.16 (q, J=7.2 Hz, 2H), 2.48 (m, 1H), 2.32 (s, 3H), 1.89 (m, 1H), 1.58 (m, 1H), 1.29 (m, 1H), 1.27 (t, J=7.2 Hz, 3H). ¹³C NMR (75 MHz, CDCl₃) trans-isomer: δ 173.4, 140.0, 138.1, 128.3, 127.2,126.9, 123.1, 60.6, 26.1, 24.1, 21.3, 17.0, 14.2.

### Example 155

### tert-Butyl 2-(3-methylphenyl)cyclopropane-1-carboxylate

Synthesized from 3-methylstyrene with t-BDA. ¹H NMR (300 MHz, CDCl₃) trans-isomer: δ 6.86-7.18 (m, 4H), 2.40 (m, 1H), 2.31 (s, 3H), 1.82 (m, 1H), 1.53 (m, 1H), 1.46 (s, 9H), 1.20 (m, 1H). ¹³C NMR (75 MHz, CDCl₃) trans-isomer: δ 172.6, 140.4, 138.0, 128.3, 127.0, 126.8, 123.0, 80.4, 28.1, 25.7, 25.2, 21.3, 17.0.

### Example 156

### Ethyl 2-(2-methylphenyl)cyclopropane-1-carboxylate

Synthesized from 2-methylstyrene with EDA. ¹H NMR (300 MHz, CDCl₃) trans-isomer: δ 7.14 (m, 3H), 6.99 (m, 1H), 4.20 (q, J=7.2 Hz, 2H), 2.51 (m, 1H), 2.38 (s, 3H), 1.78 (m, 1H), 1.57 (m, 1H), 1.31 (m, 1H), 1.29 (t, J=7.2 Hz, 3H). ¹³C NMR (75 MHz, CDCl₃) trans-isomer: δ 173.9, 138.0, 137.8, 129.8, 126.7, 125.8, 60.6, 24.6, 22.3, 19.5, 15.3, 14.3.

### Example 157

### tert-Butyl 2-(2-methylphenyl)cyclopropane-1-carboxylate

Synthesized from 2-methylstyrene with t-BDA. ¹H NMR (300 MHz, CDCl₃) trans-isomer: δ 7.11 (m, 3H), 6.97 (m, 1H), 2.42 (m, 1H), 2.38 (s, 3H), 1.68 (m, 1H), 1.50 (m, 1H), 1.48 (s, 9H), 1.25 (m, 1H). ¹³C NMR (75 MHz, CDCl₃) trans-isomer: δ 173.0, 138.1, 138.0, 129.8, 126.5, 125.8, 125.7, 80.4, 28.1, 24.3, 23.5, 19.5, 14.8.

### Example 158

### Ethyl 2-[4-(tert-butyl)phenyl]cyclopropane-1-carboxylate

Synthesized from 4-tert-butylstyrene with EDA. ¹H NMR (300 MHz, CDCl₃) trans-isomer: δ 7.31 (d, J=8.1 Hz, 2H), 7.04 (d, J=8.1 Hz, 2H), 4.16 (q, J=7.2 Hz, 2H), 2.49 (m, 1H), 1.88 (m, 1H), 1.58 (m, 1H), 1.30 (m, 1H), 1.30 (s, 9H), 1.27 (t, J=7.2 Hz, 3H). ¹³C NMR (75 MHz, CDCl₃) trans-isomer: δ 173.5, 149.4, 137.1, 125.8, 125.3, 60.6, 34.4, 31.3, 25.8, 24.1, 16.9, 14.2.

### Example 159

### tert-Butyl 2-[4-(tert-butyl)phenyl]cyclopropane-1-carboxylate

Synthesized from 4-tert-butylstyrene with t-BDA. ¹H NMR (300 MHz, CDCl₃) trans-isomer: δ 7.30 (d, J=8.1 Hz, 2H), 7.03 (d, J=8.1 Hz, 2H), 2.41 (m, 1H), 1.80 (m, 1H), 1.53 (m, 1H), 1.45 (s, 9H), 1.25 (s, 9H), 1.21 (m, 1H). ¹³C NMR (75 MHz, CDCl₃) trans-isomer: δ 172.7, 149.2, 137.5, 125.6, 125.3, 80.4, 34.3, 31.3, 28.1, 25.4, 25.3, 16.0.

### Example 160

### Ethyl 2-(4-bromophenyl)cyclopropane-1-carboxylate

Synthesized from 4-bromostyrene with EDA. ¹H NMR (300 MHz, CDCl₃) trans-isomer: δ 7.39 (d, J=8.7 Hz, 2H), 6.97 (d, J=8.7 Hz, 2H), 4.17 (q, J=7.2 Hz, 2H), 2.47 (m, 1H), 1.87 (m, 1H), 1.60 (m, 1H), 1.28 (t, J=7.2 Hz, 3H), 1.26 (m, 1H). ¹³C NMR (75 MHz, CDCl₃) trans-isomer: δ 173.1, 139.1, 131.4, 127.9, 120.1, 60.8, 25.5, 24.1, 17.0, 14.2.

### Example 161

### tert-Butyl 2-(4-bromophenyl)cyclopropane-1-carboxylate

Synthesized from 4-bromostyrene with t-BDA. ¹H NMR (300 MHz, CDCl₃) trans-isomer: δ 7.38 (d, J=8.1 Hz, 2H), 6.97 (d, J=8.1 Hz, 2H), 2.39 (m, 1H), 1.79 (m, 1H), 1.53 (m, 1H), 1.47 (s, 9H), 1.18 (m, 1H). ¹³C NMR (75 MHz, CDCl₃) trans-isomer: δ 172.2, 139.5, 131.4, 127.8, 119.9, 80.7, 28.1, 25.2, 25.1, 17.0.

### Example 162

### Ethyl 2-(4-chlorophenyl)cyclopropane-1-carboxylate

Synthesized from 4-chlorostyrene with EDA. ¹H NMR (300 MHz, CDCl₃) trans-isomer: δ 7.25 (d, J=8.4 Hz, 2H), 7.02 (d, J=8.4 Hz, 2H), 4.17 (q, J=7.2 Hz, 2H), 2.49 (m, 1H), 1.86 (m, 1H), 1.60 (m, 1H), 1.28 (t, J=7.2 Hz, 3H), 1.27 (m, 1H). ¹³C NMR (75 MHz, CDCl₃) trans-isomer: δ 173.1, 138.6, 132.1, 128.5, 127.5, 60.8, 25.5, 24.1, 17.0, 14.2.

### Example 163

### tert-Butyl 2-(4-chlorophenyl)cyclopropane-1-carboxylate

Synthesized from 4-chlorostyrene with t-BDA. ¹H NMR (300 MHz, CDCl₃) trans-isomer: δ 7.23 (d, J=8.7 Hz, 2H), 7.01 (d, J=8.7 Hz, 2H), 2.41 (m, 1H), 1.79 (m, 1H), 1.53 (m, 1H), 1.47 (s, 9H), 1.19 (m, 1H). ¹³C NMR (75 MHz, CDCl₃) trans-isomer: δ 172.2, 139.0, 131.9, 128.5, 127.4, 80.7, 28.1, 25.2, 25.0, 17.0.

### Example 164

### Ethyl 2-(4-fluorophenyl)cyclopropane-1-carboxylate

Synthesized from 4-fluorostyrene with EDA. ¹H NMR (300 MHz, CDCl₃) trans-isomer: δ 6.94-7.10 (m, 4H), 4.17 (q, J=7.2 Hz, 2H), 2.51 (m, 1H), 1.87 (m, 1H), 1.61 (m, 1H), 1.29 (m, 1H), 1.28 (t, J=7.2 Hz, 3H). ¹³C NMR (75 MHz, CDCl₃) trans-isomer: δ 173.3, 163.1, 159.9, 135.7, 127.8, 127.7, 115.4, 115.1, 60.7, 25.4, 24.0, 17.0, 14.2.

### Example 165

### tert-Butyl 2-(4-fluorophenyl)cyclopropane-1-carboxylate

Synthesized from 4-fluorostyrene with t-BDA. ¹H NMR (300 MHz, CDCl₃) trans-isomer: δ 6.93-7.08 (m, 4H), 2.43 (m, 1H), 1.76 (m, 1 H), 1.51 (m, 1H), 1.47 (s, 9H), 1.17 (m, 1H). ¹³C NMR (75 MHz, CDCl₃) trans-isomer: δ 172.4, 163.1, 159.8, 136.0, 127.5, 115.3, 115.0, 80.6, 28.1, 25.1, 24.9, 16.9.

### Example 166

### Ethyl 2-(4-trifluoromethylphenyl)cyclopropane-1-carboxylate

Synthesized from 4-trifluoromethylstyrene with EDA. ¹H NMR (300 MHz, CDCl₃) trans-isomer: δ 7.53 (d, J=8.1 Hz, 2H), 7.19 (d, J=8.1 Hz, 2H), 4.18 (q, J=7.2 Hz, 2H), 2.57 (m, 1H), 1.95 (m, 1H), 1.67 (m, 1H), 1.33 (m, 1H), 1.29 (t, J=7.2 Hz, 3H). ¹³C NMR (75 MHz, CDCl₃) trans-isomer: δ 172.9, 144.3, 126.3, 125.4, 60.9, 25.7, 24.5, 17.3, 14.2.

### Example 167

### tert-Butyl 2-(4-trifluoromethylphenyl)cyclopropane-1-carboxylate

Synthesized from 4-trifluoromethylstyrene with t-BDA. ¹H NMR (300 MHz, CDCl₃) trans-isomer: δ 7.52 (d, J=8.1 Hz, 2H), 7.18 (d, J=8.1 Hz, 2H), 2.48 (m, 1H), 1.88 (m, 1H), 1.60 (m, 1H), 1.48 (s, 9H), 1.27 (m, 1H). ¹³C NMR (75 MHz, CDCl₃) trans-isomer: δ 172.0, 144.7, 126.3, 125.3, 80.9, 28.1, 25.7, 25.3, 17.3.

### Example 168

### Ethyl 2-pentafluorophenylcyclopropane-1-carboxylate

Synthesized from pentafluorostyrene with EDA. ¹H NMR (300 MHz, CDCl₃) trans-isomer: δ 4.18 (q, J=7.2 Hz, 2H), 2.43 (m, 1H), 2.13 (m, 1H), 1.59 (m, 1H), 1.50 (m, 1H), 1.28 (t, J=7.2 Hz, 3H). ¹³C NMR (75 MHz, CDCl₃) trans-isomer: δ 172.7, 164.0, 61.1, 20.9, 15.0, 14.7, 14.2.

### Example 169

### tert-Butyl 2-pentafluorophenylcyclopropane-1-carboxylate

Synthesized from pentafluorostyrene with t-BDA. ¹H NMR (300 MHz, CDCl₃) trans-isomer: δ 2.38 (m, 1H), 2.08 (m, 1H), 1.46-1.62 (m, 2H), 1.49 (s, 9H). ¹³C NMR (75 MHz, CDCl₃) trans-isomer: δ 171.0, 158.5, 81.2, 28.1, 22.0, 14.8, 14.4.

### Example 170

### Ethyl 2-(4-acetoxyphenyl)cyclopropane-1-carboxylate

Synthesized from 4-acetoxystyrene with EDA. ¹H NMR (300 MHz, CDCl₃) trans-isomer: δ 7.11 (d, J=8.7 Hz, 2H), 6.99 (d, J=8.4 Hz, 2H), 4.17 (q, J=7.2 Hz, 2H), 2.51 (m, 1H), 2.29 (s, 3H), 1.88 (m, 1H), 1.59 (m, 1H), 1.29 (m, 1H), 1.28 (t, J=7.2 Hz, 3H). ¹³C NMR (75 MHz, CDCl₃) trans-isomer: δ 173.3, 169.6, 149.1, 137.7, 127.2, 121.5, 60.7, 25.6, 24.1, 21.1, 16.9, 14.2

### Example 171

### tert-Butyl 2-(4-acetoxyphenyl)cyclopropane-1-carboxylate

Synthesized from 4-acetoxystyrene with t-BDA. ¹H NMR (300 MHz, CDCl₃) trans-isomer: δ 7.10 (d, J=8.7 Hz, 2H), 6.99 (d, J=8.4 Hz, 2H), 2.43 (m, 1H), 2.28 (s, 3H), 1.81 (m, 1H), 1.52 (m, 1H), 1.47 (s, 9H), 1.22 (m, 1H). ¹³C NMR (75 MHz, CDCl₃) trans-isomer: δ 172.4, 169.6, 149.0, 138.1, 127.1, 121.5, 80.6, 28.1, 25.2, 21.1, 20.3, 16.9.

### Example 172

### Ethyl 2-methyl-2-phenylcyclopropane-1-carboxylate

Synthesized from a-methylstyrene with EDA. ¹H NMR (300 MHz, CDCl₃): trans-isomer: δ 7.18-7.30 (m, 5H), 4.19 (q, J=7.2 Hz, 2H), 1.96 (dd, J=8.1, 5.7 Hz, 1H), 1.52 (s, 3H), 1.42 (m, 2H), 1.29 (t, J=7.2 Hz, 3H). ¹³C NMR (75 MHz, CDCl₃) trans-isomer: δ 172.2, 145.9, 128.4, 127.3, 126.4, 60.5, 30.5, 27.8, 20.7, 19.8, 14.4.

### Example 173

### Ethyl 2,2-diphenylcyclopropane-1-carboxylate

Synthesized from 1,1-diphenylethylene with EDA. ¹H NMR (300 MHz, CDCl₃) trans-isomer: δ 7.14-7.36 (m, 10H), 3.91 (m, 2H), 2.53 (dd, J=8.1, 6.3 Hz, 1H), 2.17 (m, 1H), 1.58 (dd, J=8.1, 4.8 Hz, 1H), 1.00 (t, J=7.2 Hz, 3H). ¹³C NMR (75 MHz, CDCl₃) trans-isomer: δ 170.6, 144.8, 140.2, 129.7, 128.4, 128.2, 127.5, 126.9, 126.5, 60.4, 39.8, 29.0, 20.1, 14.0.

### Example 174

### Ethyl 2-(2-naphthalenyl)cyclopropane-1-carboxylate

Synthesized from 2-vinylnaphthalene with EDA. ¹H NMR (300 MHz, CDCl₃) trans-isomer: δ 7.77 (m, 3H), 7.56 (s, 1H), 7.43 (m, 2H), 7.19 (d, J=8.4 Hz, 1H), 4.19 (q, J=7.2 Hz, 2H), 2.69 (m, 1H), 2.00 (m, 1H), 1.67 (m, 1H), 1.43 (m, 1H), 1.31 (t, J=7.2 Hz, 3H). ¹³C NMR (75 MHz, CDCl₃) trans-isomer: δ 173.4, 137.5, 133.3, 132.2, 128.2, 127.6, 127.4, 126.2, 125.4, 124.7, 124.5, 60.7, 26.4, 24.1, 17.0, 14.2.

### Example 175

### tert-Butyl 2-(2-naphthalenyl)cyclopropane-1-carboxylate

Synthesized from 2-vinylnaphthalene with t-BDA. ¹H NMR (300 MHz, CDCl₃) trans-isomer: δ 7.76 (m, 3H), 7.55 (s, 1H), 7.43 (m, 2H), 7.17 (d, J=8.4 Hz, 1H), 2.61 (m, 1H), 1.94 (m, 1H), 1.60 (m, 1H), 1.48 (s, 9H), 1.34 (m, 1H). ¹³C NMR (75 MHz, CDCl₃) trans-isomer: δ 172.5, 137.8, 133.3, 132.3, 128.1, 127.6, 127.3, 126.2, 125.3, 124.6, 124.4, 80.6, 28.1, 26.0, 25.3, 17.0.

### Example 176

### General Procedure for Intramolecular Cyclopropanation

The cobalt porphyrin catalyst (21a, 21d, 21f, 21j, 21l, 21n, 21q, or 21r), diazoacetates (if solid), and DMAP were added to an oven dried resealable Schlenk tube (that had been previously evacuated and back filled with nitrogen). The tube was capped with a Teflon screw cap, evacuated, and backfilled with nitrogen. The screw cap was replaced with a rubber septum and the solvent was added via syringe. The tube was then purged with nitrogen for 2 min, and the septum was replaced with the Teflon screw cap. The tube was sealed and placed in an oil bath with the desired temperature with stirring. The resulting product was cooled to room temperature and its contents taken up with methylene chloride and rotary evaporated to dryness. The crude mixture was purified by passing the reaction mixture through a pipette loaded with silica gel and eluted with methylene chloride.

### Example 177

### (1R,5S)-6,6-Dimethyl-oxabicyclo[3.1.0]hexan-2-one

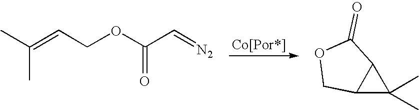

(1R,5S)-6,6-Dimethyl-oxabicyclo[3.1.0]hexan-2-one was synthesized by Co-porphyrin (21a, 21d, 21f, 21j, 21l, 21n, 21q, or 21r) catalyzed decomposition of 3-methyl-2-buten-1-yl diazoacetate as a clear liquid in up to 62% yield and 90% ee. Enantiomer separation was performed on a Chiraldex G-TA column: 22.94 min for (1S,5R)-enantiomer and 24.43 min for (1R,5S)-enantiomer. ¹H NMR (300 MHz, CDCl₃) δ 4.34 ppm (dd, J=5.4, 9.9 Hz, 1H), 4.11 ppm (d, J=9.9 Hz, 1H), 2.02 ppm (pt, J=6 Hz, 1H), 1.92 ppm (d, J=6.3 Hz, 1H), 1.15 ppm (s, 3H), 1.14 ppm (s, 3H).

### Example 178

### (1R,5S)-3-Oxabicyclo[3.1.0]hexan-2-one

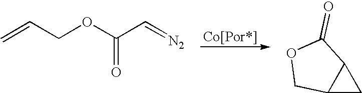

(1R,5S)-3-Oxabicyclo[3.1.0]hexan-2-one was synthesized by Co-porphyrin [21l or 21a] catalyzed decomposition of 2-propen-1-yl diazoacetate.

### Example 179

### [1S-(1α;5α,6β]-6-Phenyl-3-oxabicyclo[3.1.0]hexan-2-one

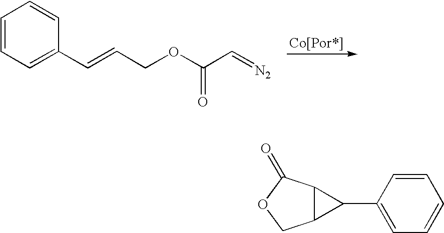

[1S-(1α;5α,6β]-6-Phenyl-3-oxabicyclo[3.1.0]hexan-2-one was synthesized by Co-porphyrin [21a, 21d, 21f, 21j, 21l, 21n, 21q, or 21r] catalyzed decomposition of trans-3-phenyl-2-propen-1-yl diazoacetate as a white solid in up to 95% yield and 71% ee. Enantiomer separation was performed on a Chiraldex G-TA column: 27.64 min for (1S)-enantiomer and 31.40 min for (1R)-enantiomer. ¹HNMR (300 MHz, CDCl₃) δ 7.28 ppm (comp,3H), 7.06 ppm (d, 2H), 4.47 ppm (dd, 1H), 4.41 ppm (d, 1H), 2.53 ppm (m, 1H), 2.32 ppm (comp, 2H).

### Example 180

### [1S-(1α;5α,6β]-6-para Chlorophenyl-3-oxabicyclo[3.1.0]hexan-2-one

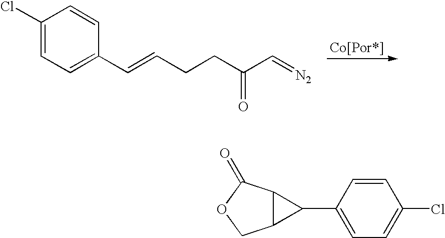

[1S-(1α;5α,6β]-6-para Chlorophenyl-3-oxabicyclo[3.1.0]hexan-2-one was synthesized by Co-porphyrin [21l or 21a] catalyzed decomposition of trans-3-(para-chlorophenyl)-2-propen-1-yl diazoacetate as a white solid in up to 50% yield and 81% ee. Enantiomer separation was performed on a Chiraldex G-TA column: 73.09 (1S)-enantiomer and 76.58 min for (1R)-enantiomer. ¹HNMR (300 MHz, CDCl₃) δ 7.28 ppm (d, 2H), 7.00 ppm (d, 2H), 4.47 ppm (dd, 1H), 4.41 ppm (d, 1H), 2.51 ppm (m, 1H), 2.29 ppm (comp, 2H).

### Example 181

### [1S-(1α;5α,6β]-6-para Bromophenyl-3-oxabicyclo[3.1.0]hexan-2-one

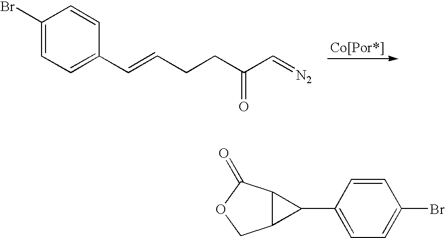

[1S-(1α;5α,6β]-6-para Bromophenyl-3-oxabicyclo[3.1.0]hexan-2-one was synthesized by Co-porphyrin [21l or 21a] catalyzed decomposition of trans-3-(para-bromophenyl)-2-propen-1-yl diazoacetate as a white solid in up to 82% yield and 81% ee. Enantiomer separation was performed on a Chiraldex G-TA column: 85.37 min (1S)-enantiomer and 90.16 min for (1R)-enantiomer. ¹HNMR (300 MHz, CDCl₃) δ 7.40 ppm (d, J=8.7 Hz, 2H), 6.91 ppm (d, J=8.4 Hz, 2H), 4.42 ppm (m, 2H), 2.49 ppm (m, 1H), 2.27 ppm (comp, 2H).

### Example 182

### [1S-(1α;5α,6β]-6-para Triflouromethylphenyl-3-oxabicyclo[3.1.0]hexan-2-one

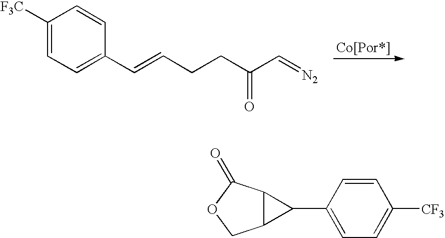

[1S-(1α;5α,6β]-6-para Triflouromethylphenyl-3-oxabicyclo[3.1.0]-hexan-2-one was synthesized by Co-porphyrin [21l or 21a] catalyzed decomposition of trans-3-(para-trifluoromethyl phenyl)-2-propen- 1-yl diazoacetate as a white solid in up to 50% ee. Enantiomer separation was performed on a Chiraldex G-TA column: 58.55 (1S)-enantiomer and 66.36 min for (1R)-enantiomer. ¹HNMR (300 MHz, CDCl₃) δ 7.55 ppm (d, J=8.1 HZ, 2H), 7.16 ppm (d, J=8.1 Hz, 2H), 4.45 ppm (m, 2H), 2.55 ppm (m, 1H), 2.36 ppm (comp, 2H).

### Example 183

### [1S-(1α;5α,6β]-6-para Methoxyphenyl-3-oxabicyclo[3.1.0]hexan-2-one

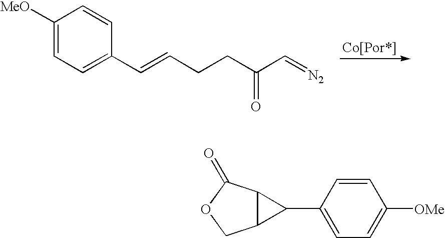

[1S-(1α;5α,6β]-6-para Methoxyphenyl-3-oxabicyclo[3.1.0]hexan-2-one was synthesized by Co-porphyrin [21l or 21a] catalyzed decomposition of trans-3-(para-methoxyphenyl)-2-propen-1-yl diazoacetate as a white solid in up to 99% yield and 68% ee. Enantiomer separation was performed on a Chiraldex G-TA column.

### Example 184

### [1S-(1α;5α,6β-6-para tert-Butylphenyl-3-oxabicyclo[3.1.0]hexan-2-one

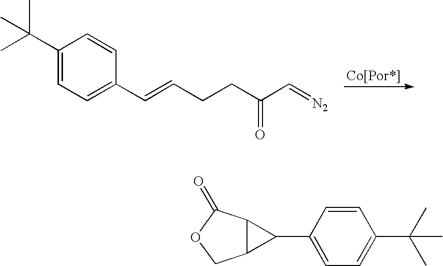

[1S-(1α;5α,6β]-6-para tert-Butylphenyl-3-oxabicyclo[3.1.0]hexan-2-one was synthesized by Co-porphyrin [21l or 21a] catalyzed decomposition of trans-3-(para-tert butylphenyl)-2-propen-1-yl diazoacetate as a white solid in up to 99% yield and 24% ee. Enantiomer separation was performed on a Chiraldex G-TA column.

### Example 185

### General Considerations for Aziridination Reactions

All reactions were carried out under nitrogen atmosphere in an oven dried Schlenk tube. All olefins were purchased from Acros or Aldrich Chemicals and used without further purification. Diphenylphosphoryl azide was purchased from Acros Chemicals. Acetonitrile and chlorobenzene were dried with calcium hydride in reflux. All metalloporphyrins were purchased from Strem or Mid-century Chemicals. Bromamine-T was prepared from Chloramine-T according to the literature procedure and dried at 80° C. in vacuum overnight before use. See Nair, C. G .R.: Indrasenan, P. Talanta 1976, 23, 239; Nair, C. G. N., et al., Talanta 1978, 25, 525; and Mahadevappa. D. S., et al., Tetrahedron 1984, 40, 1673. Proton, carbon, phosphorous, and fluorine nuclear magnetic resonance spectra (¹H NMR, ¹³C NMR, ³¹P NMR, and ¹⁹F NMR) were recorded on a Varian Mercury 300 spectrometer and referenced with respect to residual solvent. Infrared spectra were obtained using a Bomem B100 Series FT-IR spectrometer. Samples were prepared as films on a NaCl plate with chloroform as solvent. Thin layer chromatography was carried out on E. Merck Silica Gel 60 F-254 TLC plates.

### Example 186

### General Procedure for Aziridination of Alkenes with Bromamine-T

An oven dried Schlenk tube equipped with stirring bar was degassed on vacuum line and purged with nitrogen. The tube was charged with metalloporphyrin (5 mol %), Bromamine-T (0.4 mmol) and activated 5 A molecular sieves (500 mg). The tube was capped with a Teflon screw cap, evacuated on vacuum line for 30-45 min. The Teflon screw cap was replaced with a rubber septum and 3-5 mL of solvent and substrate (0.2 mmol) were then added successively. The tube was purged with nitrogen for 1-2 min and the contents were stirred overnight at ambient temperatures. After completion of the reaction, molecular sieves were removed by filtration and the filtrate was concentrated under vacuum. The solid residue was purified by flash chromatography (silica gel, ethyl acetate:hexanes (V:V)=3:7) to afford the pure product.

### Example 187

N-(p-Tolylsulfonyl)-2-phenylaziridine was synthesized from styrene as substrate and the product obtained as a yellow oil (45 mg, yield, 83%). ¹H NMR (300 MHz, CDCl₃): δ 7.83 (δ, 2H, J=8.1 Hz), 7.17-7.31 (m, 7H), 3.73 (dd, 1H, J=7.2, 4.5 Hz), 2.94 (d, 1H, J=7.2 Hz), 2.39 (s, 3H), 2.35 (d, 1H, J=4.5 Hz). ¹³C NMR (75 MHz, CDCl₃): δ 144.6, 140.5, 135.0, 134.8, 129.7, 128.5, 128.3, 127.9, 126.5, 41.0, 35.9, 21.6. FT-IR (film, cm⁻¹): 3064, 3034, 2924, 2855, 1597, 1495, 1459, 1385, 1324, 1292, 1232, 1190, 1160, 978, 909, 815, 775, 758, 715, 696, 665.

### Example 188

N-(p-Tolylsulfonyl)-2-(p-methylphenyl)aziridine was synthesized from p-methylstyrene as substrate and the product obtained as a yellow oil (43 mg, yield, 76%). ¹H NMR (300 MHz, CDCl₃): δ 7.84 (δ, 2H J=8.1 Hz), 7.31 (δ, 2H, J=8.4 Hz), 7.08 (s, 4H), 3.73 (dd, 1H, J=7.2,-4.5 Hz), 2.95 (d, 1H, J=7.2 Hz), 2.41 (s, 3H), 2.36 (d, 1H, J=4.5 Hz), 2.29 (s, 3H). FT-IR (film, cm⁻¹): 3057, 1600, 1150, 916. ¹³C NMR (75 MHz, CDCl₃): δ 144.6, 140.5, 138.1, 134.9, 131.9, 129.7, 129.2, 127.9, 126.4, 41.0, 35.8, 21.6, 21.1. FT-IR (film, cm⁻¹): 2922, 2857, 1597, 1517, 1494, 1453, 1381, 1324, 1292, 1232, 1186, 1160, 1118, 1093, 1019, 979, 911, 815, 731, 716, 693, 665.

### Example 189

N-(p-Tolylsulfonyl)-2-(p-tert-butylphenyl)aziridine was synthesized from p-tert-butylstyrene as substrate and the product obtained as a yellow oil (53 mg, yield, 81%). ¹H NMR (300 MHz, CDCl₃): δ 7.85 (d, 2H, J=8.1 Hz), 7.29-7.33 (m, 4H), 7.13 (d, 2H, J=8.1 Hz), 3.75 (dd, 1H, J=7.2, 4.5 Hz), 2.94 (d, 1H, J=7.2 Hz), 2.42 (s, 3H), 2.37 (d, 1H, J=4.8 Hz), 1.28 (s, 9H). ¹³C NMR (75 MHz, CDCl₃): δ 151.4, 147.4, 144.6, 134.9, 131.9, 129.7, 127.9, 126.2, 125.8, 40.9, 35.8, 34.5, 31.2, 21.6. FT-IR (film, cm⁻¹): 3281, 2962, 2869, 1688, 1599, 1511, 1459, 1410, 1332, 1271, 1161, 1093, 1019, 958, 834, 757, 725, 705, 663.

### Example 190

N-(p-Tolylsulfonyl)-2-(3-methylphenyl)aziridine was synthesized from 3-methylstyrene as substrate and the product obtained as a yellow oil (51 mg, yield, 89%). ¹H NMR (300 MHz, CDCl₃): δ 7.85 (δ, 2H, J=8.4 Hz), 7.31 (δ, 2H, J=8.7 Hz), 7.01-7.16 (m, 4H), 3.73 (dd, 1H, J=7.2, 4.5 Hz), 2.94 (d, 1H, J=6.9 Hz), 2.42 (s, 3H), 2.36 (d, 1H, J=4.5 Hz), 2.29 (s, 3H). ¹³C NMR (75 MHz, CDCl₃): δ 144.6, 138.3, 134.8, 129.7, 129.0, 128.4, 127.9, 127.1, 123.6, 41.0, 35.9, 21.6, 21.2. FT-IR (film, cm⁻¹): 3292, 2922, 2362, 1688, 1596, 1492, 1453, 1382, 1325, 1292, 1215, 1184, 1160, 1093, 1039, 1019, 981, 929, 867, 816, 787, 720, 692, 666.

### Example 191

N-(p-Tolylsulfonyl)-2-(p-chloromethylphenyl)aziridine) was synthesized from p-vinylbenzyl chloride as substrate and the product obtained as a yellow oil (55 mg, yield, 86%). ¹H NMR (300 MHz, CDCl₃): δ 7.85 (d, 2H, J=8.4 Hz), 7.30 (m, 4H), 7.20 (d, 2H, J=7.8 Hz), 4.54 (s, 2H), 3.76 (dd, 1H, J=4.5, 7.2 Hz), 3.00 (d, 1H, J=6.9 Hz), 2.44 (s, 3H), 2.36 (d, 1H, J=4.2 Hz). ¹³C NMR (75 MHz, CDCl₃): δ 145.3, 135.4, 129.8, 128.8, 128.0, 127.9, 126.9, 45.7, 40.5, 36.0, 21.6. FT-IR (film, cm⁻¹): 2362, 1596, 1519, 1493, 1453, 1383, 1324, 1268, 1187, 1161, 1093, 1019, 981, 909, 815, 750, 715, 674.

### Example 192

N-(p-Tolylsulfonyl)-2-(4-trifluoromethylphenyl)aziridine was synthesized from 4-(trifluoromethyl)styrene as substrate and the product obtained as a yellow oil (61 mg, yield, 90%). ¹H NMR (300 MHz, CDCl₃):δ 7.86 (d, 2H, J=8.1 Hz), 7.54 (d, 2H, J=8.1 Hz), 7.34 (d, 4H, J=8.1 Hz), 3.80 (dd, 1H, J=4.2, 7.2 Hz), 3.01 (d, 1H, J=7.2 Hz), 2.44 (s, 3H), 2.37 (d, 1H, J=4.5 Hz). ¹³C NMR (75 MHz, CDCl₃): δ 147.4, 145.0, 139.2, 134.6, 129.8, 127.9, 126.9, 125.6, 125.5, 125.4, 40.1, 36.2, 21.6. ¹⁹F NMR (75 MHz, CDCl₃): −63.1. FT-IR (film cm⁻¹): 2926, 2362, 1621, 1597, 1494, 1454, 1421, 1383, 1325, 1233, 1163, 1121, 1093, 1067, 1018, 982, 911, 840, 817, 751, 697, 661.

### Example 193

N-(p-Tolylsulfonyl)-2-(4-acetoxylphenyl)aziridine was synthesized from 4-acetoxystyrene as substrate and the product obtained as a yellow oil (61 mg, yield, 92%). ¹H NMR (300 MHz, CDCl₃): δ 7.86 (d, 2H, J=8.1 Hz), 7.33 (d, 2H, J=8.1 Hz), 7.21 (d, 4H, J=8.4 Hz), 7.01 (d, 4H, J=8.4 Hz), 3.75 (dd, 1H, J=4.2, 7.2 Hz), 2.97 (d, 1H, J=7.2 Hz), 2.43 (s, 3H), 2.35 (d, 1H, J=4.5 Hz), 2.27 (s, 3H). ¹³C NMR (75 MHz, CDCl₃): δ 169.4, 150.5, 144.7, 134.7, 132.6, 129.8, 127.9, 127.6, 121.7, 40.4, 36.0, 21.6, 21.0. FT-IR (film cm⁻¹): 3583, 2925, 1762, 1597, 1510, 1453, 1371, 1325, 1194, 1162, 1093, 1016, 982, 911, 852, 816, 724, 710, 694, 666.

### Example 194

N-(p-Tolylsulfonyl)-2-(2′,4′,6′-trimethylphenyl)aziridine was synthesized from 2,4,6-trimethylstyrene as substrate and the product obtained as a yellow oil (39 mg, yield, 61%). ¹H NMR (300 MHz, CDCl₃) δ 7.88 (d, 2H, J=8.4 Hz), 7.35 (d, 2H, J=7.8 Hz), 6.79 (s, 2H), 3.86 (t, 1H, J=4.8 Hz), 2.93 (d, 1H, J=7.5 Hz), 2.46 (s, 3H), 2.31 (s, 6H), 2.24 (s, 3H), 2.16 (d, 1H, J=4.5 Hz). ¹³C NMR (75 MHz, CDCl₃): δ 144.9, 139.8, 137.4, 135.2, 129.7, 129.1, 128.8, 128.2, 127.1, 38.9, 35.4, 21.7, 20.9, 20.1. FT-IR (film cm⁻¹): 3583, 3282, 2923, 1711, 1610, 1449, 1331, 1160, 1092, 934, 852, 814, 754, 665.

### Example 195

N-(p-Tolylsulfonyl)-2-(2-naphthyl)aziridine was synthesized from 2-vinylnapthalene as substrate and the product obtained as a yellow oil (34 mg, yield, 53%). ¹H NMR (300 MHz, CDCl₃): δ 7.87 (d, 2H, J=8.1 Hz), 7.71-7.79 (m, 4H), 7.46 (m, 2H), 7.30 (d, 2H, J=8.4 Hz), 7.26 (s, 1H), 3.80 (d, 1H, J=7.2, 4.5 Hz), 3.05 (d, 1H, J=7.2 Hz), 2.47 (d, 1H, J=4.5 Hz), 2.40 (s, 3H). ¹³C NMR (75 MHz, CDCl₃): δ 147.4, 144.7, 134.9, 133.1, 133.0, 132.4, 129.8, 128.5, 127.9, 127.8, 127.7, 126.4, 126.3, 126.1, 123.6, 41.3, 36.0, 21.6. FT-IR (film cm⁻¹): 3583, 3056, 2923, 2362, 1685, 1597, 1509, 1453, 1398, 1324, 1160, 1093, 1019, 953, 920, 858, 816, 751, 722, 666.

### Example 196

N-(p-Tolylsulfonyl)-2-(p-bromophenyl)aziridine was synthesized from 4-bromostyrene as substrate and the product obtained as a yellow oil (49 mg, yield, 70%). ¹H NMR (300 MHz, CDCl₃): δ 7.83 (d, 2H, J=8.4 Hz), 7.40 (d, J=8.4 Hz), 7.31 (d, 2H, J=8.4 Hz), 7.06 (d, 2H, J=8.4 Hz), 3.70 (dd, 1H, J=4.2, 7.2 Hz), 2.95 (d, 1H, J=7.2 Hz), 2.42 (s, 3H), 2.32 (d, 1H, J=4.5 Hz). ¹³C NMR (75 MHz, CDCl₃): δ 144.8, 134.7, 134.1, 131.7, 129.7, 128.2, 127.9, 122.3, 40.2, 35.9, 21.6. FT-IR (film cm⁻¹): 2922, 1491, 1453, 1407, 1377, 1325, 1229, 1186, 1161, 1115, 1093, 1071, 1012, 981, 910, 816, 769, 727, 705, 693, 665.

### Example 197

N-(p-Tolylsulfonyl)-2-(p-chlorophenyl)aziridine was synthesized from 4-chlorostyrene as substrate and the product obtained as a yellow oil (44 mg, yield, 71%). ¹H NMR (300 MHz, CDCl₃):δ7.85 (d, 2H, J=8.4 Hz), 7.33 (d, 2H, J=8.1 Hz), 7.27 (d, 2H, J=8.7 Hz), 7.16 (d, 2H, J=8.4 Hz), 3.73 (dd, 1H, J=4.2, 7.2 Hz), 2.97 (d, 1H, J=6.9 Hz), 2.44 (s, 3H), 2.34 (d, 1H, J=4.2 Hz). ¹³C NMR (75 MHz, CDCl₃): δ 144.8, 134.7, 134.1, 133.6, 129.8, 128.7, 127.9, 40.2, 36.0, 21.6. FT-IR (film cm⁻¹): 2924, 2361, 1597, 1494, 1454, 1414, 1378, 1325, 1306, 1230, 1188, 1162, 1092, 1016, 981, 911, 816, 776, 729, 708, 694, 669.

### Example 198

N-(p-Tolylsulfonyl)-2-(p-fluorophenyl)aziridine was synthesized from 4-fluorostyrene as substrate and the product obtained as a yellow oil (50 mg, yield, 86%). ¹H NMR (300 MHz, CDCl₃): δ 7.83 (d, 2H, J=8.1 Hz), 7.31 (d, 2H, J=8.4 Hz), 7.16 (m, 2H), 6.96 (m, 2H), 3.74 (dd, 1H, J=4.5, 7.2 Hz), 2.94 (d, 1H, J=7.2 Hz), 2.42 (s, 3H), 2.32 (d, 1H, J=4.5 Hz). ¹³C NMR (75 MHz, CDCl₃): δ 144.8, 134.7, 130.7, 129.8, 128.3, 128.2, 127.9, 115.7, 115.4, 40.2, 36.0, 21.6. FT-IR (film cm⁻¹): 3583, 3069, 2924, 1600, 1513, 1454, 1379, 1325, 1235, 1188, 1162, 1093, 1017, 982, 912, 839, 818, 720, 692, 665.

### Example 199

N-(p-Tolylsulfonyl)-2-(pentafluorophenyl)aziridine was synthesized from pentafluorostyrene as substrate and the product obtained as a yellow oil (45 mg, yield, 61%). ¹H NMR (300 MHz, CDCl₃): δ 7.83 (d, 2H, J=8.1 Hz), 7.34 (d, 2H, J=8.1 Hz), 3.77 (dd, 1H, J=4.5, 7.2 Hz), 3.01 (d, 1H, J=7.2 Hz), 2.77 (d, 1H, J=4.2 Hz), 2.44 (s, 3H). ¹³C NMR (75 MHz, CDCl₃): δ 145.2, 134.1, 129.8, 128.3, 32.4, 31.9, 21.7. ¹⁹F NMR (75 MHz, CDCl₃): −142.6, −153.1, −161.8. FT-IR (film cm⁻¹): 2926, 1656, 1597, 1525, 1504, 1456, 1379, 1333, 1307, 1230, 1186, 1164, 1131, 1093, 1023, 975, 943, 873, 816, 778, 747, 711, 696, 673.

### Example 200

N-(p-Tolylsulfonyl)-2-methyl-2-phenylaziridine was synthesized from α-methylstyrene as substrate and the product obtained as a yellow oil (42 mg, yield, 73%). ¹H NMR (300 MHz, CDCl₃): δ 7.85 (d, 2H, J=8.4 Hz), 7.28-7.36 (m, 7H), 2.95 (s, 1H), 2.51 (s, 1H), 2.42 (s, 3H), 2.03 (s, 3H). ¹³C NMR (75 MHz, CDCl₃): δ 144.0, 141.0, 137.6, 129.5, 128.4, 127.7, 127.5, 126.5, 51.8, 41.8, 21.6, 20.9. FT-IR (film cm⁻¹): 3276, 2362, 1598, 1495, 1447, 1326, 1159, 909, 815, 765, 703, 666.

### Example 201

N-(p-Tolylsulfonyl)-2,2-diphenylaziridine was synthesized from 1,1-diphenylethylene as substrate and the product obtained as a yellow oil (57 mg, yield, 81%). ¹H NMR (300 MHz, CDCl₃): δ 7.45 (d, 2H, J=7.8 Hz), 7.00-7.14 (m, 12 H), 2.83 (s, 2H), 2.18 (s, 3H). ¹³C NMR (75 MHz, CDCl₃): δ 147.4, 129.7, 129.4, 128.8, 128.6, 128.2, 128.1, 127.9, 40.8, 21.6. FT-IR (film cm⁻¹): 3273, 3061, 2362, 1722, 1597, 1557, 1542, 1492, 1448, 1398, 1327, 1279, 1159, 1090, 1020, 943, 917, 814, 756, 700, 673.

### Example 202

N-(p-Tolylsulfonyl)amino-1,2,3,4-tetrahydronapthalene-1,2-imine was synthesized from 1,2-dihydronapthalene as substrate and the product obtained as a yellow oil (20 mg, yield, 33%). ¹H NMR (300 MHz, CDCl₃): δ 7.82 (d, 2H, J=8.1 Hz), 7.30 (d, 2H, J=8.1 Hz), 7.03-7.25 (m, 4H), 3.81 (d, 1H, J=7.2 Hz), 3.55 (d, 1H, J=6.9 Hz), 2.72 (dt, 1H, J=13.5, 6.3 Hz), 2.5 (dd, 1H, J=13.5, 5.4 Hz), 2.42 (s, 3H), 2.24 (dd, 1H, J=14.8, 6.3 Hz), 1.62 (m, 1H). ¹³C NMR (75 MHz, CDCl₃): δ 144, 136.6, 135.5, 129.7, 129.4, 128.5, 128.4, 127.6, 126.3, 114.3, 42.0, 41.7, 24.7, 21.6, 19.9. FT-IR (film cm⁻¹): 3585, 3026, 2925, 2854, 1598, 1494, 1433, 1398, 1321, 1229, 1157, 1091, 1028, 989, 945, 908, 877, 814, 754, 731, 715, 670.

### Example 203

2,2-Dimethyl-1-(toluene-4-sulfonyl)-1,1a,2,7b-tetrahydro-3-oxa-1-aza-cyclopropa[a]naphthalene-6-carbonitrile was synthesized from 2,2-dimethyl-2H-1-benzopyran-6-carbonitrile (185 mg, 1.0 mmol) and bromamine-T (54.4 mg, 0.2 mmol) and the product obtained as a yellow oil (47 mg, yield, 66%). ¹H NMR (300 MHz, CDCl₃): δ 7.80 (d, 2H, J=8.1 Hz), 7.53 (d, 1H, J=1.8 Hz), 7.46 (dd, 2H, J=8.4, 1.8 Hz), 7.31 (d, 2H, J=8.1 Hz), 6.80 (d, 1H, J=8.4 Hz), 3.86 (d, 1H, J=7.2 Hz), 3.35 (d, 1H, J=7.5 Hz), 2.43 (s, 3H), 1.29 (s, 3H), 1.24 (s, 3H). ¹³C NMR (75 MHz, CDCl₃): δ 156.3, 145.2, 134.4, 134.2, 133.2, 129.8, 128.0, 119.3, 119.2, 104.7, 73.2, 49.2, 38.7, 25.8, 23.8, 21.7. FT-IR (film cm⁻¹): 2980, 2925, 2854, 2360, 2227, 1615, 1598, 1579, 1492, 1461, 1328, 1276, 1251, 1208, 1159, 1092, 1025, 962, 935, 874, 850, 833, 784, 768, 717, 674.

### Example 204

trans-N-(p-Tolylsulfonyl)-2-methyl-3-phenylaziridine was synthesized from cis-β-methystyrene as substrate and the product obtained as a yellow oil (54 mg, yield, 94%). ¹H NMR (300 MHz, CDCl₃): δ 7.84 (d, 2H, J=8.4 Hz), 7.24-7.30 (m, 5 H), 7.15-7.19 (m, 2H), 3.81 (d, 1H, J=4.2 Hz), 2.93 (dq, 1H, J=6.3, 4.8 Hz), 2.40 (s, 3H), 1.85 (d, 3H, J=5.7 Hz). ¹³C NMR: δ 143.9, 137.8, 135.5, 129.5, 128.5, 128.0, 127.1, 126.2, 49.1, 49.0, 21.5, 14.1. FT-IR (film, cm⁻¹): 3030, 1400, 1090, 970, 890. FT-IR (film cm⁻¹): 2929, 1598, 1497, 1455, 1413, 1383, 1321, 1239, 1205, 1184, 1159, 1091, 1059, 1037, 971, 890, 815, 749, 697, 685.

### Example 205

(cis-&trans)-N-(p-Tolylsulfonyl)-2,3-diphenylaziridine was synthesized with cis-stilbene (36 μL, 0.2 mmol) with bromamine-T (163.0 mg, 0.6 mmol) in the presence of 10 mole % Co(DCITPP) (19 mg, 0.2 mmol), and the products obtained as a mixture of cis- and trans-aziridine (65 mg, yield, 92%). For cis-N-(p-Tolylsulfonyl)-2,3-diphenylaziridine ¹H NMR (300 MHz, CDCl₃): δ 7.96 (d, 2H, J=8.4 Hz), 7.47 (d, 2H, J=8.7 Hz), 7.03-7.13 (m, 10H), 4.23 (s, 2H), 2.45 (s, 3H). For trans-N-(p-Tolylsulfonyl)-2,3-diphenylaziridine ¹H NMR (300 MHz, CDCl₃): δ 7.63 (d, 2H, J=8.4 Hz), 7.34-7.44 (m, 10H), 7.20 (d, 2H, J=8.4 Hz), 4.27 (s, 2H), 2.38 (s, 3H). ¹³C NMR (75 MHz, CDCl₃): δ 144.7, 143.9, 137.3, 137.0, 134.8, 133.0, 132.0, 129.8, 129.4, 128.6, 128.4, 128.2, 128.0, 127.9, 127.7, 127.6, 127.5, 126.5, 50.3, 47.4, 21.7, 21.6. FT-IR (film cm⁻¹): 3061, 3031, 2923, 2854, 1598, 14957, 1452, 1401, 1327, 1160, 1090, 1026, 908, 813, 785, 760, 697, 674.

### Example 206

trans-N-(p-Tolylsulfonyl)-2-methyl-3-phenylaziridine was synthesized from trans-β-methystyrene as substrate and the product obtained as a yellow oil (51 mg, yield, 87%). ¹H NMR (300 MHz, CDCl₃): δ 7.84 (d, 2H, J=8.4 Hz), 7.24-7.30 (m, 5 H), 7.15-7.19 (m, 2H), 3.81 (d, 1H, J=4.2 Hz), 2.93 (dq, 1H, J=6.3, 4.8 Hz), 2.40 (s, 3H), 1.85 (d, 3H, J=5.7 Hz). ¹³C NMR: δ 143.9, 137.8, 135.5, 129.5, 128.5, 128.0, 127.1, 126.2, 49.1, 49.0, 21.5, 14.1. FT-IR (film cm⁻¹): 3583, 2928, 1598, 1497, 1456, 1413, 1383, 1320, 1238, 1205, 1184, 1159, 1091, 1059, 1037, 971, 890, 815, 748, 697, 685, 665.

### Example 207

(cis-&trans)-N-(p-Tolylsulfonyl)-2,3-diphenylaziridine was synthesized with trans-stilbene (36 mg, 0.2 mmol) with bromamine-T (163.0 mg, 0.6 mmol) in the presence of 10 mole % Co(DCITPP) (19 mg, 0.2 mmol), and the products obtained as a mixture of cis- and trans-aziridine (66 mg, yield, 94%). For cis-N-(p-Tolylsulfonyl)-2,3-diphenylaziridine ¹H NMR (300 MHz, CDCl₃): δ 7.96 (d, 2H, J=8.4 Hz), 7.47 (d, 2H, J=8.7 Hz), 7.03-7.13 (m, 10H), 4.23 (s, 2H), 2.45 (s, 3H). For trans-N-(p-Tolylsulfonyl)-2,3-diphenylaziridine ¹H NMR (300 MHz, CDCl₃): δ 7.63 (d, 2H, J=8.4 Hz), 7.34-7.44 (m, 10H), 7.20 (d, 2H, J=8.4 Hz), 4.27 (s, 2H), 2.38 (s, 3H). ¹³C NMR (75 MHz, CDCl₃): δ 144.7, 143.9, 137.3, 137.0, 134.8, 133.0, 132.0, 129.8, 129.4, 128.6, 128.4, 128.2, 128.0, 127.9, 127.7, 127.6, 127.5, 126.5, 50.3, 47.4, 21.7, 21.6. FT-IR (film cm⁻¹): 3583, 3273, 3062, 3032, 1723, 1598, 1495, 1451, 1399, 1327, 1160, 1092, 1025, 906, 813, 785, 752, 699, 669.

### Example 208

N-(p-Tolylsulfonyl)-6-(azabicyclo)[3.1.0]hexane was synthesized from cyclopentene as substrate and the product obtained as a yellow oil (29 mg, yield, 61%). ¹H NMR (300 MHz, CDCl₃): δ 7.80 (d, 2H, J=8.4Hz), 7.31 (d, 2H, J=8.1 Hz), 3.33 (s, 2H), 2.44 (s, 3H), 1.95 (m, 2H), 1.61 (m, 4H). ¹³C NMR (75 MHz, CDCl₃): δ 144.1, 135.9, 129.6, 127.6, 46.7, 26.9, 21.6, 19.5. FT-IR (film cm⁻¹): 3271, 2958, 2925, 2854, 1598, 1494, 1439, 1367, 1320, 1303, 1157, 1093, 1075, 1009, 977, 931, 874, 832, 815, 723, 673.

### Example 209

N-(p-Tolylsulfonyl)-7-azabicyclo[4.1.0]heptane was synthesized from cyclohexene as substrate and the product obtained as a yellow oil (33 mg, yield, 66%). ¹H NMR (300 MHz, CDCl₃): δ 7.79 (d, 2H, J=7.8 Hz), 7.30 (d, 2 H, J=8.1 Hz), 2.96 (s, 2H), 2.42 (s, 3H), 1.77 (m, 4H), 1.39 (m, 2H), 1.20 (m, 2H). ¹³C NMR (75 MHz, CDCl₃): δ 143, 141, 129, 127, 39.3, 22.3, 21.9, 18.9. FT-IR (film, cm⁻¹): 3000, 1600, 1392, 1155, 920. FT-IR (film cm⁻¹): 3280, 2927, 2856, 2361, 2340, 1598, 1494, 1440, 1399, 1320, 1239, 1184, 1157, 1091, 1020, 965, 921, 847, 816, 793, 724, 667.

### Example 210

N-(p-Tolylsulfonyl)-9-azabicyclo[6.1.0]nonane was synthesized from cyclooctene as substrate and the product obtained as a yellow oil (44 mg, yield, 79%). ¹H NMR (300 MHz, CDCl₃): δ 7.80 (d, 2H, J=8.1 Hz), 7.31 (d, 2H, J=7.8 Hz), 2.76 (d, 1H, J=9.9 Hz), 2.43 (s, 3H), 1.99 (d, 1H, J=14.1 Hz), 1.55-1.23 (m, 12H). ¹³C NMR (75 MHz, CDCl₃): δ 144.0, 135.8, 129.6, 127.5, 43.9, 26.3, 26.1, 25.2, 21.6. FT-IR (film cm⁻¹): 2927, 2857, 1598, 1494, 1468, 1449, 1425, 1320, 1158, 1092, 1016, 985, 933, 889, 869, 856, 825, 815, 796, 764, 750, 720, 668.

### Example 211

N-(p-Tolylsulfonyl)-2-heptylaziridine was synthesized from 1-nonene as substrate and the product obtained as a yellow oil (33 mg, yield, 56%). ¹H NMR (300 MHz, CDCl₃): δ 7.82 (d, 2H, J=6.6 Hz), 7.33 (d, 2H, J=8.1 Hz), 2.68 (m, 1H), 2.63 (d, 1H, J=6.9 Hz), 2.44 (s, 3H), 2.05 (d, 1H, J=4.2 Hz), 1.2 (m, 12H), 0.87 (t, 3H, J=6.3 Hz). ¹³C NMR (75 MHz, CDCl₃): δ 144.4, 129.6, 128.0, 40.5, 33.8, 31.6, 31.3, 29.1, 28.9, 26.8, 22.6, 21.6, 14.1. FT-IR (film cm⁻¹): 2926, 2856, 1725, 1598, 1494, 1458, 1401, 1325, 1232, 1161, 1092, 1020, 929, 869, 815, 768, 747, 715, 694, 662.

### Example 212

N-(p-Tolylsulfonyl)-1-azaspiro[2.5]octane was synthesized from methylene cyclohexane as substrate and the product obtained as a yellow oil (36 mg, yield, 67%). ¹H NMR (300 MHz, CDCl₃): δ 7.82 (d, 2H, J=7.8 Hz), 7.30 (d, 2H, J=8.1 Hz), 2.43 (s, 3H), 2.40 (s, 2H), 1.72-1.97 (m, 6H), 1.50 (m, 4H). ¹³C NMR (75 MHz, CDCl₃). δ 143.7, 137.7, 129.4, 127.3, 54.0, 41.0, 33.0, 25.4, 25.2, 21.6. FT-IR (film cm⁻¹): 3287, 2934, 2857, 1598, 1495, 1448, 1386, 1318, 1252, 1209, 1158, 1129, 1092, 1002, 943, 867, 840, 816, 793, 723, 664.

### Example 213

### General Procedure for Aziridination of Alkenes with Diphenylphosphoryl Azide (DPPA)

An oven dried Schlenk tube equipped with a stirring bar was degassed on a vacuum line and purged with nitrogen. The tube was charged with metalloporphyrin (10 mol %) and activated 5 Å molecular sieves (200 mg). The tube was capped with a Teflon screw. cap, evacuated on a vacuum line for 30-45 min. The Teflon screw cap was replaced with a rubber septum and 2 mL of solvent, diphenylphosphoryl azide (0.2 mmol) and substrate (1.0 mmol) were then added successively. The tube was purged with nitrogen for 1-2 min and the contents were stirred and heated at 80-100° C., overnight. After completion of the reaction, the mixture was cooled down to room temperature. The molecular sieves were removed by filtration and the filtrate was concentrated under vacuum. The solid residue was purified by flash chromatography (silica gel, ethyl acetate:hexanes (V:V)=3:7) to afford the pure product.

### Example 214

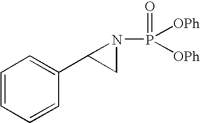

(2-Phenyl-aziridin-1-yl)-phosphonic acid diphenyl ester was synthesized from the reaction of styrene with DPPA and obtained as a yellow oil. ¹H NMR (300 MHz, CDCl₃): δ 7.27-7.00 (m, 15H), 3.63 (ddd, 1H, J=16.5, 6.3, 3.6 Hz), 2.82 (ddd, 1H, J=19.5, 6.3, 1.2 Hz), 2.24 (ddd, 1H, J=15.6, 3.6, 1.2 Hz). ¹³C NMR (75 MHz, CDCl₃): δ 129.7, 129.6, 128.5, 128.1, 126.2, 125.2, 120.5, 120.4, 120.3, 39.0, 38.9, 35.0, 34.9. ³¹P NMR (121 MHz, CDCl₃): δ 6.11 (s). FT-IR (film, cm⁻¹): 1590, 1191, 941, 669. HRMS-EI ([M]⁺) for C₂₀H₁₈NO₃P, calcd 351.1024, found 351.1022.

### Example 215

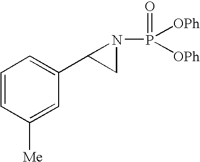

(2-m-Tolyl-aziridin-1-yl)-phosphonic acid diphenyl ester was synthesized from the reaction of 3-methylstyrene with DPPA and obtained as a yellow oil. ¹H NMR (300 MHz, CDCl₃): δ 7.28-6.95 (m, 14H), 3.61 (ddd, 1H, J=16.5, 6.0, 3.3 Hz), 2.80 (dd, 1H, J=19.2, 6.0 Hz), 2.22 (m, 4H). ¹³C NMR (75 MHz, CDCl₃): δ 129.7, 129.6, 128.8, 128.3, 126.8, 125.2, 123.4, 120.5, 120.4, 120.3, 39.0, 38.9, 34.9, 34.8, 21.3. ³¹P NMR (121 MHz, CDCl₃): δ 6.19 (s). FT-IR (film, cm⁻¹): 1711, 1585, 1482, 1190, 1010, 932, 762. HRMS-EI ([M]⁺) for C₂₁H₂₀NO₃P, calcd 365.1181, found 365.1174.

### Example 216

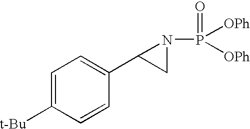

[2-(4-tert-Butyl-phenyl)-aziridin-1-yl]-phosphonic acid diphenyl ester was synthesized from the reaction of p-tert-butylstyrene with DPPA and obtained as a yellow oil. ¹H NMR (300 MHz, CDCl₃): δ 7.28-7.00 (m, 14H), 3.62 (ddd, 1H, J=16.5, 6.0, 3.6 Hz), 2.80 (ddd, 1H, J=19.8, 6.6, 1.2 Hz), 2.24 (ddd, 1H, J=15.3, 3.6, 1.2 Hz), 1.24 (s, 9H). ¹³C NMR (75 MHz, CDCl₃): δ 129.7, 129.6, 125.9, 125.4, 125.2, 120.5, 120.4, 120.3, 38.9, 38.8, 34.9, 34.8, 34.5, 31.3. ³¹P NMR (121 MHz, CDCl₃): δ 6.24 (s). FT-IR (film, cm⁻¹): 1592, 1490, 1193, 943, 773, 689. HRMS-EI ([M]⁺) for C₂₄H₂₆NO₃P, calcd 407.1650, found 407.1658.

### Example 217

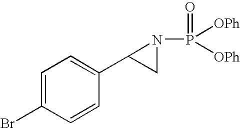

[2-(4-Bromo-phenyl)-aziridin-1-yl]-phosphonic acid diphenyl ester was synthesized from the reaction of p-bromostyrene with DPPA and obtained as a yellow oil. ¹H NMR (300 MHz, CDCl₃): δ 7.35 (d, 2H, J=8.4 Hz), 7.28-7.07 (m, 10H), 7.01 (d, 2H, J=8.4 Hz), 3.57 (ddd, 1H, J=16.2, 6.0, 3.3 Hz), 2.80 (ddd, 1H, J=18.9, 6.0, 1.2 Hz), 2.19 (ddd, 1H, J=15.3, 3.3, 1.2 Hz). ¹³C NMR (75 MHz, CDCl₃): δ 129.7, 129.6, 127.9, 125.3, 120.4, 120.3, 120.3, 38.3, 38.2, 35.0, 34.9. ³¹P NMR (121 MHz, CDCl₃): δ 5.76 (s). FT-IR (film, cm⁻¹): 1591, 1489, 1283, 1192, 1163, 1072, 1006, 945, 827, 774. HRMS-EI ([M]⁺) for C₂₀H₁₇BrNO₃P, calcd 429.0129, found 429.0127.

### Example 218

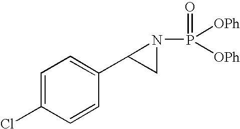

[2-(4-Chloro-phenyl)-aziridin-1-yl]-phosphonic acid diphenyl ester was synthesized from the reaction of p-chlorostyrene with DPPA and obtained as a yellow oil. ¹H NMR (300 MHz, CDCl₃): δ 7.28-7.07 (m, 14H), 3.57 (ddd, 1H, J=16.2, 6.0, 3.3 Hz), 2.80 (ddd, 1H, J=19.2, 6.0, 1.2 Hz), 2.19 (ddd, 1H, J=15.3, 3.3, 1.2 Hz). ¹³C NMR (75 MHz, CDCl₃): δ 129.7, 129.6, 128.6, 127.5, 125.3, 120.4, 120.3, 120.2, 38.3, 38.2, 35.0, 34.9. ³¹P NMR (121 MHz, CDCl₃): δ 5.79 (s). FT-IR (film, cm⁻¹): 1592, 1490, 1193, 943, 773, 689. HRMS-EI ([M]⁺) for C₂₀H₁₇ClNO₃P, calcd 385.0635, found 385.0629.

### Example 219

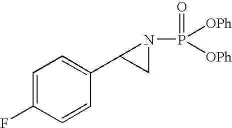

[2-(4-Fluoro-phenyl)-aziridin-1-yl]-phosphonic acid diphenyl ester was synthesized from the reaction of p-fluorostyrene with DPPA and obtained as a yellow oil. ¹H NMR (300 MHz, CDCl₃): δ 7.28- 7.04 (m, 12H), 6.92 (t, 2H, J=8.7 Hz), 3.59 (ddd, 1H, J=16.5, 6.0, 3.6 Hz), 2.80 (ddd, 1H, J=19.5, 6.3, 1.2 Hz), 2.19 (ddd, 1H, J=15.0, 3.3, 0.6 Hz). ¹³C NMR (75 MHz, CDCl₃): δ 129.7, 129.6, 127.8, 127.7, 125.3, 120.4, 120.3, 120.2, 115.6, 115.3, 38.3, 38.2, 35.0, 34.9. ³¹P NMR (121 MHz, CDCl₃): δ 5.96 (s). FT-IR (film, cm⁻¹): 1592, 1490, 1224, 1192, 932, 835, 689. HRMS-EI ([M]⁺) for C₂₀H₁₇FNO₃P, calcd 369.0930, found 369.0946.

### Example 220

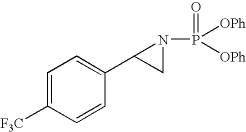

[2-(4-Trifluoromethyl-phenyl)-aziridin-1-yl]-phosphonic acid diphenyl ester was synthesized from the reaction of p-trifluoromethylstyrene with DPPA and obtained as a yellow oil. ¹H NMR (300 MHz, CDCl₃); δ 7.48 (d, 2H, J=7.80 Hz), 7.26 (d, 2H, J=7.5 Hz), 7.23-7.04 (m, 10H), 3.65 (ddd, 1H, J=16.2, 6.0, 3.6 Hz), 2.84 (dd, 1H, J=19.2, 6.3 Hz), 2.22 (dd, 1H, J=15.3, 3.3 Hz). ¹³C NMR (75 MHz, CDCl₃): δ 129.8, 129.7, 126.5, 125.5, 125.4, 125.3, 120.3, 120.3, 120.2, 38.3, 38.2, 35.0, 35.0. ³¹P NMR (121 MHz, CDCl₃): δ 5.63 (s). ¹⁹F NMR (280 MHz, CDCl₃): δ −62.95. FT-IR (film, cm⁻¹): 1621, 1592, 1490, 1326, 1193, 1165, 1068, 1005, 947, 904, 774, 689. HRMS-EI ([M]⁺) for C₂₁H₁₇F₃NO₃P, calcd 419.0898, found 419.0894.

### Example 221

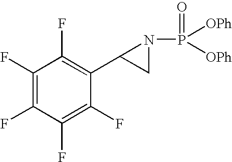

(2-Pentafluorophenyl-aziridin-1-yl)-phosphonic acid diphenyl ester was synthesized from the reaction of pentafluorostyrene with DPPA and obtained as a yellow oil. ¹H NMR (300 MHz, CDCl₃): δ 7.29-7.07 (m, 10H), 3.71 (ddd, 1H, J=16.5, 6.3, 3.6 Hz), 2.86 (dd, 1H, J=18.9, 6.3 Hz), 2.76 (dd, 1H, J=15.6, 3.6 Hz). ¹³C NMR (75 MHz, CDCl₃): δ 129.8, 129.7, 125.4, 125.3, 120.2, 120.1, 120.0, 30.9, 30.0. ³¹P NMR (121 MHz, CDCl₃): δ 5.35 (s). ¹⁹F NMR (280 MHz, CDCl₃): δ −140, −152, −160. FT-IR (film, cm⁻¹): 1687, 1520, 1476, 1176, 980. HRMS-EI ([M]⁺) for C₂₀H₁₃F₅NO₃P, calcd 441.0553, found 441.0559.

### Example 222

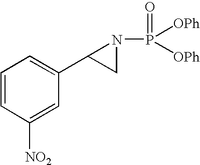

[2-(3-Nitro-phenyl)-aziridin-1-yl]-phosphonic acid diphenyl ester was synthesized from the reaction of 3-nitrostyrene with DPPA and obtained as a yellow oil. ¹H NMR (300 MHz, CDCl₃): δ 8.05 (dd, 1H, J=8.1, 0.9 Hz), 7.98 (s, bro., 1H), 7.49-7.04 (m, 10H), 3.70 (ddd, 1H, J=16.2, 6.0, 3.3 Hz), 2.89 (dd, 1H, J=18.6, 6.0 Hz), 2.25 (dd, 1H, J=15.3, 3.3 Hz). ¹³C NMR (75 MHz, CDCl₃): δ 132.3, 129.8, 129.7, 129.5, 125.4, 123.0, 121.1, 120.3, 120.2, 37.9, 37.8. ³¹P NMR (121 MHz, CDCl₃): δ 5.27 (s). FT-IR (film, cm⁻¹): 1591, 1531, 1488, 1350, 1190, 950. HRMS-EI ([M−H]⁺) for C₂₀H₁₇N₂O₅P, calcd 395.0797, found 395.0789.

### Example 223

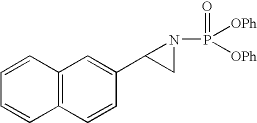

(2-Naphthalen-2-yl-aziridin-1-yl)-phosphonic acid diphenyl ester was synthesized from the reaction of 2-vinylnaphthalene with DPPA and obtained as a yellow oil. ¹H NMR (300 MHz, CDCl₃): δ 7.77-7.64 (m, 4H), 7.42-7.39 (m, 2H), 7.28-7.05 (m, 10H), 3.80 (ddd, 1H, J=16.5, 6.0, 3.6 Hz), 2.89 (ddd, 1H, J=19.2, 6.0, 1.2 Hz), 2.34 (ddd, 1H, J=15.3, 3.3, 1.2 Hz). ¹³C NMR (75 MHz, CDCl₃): δ 129.7, 129.6, 128.3, 127.7, 127.7, 126.3, 126.1, 125.8, 125.2, 123.4, 120.5, 120.4, 120.3, 39.2, 34.9. ³¹P NMR (121 MHz, CDCl₃): δ 6.13 (s). FT-IR (film, cm⁻¹): 1593, 1489, 1193, 936, 689. HRMS-EI ([M]⁺) for C₂₄H₂₀NO₃P, calcd 401.1 181, found 401.1183.

### Results

I. Synthesis of Chiral Porphyrins

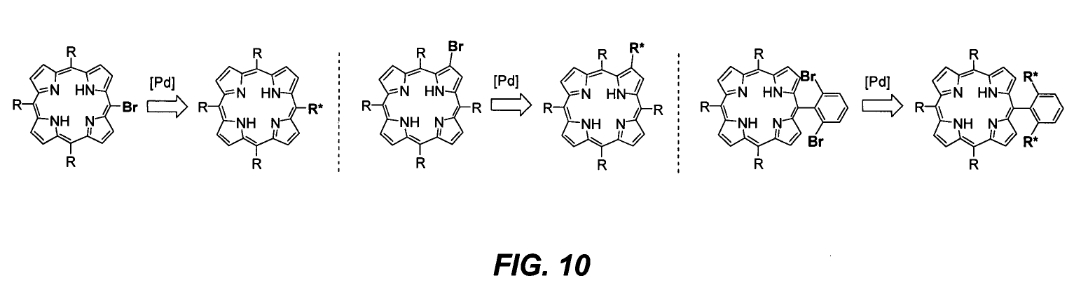

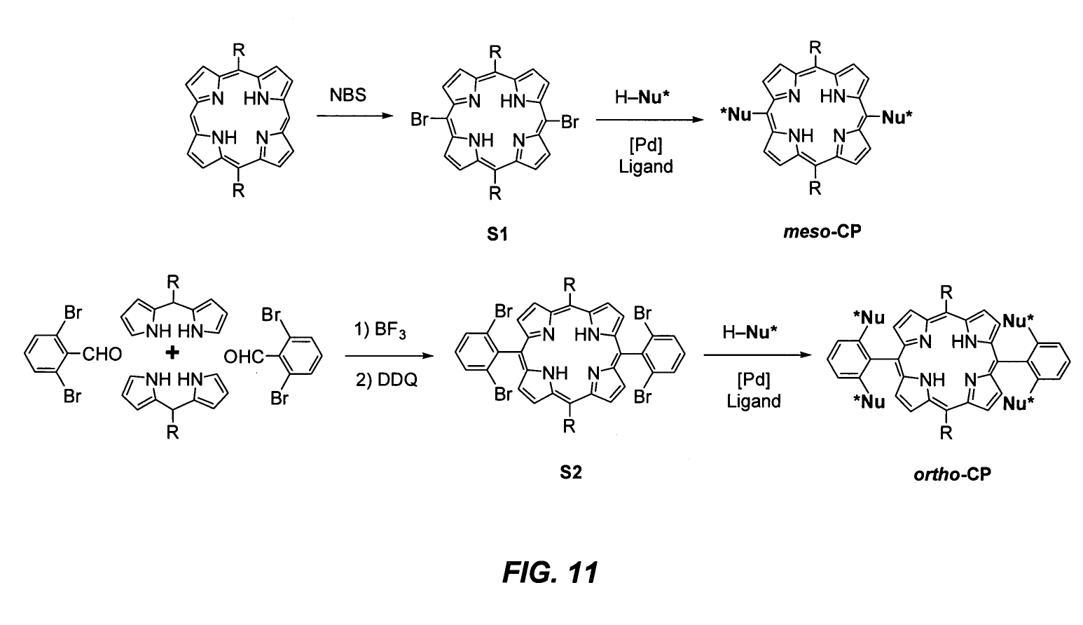

A generalized schematic representation of the use of haloporphyrins, e.g., bromoporphyrins, as synthons for the modular construction of chiral porphyrins is provided in FIG. 10. More particularly, as shown in FIG. 11, in some embodiments, the presently disclosed subject matter describes the use of two types of brominated porphyrin synthons, e.g., 5,15-dibromoporphyrins (S1) and 5,15-bis(2,6-dibromophenyl)porphyrins (S2), which, in some embodiments, bear R substituents at the 10,20-positions, for the synthesis of chiral porphyrins.

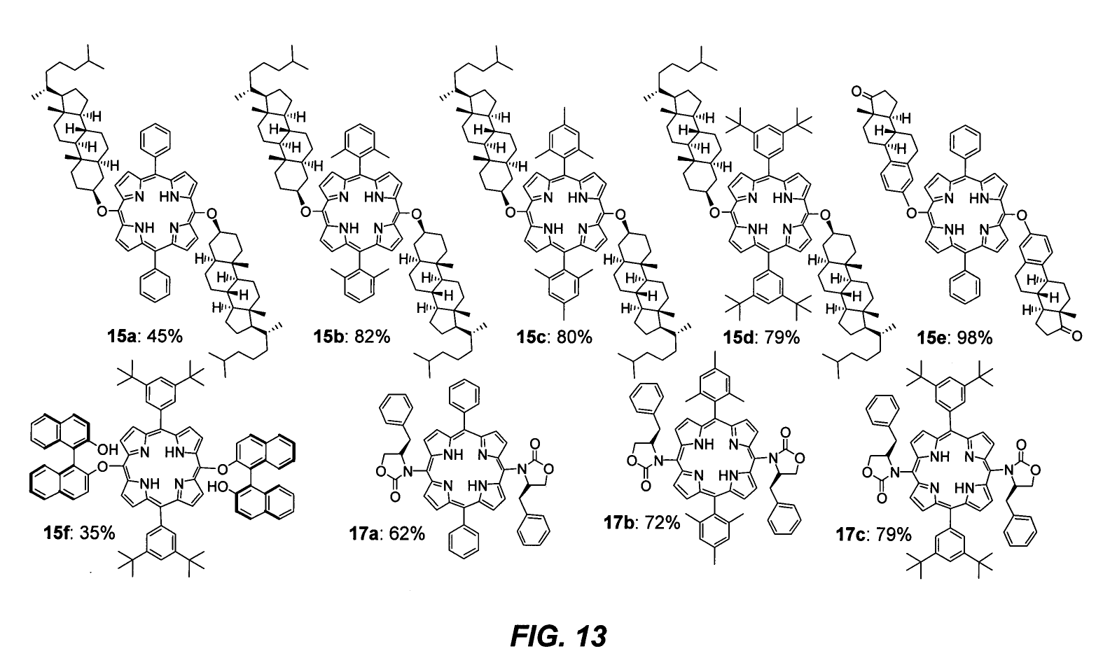

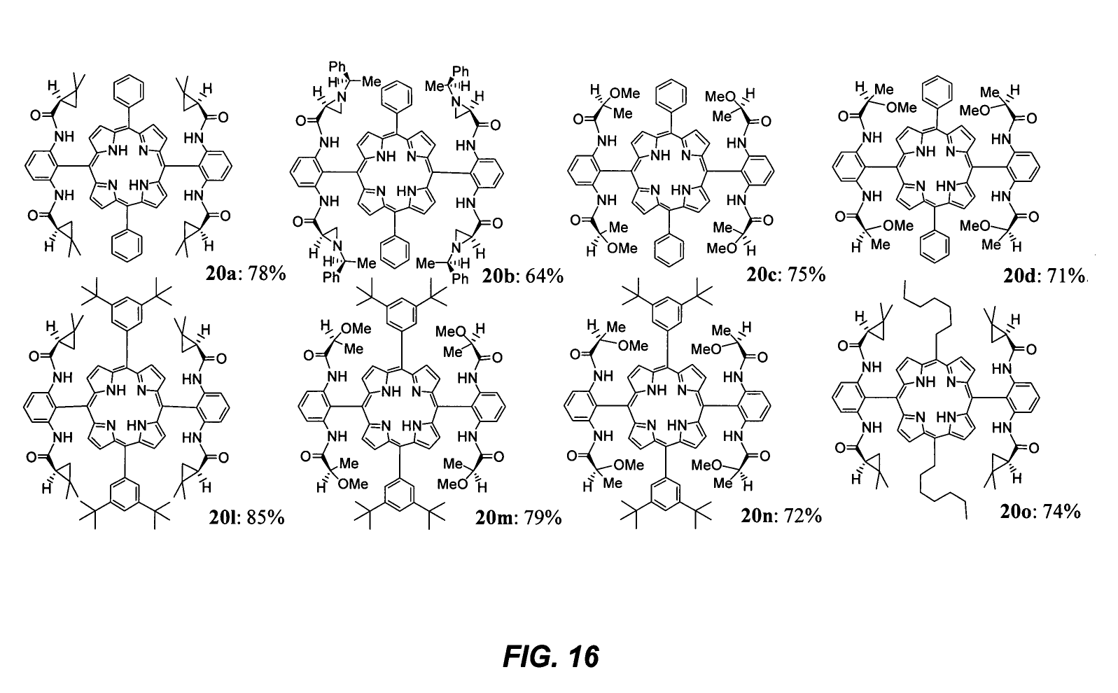

The bromoporphyrin synthons, e.g., bromoporphyrin synthons S1 and S2 as shown in FIG. 11, used in the presently disclosed subject matter are prepared by selective bromination of preformed porphyrins with N-bromosuccinimide (NBS) (first step in the upper reaction sequence of FIG. 11) and by MacDonald [2+2] porphyrin synthesis using Lindsey's condition, see Lindsey, (2000) in The Porphyrin Handbook; Kadish, K. M., Smith, K. M., Guilard, R., Eds., Academic Press: San Diego, Calif.; Vol. 1; pp 45-118 (first step in the lower reaction sequence of FIG. 11). Commercially available chiral building blocks are used as nucleophiles (designated as H-Nu* in FIG. 11) to couple with synthons S1 and S2 via palladium-mediated multiple crosscoupling reactions, yielding a series of D₂-or pseudo-D₂-symmetric meso-chiral porphyrins, e.g., compounds 15a-15f and compounds 17a-17c of FIG. 13, and ortho chiral porphyrins, e.g., compounds 20a-20d and compounds 20l-20o of FIG. 16, respectively. The R substituents at the 10,20-positions can be aromatic, substituted aromatic, aliphatic, substituted aliphatic, aralalkyl or heteroatom-containing groups, which allows fine-tuning of the electronic and steric environments and the manipulation of product solubility.

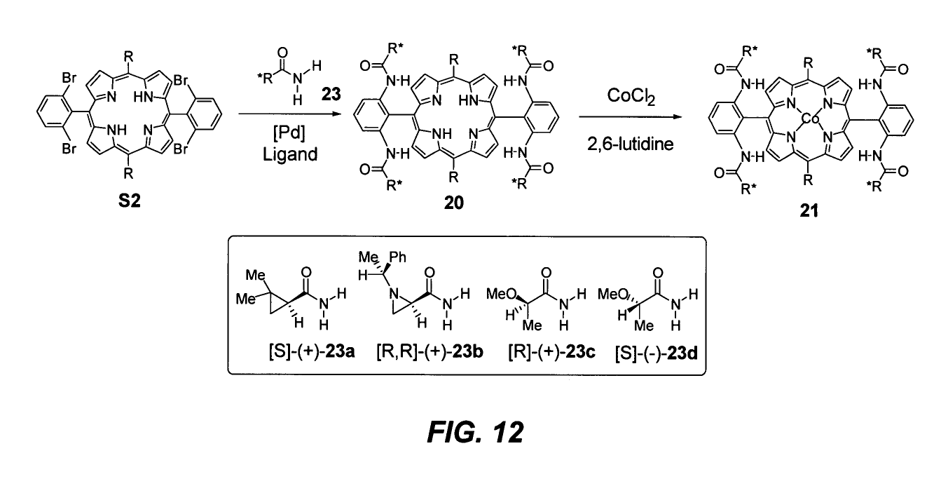

In some embodiments, the chiral nucleophile H-Nu* comprises a chiral amide. As shown in FIG. 12, the quadruple carbon-nitrogen bond formation reactions can be accomplished in high yields with different chiral amide building blocks, e.g., compounds 23a-23d, under mild conditions, forming a family of ortho-chiral porphyrins, e.g., compounds 20a-20p, and their corresponding Co complexes, e.g., compounds 21a-21p, of Table 4.

As summarized in Table 4, a series of 5,15-bis(2,6-dibromophenyl)porphyrins, 19a-19k, comprising different meso-aryl and meso-alkyl R groups at the 10,20-positions, were coupled with several optically pure amides 23a-23d under palladium-catalyzed amidation conditions. The combination of Pd(OAc)₂ and XantPhos mediates the quadruple amidation reactions of synthons 19a-19k with chiral amides 23a-23d to deliver a family of D₂-symmetric chiral porphyrins 20a-20p in high yields.

Without being bound to any particular theory, the near perpendicular arrangement between the meso-phenyl ring and the porphyrin plane, in combination with the trans-amide conformation, appears to direct the ortho-chiral R* units toward the center of porphyrins, as suggested from the observed large high-field NMR chemical shifts of the chiral R* units. As a result, as will be described in more detail herein below, high asymmetric induction can be achieved for catalytic reactions with metal complexes of these chiral porphyrins. Accordingly, through the combined use of the chiral R* and meso-R groups, it is possible to control diastereoselectivity as well as enantioselectivity in catalytic reactions with metal complexes of these chiral porphyrins.

TABLE 4
Synthesis of Chiral Porphyrins 20 and Cobalt Complexes 21.^{a}

| Entry | R | 19 | 23 | 20: yield^{a} | 21: yield^{a} |
|---|---|---|---|---|---|
| 1 | Ph | 19a | 23a | 20a: 78% | 21a: 88% |
| 2 | Ph | 19a | 23b | 20b: 64% | 21b: 86% |
| 3 | Ph | 19a | 23c | 20c: 75% | 21c: 95% |
| 4 | Ph | 19a | 23d | 20d: 71% | 21d: 95% |
| 5 | 4-t-BuPh | 19b | 23a | 20e: 86% | 21e: 72% |
| 6 | 4-CF₃Ph | 19c | 23a | 20f: 77% | 21f: 95% |
| 7 | PentaFPh | 19d | 23a | 20g: 46% | 21g: 86% |
| 8 | 4-AcetylPh | 19e | 23a | 20h: 66% | 21h: 83% |
| 9 | 2,4,6-triMePh | 19f | 23a | 20i: 84% | 21i: 91% |
| 10 | 2,6-diMeOPh | 19g | 23a | 20j: 59% | 21j: 95% |
| 11 | 3,5-diMeOPh | 19h | 23a | 20k: 88% | 21k: 96% |
| 12 | 3,5-di-t-BuPh | 19i | 23a | 20l: 85% | 21l: 91% |
| 13 | 3,5-di-t-BuPh | 19i | 23c | 20m: 79% | 21m: 96% |
| 14 | 3,5-di-t-BuPh | 19i | 23d | 20n: 72% | 21n: 92% |
| 15 | 4-n-heptyl | 19j | 23a | 20o: 74% | 21o: 95% |
| 16 | H | 19k | 23a | 20p: 79% | 21p: 91% |

^{a}Yields represent isolated yields of >95% purity as determined by ¹H NMR.

As also will be described in more detail herein below cobalt complexes of chiral porphyrins 21a-21p, which were prepared in high yields, were applied as catalysts for cyclopropanation using styrene as a model substrate (see Table 5). Among other options, metallation reactions can be performed with either CoCl₂ in THF in the presence of 2,6-lutidine or with Co(OAc)₂ in DMF at higher temperature.

Representative meso-chiral porphyrins of the presently disclosed subject matter are provided in FIG. 13. By way of example, four meso-porphyrins dibromoporphyrins S1 comprising different meso-aryl groups (e.g., R=phenyl; R=2,6-dimethylphenyl; R=2,4,6-trimethylphenyl; R=3,5-di-tert-enyl), butylphenyl), were prepared via selective bromination of the corresponding 5,15-diarylporphyrins. These meso-dibromoporphyrins were successfully coupled with chiral alcohols and chiral amides under palladium-catalyzed etheration and amidation conditions, affording a series of novel meso-chiral porphyrins 15a-15f and 17a-17c in 35-98% yields (see FIG. 13 and Table 5).

TABLE 5
Synthetic Conditions and Yields for meso-Chiral
Porphyrins and Their Cobalt Complexes.

| entry | Ar group of 1 | *ROH/*RNH | coupling | product:^{a} | metallation | product:^{a} |
|---|---|---|---|---|---|---|
| 1 | 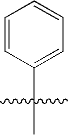 | 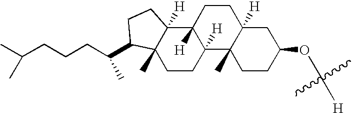 | Pd₂(dba)₃/ DPEphos/Cs₂CO₃toluene/100° C./17 h | 15a 45% | CoCl₂/2,6-lutidine THF/70° C./15 h | 15a: 71% |
| 2 | 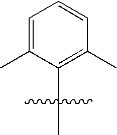 | 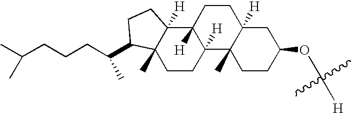 | Pd₂(dba)₃/ DPEphos/Cs₂CO₃toluene/100° C./20 h | 15b 82% | CoCl₂/2,6-lutidine THF/70° C./14 h | 15b: 96% |
| 3 | 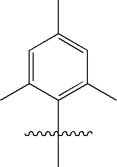 | 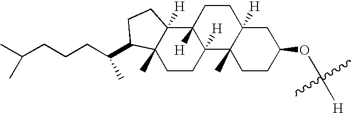 | Pd₂(dba)₃/ DPEphos/Cs₂CO₃toluene/100° C./20 h | 15c 80% | CoCl₂/2,6-lutidine THF/70° C./14 h | 15c: 88% |
| 4 | 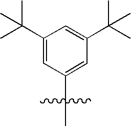 | 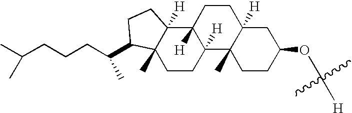 | Pd₂(dba)₃/ DPEphos/Cs₂CO₃toluene/100° C./18 h | 15d 79% | COCl₂/2,6-lutidine THF/70° C./14 h | 15d: 89% |
| 5 | 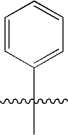 | 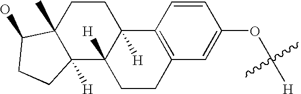 | Pd₂(dba)₃/ DPEphos/Cs₂CO₃toluene/100° C./40 h | 15e 98% | Co(OAc)₂.4H₂O DMF/160° C./2 h | 15e: 77% |
| 6 | 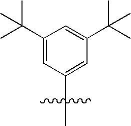 | 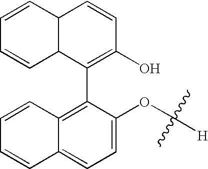 | Pd₂(dba)₃/ DPEphos/Cs₂CO₃toluene/100° C./20 h | 15f 35%^{b} | CoCl₂/2,6-lutidine THF/70° C./14 h | 15f: 96%^{b} |
| 7 | 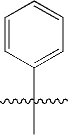 | 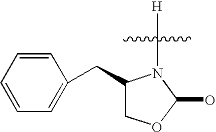 | Pd₂(dba)₃/ Xantphos/Cs₂CO₃THF/68° C./22 h | 17a: 62%^{b} | CoCl₂/2,6-lutidine THF/70° C./14 h | 17a: 87%^{b} |
| 8 | 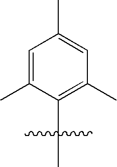 | 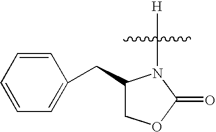 | Pd₂(dba)₃/ Xantphos/Cs₂CO₃THF/80° C./20 h | 17b: 72%^{b} | CoCl₂/2,6-lutidine THF/70° C./14 h | 17b: 94%^{b} |
| 9 | 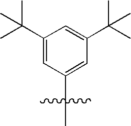 | 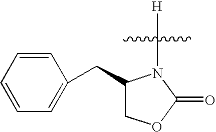 | Pd₂(dba)₃/ Xantphos/Cs₂CO₃THF/80° C./22 h | 17c: 79%^{b} | CoCl₂/2,6-lutidine THF/70° C./14 h | 17c: 95%^{b} |

^{a}Yields represent isolated yields of > 95% purity as determined by ¹H NMR.
^{b}Products existed as a mixture of two astropisomers (α,α- and α,β-isomers) in approximately equal amounts.

Only one set of resonances was observed in both ¹H and ¹³C spectra of the products 15a-15e, suggesting that there is a free rotation around the O—C bond at ambient temperature in these meso-chiral porphyrins. When R-(+)-BINOL was used in an excess amount, the double etheration reaction could be controlled to give meso-chiral porphyrin 15f where only one of the two hydroxyl groups was reacted, although the yield was low (Table 5, entry 6). The observation of multiple ¹H NMR resonances for the tert-butyl groups suggests the product 15f existed as a mixture of two atropisomers (α,α- and α,β-isomers), presumably due to increased rotation barrier around the O—C bond. As evidenced by the well-separated two sets of ¹H NMR resonances in approximately equal intensities, the products 17a-17c all existed as a mixture of two atropisomers in near same amounts, resulting from a high rotation barrier around the bond between the porphyrin meso-carbon atom and the amide nitrogen atom. Attempts to separate these atropisomers have been unsuccessful. The same or similar R_{f} values were obtained for two atropisomers using different solvent systems.

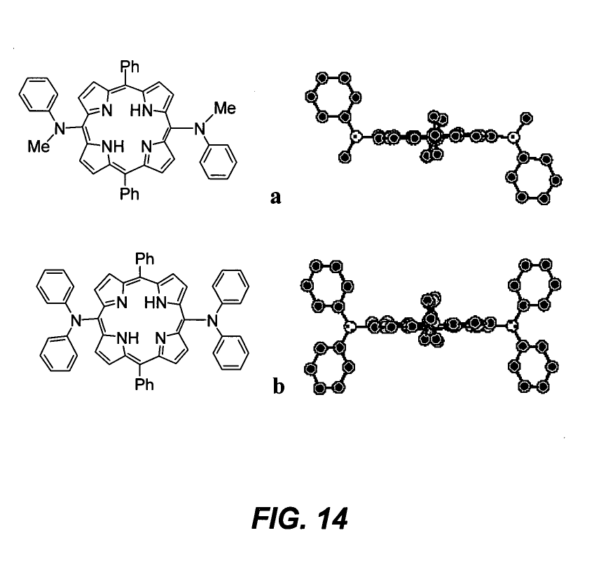

X-ray structural studies of several meso-aminoporphyrins (for example, see FIG. 14a and 14b) revealed that all the amino nitrogen atoms adopt an almost perfect planar geometry to minimize steric interactions with the β-hydrogen atoms and that the plane is nearly perpendicular to the porphyrin ring. These data suggest that chiral secondary amines also could be suitable building blocks for the construction of meso-chiral porphyrins.

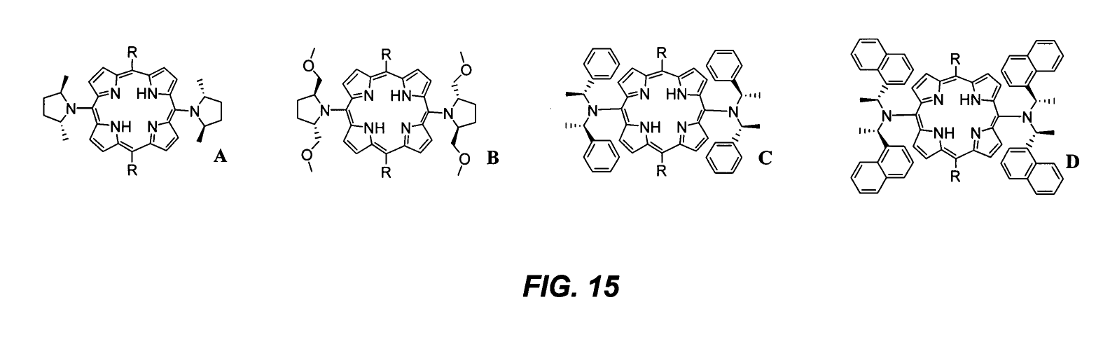

Accordingly, a series of meso-chiral porphyrins (represented generally as A, B, C, and D in FIG. 15) from reactions of synthon S1 with a selection of C2-symmetric chiral secondary amines via palladium-catalyzed double amination are provided by the presently disclosed subject matter. The meso-chiral porphyrins provided in FIG. 15 contain more rigid chiral appendages with desirable geometries and orientations and therefore also should be good ligands for asymmetric catalysis.

Further, representative ortho-chiral porphyrins of the presently disclosed subject matter are provided in FIG. 16. By way of example, three 5,15-bis(2,6-dibromophenyl)porphyrins, S2, comprising different meso-aryl and meso-alkyl groups (e.g., R=phenyl; R=2,6-di-tert-butylphenyl; and R=n-heptyl), were prepared by MacDonald [2+2] porphyrin synthesis under Lindsey's condition from 2,6-dibromobenzaldhyde and corresponding dipyrromethanes. These dibromoporphyrins were coupled with chiral amides 23a-23d under palladium-catalyzed amidation conditions to produce ortho-chiral porphyrins 20 (see, e.g., the reaction schemes provided in FIG. 12).

Accordingly, in some embodiments, the combination of the palladium catalyst Pd(OAc)₂ and the ligand XantPhos mediates the quadruple amidation reactions of synthons S2 with building blocks 23a-23d to yield a series of D₂-symmetric ortho-chiral porphyrins, e.g., compounds 20a-20d and 20l-20o, in 64-85% yields (see FIG. 16). Without being bound to any one particular theory, the near perpendicular arrangement between the meso-phenyl ring and the porphyrin plane, in combination with the trans-amide conformation, appears to direct the ortho-chiral units toward the center of the porphyrins. The observed large high-field NMR chemical shifts (Δδ˜1.0-1.5 ppm) of the chiral units are consistent with this conclusion. As a result, high asymmetric induction can be achieved with these chiral porphyrins during catalysis. Using a variety of meso-R groups, it also is possible to control diastereoselectivity. As described in more detail herein below, the asymmetric cyclopropanation results demonstrate that both high enantioselectivity and high diastereoselectivity, as well as high chemical yields, can be achieved with an appropriate combination of chiral R* units and meso-R groups.

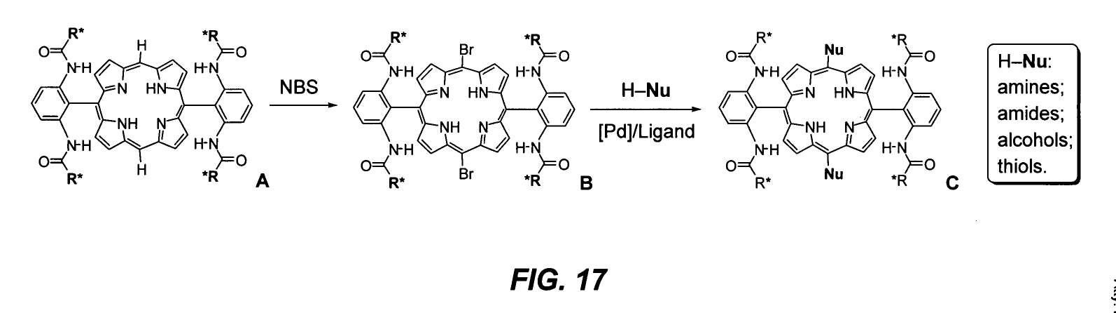

Further, the yields of the multiple amidation reactions can be improved by using different combinations of phosphine ligands and palladium precursors. Thus, syntheses using synthons S2 that bear a series of other aromatic and aliphatic groups, allowing fine-tuning of electronic and steric properties of the resulting chiral porphyrins, can be prepared by the method of the presently disclosed subject matter. Compared to carbon-based groups, heteroatom substituents are expected to have very different electronic and steric effects. Therefore, chiral porphyrins (represented by C in FIG. 17) bearing various meso-heteroatom substituents, including amino, amido, alkoxy/aryloxy, and alkylsulfanyl/arylsulfanyl groups are provided by the presently disclosed subject matter.

Further, chiral porphyrins containing hydrogen atoms at meso-positions (represented by A in FIG. 17) can be prepared in a similar way as described for chiral porphyrins 15a-15f, 17a-17c, 20a-20d, and 20l-20o (see FIG. 11 and FIG. 12), and can be converted to meso-dibromoporphyrins (represented by B in FIG. 17) by selective bromination (see FIG. 17). The methods provided in FIG. 8 and described herein above allow the conversion of meso-dibromoporphyrins B to the desired meso heteroatom-substituted ortho-chiral porphyrins C. In addition to the achiral nucleophiles commonly employed, chiral nucleophiles can be attached in a similar manner in the construction, resulting in an array of meso-/ortho-heterochiral porphyrins, which provides a new dimension in the tuning of electronic, steric, and chiral environments.

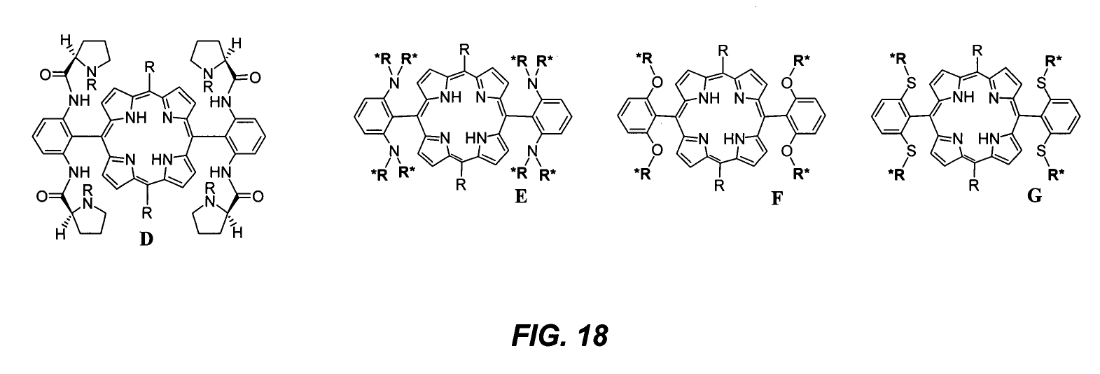

In addition to chiral amides 23a-23d, other primary and secondary chiral amides also can be employed in the construction of chiral porphyrins to produce diverse chiral environments. In particular, amides of natural α-amino acids and of short peptides can be attached to synthons S2. For example, primary amides of proline derivatives can be coupled with synthons S2 to afford chiral porphyrins of the presently disclosed subject matter (see, e.g., D of FIG. 18). Similarly, the strategy can be expanded to include chiral amines, chiral alcohols, and chiral thiols for the construction of ortho-chiral porphyrins E, F, and G of FIG. 18, respectively.

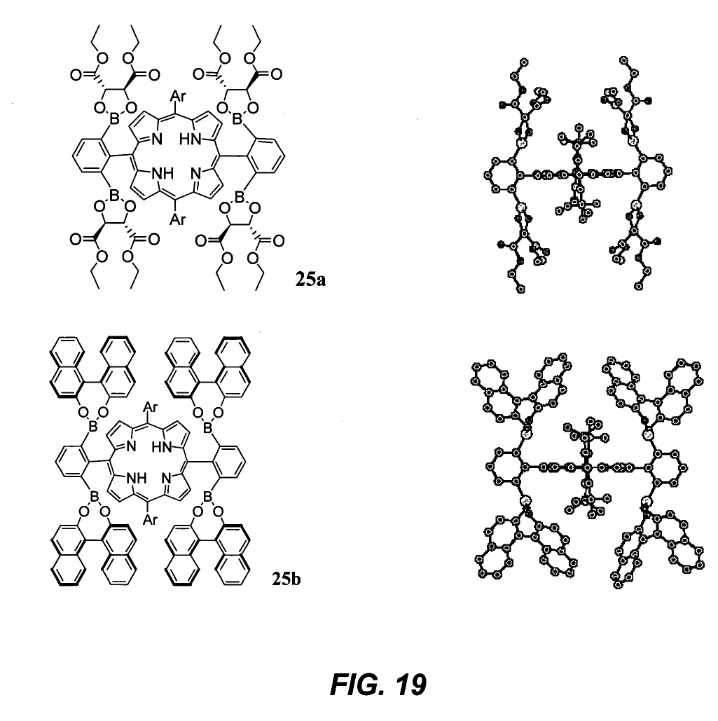

Further, in view of the availability of chiral diborate esters, the palladium-mediated carbon-boron bond formation reactions can be employed to synthesize borate ester-containing chiral porphyrins. Two types of borate ester-containing chiral porphyrins 25a and 25b, along with their 3D structures generated from computer modeling, are shown in FIG. 19.

Further, in combination with computer modeling, the correlation between chiral porphyrin structure and observed catalytic activity in asymmetric cyclopropanation and aziridination can be used to guide the design and syntheses of new chiral porphyrins with improved reactivity and selectivity.

II. Asymmetric Cyclopropanation by Metalloporphyrins

Transition metal complex-mediated cyclopropanation of alkenes with diazo compounds as shown in Equation 1 is an efficient and selective method for constructing synthetically and biologically important cyclopropanes.

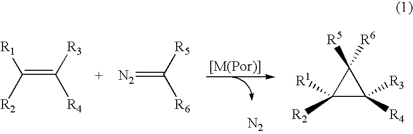

For representative examples of metal-catalyzed cyclopropanation, see Niimi et al., (2001) Adv. Synth. Catal. 343: 79; Ikeno et al., (2001) Bull. Chem. Soc. Jpn. 74: 2139; Che et al., (2002) Coord. Chem. Rev. 231: 151; Berkessel et al., (2003) Chem Eur. J. 9: 4746; Gross et al., (1999) Tetrahedron Lett. 40: 1571; Du et al., (2002) Organometallics 21: 4490; and Huang et al., (2003) J. Org. Chem. 68: 8179.

Among the various catalysts used in cyclopropanation reactions, metalloporphyrins are unique in their unusual selectivity and high catalytic turnover, as well as in their biological relevance. Several metalloporphyrins have been found to catalyze cyclopropanation, including Rh, Os, Fe, and Ru porphyrins. Despite the close periodic relationship of Co to Rh, until recently Co porphyrins had not been demonstrated to have catalytic carbene transfer activity. See Huang et al., (2003) J. Org. Chem., 68: 8179; Penoni et al., (2003) Eur. J. Inorg. Chem. 1452; Niimi et al., Adv. Synth. Catal. (2001), 342: 79; and Ikeno et al., Bull. Chem. Soc. Jpn. (2001), 74: 2139. Although Co porphyrins have been shown to be better catalysts than previously known metalloporphyrins only moderate diastereoselectivity and enantioselectivity were achieved.

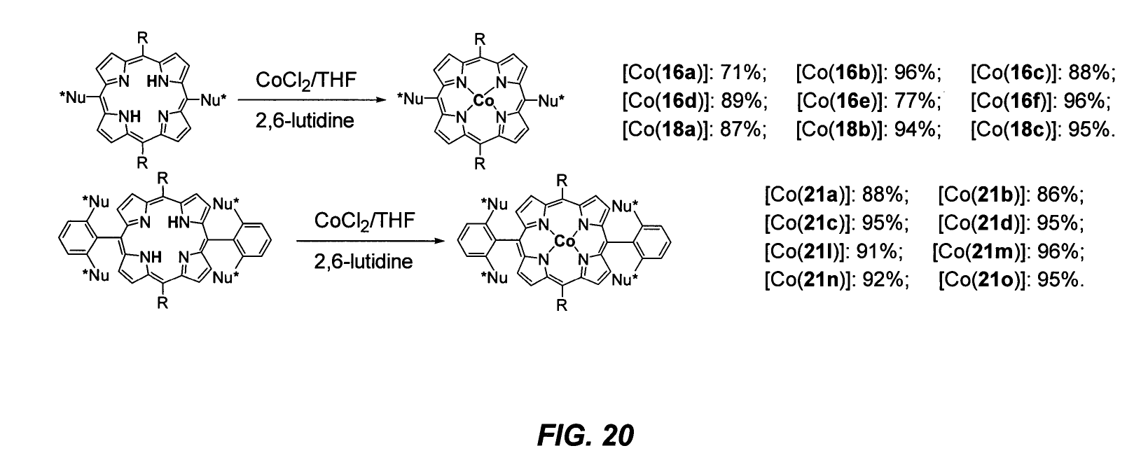

The family of chiral porphyrins described by the presently disclosed subject matter improves cobalt-based, as well as other metal-based, catalytic systems for cyclopropanation. Accordingly, metal complexes, e.g., Co complexes, of meso-chiral and ortho-chiral porphyrins were prepared. By way of example, both meso-chiral porphyrins (e.g., compounds 15a-15f and compounds 17a-17c of FIG. 13) and ortho-chiral porphyrins (e.g., compounds 20a-20d and compounds 20l-20o of FIG. 16) were converted to their Co(II) complexes in high to excellent yields (see FIG. 20).

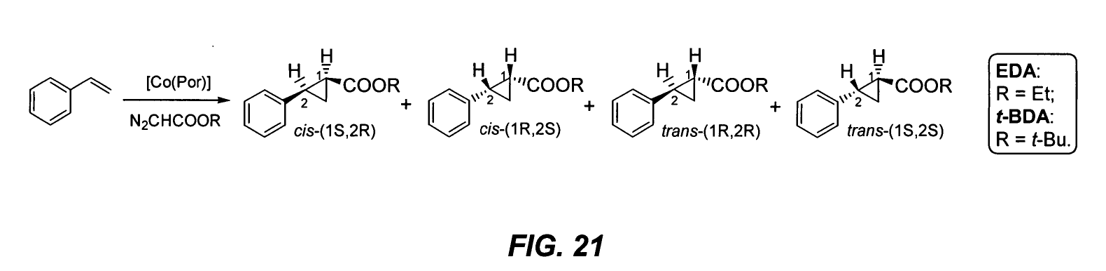

Cobalt complexes of meso-chiral porphyrins, e.g., compounds 15a-15f and compounds 17a-17c, were found to be effective catalysts for the cyclopropanation of styrene as illustrated in FIG. 21. Using 2 mol % meso-chiral cobalt porphyrin catalysts, e.g., compounds 16a-16f and compounds 18a-18c, the reactions proceeded successfully at room temperature, 80° C. or 0° C. with styrene as the limiting reagent and did not require slow-addition of EDA, affording the desired product in up to 99% yield (Table 6, below). While moderate trans selectivities (trans:cis ˜70:30) were observed, the enantioselectivities (<12% ee) were low. Without being bound to any particular theory, the orientation and flexibility of the chiral appendages (FIG. 13) are likely responsible for the low enantioselectivities observed for these meso-chiral porphyrins. For complexes 16f and 18a-c, the existence of atropisomers might cause additional problems for obtaining high enantioselectivity. It is provided that meso-chiral porphyrins containing more rigid chiral appendages with desirable geometry and orientation can improve diastereoselectivity and enantioselectivity.

TABLE 6
Cyclopropanation of Styrene with EDA Catalyzed by Cobalt
Complexes of meso-Chiral Porphyrins.^{a}

| entry | [Co(por)]^{b} | temp (° C.) | yield (%)^{c} | cis:trans^{c} | ee (%)^{d} |
|---|---|---|---|---|---|
| 1 | 16a | 80 | 97 | 28:72 | 9(1) |
| 2 | 16a | 23 | 92 | 27:73 | 11(2) |
| 3 | 16a | 0 | 88 | 26:74 | 12(2) |
| 4 | 16b | 80 | 95 | 32:68 | 8(5) |
| 5 | 16c | 80 | 95 | 31:69 | 9(5) |
| 6 | 16d | 0 | 87 | 27:73 | 12(1) |
| 7^{e} | 16e | 80 | 99 | 32:68 | 1(1) |
| 8 | 16^{e} | 23 | 98 | 30:70 | 4(1) |
| 9^{e} | 16e | 0 | 92 | 30:70 | 3(1) |
| 10 | 16f | 80 | 79 | 36:64 | 1(1) |
| 11 | 16f | 23 | 73 | 35:65 | 1(0) |
| 12 | 18a | 80 | 80 | 34:66 | 6(6) |
| 13 | 18a | 0 | 83 | 32:68 | 5(8) |
| 14 | 18b | 80 | 99 | 37:63 | 6(6) |
| 15 | 18c | 80 | 82 | 32:68 | 5(4) |
| 16 | 18c | 23 | 79 | 32:68 | 6(5) |
| 17 | 18c | 0 | 73 | 32:68 | 6(6) |

^{a}Reactions were carried out in toluene for 24 h under N₂ with 1.0 equiv of styrene, 1.2 equiv of EDA and 2 mol % [Co(por)]. Concentration: 0.5 mmol styrene/2 mL toluene.
^{b}See FIG. 1 for structures.
^{c}Determined by GC.
^{d}Determined by chiral GC: trans(cis).
^{e}Reaction time was 15 h.

Further, cobalt complexes of ortho-chiral porphyrins, e.g., compounds 21-a-20d and compounds 21l-21o, also were examined as catalysts for the model cyclopropanation reaction provided in FIG. 21. Using 1 mol % ortho-chiral porphyrin catalyst, the reactions proceeded effectively at room temperature in a one-pot synthesis method with styrene as the limiting reagent, producing the desired cyclopropanes in high yields (see Table 7).

Each of the four possible stereoisomers (trans-(1R,2R), trans-(1S,2S), cis-(1S,2R), or cis-(1R,2S) as provided in FIG. 21) could be produced as the dominant product when 21a, 21b, 21c or 21d was used as the catalyst, respectively, (Table 7, entries 1-4). This result indicates a high dependence of catalytic selectivity on the structure of the chiral R* units. The moderate enantioselectivities were doubled when 0.5 equivalents of 4-dimethylaminopyridine (DMAP) were added (Table 7, entries 5-7), suggesting significant trans influence of potential coordinate ligands on the metal center. The DMAP additive also boosted the production of the trans isomer (Table 7, entries 5-7). Further improvements in diastereoselectivity and enantioselectivity were observed when 21a was replaced with 21l wherein the two meso-groups are 3,5-di-tert-butylphenyl instead of phenyl (Table 7, entry 8). When t-BDA was used, the same catalyst produced trans-(1R,2R)-isomer as the only diastereomer in 95% ee, which was further improved to 98% ee at −20° C. (Table 7, entries 9 and 10). It is reasonable to expect that the same results would be obtained for trans-(1S,2S)-isomer if the enantiomer of 21l is employed as a catalyst.

TABLE 7
Asymmetric Cyclopropanation of Styrene Catalyzed by 21.^{a}

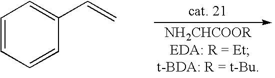

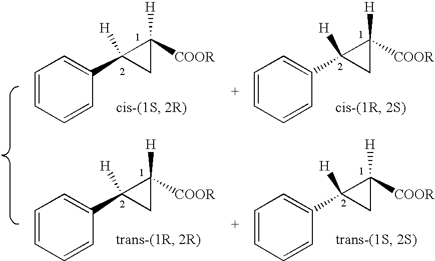

| Entry | 21 | diazo | Additive | Yield (%)^{b} | Trans: cis^{b} | ee^{c} | config^{d} |
|---|---|---|---|---|---|---|---|
| 1 | 21a | EDA | — | 92 (—) | 87:13 | 31% | 1R, 2R |
| 2 | 21b | EDA | — | 77 (—) | 66:34 | 35% | 1S, 2S |
| 3 | 21c | EDA | — | 92 (—) | 32:68 | 48% | 1S, 2R |
| 4 | 21d | EDA | — | 95 (—) | 32:68 | 51% | 1R, 2S |
| 5 | 21a | EDA | DMAP | 91 (—) | 96:04 | 67% | 1R, 2R |
| 6 | 21c | EDA | DMAP | 52 (—) | 44:56 | 88% | 1R, 2R |
| 7 | 21d | EDA | DMAP | 57 (—) | 42:58 | 89% | 1R, 2R |
| 8 | 21l | EDA | DMAP | 86 (82) | 97:03 | 78% | 1R, 2R |
| 9 | 21l | t-BDA | DMAP | 88 (84) | >99:01 | 95% | 1R, 2R |
| 10^{e} | 21l | t-BDA | DMAP | 84 (85) | >99:01 | 98% | 1R, 2R |
| 11 | 21m | EDA | DMAP | 65 (59) | 31:69 | 92% | 1S, 2R |
| 12^{f} | 21m | t-BDA | DMAP | 78 (75) | 37:63 | 96% | 1S, 2R |
| 13 | 21n | EDA | DMAP | 68 (—) | 30:70 | 95% | 1R, 2S |
| 14^{f} | 21n | t-BDA | DMAP | 76 (—) | 38:62 | 95% | 1R, 2S |
| 15 | 21o | EDA | DMAP | 80 (—) | 96:04 | 59% | 1R, 2R |
| 16 | 21o | t-BDA | DMAP | 73 (—) | 99:01 | 78% | 1R, 2R |

^{a}Reactions were carried out at room temperature in toluene for 20 h under N₂ with 1.0 equiv of styrene, 1.2 equiv of diazo reagent and 1 mol % 21 in the presence of 0.5 equiv of additive. Concentration: 0.25 mmol styrene/1 mL toluene.
^{b}Determined by GC. Yields in parentheses represent isolated yields.
^{c}ee of major diastereomer determined by chiral GC.
^{d}Absolute configuration of major enantiomer determined by optical rotation.
^{e}Carried out at −20° C. for 8 h.
^{f}5 mol % 21 was used.

The same structure modification resulted in 96% ee for cis-(1S,2R)-isomer with 21m and 95% ee for cis-(1R,2S)-isomer with 21n (entries 12 and 14). The results obtained with 210 bearing meso-n-heptyl groups (Table 5, entries 15 and 16) further underline the importance of both R and R* groups of the chiral porphyrins (see, e.g., FIG. 18) in achieving high selectivities.

The prototypical cyclopropanation reaction (FIG. 21) also can be used to evaluate the catalytic activities of meso- and ortho-chiral porphyrins as provided in hereinabove. The C2-symmetric secondary chiral amine units of meso-chiral porphyrins as provided in FIG. 15 should avoid the unfavorable orientation and flexibility that are associated with the chiral alcohol and amide constituents of compounds 15a-15f and 17a-17c provided in FIG. 13, and should provide improved asymmetric induction. In combination with the tuning of the meso-R group, diastereomer differentiation also can be achieved. Given the results presented in Table 5, provided with cobalt complexes of ortho-chiral porphyrins 20a-20d and 20l-20o, systematic catalytic studies can be performed with ortho-chiral porphyrins (see, e.g., FIG. 16), C (FIG. 17) and D, E, F, and G (FIG. 18) that possess diverse electronic, steric, and chiral environments. In addition to DMAP, common nitrogen, phosphine, and sulfur coordinating ligands can be used as additives.

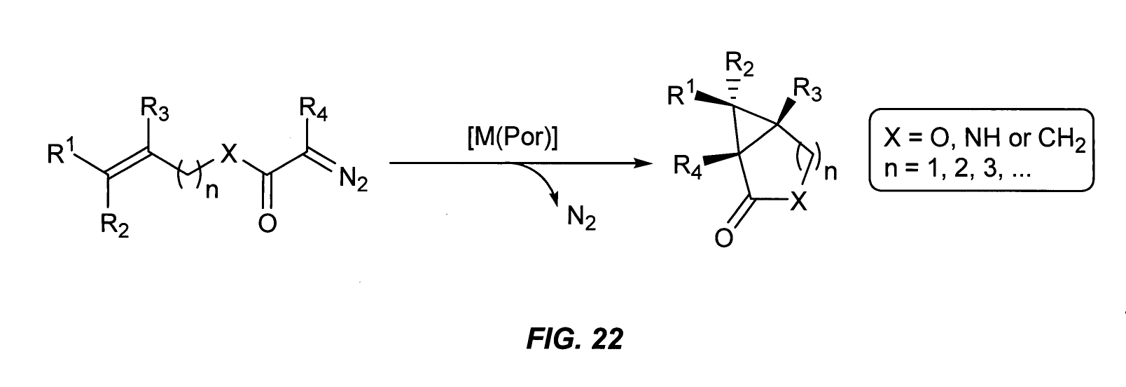

Efficient catalytic systems that allow exclusive formation of each one of the four possible isomers (FIG. 21) also can be developed, as achieved for the (1R,2R)-isomer with 21l (Table 7, entry 10). Thus, the cobalt-based catalytic system can be applied to cyclopropanation reactions comprising a wide range of alkenes. In addition to styrene derivatives, substrates can include nonaromatic alkenes, including but not limited to di-, tri- and tetra-substituted alkenes; cis- and trans-alkenes; and cyclic and non-cyclic alkenes. In addition to EDA and t-BDA, other diazo reagents can be used as potential carbene sources to further expand the utility of the catalytic reaction, including, but not limited to 2,6-di-tert-butyl-4-methylphenyl diazoacetate, methyl phenyldiazoacetate, ethyl diazoacetate, diethyl diazomalonate, and trimethylsilyldiazomethane. A series of diazo compounds bearing a pendant alkene C═C bond, including allylic diazoacetates (X═O, n=1), also can be employed for the development of intramolecular asymmetric cyclopropanation, leading to the construction of fused ring structures (see FIG. 22).

Further, ortho-chiral cobalt porphyrin catalysts 21a, 21d, 21f, 21j, 21l, 21m, 21q, and 21r catalyzed the intramolecular cyclopropanation of a variety of aromatic and non-aromatic diazo alkenes (Table 8). Yields of the reactions ranged from 50 to 99% with 24-90% ee.

Thus, the presently disclosed subject matter demonstrates that porphyrins with different electronic, steric, and chiral environments can be combined with different substrates to achieve high activity and selectivity. Accordingly, a chemical library or “toolbox” of effective metalloporphyrins for the asymmetric and symmetric cyclopropanation of a broad scope of substrates can be assembled.

TABLE 8
Cobalt Porphyrin Catalyzed Intramolecular Cyclopropanation
Reactions

| Reactant | Product |
|---|---|
| 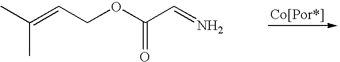 | 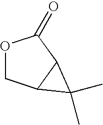 |
| 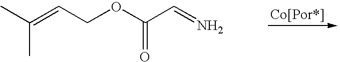 | 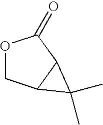 |
| 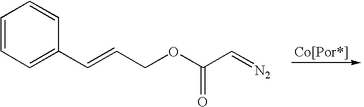 | 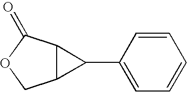 |
| 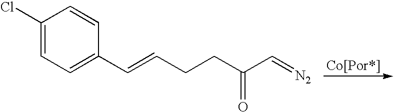 | 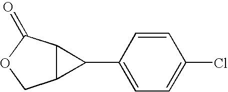 |
| 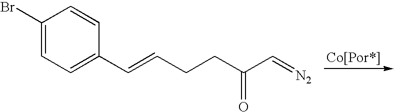 | 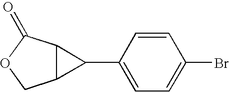 |
| 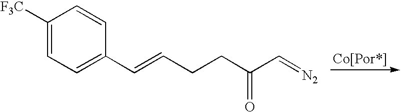 | 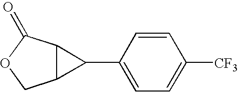 |
| 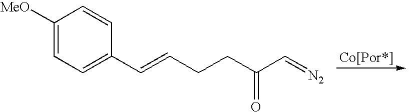 | 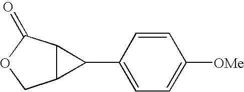 |
| 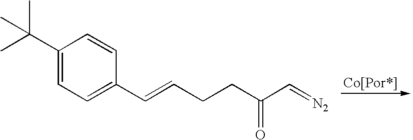 | 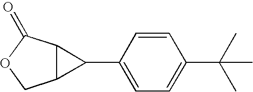 |

In comparative studies of cobalt, iron, ruthenium and rhodium porphyrins for catalytic cyclopropanation of styrene, it was found that the catalytic conversion of styrene to the desired cyclopropane ester increased in the order of Ru(TPP)(CO), Fe(TPP)Cl, Rh(TPP)I and Co(TPP) (TPP: tetraphenylporphyrin) using a practical one-pot protocol (alkenes as limiting reagents and no slow addition of diazo reagents) because the formation of the dimerization products (ethyl maleate and ethyl fumarate) from EDA decreased in the same order. See Huana et al., supra. It was also observed that the trans:cis isomer ratios of the desired product increased from 64:34 with Rh(TPP)I to 75:25 with Co(TPP) to 88:12 with Fe(TPP)Cl to 94:06 with Ru(TPP)(CO). See Huang et al., supra. In addition to the foregoing ligand tuning, the observed metal ion effect should provide an additional element for controlling the catalytic activity and selectivity. In addition to cobalt, complexes of iron, ruthenium and rhodium with these chiral porphyrins can be prepared to further characterize the metal ion effect.

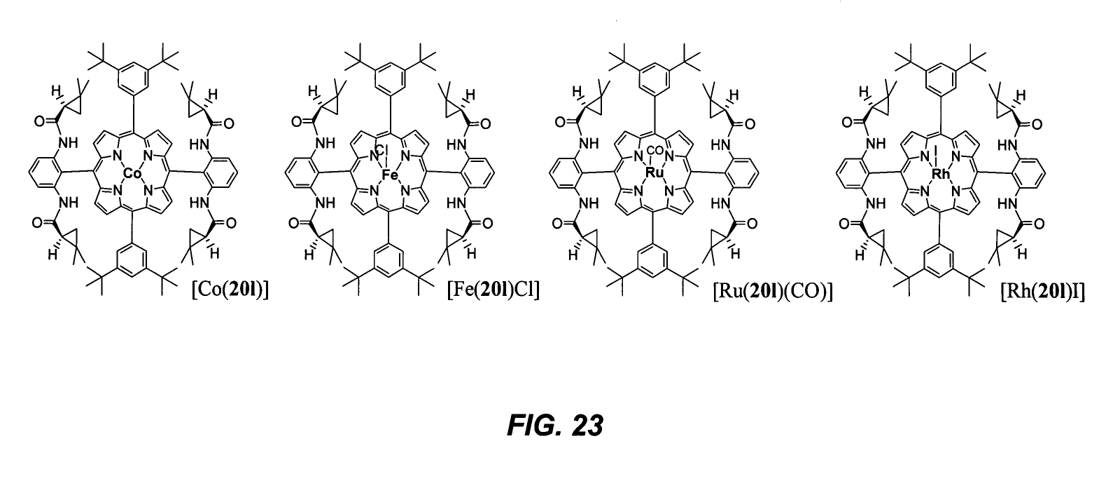

Accordingly, different metal complexes of ortho-chiral porphyrin 201 (see FIG. 23), the cobalt complex of which displayed remarkable diastereoselectivity and enantioselectivity (Table 7), can be used in the presently disclosed subject matter.

Given that the trans:cis isomer ratio was dramatically improved from 88:12 with Co(TPP) to >99:01 with [Co(20l)], it is of interest to determine the diastereoselectivities and enantioselectivities of [Fe(20l)Cl], [Ru(20l)(CO)], [Rh(20l)I] (see FIG. 23). Because Rh(TPP)I gave the highest cis:trans isomer ratio, [Rh(20f)I] and Rh(20g)I] also can be prepared by the presently disclosed subject matter in an effort to further increase the cis-diastereoselectivity observed for [Co(20m)] and [Co(20n)] (see Table 7).

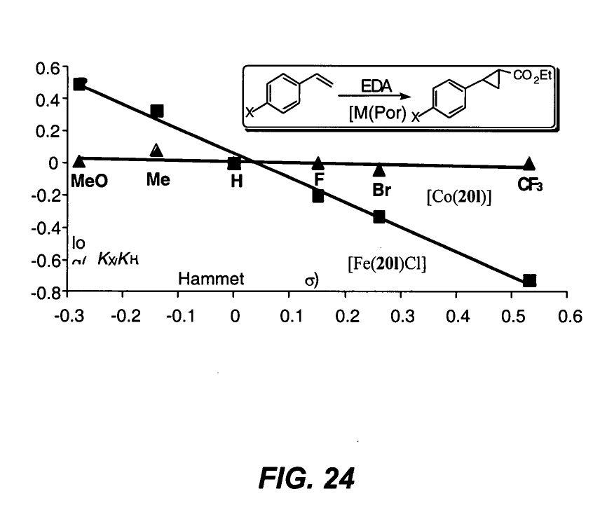

The electronic and steric effects of substrates on asymmetric cyclopropanation reactions catalyzed by [Co(20l)] were compared with those effects in reactions catalyzed by [Fe(20l)Cl]. Unlike in other metal complex-based systems, an unusual electronic insensitivity but a pronounced steric influence was observed in the [Co(20l)] catalytic system (Table 9). The variety of styrene substrates includes derivatives with electron-donating, neutral and electron-withdrawing substituents. In most cases, the corresponding trans-cyclopropane ester was produced with excellent diastereoselectivity and enantioselectivity. The Co center is believed to be responsible for the observed insensitivity since, as shown in FIG. 24, cyclopropanation by [Fe(20l)Cl] exhibited the expected linear Hammett plot with a large negative slope of −1.53.

TABLE 9
Asymmetric Cyclopropanation of Alkenes by [Co(201)].^{a}

| | | | temp | yield^{b} | | ee (%)^{d} |
|---|---|---|---|---|---|---|
| entry | product | R | (° C.) | (%) | trans:cis^{c} | trans |
| 1 2 3 4 | 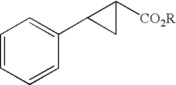 | Et Et t-Bu t-Bu | RT −20 RT −20 | 82 86^{e}84 85^{e} | 97:3 98:2 99:1 99:1 | 78 80 95 98 |
| 5 6 7 | 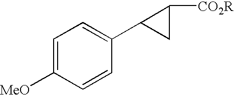 | Et t-Bu t-Bu | RT RT −20 | 82 86 76^{e} | 93:7 99:1 99:1 | 84 96 98 |
| 8 9 10 | 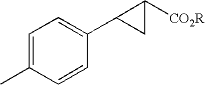 | Et t-Bu t-Bu | RT RT −20 | 71 91 66^{e} | 96:4 99:1 >99:1 | 70 94 92 |
| 11 12 13 14 | 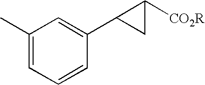 | Et Et t-Bu t-Bu | RT −20 RT −20 | 87^{c}82^{e}92 54^{e} | 97:3 99:1 99:1 99:1 | 79 87 94 98 |
| 15 16 17 | 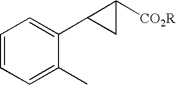 | Et t-Bu t-Bu | RT RT −20 | 61 84 76^{e} | 97:3 >99:1 >99:1 | 79 94 97 |
| 18 19 20 | 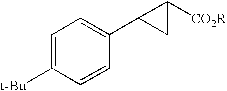 | Et t-Bu t-Bu | RT RT −20 | 95 92 86^{e} | 96:4 99:1 >99:1 | 89 93 91 |
| 21 22 23 | 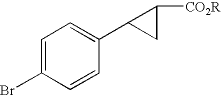 | Et t-Bu t-Bu | RT RT −20 | 81 92 78^{e} | 95:5 99:1 >99:1 | 73 93 97 |
| 24 25 26 | 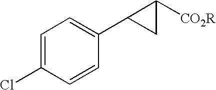 | Et t-Bu t-Bu | RT RT −20 | 71 69 52^{e} | 93:7 98:2 98:2 | 68 91 96 |
| 27 28 29 | 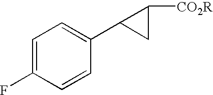 | Et t-Bu t-Bu | RT RT −20 | 60 88 29^{e} | 93:7 98:2 97:3 | 72 86 94 |
| 30 31 32 | 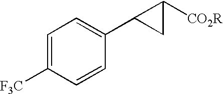 | Et t-Bu t-Bu | RT RT −20 | 79 64 65^{e} | 95:5 99:1 99:1 | 77 92 87 |
| 33 34 35 | 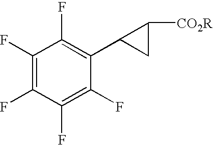 | Et t-Bu t-Bu | RT RT −20 | 40 74^{f}75^{e,f} | 93:7 97:3 99:1 | 65 84 90 |
| 36 37 38 | 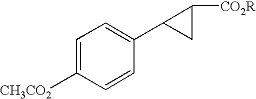 | Et t-Bu Et | 40 40 RT | 63 87 83^{c} | 92:8 98:2 94:6 | 72 88 75 |
| 39 40 41 | 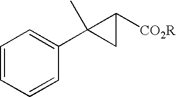 | Et Et t-Bu | 0 −20 RT | 69^{c}67^{e}31^{c} | 96:4 98:2 96:4 | 82 87 nr^{h} |
| 42 43 44 | 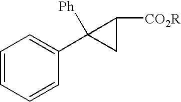 | Et Et t-Bu | RT RT RT | 46^{c}85^{f}10^{c} | nd^{g} nd^{g} nd^{g} | nr^{h}nr^{h}nr^{h} |
| 45 46 47 | 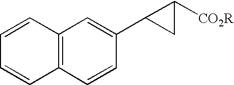 | Et t-Bu t-Bu | RT RT −20 | 72 84 58^{e} | 96:4 99:1 99:1 | 85 95 98 |

¹Performed in toluene for 20 h under N₂ with 1.0 equiv of alkene, 1.2 equiv of EDA or t-BDA and 1 mol % [Co(201)] in the presence of 0.5 equiv of DMAP. [alkene]: 0.25 M.
^{b}Isolated yields.
^{c}Determined by GC.
^{d}Determined by chiral GC or chiral HPLC.
^{e}Carried out for 8 h.
^{f}Used 5 mol % [Co(201)].
^{g}No diastereomers;
^{h}Not resolved.

Better stereoselectivities were generally seen with t-BDA than EDA and lowering the reaction temperature is believed to further improve enantioselectivity. Competition experiments with sterically different substrates showed that styrene reacted with EDA 1.3 and 3.0 times faster than α-methyl and α-phenyl styrenes, respectively, and that bulkier t-BDA was 3.8 times less reactive than EDA. Also, 2-vinylnapthalene was more reactive than styrene. Experiments with styrene and d₈-styrene found no secondary kinetic isotope effect.

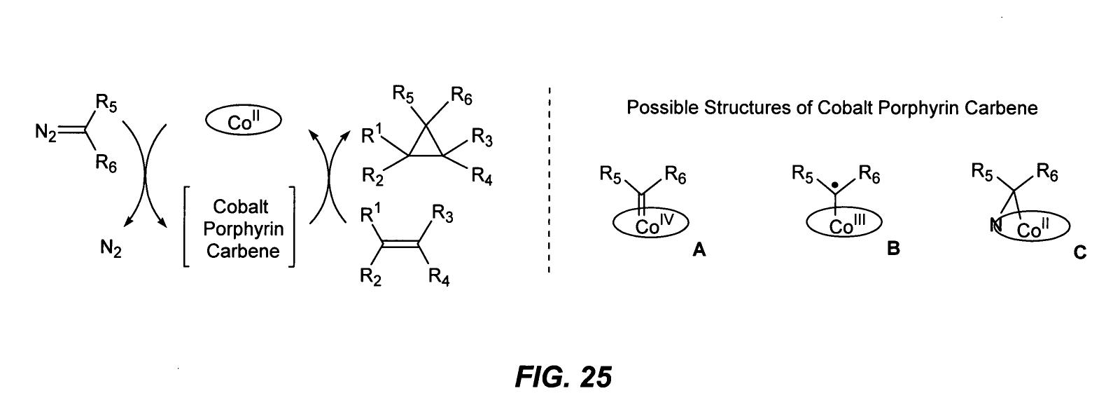

Understanding the reaction mechanism plays a role in further improvement of the metalloporphyrin-catalyzed cyclopropanation systems. Without being bound to any particular theory, on the basis of the proposed mechanisms for other metalloporphyrin systems, it is reasonable to assume the catalytic cyclopropanation by cobalt porphyrins proceeds via a similar mechanism involving a metal-carbene intermediate, although a noncarbene mechanism cannot be excluded. As shown in FIG. 25, reaction of Co(II) porphyrin with the diazo reagent generates a cobalt porphyrin carbene intermediate with the expulsion of nitrogen.

Carbene transfer from the intermediate to the alkene substrate affords the cyclopropane product and regenerates Co(II) porphyrin which continues the catalytic cycle. Three possible structures could be proposed for the cobalt porphyrin carbene intermediate (FIG. 25). Structure A of FIG. 25 represents a normal metal-carbene complex with a Co—C double bond that requires an uncommon Co(IV) high oxidation state. Stable carbene complexes of rhodium, iron, ruthenium, and osmium porphyrins have been characterized. Structure B of FIG. 25 illustrates an unusual metal-carbene complex with a Co—C single bond between a Co(III) center and a carbon-based radical. A similar structure was recently proposed for 3-oxobutylideneaminato-cobalt and salen-cobalt-based cyclopropanation system. While detection of such an intermediate remains elusive, the radical character of B appears to be consistent with the substrate electronic insensitivity observed with [Co(20l)] and could rationalize the lack of olefin side products even in the presence of excess diazo reagents. Combined with the stereochemical outcome and absence of kinetic isotope effect, the alkene presumably approaches B in a parallel, end-on fashion via an early transition state in that there is no charge build-up and the alkene rehybridization is insignificant. Cyclopropanation is then completed in either a concerted mode or a stepwise manner followed by rapid ring closure, as suggested by the high stereoselectivities. Structure C of FIG. 25 contains a carbene unit bridging a Co(II) center and one of the pyrrole nitrogen atoms. This bonding mode was previously observed in Co(III) porphyrins, but not in Co(II) porphyrins.

III. Aziridination by Metalloporphyrins

Aziridines are a class of synthetically and biologically important compounds that have found many applications. See Hu. X. E. Tetrahedron 2004, 60, 2701; Sweeney. J. B. Chem. Soc. Rev. 2002, 31, 247; Zwanenburq, B.; ten Holte. P. Top. Curr. Chem. 2001, 216, 93; McCoull. W.; Davis. F. A. Synthesis 2000, 1347; and Tanner, D. Angew. Chem., Int. Edit. Engl. 1994, 33, 599. In addition to being an important motif in many biologically and pharmaceutically interesting compounds, aziridines are notably known as a class of versatile synthons for preparation of functionalized amines. Among synthetic methodologies, transition metal complex-mediated aziridination of alkenes with a nitrene source represents a direct and powerful approach for the construction of the aziridine rings. See Muller, P.; Fruit, C. Chem. Rev. 2003, 103, 2905; Jacobsen. E. N. In Comprehensive Asymmetric Catalysis; Jacobsen, E. N., Pfaltz, A., Yamamoto, H., Eds.; Springer: Berlin, 1999, 2, 607; and Osborn. H. M. I.; Sweeney, J. Tetrahedron: Asymmetry 1997, 8, 1693.

[N-(p-toluenesulfonyl)imino]phenyliodinane (Phl=NTs) has been extensively used as a primary nitrene source for catalytic aziridination. See Dauban. P.: Dodd, R. H. Synlett 2003, 1571; Koser. G. F. Top. Curr. Chem. 2003, 224, 137; and Yamada. Y. et al., Chem. Lett. 1975, 361. While significant progress has been made with Phl=NTs in a number of metal-catalyzed systems, this nitrene source suffers from several drawbacks: commercial unavailability, high cost, short shelf life, insolubility in common solvents, and the generation of Phl as a by-product. To overcome these limitations, it is desirable to develop catalytic systems capable of employing alternative nitrene sources.

Recently, there has been growing interest in using chloramine-T (CT) (see Simkhovich. L.; Gross, Z. Tetrahedron Lett. 2001, 42, 8089; Albone, D., et al., J. Org. Chem. 1998, 63, 9569; and Mairena. M. A., et al., Organometallics 2004, 23, 253), bromamine-T (BT) (see Chanda. B. M. et al., J. Org. Chem. 2001, 66, 30; Antunes. A. M. M., et al., Chem. Comm. 2001, 405), and organic azides (see Omura. K. et al., Chem. Commun. 2004, 2060; Li. Z. et al., J. Am. Chem. Soc. 1995, 117, 5889) as alternative nitrene sources for metal-catalyzed aziridination, because of their attractive properties. The commercially available CT is inexpensive and has excellent stability. The analogous BT, which can be easily prepared from CT, has the same characteristics but exhibits different reactivity. Organic azides represent a class of compounds with diverse structures that can be synthesized in straightforward reactions of the corresponding halides with sodium azide. Many low cost organic azides are also commercially available. Furthermore, catalytic aziridination processes with these nitrene sources would generate more environmentally benign by-products (sodium halides or dinitrogen).

Owing to their unusual selectivity and excellent stability, as well as their biological relevance, metalloporphyrins have played a pivotal role in the development of several important catalytic atom/group transfer reactions, including aziridination. In fact, metalloporphyrins were the first transition metal complexes that demonstrated catalytic aziridination activity. See Groves. J. T.: Takahashi, T. J. Am. Chem. Soc. 1983, 105, 2073; and Mansuy. D., et al., J. Chem. Soc., Chem. Commun. 1984, 1161. Following this breakthrough, porphyrin complexes of several metal ions (Mn, Fe, and Ru) were reported to be effective with Phl=NTs. See Mahv, J.-P., et al., J. Chem. Soc. Perkin Trans. II 1988, 1517; Lai. T.-S., et al., Chem. Commun. 1997, 2373; Simonato, J.-P. et al., Chem. Commun. 1997, 989; Au, S.-M et al., J. Am. Chem. Soc. 1999, 121, 9120; Liang. J.-L. et al., Chem. Eur. J. 2002, 8, 1563. While CT, BT, and organic azides have been pursued using several transition metal complex systems, the catalytic activity of metalloporphyrins with these attractive nitrene sources has not been explored.

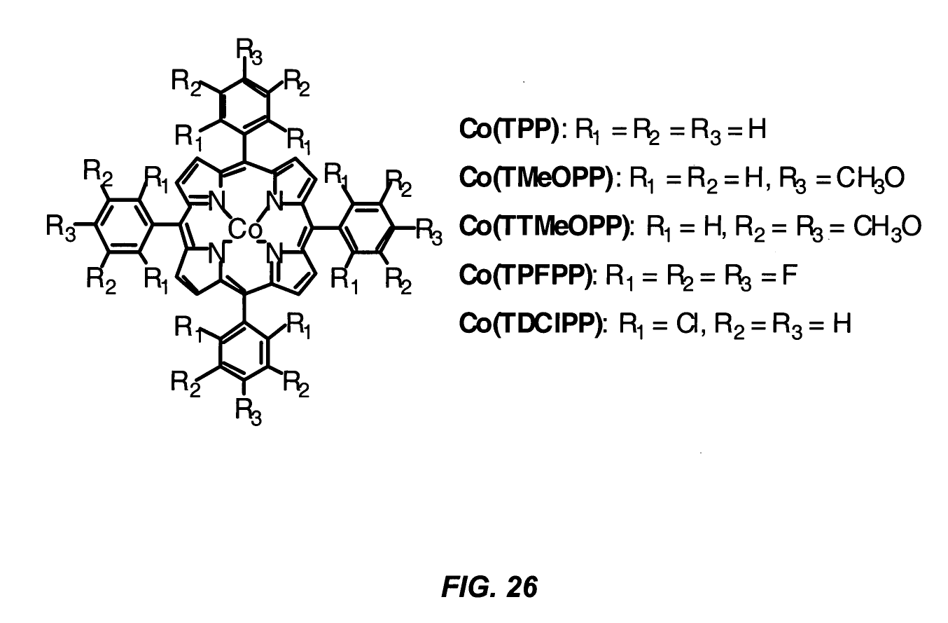

Accordingly, the presently disclosed subject matter identifies suitable alternative nitrene sources for catalytic aziridination by metalloporphyrins, and provides the cobalt-based catalytic systems that are efficient for aziridination of a wide variety of alkenes, including aromatic alkenes, aliphatic alkenes, acyclic alkenes, cyclic alkenes, electronically deficient alkenes, and sterically hindered alkenes. Representative, non-limiting examples of Cobalt(II) porphyrin catalysts useful for aziridination are provided in FIG. 26. Among cobalt complexes of different porphyrins, Co(TDCIPP) is an effective catalyst that can aziridinate a wide variety of alkenes, using BT as the nitrene source. The catalytic system can operate at room temperature in one-pot fashion with alkenes as limiting reagents, forming the desired N-sulfonylated aziridine derivatives in high to excellent 10 yields with NaBr as the by-product. Employing the family of new chiral porphyrins described hereinabove asymmetric versions of the aziridination processes shown in Equations 4 and 5 are possible.

Bromamine-T can be an effective nitrene source for aziridination of alkenes by metalloporphyrins (see Equation 4).

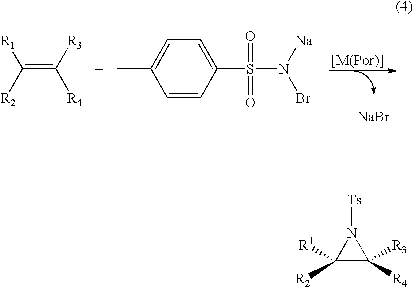

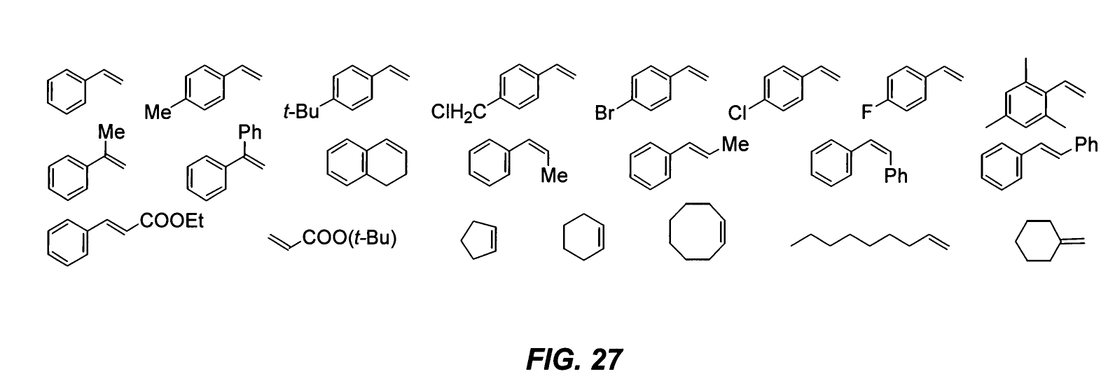

The combination of Fe(TPP)Cl and BT can effect aziridination reactions under mild and practical conditions with alkenes as limiting agents. The catalytic system is general and suitable for a wide range of substrates, including aromatic, aliphatic, cyclic, and acyclic alkenes, and α,β-unsaturated esters (see FIG. 27), producing the corresponding N-sulfonylated aziridines. The isolated yields ranged from 35% for aliphatic alkenes to 80% for styrene derivatives.

For 1,2-disubstituted alkenes, only moderate stereospecificities were achieved. A notable porphyrin ligand dependence was uncovered for the Fe(Por)Cl/BT catalytic system. While no catalytic activity was observed with Fe(OEP)Cl (OEP: octaethylporphyrin), the electron deficient Fe(TPFPP)Cl (TPFPP: tetrakis(pentafluorophenyl)porphyrin) improved the yield of styrene reaction significantly. It is also worth pointing out that other metalloporphyrins could aziridinate styrene with BT, including Mn(TPP)Cl, Ru(TPP)(CO), and Co(TPP), albeit at lower yields.

To explore the catalytic aziridination activities of Co complexes supported by various porphyrins under practical conditions (room temperature (20° C.-25° C.), one-pot protocol, and styrene as the limiting reagent), a series of achiral porphyrins with varied electronic and steric properties including porphyrins with various heteroatom substituents, were prepared. The results of aziridination reactions using styrene as a model substrate are summarized in Table 10. Although the Co complex of the most common porphyrin Co(TPP) could aziridinate styrene in a low yield, the Co complexes of electron-rich porphyrins, such as Co(TTMeOPP) and Co(TMeOPP) furnished no or only a trace amount of the desired product (see Table 10, entries 1-3). The production of aziridine, however, was tripled when the reaction was catalyzed by the Co complex of an electron-deficient porphyrin Co(TPFPP) (see Table 10, entry 4). Significant further improvement was achieved with the Co complex of an electron-deficient and sterically-hindered porphyrin Co(TDCIPP) as the catalyst, producing the desired aziridine in 83% isolated yield (see Table 10, entry 5).

Although a change in the ratio of styrene to bromamine-T from 1:2 to 1:1.2 had no significant influence on the catalytic reaction, excess of styrene resulted in a relatively lower yield (see Table 10, entries 6 and 7). Further, acetonitrile appeared to be the solvent of choice for the catalytic reaction, as the use of other solvents, such as tetrahydrofuran, methylene chloride, and toluene, gave no or only a trace amount of the desired product (see Table 10, entries 8-10). Although a slightly better yield was obtained at 40° C., a further increase in the reaction temperature caused a lower yield (see Table 10, entries 11 and 12). The room temperature reaction could be carried out effectively at a lower catalyst loading without affecting the yield (see Table 10, entry 13). A relatively lower yield was observed when the reaction time was shortened (see Table 10, entry 14). The use of a higher catalyst loading, however, could allow the reaction to be finished in a short time without a decrease of the yield (see Table 10, entry 15).

TABLE 10
Aziridination of Styrene by Cobalt Porphyrins.^{a}

| | | | mol | | temp | time | yield |
|---|---|---|---|---|---|---|---|
| entry | S:BT^{b} | [Co(Por)]^{c} | (%) | solvent | (° C.) | (h) | (%)^{d} |
| 1 | 1:2 | Co(TPP) | 5 | CH3CN | 23 | 18 | 18 |
| 2 | 1:2 | Co(TMeOPP) | 5 | CH3CN | 23 | 16 | <5 |
| 3 | 1:2 | Co(TTMeOPP) | 5 | CH3CN | 23 | 20 | 0 |
| 4 | 1:2 | Co(TPFPP) | 5 | CH3CN | 23 | 17 | 53 |
| 5 | 1:2 | Co(TDClPP) | 5 | CH3CN | 23 | 18 | 83 |
| 6 | 1:1.2 | Co(TDClPP) | 5 | CH3CN | 23 | 18 | 75 |
| 7 | 5:1 | Co(TDClPP) | 5 | CH3CN | 23 | 17 | 67 |
| 8 | 1:2 | Co(TDClPP) | 5 | THF | 23 | 20 | 0 |
| 9 | 1:2 | Co(TDClPP) | 5 | CH₂Cl₂ | 23 | 17 | <5 |
| 10 | 1:2 | Co(TDClPP) | 5 | CH₃C₆H₅ | 23 | 19 | 0 |
| 11 | 1:2 | Co(TDClPP) | 5 | CH₃CN | 40 | 17 | 84 |
| 12 | 1:2 | Co(TDClPP) | 5 | CH₃CN | 82 | 18 | 66 |
| 13 | 1:2 | Co(TDClPP) | 2 | CH₃CN | 23 | 17 | 80 |
| 14 | 1:2 | Co(TDClPP) | 5 | CH₃CN | 23 | 7 | 71 |
| 15 | 1:2 | co(TDClPP) | 10 | CH₃CN | 23 | 7 | 82 |

^{a}Carried out under N₂ in the presence of 5 Å molecular sieves with a concentration of 0.1 mmol styrene/2 mL solvent.
^{b}The mole ratio of styrene substrate to Bromamine-T.
^{c}See FIG. 1.
^{d}Isolated yields.

Using the reaction conditions described hereinabove, the Co(TDCIPP)-based catalytic system was found to be suitable for the aziridinated of several different types of alkene substrates (see Table 11). In addition to styrene, derivatives of styrene with alkyl substituents could be aziridinated to afford the desired products in high yields (see Table 11, entries 1-4). Further, functional groups in styrene derivatives could be well tolerated to generate the corresponding aziridines (see Table 11, entries 5-7). Sterically hindered derivatives, such as 2,4,6-trimethylstyrene, as well as 2-vinylnaphthalene, also could be catalytically aziridinated, albeit in lower yields (see Table 11, entries 8-9). Halogenated styrenes, including highly electron-deficient pentafluorostyrene, could be successfully converted to the desired aziridines in good to high yields (see Table 11, entries 10-13).

In addition, both α-substituted and β-substituted (cyclic and acyclic) styrenes were suitable substrates for the presently disclosed catalytic process (Table 11, entries 14-21). Without wishing to be bound to any particular theory, it appears that a competitive amidation reaction likely contributed to the low aziridination yield of 1,2-dihydronapthalene (see Table 11, entry 16), excellent yields were obtained for acyclic β-substituted styrenes in both cis and trans forms (see Table 11, entries 18-21). For the latter substrates, the reactions catalyzed by Co(TDCIPP) appeared to lack stereospecificity, although a high trans-stereoselectivity was observed for both cis- and trans-β-methylstyrenes. In addition to aromatic and conjugated alkenes, acyclic and cyclic aliphatic alkenes with different ring sizes are suitable substrates for the catalytic system (see Table 11, entries 22-25). Under similar conditions, exo-methylene carbocycles, such as methylenecyclohexane, also could be aziridinated to afford the desired spirocyclic aziridine in 67% isolated yield (see Table 11, entry 26).

TABLE 11
Aziridination of Different Alkenes by Co(TDCIPP).^{a}

| entry | substrate | product | yield (%)^{b} |
|---|---|---|---|
| 1 2 3 | 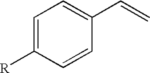 | 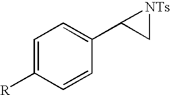 | R = H: 83 R = Me: 76 R = t-Bu: 81 |
| 4 | 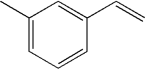 | 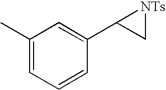 | 89 |
| 5 6 7 | 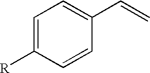 | 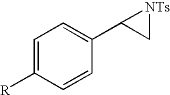 | R = ClCH₂: 86 R = CF₃: 90 R = CH₃CO₂: 91 |
| 8 | 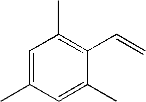 | 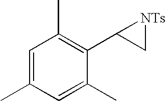 | 61 |
| 9 | 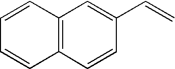 | 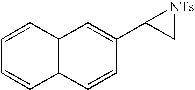 | 53 |
| 10 11 12 | 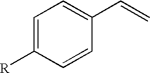 | 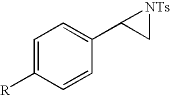 | R = Br: 70 R = Cl: 71 R = F: 86 |
| 13 | 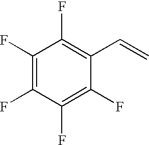 | 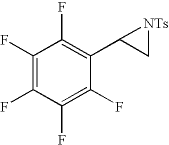 | 61 |
| 14 15 | 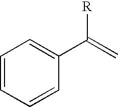 | 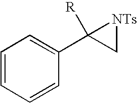 | R = Me: 70 R = Ph: 81 |
| 16 | 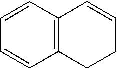 | 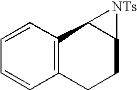 | 33 |
| 17^{c} | 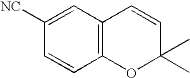 | 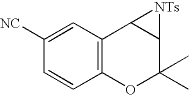 | 68 |
| 18 19^{d} | 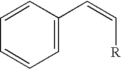 | 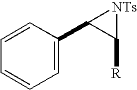 | R = Me: 94^{e}R = Ph: 92^{f} |
| 20 21^{d} | 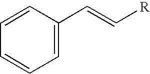 | 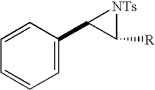 | R = Me: 87^{g}R = Ph: 94^{h} |
| 22 23 24 | 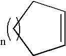 | 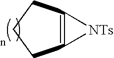 | n = 1: 61 n = 2: 66 n = 3: 79 |
| 25 | 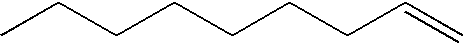 | 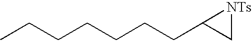 | 56 |
| 26 | 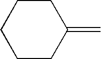 | 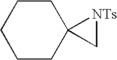 | 67 |

^{a}Carried out at RT in CH₃CN overnight under N_{2 with alkenes as limiting reagent (alkene:bromamine-T = 1:2) using 5 mol % Co(TDCIPP) in the presence of 5Å molecular sieves at concentration of 0.2 mmol alkene/4-5 mL CH}₃CN.
^{b}Isolated yields.
^{c}Performed with alkene:bromamine-T = 5:1.
^{d}Performed with alkene:bromamine-T = 1:3 using 10 mol % Co(TDCIPP).
^{e}cis:trans = 9:91.
^{f}cis:trans = 47:53.
^{g}cis:trans = 8:92.
^{h}cis:trans = 58:42.

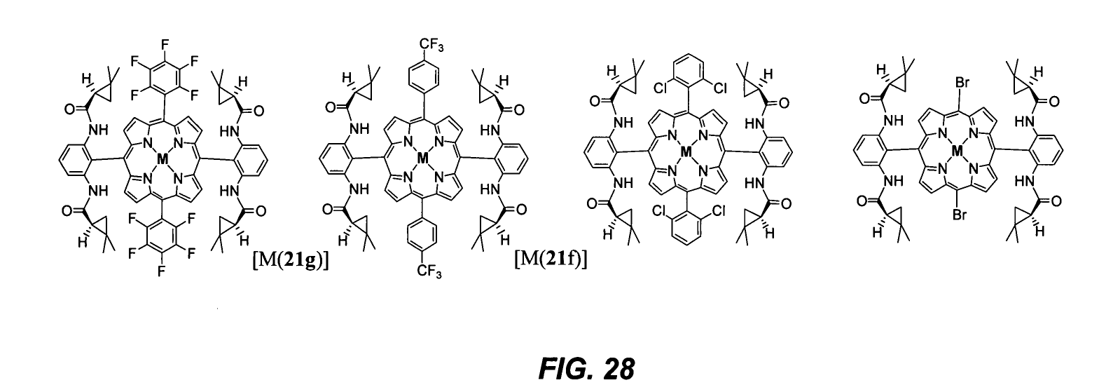

In light of the aziridination activities herein described for achiral porphyrins, electron-deficient chiral metalloporphyrins also can have enhanced catalytic activity and allow asymmetric induction for aziridination of alkenes with BT. In addition to the potential asymmetric aziridination activities of the meso- and ortho-chiral porphyrins, D₂-symmetric meso-chiral porphyrins that bear electron-withdrawing groups can be prepared, along with their iron, cobalt, manganese, and ruthenium complexes (FIG. 28) Similar approaches to those outlined hereinabove involving asymmetric cyclopropanation can be applied to gain a mechanistic understanding of asymmetric aziridination catalyzed by metalloporphyrins with BT, including the characterization of potential metalloporphyrin nitrene intermediates.

In comparison with N-sulfonylated aziridines, N-phosphorylated and N-phosphinylated aziridines have been shown to be advantageous as synthetic building blocks, since the phosphoryl and phosphinyl groups bring suitable activation to the aziridine ring and can be easily deprotected. See Hu. X. E. Tetrahedron Lett. 2002, 43, 5315; Hu, X. E.; Kim. N. K.: Ledoussal. B.: Colson. A.-O. Tetrahedron Lett. 2002, 43, 4289; Osowska-Pacewicka. K.; Zwierzak. A. Syn. Comm. 1998, 28, 1127; Gaida. T. et al., Tetrahedron 1997, 53, 4935; Osowska-Pacewicka. K.; Zwierzak. A. Synthesis 1996, 333; Osowska-Pacewicka. K.; Zwierzak. A. Polish J. Chem. 1994, 68, 1263; Sweeney, J. B.; Cantrill. A. A. Tetrahedron 2003, 59, 3677; Cantrill, A. A. et al., Tetrahedron 1998, 54, 2181; Osborn. H. M. I., et al., Tetrahedron Lett. 1994, 35, 2739; Osborn. H., et al., Synlett 1994, 145. For examples of biomedical applications of N-phosphorus-substituted aziridines, see Perlman. M. E.; Bardos. T. J. J. Org. Chem. 1988, 53,1761; Borkovec, A. B., et al., J. Med. Chem. 1966, 9, 522.

Although methods are available for the preparation of N-phosphorous-substituted aziridines, see Hu, X. E. Tetrahedron Lett. 2002, 43, 5315; Hu, X. E., et al., Tetrahedron Lett. 2002, 43, 4289; Osowska-Pacewicka. K.; Zwierzak, A. Syn. Comm. 1998, 28, 1127; Gaida. T. et al., Tetrahedron 1997, 53, 4935; Osowska-Pacewicka. K.; Zwierzak. A. Synthesis 1996, 333; and Osowska-Pacewicka, K.; Zwierzak. A. Polish J. Chem. 1994, 68, 1263; Sweeney, J. B.; Cantrill, A. A. Tetrahedron 2003, 59, 3677; Cantrill. A. A. et al., Tetrahedron 1998, 54, 2181; Osborn. H. M. I. et al. Tetrahedron Lett 1994, 35, 2739; Osborn. H. M. I. et al., Synlett 1994, 145, their direct synthesis via metal-mediated aziridination of alkenes has not been fully developed. A N-phosphorylated aziridine was recently synthesized in 33% yield via Rh-catalyzed aziridination. See Guthikonda. K.; Du Bois. J. J. Am. Chem. Soc. 2002, 124, 13672.

In some embodiments, the presently disclosed subject matter provides the application of diphenylphosphoryl azide (DPPA) as a new nitrene source for aziridination by metalloporphyrins (Equation 5). In particular, In some embodiments, the presently disclosed subject matter demonstrates that the cobalt(II) porphyrin complex Co(TPP) can catalyze aziridination of alkenes using diphenylphosphoryl azide (DPPA) as a convenient new nitrene source, leading to the formation of N-phosphorylated aziridines with dinitrogen as the by-product. The commercially available, low cost diphenylphosphoryl azide is a stable and distillable liquid that has been widely used in various organic syntheses. For selected examples, see Shioiri. T. et al., J. Am. Chem. Soc. 1972, 94, 6203; Yamada. S. et al., J. Am. Chem. Soc. 1975, 97, 7174; Lai. B., et al., Tetrahedron Lett. 1977, 1977; and Qian, L. et al., Tetrahedron Lett. 1990, 45, 6469.

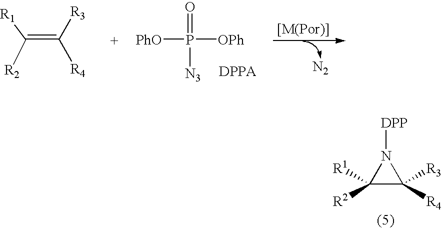

The results of the catalytic aziridination of styrene with DPPA by metal complexes of the common TPP under different conditions are provided in Table 12 and Table 13.

TABLE 12
Aziridination of Styrene with DPPA Catalyzed by Metalloporphyrins.

| | | Loading | | | Temp | Time | |
|---|---|---|---|---|---|---|---|
| Entry | Catalyst | (mol %) | S:A | Solvent | (° C.) | (h) | Yield (%) |
| 1 | Co(TPP) | 5 | 5:1 | Dichloromethane | 40 | 12 | 0 |
| 2 | Co(TPP) | 5 | 5:1 | Tetrahydrofuran | 65 | 12 | 0 |
| 3 | Co(TPP) | 5 | 5:1 | Toluene | 110 | 12 | 32 |
| 4 | Co(TPP) | 5 | 5:1 | Dimethylformamide | 150 | 12 | 0 |
| 5 | Co(TPP) | 5 | 5:1 | Chlorobenzene | 120 | 12 | 74 |
| 7 | Co(TPP) | 5 | 5:1 | Chlorobenzene | 120 | 6 | 60 |
| 8 | Co(TPP) | 10 | 5:1 | Chlorobenzene | 120 | 12 | 76 |
| 9 | Co(TPP) | 5 | 2:1 | Chlorobenzene | 120 | 12 | 54 |
| 10 | Co(TPP) | 5 | 1:2 | Chlorobenzene | 120 | 12 | 0 |
| 11 | Mn(TPP)Cl | 5 | 5:1 | Chlorobenzene | 120 | 12 | 0 |
| 12 | Fe(TPP)Cl | 5 | 5:1 | Chlorobenzene | 120 | 12 | 0 |
| 13 | Ru(TPP)(CO) | 5 | 5:1 | Chlorobenzene | 120 | 12 | 5 |

TABLE 13
Aziridination of Styrene with DPPA by Cobalt(II)
Tetraphenylporphyrin Complex under Various Conditions^{a}

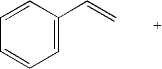

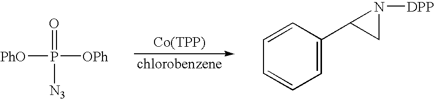

| | cat | | | temp | | |
|---|---|---|---|---|---|---|
| entry | (mol %) | S:A^{b} | additv (mol %) | (° C.) | time (h) | yield (%)^{c} |
| 1 | 10 | 1:2 | none (0) | 100 | 17 | 0 |
| 2 | 10 | 3:1 | none (0) | 100 | 17 | 32 |
| 3 | 10 | 5:1 | none (0) | 100 | 17 | 50 |
| 4 | 10 | 10:1 | none (0) | 100 | 17 | 33 |
| 5 | 10 | 5:1 | none (0) | 100 | 40 | 29 |
| 6 | 10 | 5:1 | none (0) | 120 | 17 | 54 |
| 7 | 10 | 5:1 | none (0) | 120 | 7 | 17 |
| 8 | 10 | 5:1 | none (0) | 120 | 40 | 0 |
| 9 | 10 | 5:1 | none (0) | 80 | 17 | 19 |
| 10 | 10 | 5:1 | none (0) | 80 | 46 | 56 |
| 11 | 5 | 5:1 | none (0) | 100 | 17 | 20 |
| 12 | 5 | 5:1 | none (0) | 120 | 17 | 27 |
| 13 | 10 | 5:1 | DMAP (10) | 100 | 17 | 0 |
| 14 | 10 | 5:1 | DMAP (10) | 80 | 17 | 0 |
| 15 | 10 | 5:1 | DMAP (10) | 100 | 6 | 0 |
| 16 | 10 | 5:1 | THF (100) | 100 | 17 | 35 |
| 17 | 10 | 5:1 | CH₃CN (100) | 100 | 17 | 43 |
| 18 | 10 | 5:1 | Ph₃P (10) | 100 | 17 | 42 |

^{a}Reactions were carried out in chlorobenzene under N₂ in the presence of 5Å molecular sieves using Co(TPP) as the catalyst with or without additives.
^{b}Mole ratio of styrene to DPPA.
^{c}Isolated yields.

As summarized in the Tables 12 and 13, the best results were obtained with the cobalt catalyst using chlorobenzene as a solvent. None or only a trace of the desired product was formed with other metalloporphyrins or without a catalyst. Decreasing the styrene/DPPA ratio reduced the yield. No aziridine was observed when styrene was used as the limiting reagent. A styrene to DPPA ratio of 5:1 gave the best result for the Co(TPP)-catalyzed aziridination in chlorobenzene (see Table 13, entries 14).

Overall, the best yields were obtained at temperatures of 120° C. for 12 hours (Table 12, entries 5 and 8). The yield was reduced with shorter reaction times (Table 12, entry 7). Although the catalytic reaction could proceed with a nearly complete conversion of DPPA after 17 hours at 100° C., the N-phosphoylated aziridine was isolated in only 50% yield (Table 13, entry 3) due to formation of some unidentified side products during the reaction (and possibly during product isolation with silica gel). Although a slight increase in yield was obtained at a higher temperature (Table 13, entry 6), prolonged heating resulted in yield reduction (Table 13, entries 5 and 8). A reduced yield also was observed in a shorter reaction time (Table 13, entry 7) or at a lower reaction temperature (Table 13, entry 9). A reaction that was carried out at a lower temperature for a longer time gave the desired product in an improved yield (Table 13, entry 10). A reduction in catalyst loadings dropped the yields for overnight reactions (Table 13, entries 11 and 12).

Strong solvent effects were noticed for the aziridination process. Uses of common solvents other than chlorobenzene, including acetonitrile, dichloromethane, dimethylformamide, tetrahydrofuran and toluene, gave no or a small amount of the desired product. A negative additive effect was also observed for the catalytic process. Addition of a small amount of DMAP appeared to completely shut down the reaction (Table 13, entries 13-15). The negative effect was reduced with weaker coordinative additives, such as Ph₃P, THF, and CH₃CN.

Co(TPP)-based aziridination with DPPA was then investigated with different alkenes. The results of the Co(TPP)-based aziridination with DPPA of a series of styrene derivatives are summarized in Table 14. Under the abovementioned typical reaction conditions, while aziridination of p-tert-butyl styrene gave a slightly lower yield, m-methyl styrene was a better substrate than styrene (Table 14, entries 1-3). The Co-based aziridination system appeared to be equally suitable to styrene derivatives having electron-withdrawing substituents, such as halogen and trifluoromethyl groups (Table 14, entries 4-7). Even the highly electron-deficient pentafluorostyrene could be aziridinated with DPPA, albeit in lower yields (Table 14, entry 8). Although a low yield was obtained for the aziridination of 2-vinyinaphthalene (Table 14, entry 9), the reaction of m-nitrostyrene produced the desired N-phosphorylated aziridine in highest yield (Table 14, entry 10).

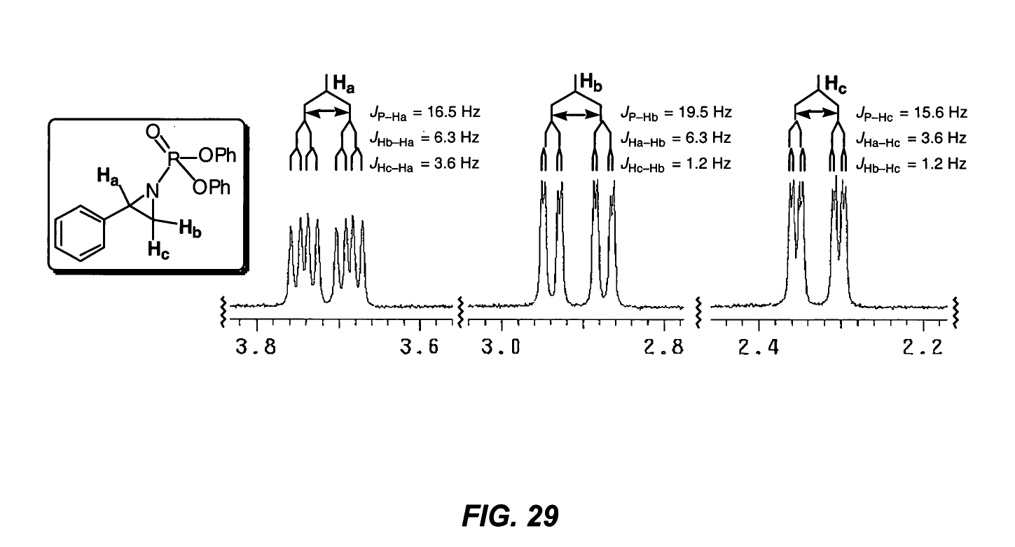

All the aziridination products were isolated in high purity and characterized by ¹H, ¹³C, and ³¹P NMR, FT-IR, and high-resolution MS spectroscopy. As exemplified with the N-phosphorylated aziridine from styrene (FIG. 29), each of the three aziridine-ring hydrogens exhibits a characteristic doublet of doublet of doublets (ddd) peak pattern in the ¹H NMR spectrum between 2.2-3.8 ppm, which results from the coupling among them and further split by the phosphorus atom.

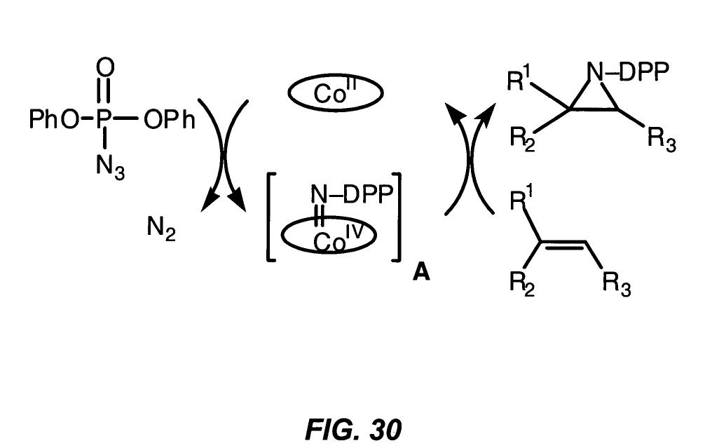

The catalytic aziridination by Co(TPP) with DPPA can be assumed to proceed via a similar mechanism to that proposed for other metalloporphyrin-based systems with Phl=NTs. See Vyas, R., et al., Org. Lett., 2004, 6, 1907; Groves. J. T.: Takahashi, T. J. Am. Chem. Soc., 1983, 105, 2073; Mansuy, D. et al., J. Chem. Soc., Chem Commun., 1984, 1161; Mahy, J.-P., et al., J. Chem. Soc. Perkin Trans. II 1988, 1517; Lai. T.-S., et al., Chem. Commun. 1997, 2373; Simonato, J.-P. et al., Chem. Commun. 1997, 989; Au. S.-M. et al., J. Am. Chem. Soc. 1999, 121, 9120; Lianq, J.-L., et al., Chem. Eur. J. 2002, 8, 1563. As illustrated in FIG. 30, this mechanism includes the involvement of a cobalt-nitrene intermediate A, which has not been known previously.

TABLE 14
Aziridination of Styrene Derivatives with DPPA Catalyzed by
Cobalt(II) Tetraphenylporphyrin Complex^{a}

| entry | substrate | product | yield (%)^{b} |
|---|---|---|---|
| 1 | 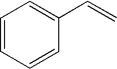 | 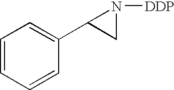 | 50 |
| 2 | 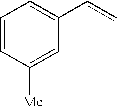 | 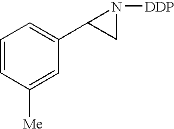 | 64 |
| 3 | 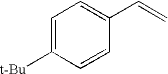 | 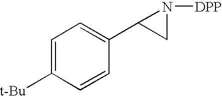 | 43 |
| 4 | 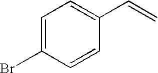 | 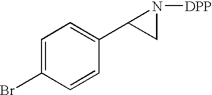 | 54 |
| 5 | 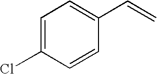 | 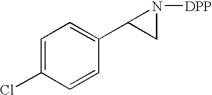 | 52 |
| 6 | 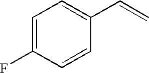 | 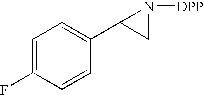 | 45 |
| 7 | 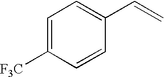 | 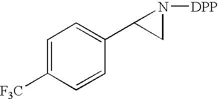 | 60 |
| 8 | 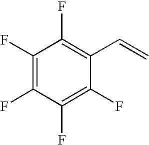 | 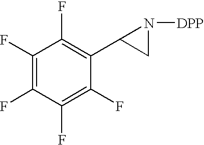 | 36 |
| 9 | 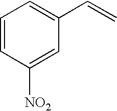 | 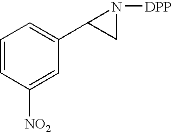 | 68 |
| 10 | 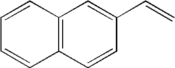 | 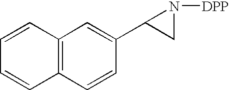 | 24 |

^{a}Reactions were carried out overnight at 100° C. in chlorobenzene under N₂ in the presence of 5Å molecular sieves using 10 mol %. Co(TPP). Concentration: 0.2 mmol DPPA/2 mL chlorobenzene; alkene:DPPA = 5:1.
^{b}Isolated yields.

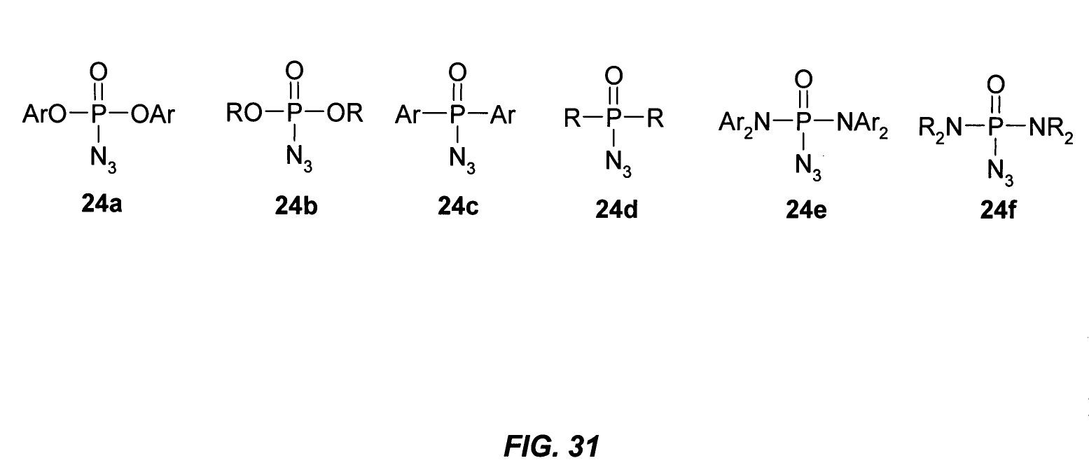

Cobalt porphyrins are capable of catalyzing aziridination with DPPA, forming synthetically useful N-phosphorylated aziridines. These results, together with the results of Co(TPP)/BT mediated aziridination, represents the first examples of cobalt-catalyzed aziridination and one of only a few catalytic aziridination systems that employs azides as nitrene sources. In addition to the further optimization of various reaction parameters, including the examination of possible trans effects of potential coordinating ligands, porphyrins containing different electronic and steric substituents can be employed to improve the catalytic efficiency, to expand the substrate scope, and to achieve high stereospecificity. Meanwhile, known derivatives of phosphoryl azides and related phosphinyl and phosphorodiamidic azides, e.g., compounds 24a-24f of FIG. 31, also can be employed as potential nitrene sources.

IV. Asymmetric Epoxidation by Metalloporphvrins

The chiral porphyrins of the presently disclosed subject matter also can be used as catalysts in asymmetric epoxidation reactions (see Equation 3).

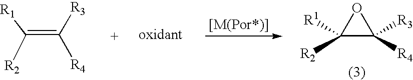

For representative metal-catalyzed epoxidation reactions, see Boschi, (1994) “Asymmetric Syntheses” In Metalloporphyrins Catalyzed Oxidations; Montanari, F., Casella, L., Eds.; Kluwer Academic Publishers: Boston, 1994; pp 239-267; Naruta, (1994) “Asymmetric Oxidation with Chiral Porphyrin Catalysts” In Metalloporphyrins in Catalytic Oxidations; Sheldon, R. A., Ed.; Marcel Dekker: New York, 1994; pp 241-259; Groves et al., (1983) J. Am. Chem. Soc. 105: 5791; Marchon et al., (2003) “Chiral Metalloporphyrins and Their Use in Enantiocontrol” in The Porphyrin Handbook; Kadish, K. M., Smith, K. M., Guilard, R., Eds.; Academic Press: San Diego, Calif., Vol. 11; pp 75-132; Rose et al., (2000) Polyhedron 19: 581; and Collman et al., (1993) Science 261: 1404-1411.

In some embodiments, the oxidant is selected from the group consisting of sodium hypochlorite, potassium monopersulfate, hydrogen peroxide, alkylhydroperoxides, m-chloroperbenzoic acid, amines N-oxides, iodosylbenzene, and dioxygen.

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. A method of synthesizing an aziridine compound, the method comprising reacting an alkene with a nitrene source in the presence of a cobalt-containing catalyst.

2. A method of synthesizing an aziridine compound, the method comprising reacting an alkene with a nitrene source in the presence of a porphyrin metal complex, wherein the porphyrin metal complex has the structure of Formula (I): 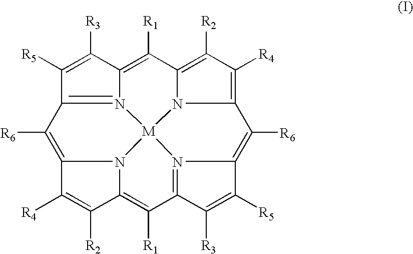
wherein:
M is a transition metal ion selected from the group consisting of zinc, rhodium, and cobalt; and
R₁, R₂, R₃, R₄, R₅ and R₆ are each independently selected from the group consisting of H, alkyl, substituted alkyl, arylalkyl, aryl, and substituted aryl and Y, wherein Y is a heteroatom-containing chiral moiety.

3. The method of claim 2, wherein the transition metal ion is cobalt.

4. The method of claim 2, wherein the alkene is selected from one of an aromatic alkene and a non-aromatic alkene.

5. The method of claim 4, wherein the one of an aromatic alkene and a non-aromatic alkene is selected from the group consisting of a di-substituted alkene, a tri-substituted alkene, and a tetra-substituted alkene.

6. The method of claim 4, wherein the one of an aromatic alkene and a non-aromatic alkene is selected from one of a cis-alkene and a trans alkene.

7. The method of claim 4, wherein the non-aromatic alkene is selected from one of a cyclic alkene and a non-cyclic alkene.

8. The method of claim 2, wherein the nitrene source is selected from the group consisting of bromamine-T, chloramine-T, and an organic azide.

9. The method of claim 8, wherein the nitrene source is bromamine-T.

10. The method of claim 8, wherein the organic azide is diphenylphosphoryl azide (DPPA).

11. The method of claim 2, wherein R₁ and R₆ are independently selected from the group consisting of aryl and substituted aryl.

12. The method of claim 11, wherein the substituted aryl is substituted with an electron-withdrawing group.

13. The method of claim 12, wherein the electron-withdrawing group is halogen.

14. The method of claim 2, wherein the porphyrin metal complex is selected from the group consisting of [Fe(TPP)Cl], [Fe(TPFPP)Cl], [Co(TDCIPP)] and [Co(TPFPP)].

15. The method of claim 14, wherein the porphyrin metal complex is [Co(TPP)].

16. The method of claim 2, wherein the porphyrin is present in a concentration ranging from about 2 mol % to about 10 mol %.

17. The method of claim 2, wherein the porphyrin is present in a concentration ranging from about 5 mol % to about 10 mol %.

18. The method of claim 2, wherein the alkene and the nitrene source are present in a ratio of about 1:2 alkene:nitrene.

19. The method of claim 2, wherein the alkene and the nitrene source are present in a ratio of about 5:1 alkene:nitrene.

20. The method of claim 2, wherein the reacting of the alkene with the nitrene source takes place in an aprotic solvent.

21. The method of claim 20, wherein the aprotic solvent is selected from the group consisting of acetonitrile and chlorobenzene.

22. The method of claim 2, wherein the reacting of the alkene with the nitrene source takes place at about room temperature.

23. The method of claim 2, wherein the reacting of the alkene with the nitrene source takes place at a temperature of between about 80° C. and about 120° C.

24. The method of claim 2, wherein the reacting of the alkene with the nitrene source takes place for between about 6 hours and about 46 hours.

25. A method for the cobalt-catalyzed intramolecular cyclopropanation of an alkene-substituted diazo compound, the method comprising reacting an alkene-substituted diazo compound with a cobalt-containing catalyst.

26. A method of synthesizing a cyclopropane compound, the method comprising reacting an alkene-substituted diazo compound with a porphyrin metal complex to form a cyclopropane compound, wherein the porphyrin metal complex has the structure of Formula (I): 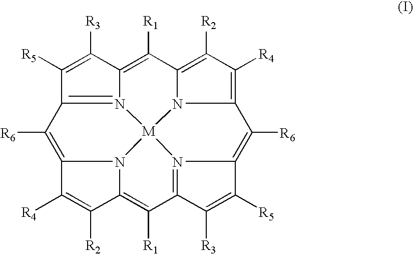
wherein:
M is Co;
R₁, R₂, R₃, R₄, R₅ and R₆ are each independently selected from the group consisting of H, alkyl, substituted alkyl, arylalkyl, aryl, substituted aryl, and Y, wherein Y is a heteroatom-containing chiral moiety.

27. The method of claim 26, wherein the alkene-substituted diazo compound comprises an alkene-substituted diazoacetate compound.

28. The method of claim 27, wherein the alkene-substituted diazoacetate compound comprises an allylic diazoacetate compound.

29. The method of claim 27, wherein the alkene-substituted diazo compound is selected from the group consisting of 3-methyl-2-buten-1-yl diazoacetate, 2-propen-1-yl diazoacetate, trans-3-phenyl-2-propen-1-yl diazoacetate, trans-3-(para-chlorophenyl)-2-propen-1-yl diazoacetate, trans-3-(para-bromophenyl)-2-propen-1-yl diazoacetate, trans-3-(para-trifluoromethylphenyl)-2-propen-1-yl diazoacetate, trans-3-(para-methoxyphenyl)-2-propen-1-yl diazoacetate, trans-3-(para-tert-butylphenyl)-2-propen-1-yl diazoacetate, and trans-3-phenyl-2-buten-1-yl diazoacetate.

30. The method of claim 26, wherein the reacting of the alkene-substituted diazo compound with the porphyrin metal complex takes place in the presence of an additive.

31. The method of claim 30, wherein the additive is selected from the group consisting of 4-dimethylaminopyridine (DMAP), nitrogen, phosphine, and sulfur coordinating ligands.

32. The method of claim 26, wherein the cyclopropane compound has an enantiomeric purity ranging from about 25% enantiomeric excess to about 99% enantiomeric excess.

33. The method of claim 32, wherein the cyclopropane compound has an enantiomeric purity ranging from about 50% enantiomeric excess to about 99% enantiomeric excess.

34. The method of claim 33, wherein the cyclopropane compound has an enantiomeric purity ranging from about 80% enantiomeric excess to about 99% enantiomeric excess.

35. The method of claim 34, wherein the cyclopropane compound has an enantiomeric purity ranging from about 90% enantiomeric excess to about 99% enantiomeric excess.
